(19) United States
(12) Patent Application Publication
(10) Pub. No.: US 2026/0077051 A1
(43) Pub. Date: Mar. 19, 2026

(54) COMPOUNDS FOR THE DEGRADATION OF EGFR KINASE

(71) Applicant: BeOne Medicines I GmbH, Basel (CH)

(72) Inventors: Huaqing LIU, Beijing (CN); Bailin LEI, Beijing (CN); Yizhou ZHAO, Beijing (CN); Jia TANG, Beijing (CN); Shaohan LIAO, Beijing (CN); Zhiwei WANG, Beijing (CN)

(73) Assignee: BeOne Medicines I GmbH, Basel (CH)

(21) Appl. No.: 19/402,615

(22) Filed: Nov. 26, 2025

Related U.S. Application Data
(63) Continuation of application No. PCT/CN2024/096861, filed on May 31, 2024.

(30) Foreign Application Priority Data
May 31, 2023 (WO) PCT/CN2023/097325

(51) Int. Cl. A61K 47/55; A61K 47/54; C07D 498/22

(52) U.S. Cl. CPC A61K 47/55; A61K 47/545; C07D 498/22

(57) ABSTRACT

Disclosed herein are novel bifunctional compounds formed by conjugating EGFR inhibitor moieties with E3 ligase Ligand moieties, which function to recruit targeted proteins to E3 ubiquitin ligase for degradation, and methods of preparation and uses thereof.

### FIELD OF THE INVENTION

Disclosed herein are novel bifunctional compounds formed by conjugating EGFR inhibitor moieties with E3 ligase Ligand moieties, which function to recruit targeted proteins to E3 ubiquitin ligase for degradation, and methods of preparation and uses thereof.

### BACKGROUND OF THE INVENTION

Proteolysis targeting chimera (PROTAC) consists of two covalently linked protein-binding molecules: one capable of engaging an E3 ubiquitin ligase, and another that binds to the protein of interest (POI) a target meant for degradation (Sakamoto K M et al., Proc. Natl. Acad. Sci. 2001, 98: 8554-9; Sakamoto K. M. et al., Methods Enzymol. 2005; 399:833-847). Rather than inhibiting the target protein's enzymatic activity, recruitment of the E3 ligase to the specific unwanted proteins results in ubiquitination and subsequent degradation of the target protein by the proteasome. The whole process of ubiquitination and proteasomal degradation is known as the ubiquitin-proteasome pathway (UPP) (Ardley H. et al., Essays Biochem. 2005, 41, 15-30; Komander D. et al., Biochem. 2012, 81, 203-229; Grice G. L. et al., Cell Rep. 2015, 12, 545-553; Swatek K. N. et al., Cell Res. 2016, 26, 399-422). Proteasomes are protein complexes which degrade unneeded, misfolded or abnormal proteins into small peptides to maintain health and productivity of the cells. Ubiquitin ligases, also called an E3 ubiquitin ligase, directly catalyze the transfer of ubiquitin from the E2 to the target protein for degradation. Although the human genome encodes over 600 putative E3 ligases, only a limited number of E3 ubiquitin ligases have been widely applied by small molecule PROTAC technology: cereblon (CRBN), Von Hippel-Lindau (VHL), mouse double minute 2 homologue (MDM2) and cellular inhibitor of apoptosis protein (cIAP) (Philipp O. et al., Chem. Biol. 2017, 12, 2570-2578), recombinant Human Ring Finger Protein 114 (RNF114) (Spradlin, J. N. et al. Nat. Chem. Biol. 2019, 15, 747-755) and DDB1 And CUL4 Associated Factor 16 (DCAF16) (Zhang, X. et al. Nat. Chem. Biol. 2019, 15, 737-746). For example, cereblon (CRBN) forms an E3 ubiquitin ligase complex with damaged DNA binding protein 1 (DDB1) and Cullin-4A (CUL4A) to ubiquitinate a number of other proteins followed by the degradation via proteasomes. (Yi-An Chen, et al., Scientific Reports 2015, 5, 1-13). Immunomodulatory drugs (IMiDs), including thalidomide, lenalidomide, and pomalidomide, function as monovalent promoters of PPIs by binding to the cereblon (CRBN) subunit of the CRL4ACRBN E3 ligase complex and recruiting neosubstrate proteins. (Matyskiela, M. E. et al., Nat Chem Biol 2018, 14, 981-987.) As a consequence, the ability of thalidomide, and its derivatives, to recruit CRBN has been widely applied in proteolysis-targeting chimeras (PROTACs) related studies (Christopher T. et al. ACS Chem. Biol. 2019, 14, 342-347; Honorine L. et al, ACS Cent. Sci. 2016, 2, 927-934). PROTACs have great potential to eliminate protein targets that are “undruggable” by traditional inhibitors or are non-enzymatic proteins. (Chu T T. et al., Cell Chem Biol. 2016; 23:453-461. Qin C. et al., J Med Chem 2018; 61: 6685-6704. Winter G E. et al., Science 2015; 348:1376-1381.) In the recent years, PROTACs as useful modulators promote the selective degradation of a wide range of target proteins have been reported in antitumor studies. (Lu J. et al., Chem Biol. 2015; 22(6):755-763; Ottis P. et al., Chem Biol. 2017; 12(4):892-898; Crews C. M. et al., J Med Chem. 2018; 61(2):403-404; Neklesa T. K. et al., Pharmacol Ther. 2017, 174:138-144; Cermakova K. et al., Molecules, 2018.23 (8); An S. et al., EBioMedicine, 2018; Lebraud H. et al., Essays Biochem. 2017; 61(5): 517-527; Sun Y H. et al., Cell Res. 2018; 28:779-81; Toure M. et al., Angew Chem Int Ed Engl. 2016; 55(6):1966-1973; Yonghui Sun et al., Leukemia, volume 33, pages 2105-2110 (2019); Shaodong Liu et al., Medicinal Chemistry Research, volume 29, pages 802-808 (2020); and has been disclosed or discussed in patent publications, e.g., US20160045607, US20170008904, US20180050021, US20180072711, WO2002020740, WO2014108452, WO2016146985, WO2016149668, WO2016197032, WO2016197114, WO2017011590, WO2017030814, WO2017079267, WO2017182418, WO2017197036, WO2017197046, WO2017197051, WO2017197056, WO2017201449, WO2018071606, WO2021178920, WO2021127283, WO2021127190, WO202111871 and WO202111913.

Epidermal growth factor receptor (EGFR) that belongs to the ErbB family is a transmembrane receptor tyrosine kinase (RTK), which plays a fundamentally key role in cell proliferation, differentiation, and motility (Y. Yarden, et al., Nat. Rev. Mol. Cell Biol. 2001; 2:127-137). Homo- or heterodimerization of EGFR and other ErbB family members activates cytoplasmic tyrosine kinase domains to initiate intracellular signaling. Overexpression or activating mutations of EGFR are associated the development of many types of cancers, such as pancreatic cancer, breast cancer, glioblastoma multiforme, head and neck cancer, and non-small cell lung cancer (Yewale C., et al. Biomaterials. 2013, 34 (34): 8690-8707). The activating mutations in the EGFR tyrosine kinase domain (L858R mutation and exon-19 deletion) have been identified as oncogenic drivers for NSCLC (Konduri, K., et al. Cancer Discovery 2016, 6 (6), 601-611). The first-generation EGFR tyrosine kinase inhibitors (EGFR-TKIs) gefitinib and erlotinib have approved for NSCLC patients with EGFR activation mutations (M. Maemondo, N. Engl. J. Med. 362 (2010) 2380-2388). Although most patients with EGFR mutant NSCLC respond to these therapies, patients typically develop resistance after an average of one year on treatment. There are several mechanisms of acquired resistance to gefitinib and erlotinib, including a secondary threonine 790 to methionine 790 mutation (T790M), is also called “gatekeeper” T790M mutation (Xu Y., et al. Cancer Biol Ther. 2010, 9 (8): 572-582). Therefore, the second-generation EGFR-TKIs afatinib and the third-generation EGFR-TKIs osimertinib (AZD9291) were developed as irreversible EGFR inhibitors that bind to Cys797 for the treatment of patients with T790M mutation. In particular, osimertinib that largely spares WT EGFR has achieved greater clinical response rate in NSCLC patients with EGFR T790M. However, several recent studies have reported a tertiary Cys797 to Ser797 (C797S) point mutation with osimertinib clinical therapy (Thress K S, et al. Nat. Med. 2015, 21 (6): 560-562). There is a need for drugs which can overcome EGFR (C797S) resistance obstacle in non-small cell lung cancer (NSCLC). EGFR-Targeting PROTACs serve as a potential strategy to overcome drug resistance mediated by these mutants, which has been disclosed or discussed in patent publications, e.g. WO2018119441, WO2019149922, WO2019183523, WO2019121562, US20190106417, WO202157882, WO2021123087, WO2021133809, WO2021168074, WO2021208918 and WO2021216440.

Although, a number of EGFR-targeting PROTACs which were designed to degrade EGFR mutant proteins have been published (Zhang X., et al. Eur. J. Med Chem. 2020, 192, 112199; Zhang H, et al. Eur. J. Med Chem. 2020, 189, 112061; Lu X, Med Res. Rev. 2018, 38(5):1550-1581. He K., et al. Bioorg. Med Chem. Lett. 2020, 15, 127167). Most of the published molecules are based on first, second, and third generation of EGFR inhibitors (WO2021023233, WO2019121562 and WO2018119441) or allosteric EGFR inhibitors (WO2021127561). However, there were no data which showed those EGFR-Targeting PROTACs degrading all the main EGFR mutations, Such as Del19, L858R, Del19/T790M, L858R/T790M, Del19/T790M/C797S, L858R/T790M/C797S.

The present application provides novel bifunctional compounds and compositions for the treatment of serious diseases.

### SUMMARY OF THE INVENTION

One objective of the present invention is to provide compounds and derivatives formed by conjugating EGFR inhibitor moieties with E3 ligase Ligand moieties, which function to recruit targeted proteins to E3 ubiquitin ligase for degradation, and methods of preparation and uses thereof.

The compounds described herein or salts thereof are useful in the treatment of a disease that can be affected by EGFR modulation. The present invention provides the use of the compounds described herein or a pharmaceutically acceptable salt thereof in the manufacture of a medicament for the treatment of a disease that can be affected by EGFR modulation. The present invention further provides a compound described herein or a pharmaceutically acceptable salt thereof, for use in the treatment of a disease that can be affected by EGFR modulation. The present application further provides a method of treating a proliferative disorder, comprising administering to a subject in need thereof a therapeutically effective amount of the compounds described herein or a pharmaceutically acceptable salt thereof.

Aspect 1. A compound of Formula (I):

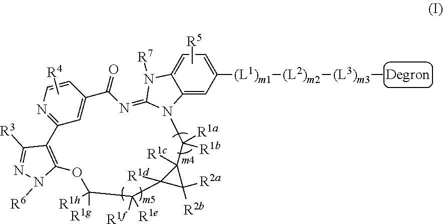

or a N-oxide thereof, or a pharmaceutically acceptable salt thereof, or a stereoisomer thereof, or a deuterated analog thereof, or a prodrug thereof,
wherein:
R^{1a}, R^{1b}, R^{1c}, R^{1d}, R^{1e}, R^{1f}, R^{1g}, R^{1h}, R^{2a}, R^{2b}, R³, R⁴ and R⁵ are each independently hydrogen, halogen, —C₁₋₄alkyl, —C₁₋₄alkoxy, —C₃₋₆cycloalkyl or —CN; each said —C₁₋₄alkyl, —C₁₋₄alkoxy, or —C₃₋₆cycloalkyl is optionally substituted with at least one substituent selected from hydrogen, halogen, —C₁₋₈alkoxy, —C₃₋₈cycloalkyl or —CN;
R⁶ and R⁷ are each independently hydrogen, —C₁₋₃alkyl or —C₃₋₆cycloalkyl; each said —C₁₋₃alkyl —C₃₋₆cycloalkyl is optionally substituted with at least one substituent selected from hydrogen, halogen, —C₁₋₈alkoxy, —C₃₋₈cycloalkyl or —CN;
m1, m2 and m3 are each independently 0, 1 or 2, provided that m1+m2+m3≤4 and m1+m2+m3≥1;
m4 and m5 are each independently 0, 1 or 2, provided that m4+m5=2;
L¹ is independently selected from —C₁₋₃alkylene-, —O—, —NR^{a}—, —C(O)—, *^{L1}—C(O)NR^{a}—**^{L1}, *^{L1}—C(O)O—**^{L1}, *^{L1}—NR^{a}C(O)—**^{L1}, *^{L1}—OC(O)—**^{L1},

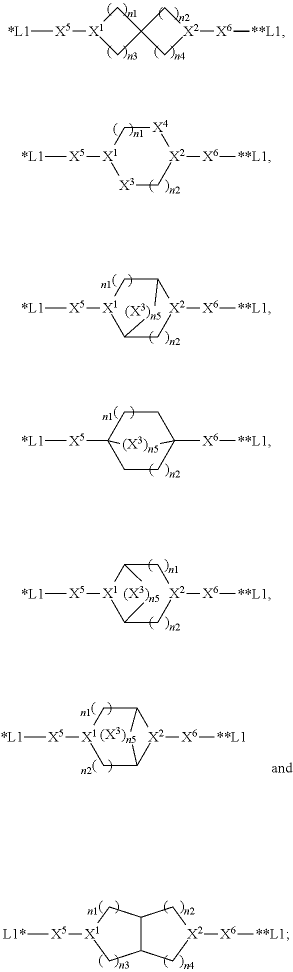

wherein each of said —C₁₋₃alkylene-,

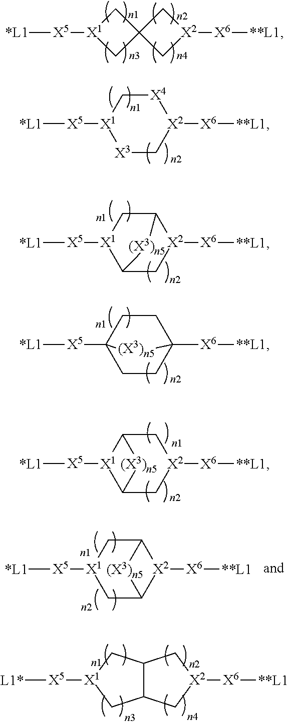

is optionally substituted with at least one R^{L1c};
wherein *^{L1} refers to the position attached to the

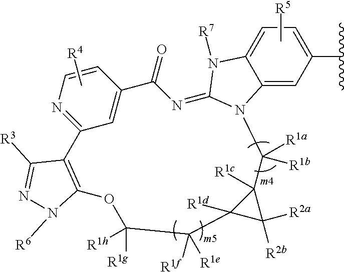

moiety, and **^{L1} refers to the position attached to the

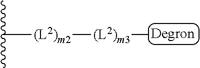

moiety;
L² is independently selected from —C₁₋₃alkylene-, —O—, —NR^{a}—, —C(O)—, *^{L2}—C(O)NR^{a}—**^{L2}, *^{L2}—C(O)O—**^{L2}, *^{L2}—NR^{a}C(O)—**^{L2}, *^{L2}—OC(O)—**^{L2},

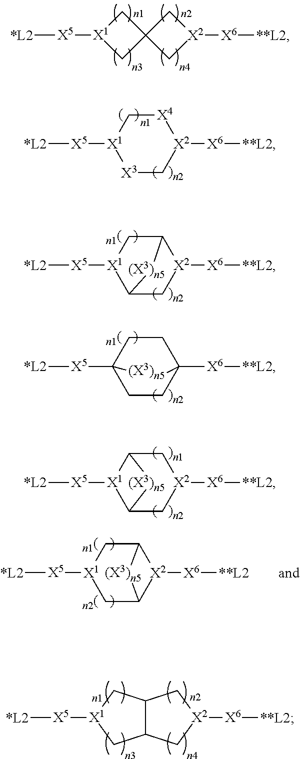

wherein each of said —C₁₋₃alkylene-,

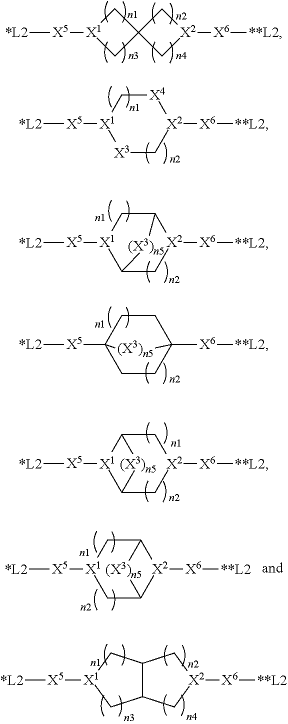

is optionally substituted with at least one R^{L2c};
wherein *^{L2} refers to the position attached to the

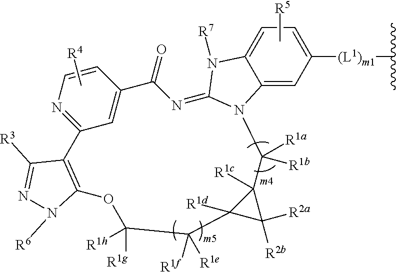

moiety, and **^{L2} refers to the position attached to the

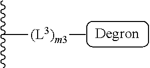

moiety;
L³ is independently selected from —C₁₋₃alkylene-, —O—, —NR^{a}—, —C(O)—, *^{L3}—C(O)NR^{a}—**^{L3}, *^{L3}—C(O)O—**^{L3}, *^{L3}—NR^{a}C(O)—**^{L3}, *^{L3}—OC(O)—**^{L3},

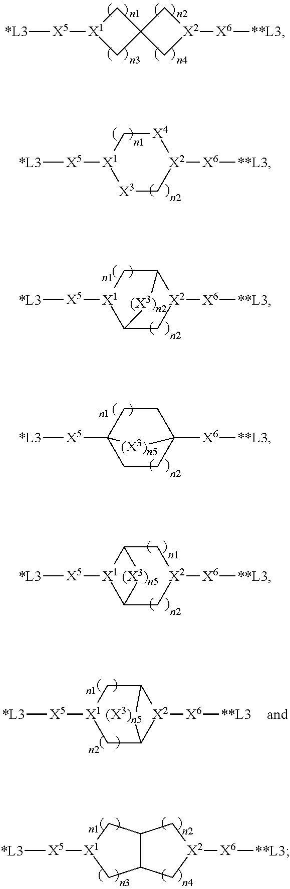

wherein each of said —C₁₋₃alkylene-,

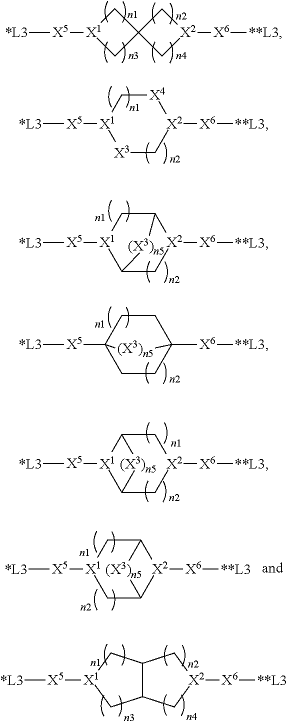

is optionally substituted with at least one R^{L3c}.
wherein *^{L3} refers to the position attached to the

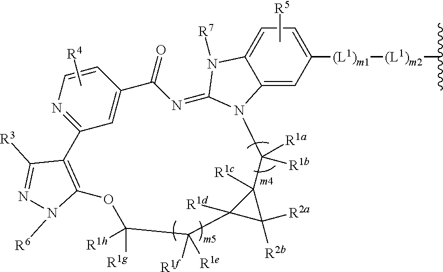

moiety,
and **^{L3} refers to the position attached to the

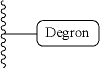

moiety;
each of said R^{L1c}, R^{L2c} and R^{L3c} are independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; each of said —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl and 5- to 12-membered heteroaryl is optionally substituted with at least one R^{Lca}
R^{Lca} is independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L2c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L3c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;

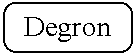

is selected from

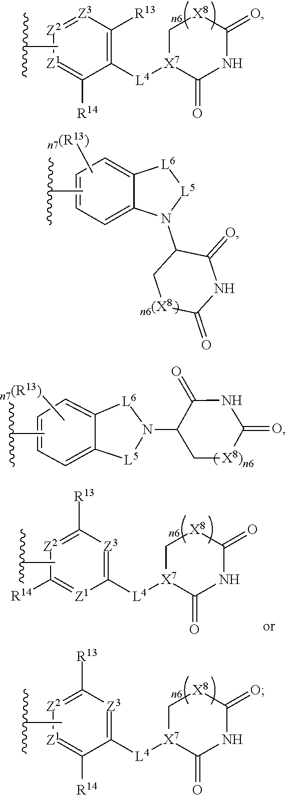

Z¹, Z² and Z³ are each independently N or CR^{z}, provided that Z¹, Z² and Z³ are not N at the same time;
R^{z}, at each occurrence, is independently selected from absence, hydrogen, halogen, —C₁₋₈alkyl, —N^{Za}R^{Za}R^{Zb}, —OR^{Za}, —SR^{Za}, C₃-C₈cycloalkyl, 3- to 8-membered heterocyclyl or CN; each of —C₁₋₈alkyl, C₃-C₈cycloalkyl, 3- to 8-membered heterocyclyl is optionally substituted with at least one R^{Zc};
the

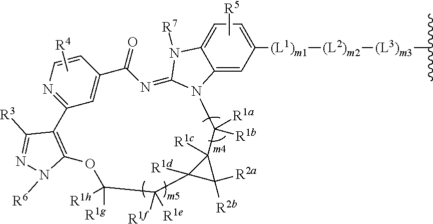

moiety is linked to the

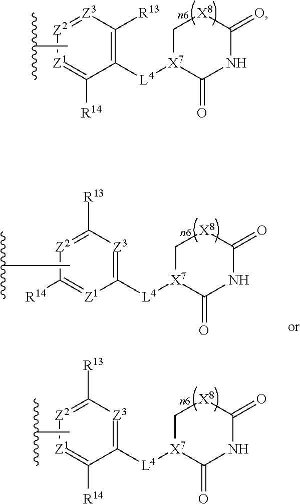

moiety via any one of Z¹, Z² or Z³ which is CR^{Z} and R^{Z} is absence;
R^{Za} and R^{Zb} are each independently selected from absence, hydrogen, —C₁-C₈alkyl, C₃-C₈cycloalkyl, 3- to 8-membered heterocyclyl, C₆-C₁₂aryl, or 5- to 12-membered heteroaryl, each of said —C₁₋₈alkyl, C₃-C₈cycloalkyl, 3- to 8-membered heterocyclyl, C₆-C₁₂aryl, or 5- to 12-membered heteroaryl is optionally substituted with at least one substituent R^{Zd};
R^{Zc} and R^{Zd} are each independently halogen, hydroxy, —C₁-C₈alkyl, —C₁₋₈alkoxy, C₃-C₈cycloalkyl, 3- to 8-membered heterocyclyl, C₆-C₁₂aryl, or 5- to 12-membered heteroaryl;
R¹³ and R¹⁴ are each independently selected from absence, hydrogen, halogen, —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}CO₂R^{13b}, or —NR^{13a}SO₂R^{13b}; each of —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, NR^{13c}COR^{13d}, —NR^{13c}CO₂R^{13d}, or —NR^{13c}SO₂R^{13d};
at each occurrence, R^{13a}, R^{13b}, R^{13c} and R^{13d} are each independently absence, hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl;
L⁴, L⁵ and L⁶ are each independently selected from a absence, single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
at each occurrence, X¹, X² and X⁷ are each independently selected from —CR^{a}, or N; at each occurrence, X³, X⁴ and X⁸ are each independently selected from —NR^{a}—, —O—, —S— and —CR^{a}R^{b}—;
at each occurrence, X⁵ and X⁶ are each independently selected from absence, single bond, —C(O)—, —NR^{a}— and —O—;
at each occurrence, Ra and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;
n1, n2, n3, n4 and n5 are each independently 0, 1, 2 or 3;
n6, n7 and n8 are each independently 0, 1, 2, 3 or 4;
provided that:
for any one of L¹, L² or L³, when X¹ is N, X⁵ is single bond, absence, —C(O)—; and/or when X² is N, X⁶ is single bond, absence, —C(O)—;
when L¹ is

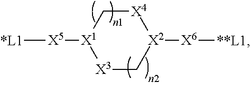

when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—;
when L² is

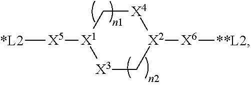

when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—;
when L³ is

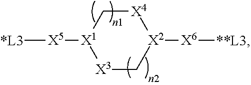

when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—.

Aspect 2. The compound of Aspect 1, wherein the compound is selected from formula (IIa):

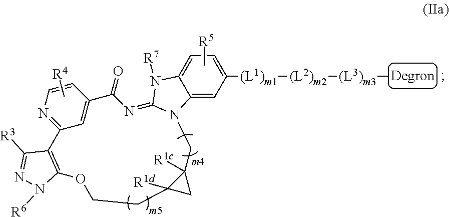

preferably, the compound is selected from formula (IIb):

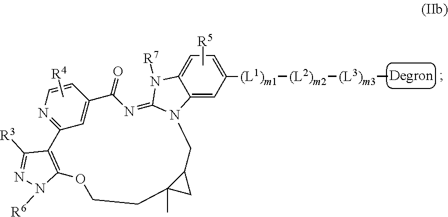

more preferably, the compound is selected from formula (IIc)

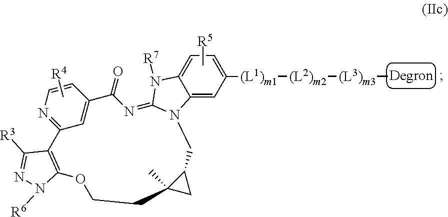

even more preferably, the compound is selected from formula (IId)

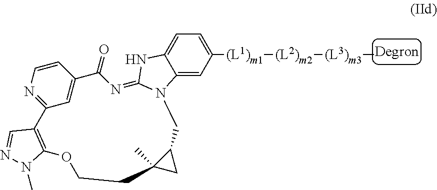

wherein R^{1c} and R^{1d} are each independently H, methyl or ethyl, provided that at most one of R^{1c} and R^{1d} is H;
R³, R⁴, R⁵, R⁶, R⁷, L¹, L², L³, m1, m2, m3, m4, m5 and Degron are defined as Aspect 1.

Aspect 3. The compound of Aspect 1, wherein the compound is selected from formula (IIIa)

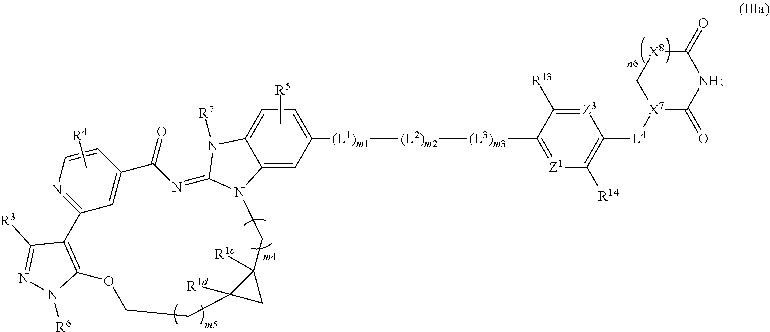

wherein R^{1c} and R^{1d} are each independently H, methyl or ethyl, provided that at most one of R^{1c} and R^{1d} is H;
R³, R⁴, R⁵, R⁶, R⁷, R¹³, R¹⁴, L¹, L², L³, L⁴, m1, m2, m3, m4, m5, Z¹, Z³, X⁷, X⁸ and n6 are defined as Aspect 1;
preferably, the compound is selected from formula (IIIb)

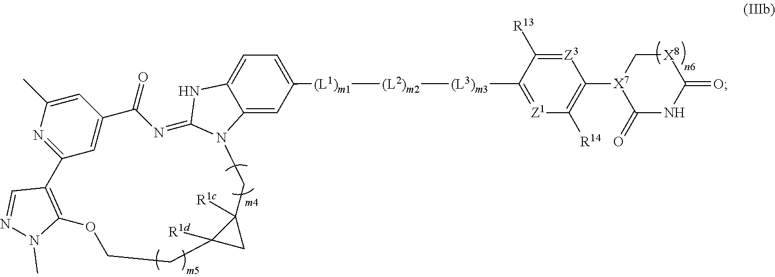

wherein R^{1c} and R^{1d} are each independently H, methyl or ethyl, provided that at most one of R^{1c} and R^{1d} is H;
R¹³, R¹⁴, L¹, L², L³, L⁴, m1, m2, m3, m4, m5, Z¹, Z³, X⁷, X⁸ and n6 are defined as Aspect 1; more preferably, the compound is selected from formula (IIIc) or (IIId)

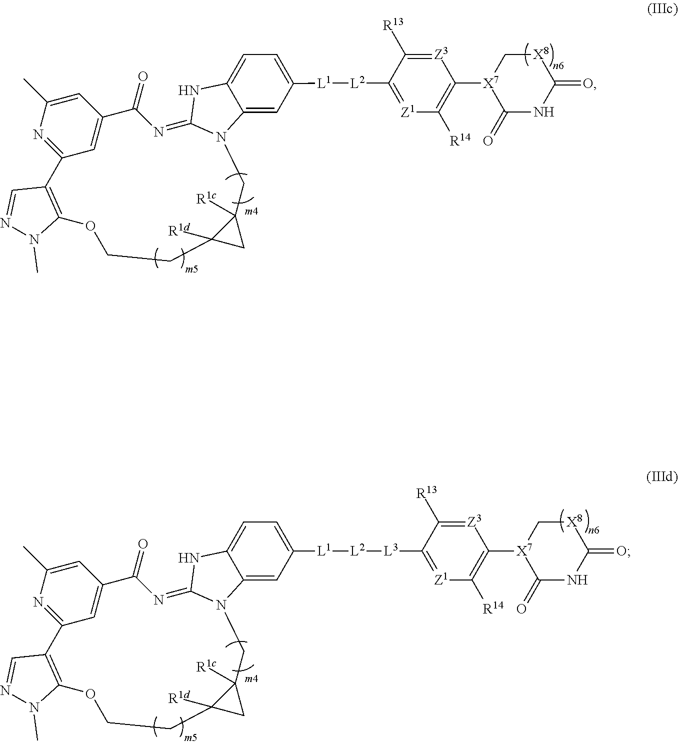

wherein R^{1c} and R^{1d} are each independently H, methyl or ethyl, provided that at most one of R^{1c} and R^{1d} is H;
R¹³, R¹⁴, L¹, L², L³, L⁴, m4, m5, Z¹, Z³, X⁷, X⁸ and n6 are defined as Aspect 1.

Aspect 4. The compound of Aspect 1, wherein the compound is selected from formula (IVa)

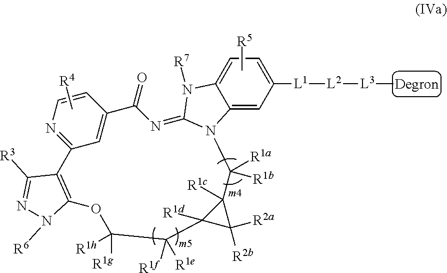

or a N-oxide thereof, or a pharmaceutically acceptable salt thereof, or a stereoisomer thereof, or a deuterated analog thereof, or a prodrug thereof,
wherein:
R^{1a}, R^{1b}, R^{1c}, R^{1d}, R^{1e}, R^{1f}, R^{1g}, R^{1h}, R^{2a}, R^{2b}, R³, R⁴ and R⁵ are each independently hydrogen, halogen, —C₁₋₄alkyl, —C₁₋₄alkoxy, —C₃₋₆cycloalkyl or —CN; each said —C₁₋₄alkyl, —C₁₋₄alkoxy, or —C₃₋₆cycloalkyl is optionally substituted with at least one substituent selected from hydrogen, halogen, —C₁₋₈alkoxy, —C₃₋₈cycloalkyl or —CN;
R⁶ and R⁷ are each independently hydrogen, —C₁₋₃alkyl or —C₃₋₆cycloalkyl; each said —C₁₋₃alkyl —C₃₋₆cycloalkyl is optionally substituted with at least one substituent selected from hydrogen, halogen, —C₁₋₈alkoxy, —C₃₋₈cycloalkyl or —CN;
m4 and m5 are each independently 0, 1 or 2, provided that m4+m5=2;
wherein
L¹ is independently selected from

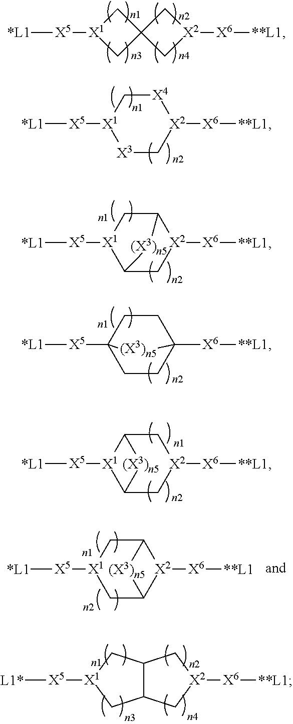

wherein each of said

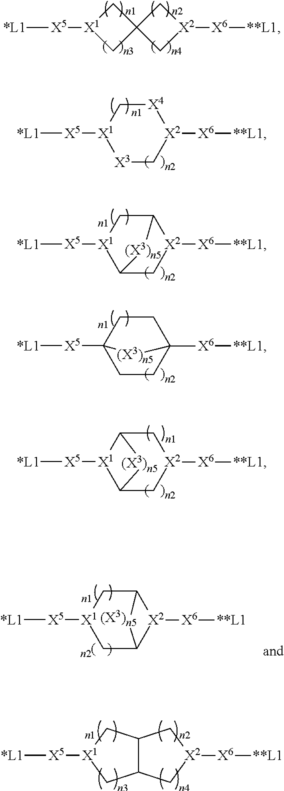

is optionally substituted with at least one R^{L1c};
wherein *^{L1} refers to the position attached to the

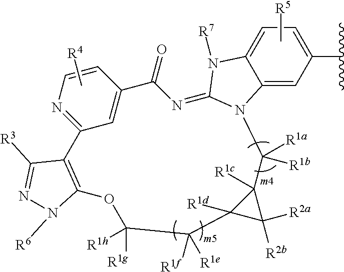

moiety, and **^{L1} refers to the position attached to the

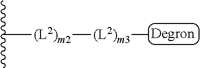

moiety;
L² is independently selected from

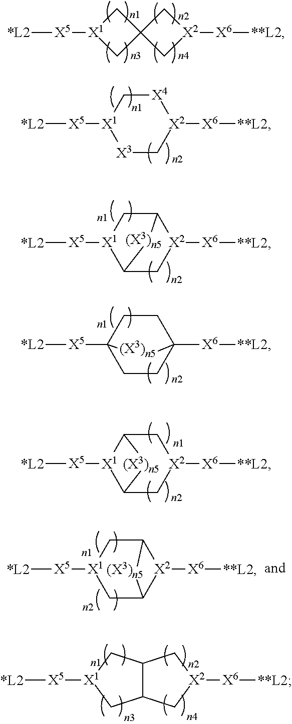

wherein each of said

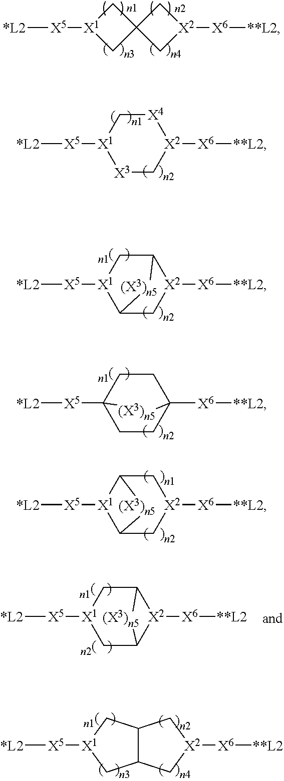

is optionally substituted with at least one R^{L2c}.
wherein *^{L2} refers to the position attached to the

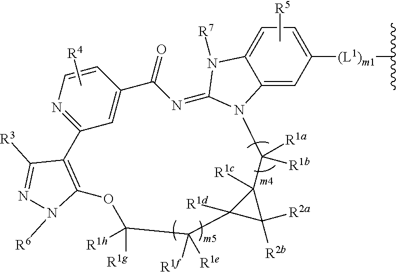

moiety, and **^{L2} refers to the position attached to the

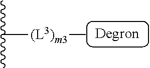

moiety;
L³ is independently selected from

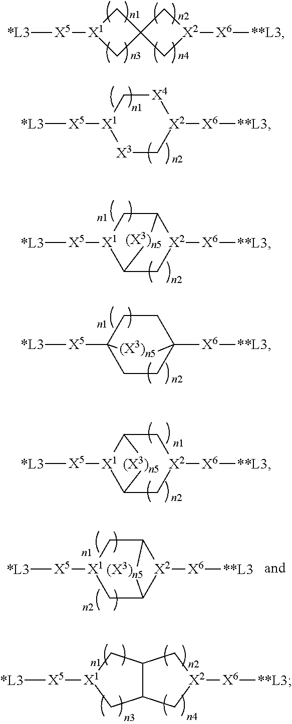

wherein each of said

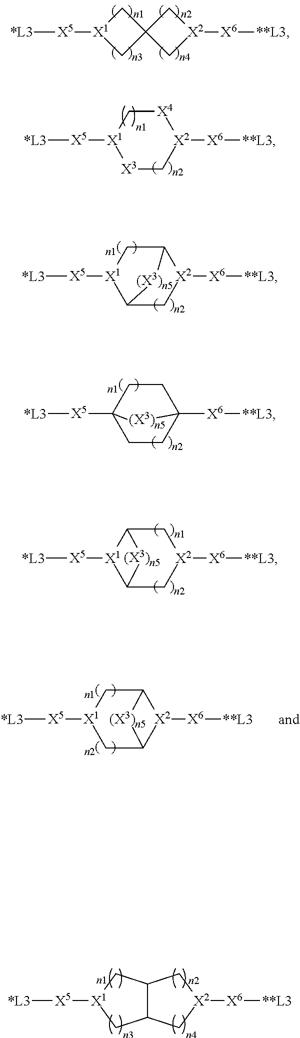

is optionally substituted with at least one R^{L3c}.
wherein *^{L3} refers to the position attached to the

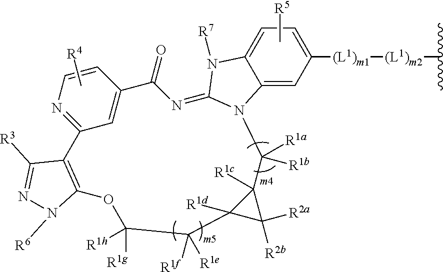

moiety,
and **^{L3} refers to the position attached to the

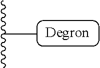

moiety;
each of said R^{L1c}, R^{L2e} and R^{L3e} are independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; each of said —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl and 5- to 12-membered heteroaryl is optionally substituted with at least one R^{Lca}
R^{Lca} is independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L2e} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L3e} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;

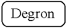

is selected from

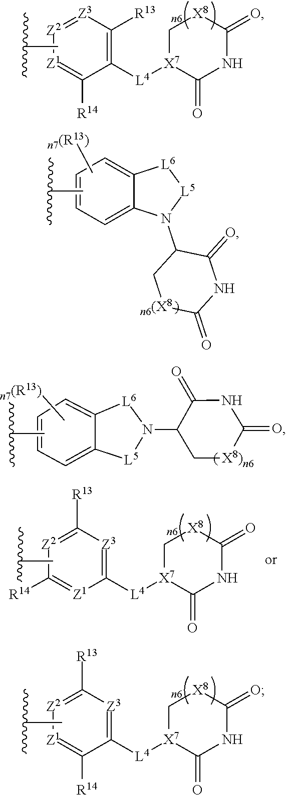

Z¹, Z² and Z³ are each independently N or CR^{z}, provided that Z¹, Z² and Z³ are not N at the same time;
R^{z}, at each occurrence, is independently selected from absence, hydrogen, halogen, —C₁₋₈alkyl, —NR^{Za}R^{Zb}, —OR^{Za}, —SR^{Za}, C₃-C₈cycloalkyl, 3- to 8-membered heterocyclyl or CN; each of —C₁₋₈alkyl, C₃-C₈cycloalkyl, 3- to 8-membered heterocyclyl is optionally substituted with at least one R^{Zc};
the

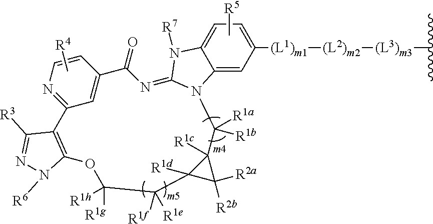

moiety is linked to the

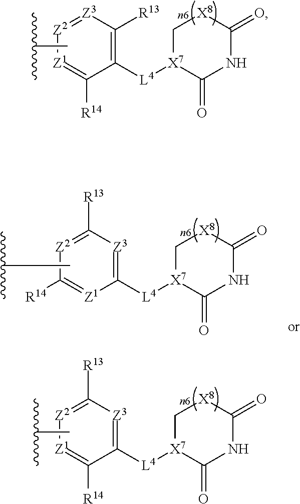

moiety via any one of Z¹, Z² or Z³ which is CR^{z} and R^{z} is absence;
R^{Za} and R^{Zb} are each independently selected from absence, hydrogen, —C₁-C₈alkyl, C₃-C₈cycloalkyl, 3- to 8-membered heterocyclyl, C₆-C₁2aryl, or 5- to 12-membered heteroaryl, each of said —C₁₋₈alkyl, C₃-C₈cycloalkyl, 3- to 8-membered heterocyclyl, C₆-C₁₂aryl, or 5- to 12-membered heteroaryl is optionally substituted with at least one substituent R^{Zd}.
R^{Ze} and R^{Zd} are each independently halogen, hydroxy, —C₁-C₈alkyl, —C₁-C₈alkoxy, C₃-C₈cycloalkyl, 3- to 8-membered heterocyclyl, C₆-C₁2aryl, or 5- to 12-membered heteroaryl;
R¹³ and R¹⁴ are each independently selected from absence, hydrogen, halogen, —C₁₋₈alkyl, —C₂₋₈ alkenyl, —C₂₋₈alkynyl, —C₁₋₈alkoxy, —C₃-C₈cycloalkyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, NR^{13a}CO₂R^{13b}, or —NR^{13a}SO₂R^{13b}; each of —C₁₋₈alkyl, —C₂₋₈alkenyl, —C₂₋₈alkynyl, —C₁₋₈alkoxy, —C₃₋₈cycloalkyl, 3- to 8-membered heterocyclyl, —C₆-C₁₂aryl or 5- to 12-membered heteroaryl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, NR^{13c}COR^{13d}, —NR^{13c}CO₂R^{13d}, or —NR^{13c}SO₂R^{13d};
at each occurrence, R^{13a}, R^{13b}, R^{13c} and R^{13d} are each independently absence, hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl;
L⁴, L⁵ and L⁶ are each independently selected from a absence, single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
at each occurrence, X¹, X² and X⁷ are each independently selected from —CR^{a}, or N;
at each occurrence, X³, X⁴ and X⁸ are each independently selected from —NR^{a}—, —O—, —S— and —CR^{a}R^{b}—;
at each occurrence, X⁵ and X⁶ are each independently selected from absence, single bond, —C(O)—, —NR^{a}— and —O—;
at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;
n1, n2, n3, n4 and n5 are each independently 0, 1, 2 or 3;
n6, n7 and n8 are each independently 0, 1, 2, 3 or 4;
provided that:
for any one of L¹, L² or L³, when X¹ is N, X⁵ is single bond, absence, —C(O)—; and/or when X² is N, X⁶ is single bond, absence, —C(O)—;
when L¹ is

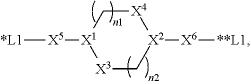

when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—;
when L² is

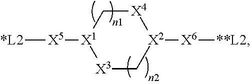

when X¹ is N, X⁵ is single bond, absence, —C(O)—; and/or X³ is —CR^{a}R^{b}—; when X² is N, X⁶ is single bond absence, —C(O)—, and/or X⁴ is —CR^{a}R^{b}—;
when L³ is

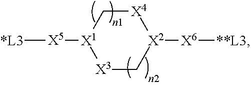

when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—.

Aspect 5. The compound of Aspect 3, wherein the compound is selected from formula (Va):

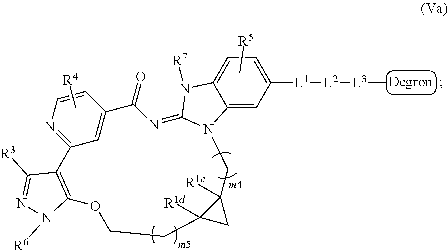

preferably, the compound is selected from formula (Vb):

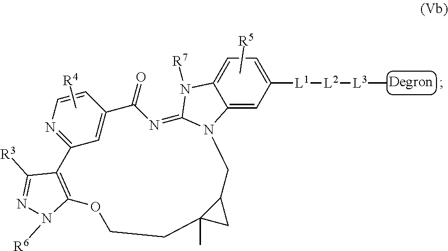

more preferably, the compound is selected from formula (Vc):

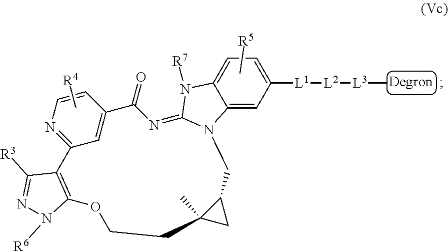

even more preferably, the compound is selected from formula (Vd):

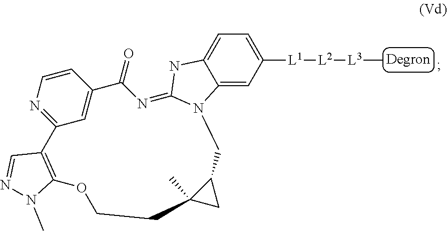

even more preferably, the compound is selected from formula (Ve):

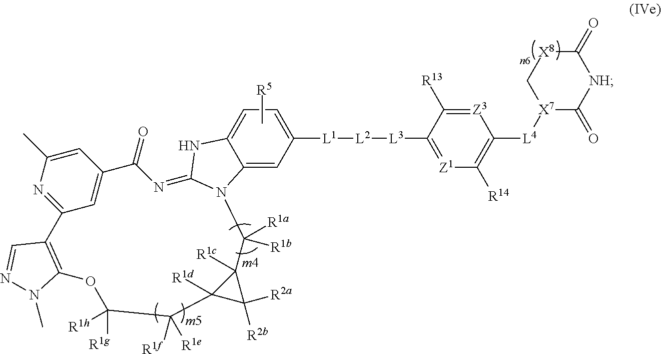

even more preferably, the compound is selected from formula (Vf):

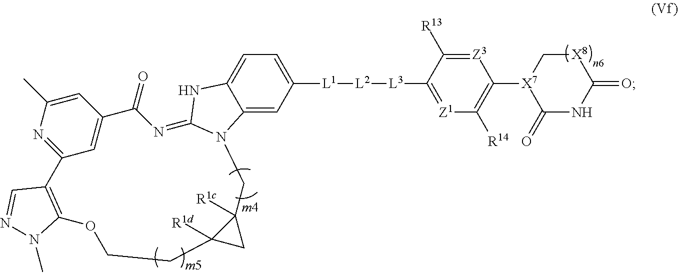

R^{1a}, R^{1b}, R^{1c}, R^{1d}, R^{1e}, R^{1f}, R^{1h}, R^{2a}, R^{2b}, R¹³, R¹⁴, L¹, L², L³, L⁴, m4, m5, Z¹, Z³, X⁷, X⁸ and n6 are defined as Aspect 3.

Aspect 6. The compound of any one of the preceding Aspects, wherein R⁶ and R⁷ are each independently hydrogen, methyl, ethyl, propyl, cyclopropyl, cyclobutyl, cyclopentyl or cyclohexyl; each of said methyl, ethyl, propyl, cyclopropyl, cyclobutyl, cyclopentyl or cyclohexyl is optionally substituted with at least one substituent selected from hydrogen, F, Cl, Br, I, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN;
preferably, R⁶ and R⁷ are each independently hydrogen, methyl, ethyl, propyl, cyclopropyl, cyclobutyl, cyclopentyl or cyclohexyl;
more preferably R⁶ is independently methyl or cyclopropyl, and R⁷ is hydrogen.

Aspect 7. The compound of any one of the preceding Aspects, wherein R^{1a}, R^{1b}, R^{1c}, R^{1d}, R^{1e}, R^{1f}, R^{1g}, R^{1h}, R^{2a}, R^{2b}, R³, R⁴ and R⁵ are each independently hydrogen, F, Cl, Br, I, methyl, ethyl, propyl, butyl, methoxy, ethoxy, propoxy, butoxy, cyclopropyl, cyclobutyl, cyclopentyl, cyclohexyl or —CN; wherein each said methyl, ethyl, propyl, butyl, methoxy, ethoxy, propoxy, butoxy, cyclopropyl, cyclobutyl, cyclopentyl or cyclohexyl is optionally substituted with at least one substituent selected from hydrogen, F, Cl, Br, I, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN;
preferably, R^{1a}, R^{1b}, R^{1c}, R^{1d}, R^{1e}, R^{1f}, R^{1g}, R^{1h}, R^{2a}, R^{2b}, R³, R⁴ and R⁵ are each independently hydrogen, F, Cl, Br, I, methyl, ethyl, propyl, butyl, methoxy, ethoxy, propoxy, butoxy, cyclopropyl, cyclobutyl, cyclopentyl, cyclohexyl, —CF₃, —CHF₂, —CN, —CH₂OCH₃, —CH₂OCH₂CH₃, —CH₂CH₂OCH₃, —CH₂CH₂OCH₂CH₃;
more preferably R^{1a}, R^{1b}, R^{1c}, R^{1d}, R^{1e}, R^{1f}, R^{1g}, R^{1h}, R^{2a}, R^{2b}, R³, R⁴ and R⁵ are each independently hydrogen, F, Cl, methyl, methoxy, cyclopropyl, —CF₃ or —CHF₂, —CH₂OCH₃; even more preferably, R⁴ is hydrogen, F, methyl, methoxy, cyclopropyl or —CF₃; R³ is hydrogen, F, methyl, —CF₃ or —CHF₂; R⁵ is hydrogen, F, Cl, methyl or —CH₂OCH₃; R^{1a}, R^{1b}, R^{1c}, R^{1d}, R^{1e}, R^{1f}, R^{1g}, R^{1h}, R^{2a} and R^{2b} are each independently hydrogen, F or methyl.

Aspect 8. The compound of any one of the preceding Aspects, wherein the

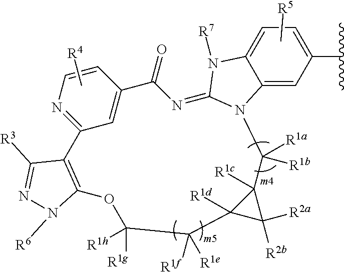

moiety is selected from

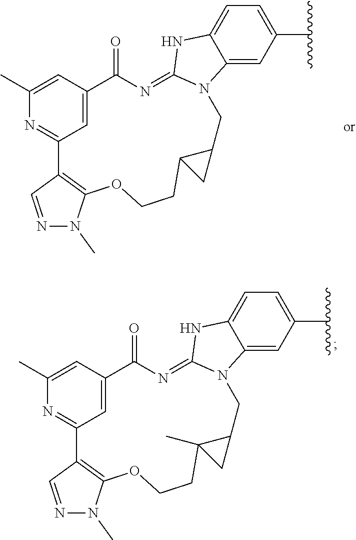 [SMILES: *c1ccc2c(c1)N1CC3CC3CCOc3c(cnn3C)-c3cc(cc(C)n3)C(=O)/N=C/1N2 ; *c1ccc2c(c1)N1CC3CC3(C)CCOc3c(cnn3C)-c3cc(cc(C)n3)C(=O)/N=C/1N2]

preferably,

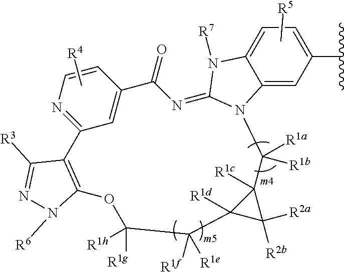

moiety is selected from

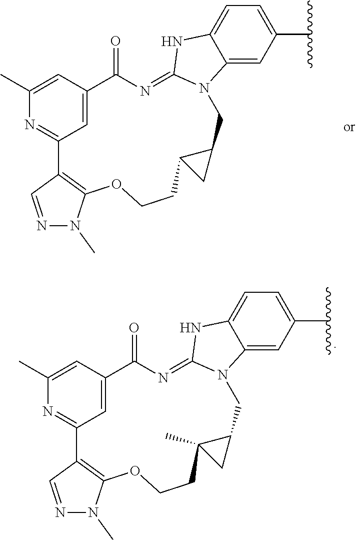 [SMILES: *c1ccc2c(c1)N1C[C@H]3C[C@@H]3CCOc3c(cnn3C)-c3cc(cc(C)n3)C(=O)/N=C/1N2 ; *c1ccc2c(c1)N1C[C@@H]3C[C@@]3(C)CCOc3c(cnn3C)-c3cc(cc(C)n3)C(=O)/N=C/1N2]

Aspect 9. The compound of any one of the preceding Aspects, wherein L¹ is selected from —O—, —C(O)—, —N(R^{a})—*^{L1}—C(O)N(R^{a})—**^{L1}, *^{L1}—C(O)O—**^{L1}, *^{L1}—N(R^{a})C(O)—**^{L1}, *^{L1}—OC(O)—**^{L1},

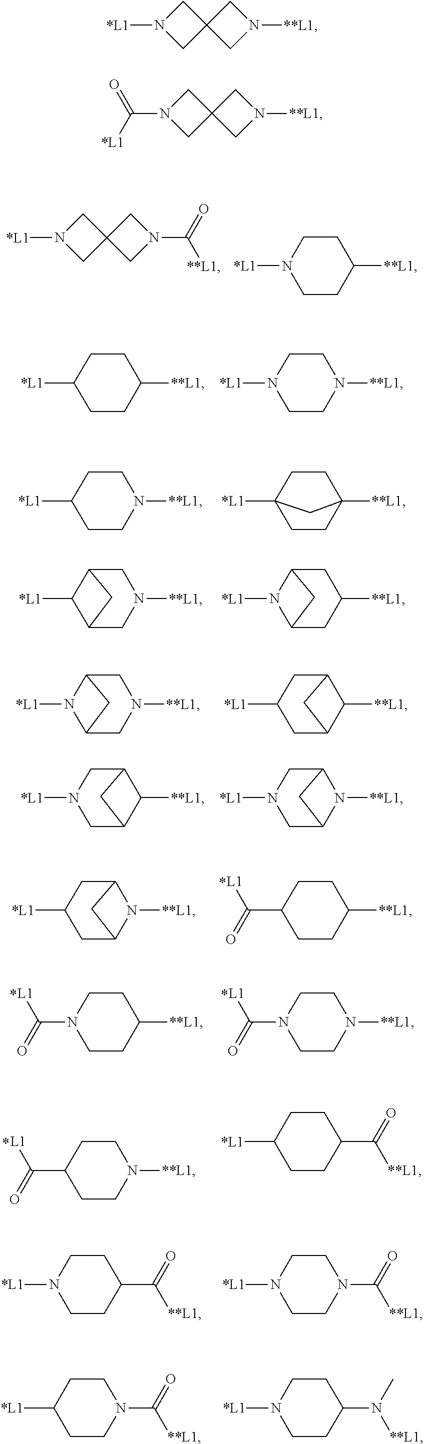

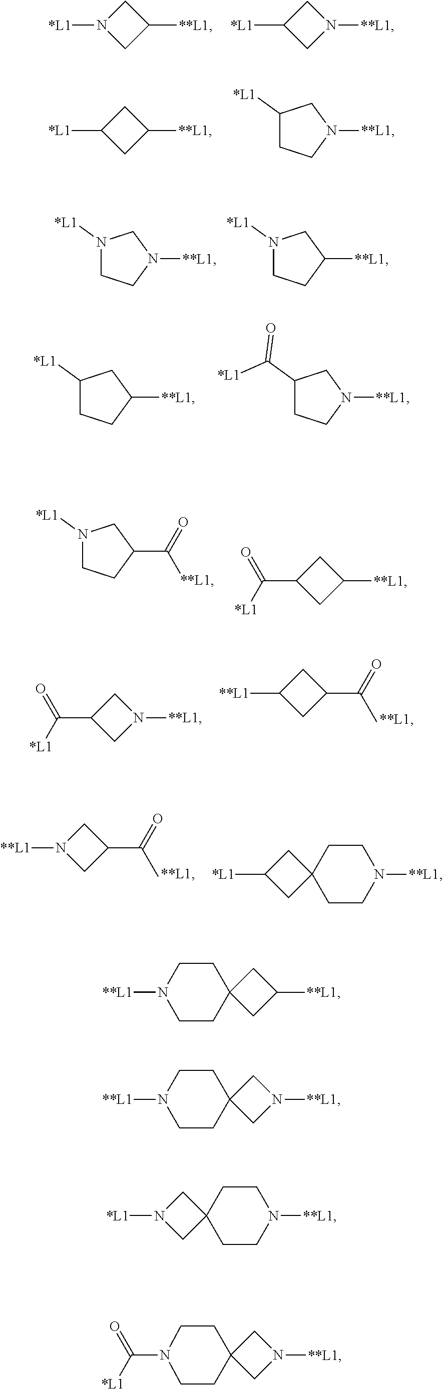

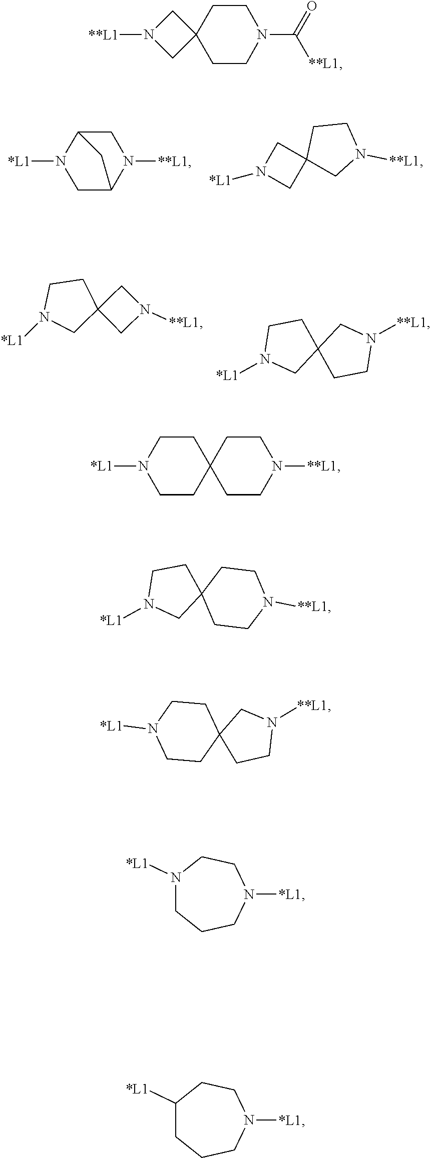

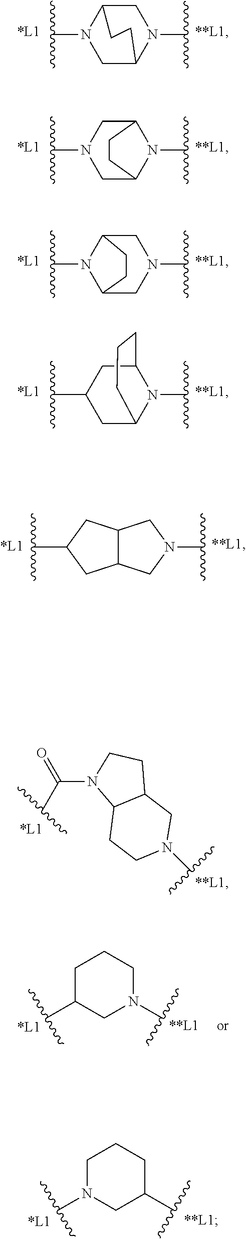

wherein each of said

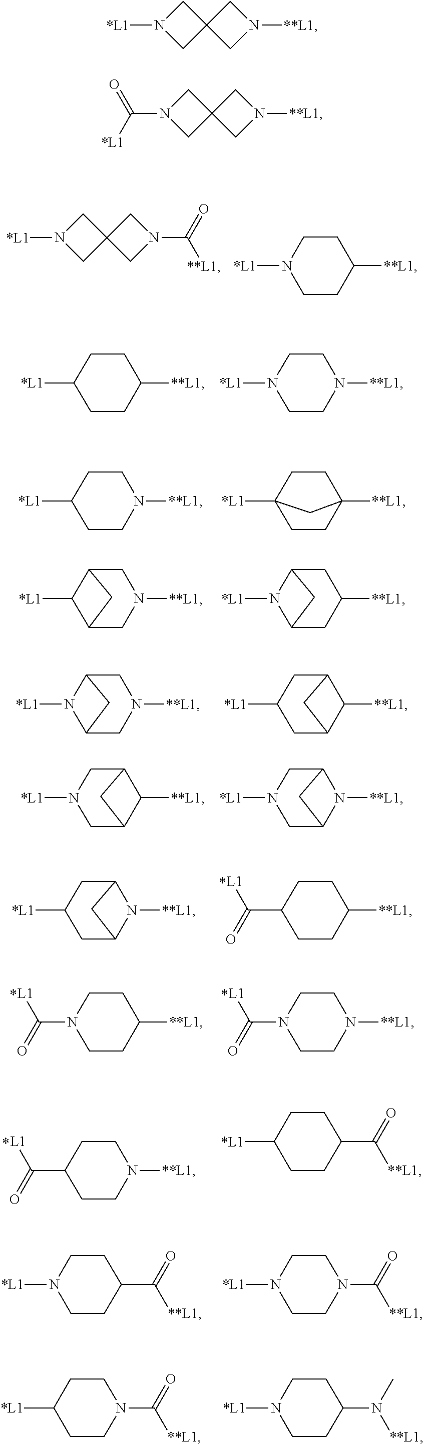

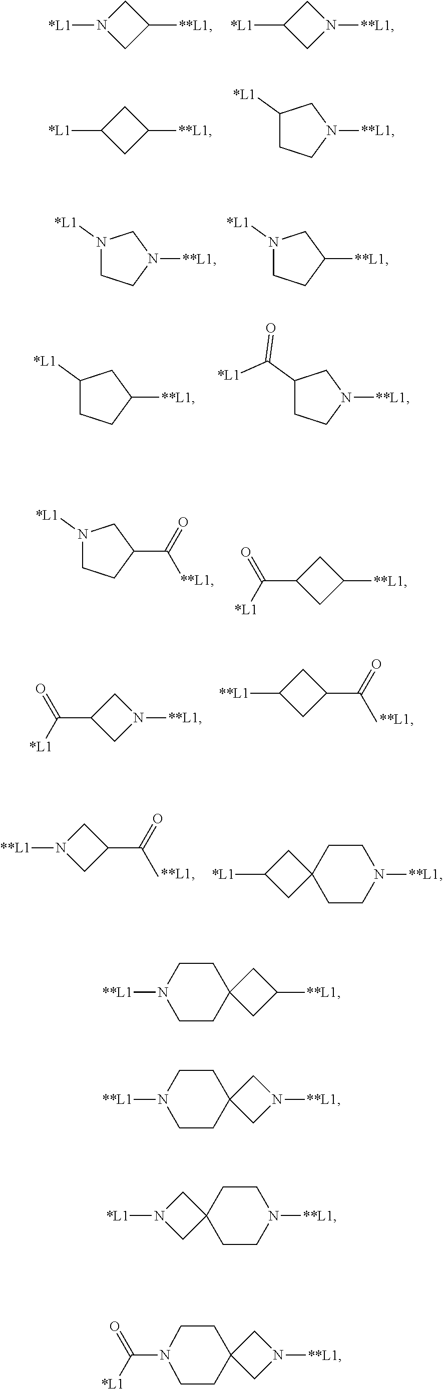

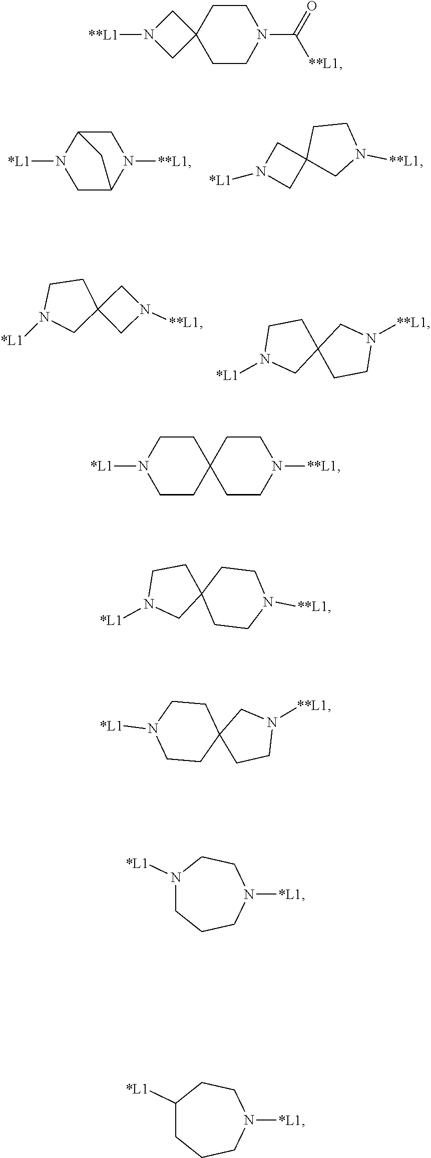

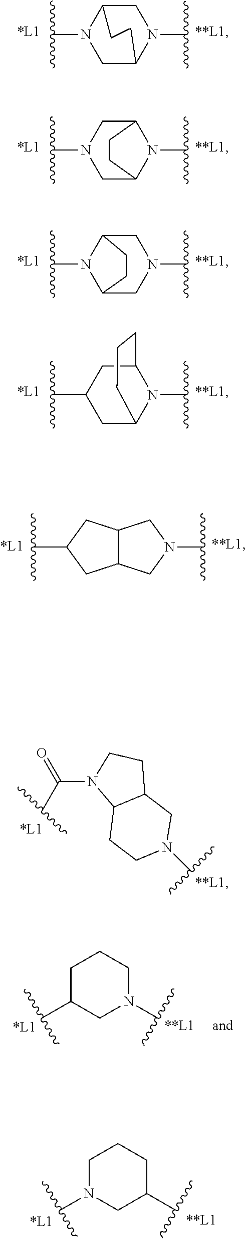

is optionally substituted with at least one R^{L1c};
each of said R^{L1c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca}
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L1c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, or octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 10. The compound of any one of the preceding Aspects, wherein L¹ is selected from —O—, —N(CH₃)—, —C(O)—, —NH—, *^{L1}—C(O)N(CH₃)—**^{L1}, *^{L1}—C(O)NH—**^{L1}, *^{L1}—C(O)O—**^{L1}, *^{L1}—C(O)N(C₂H₅)—**^{L1}, *^{L1}—C(O)N(C₃H₇)—**^{L1}, *^{L1}—N(CH₃)C(O)—**^{L1}, *^{L1}—NHC(O)—**^{L1}, *^{L1}—OC(O)—**^{L1}, *^{L1}—N(C₂H₅)C(O)—**^{L1}, *^{L1}—N(C₃H₇)C(O)—**^{L1},

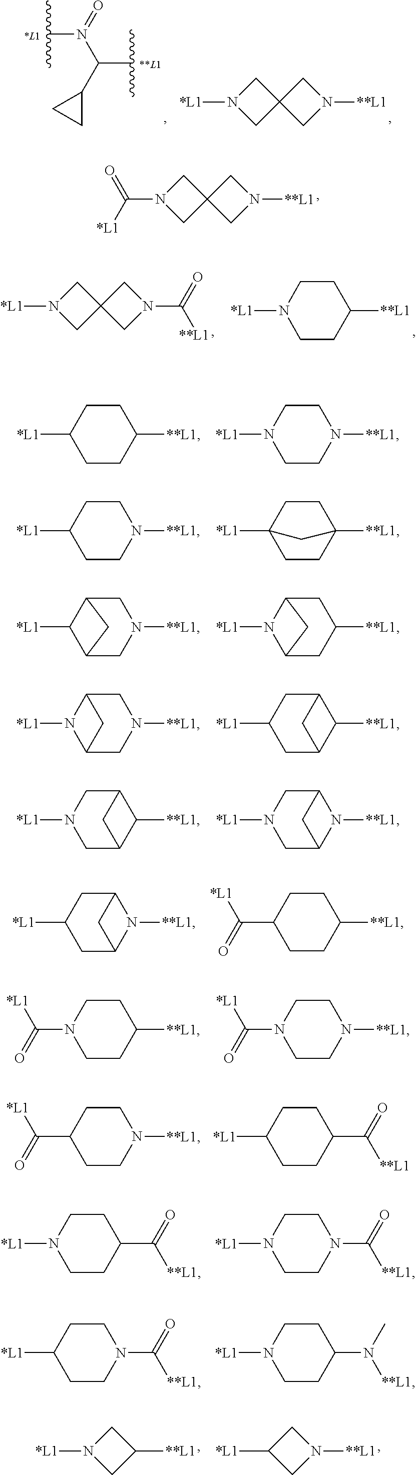

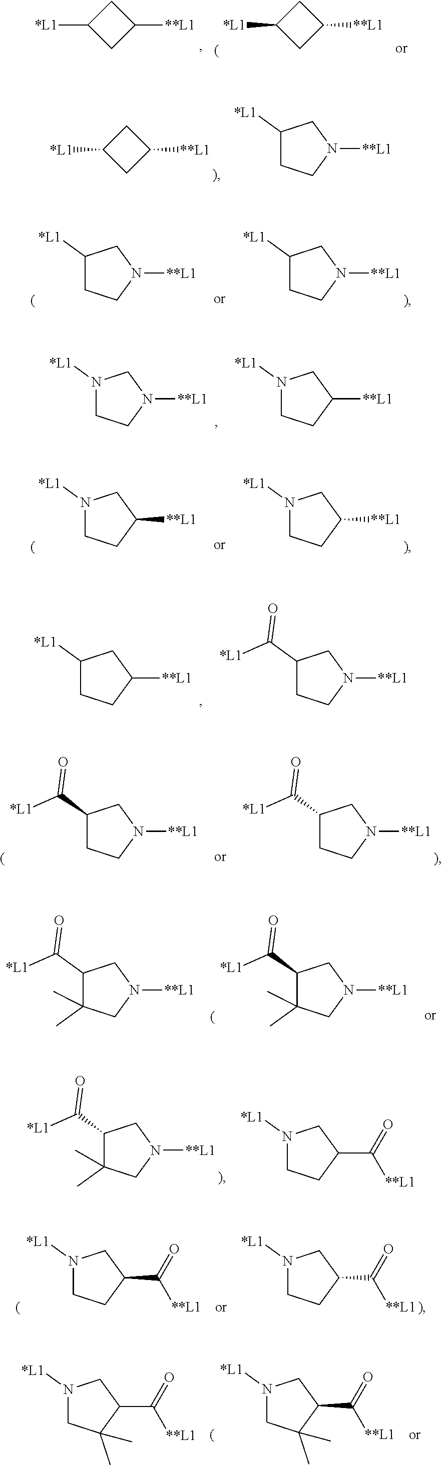

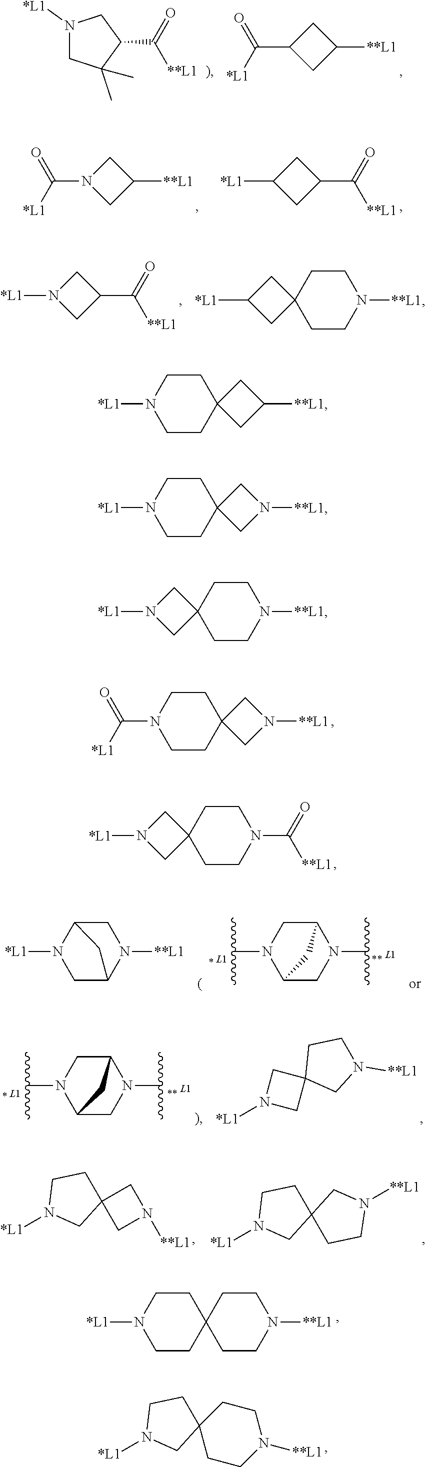

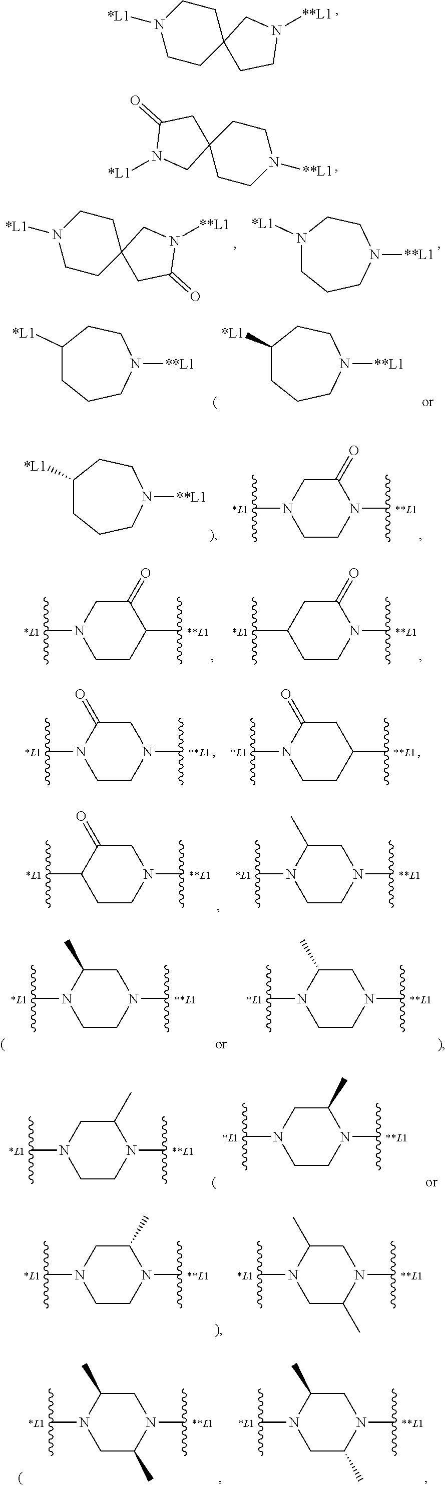

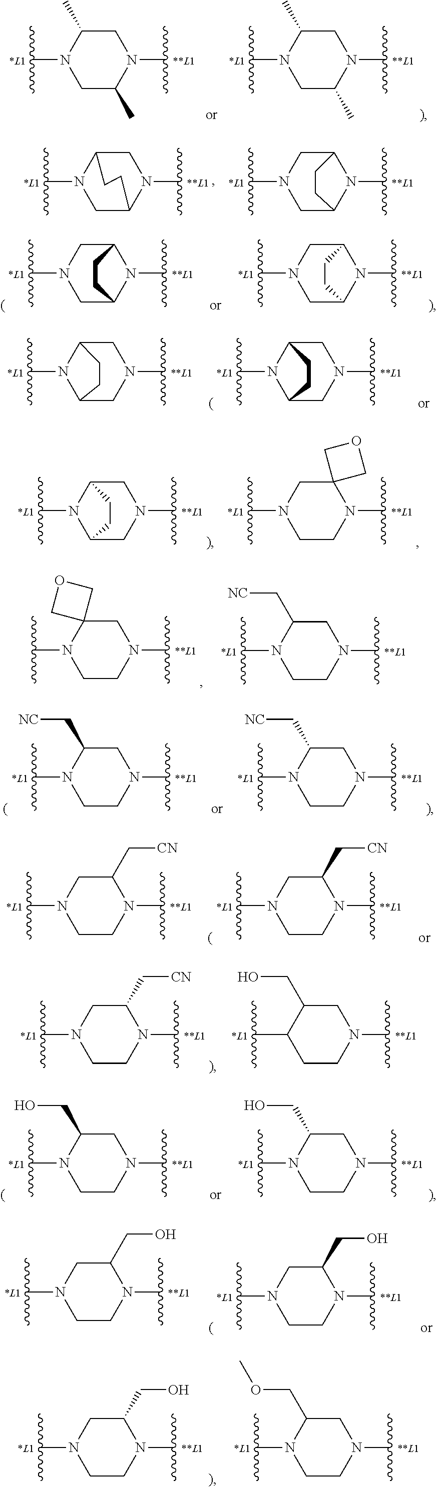

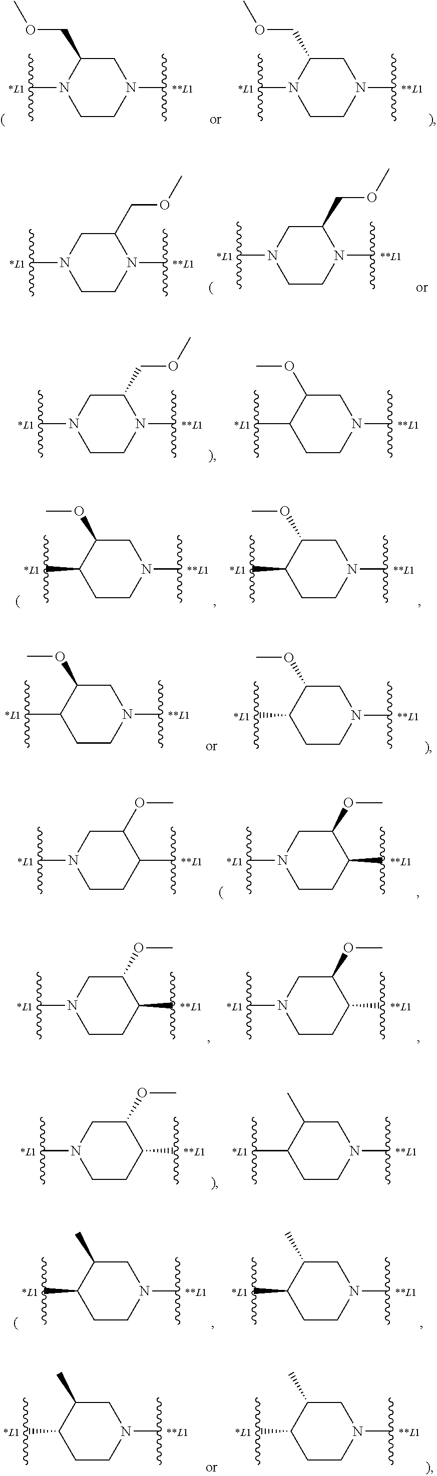

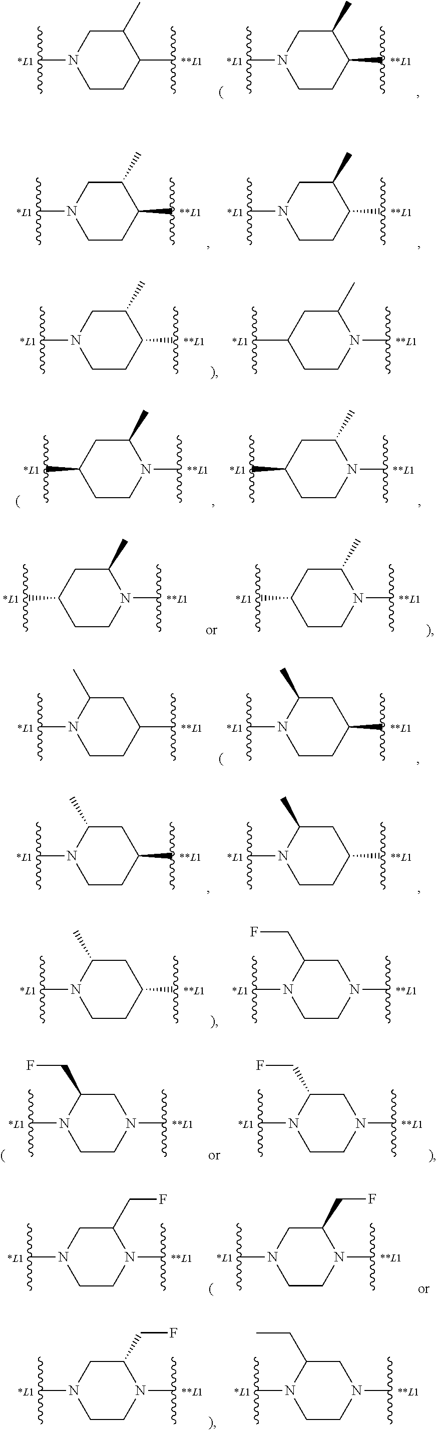

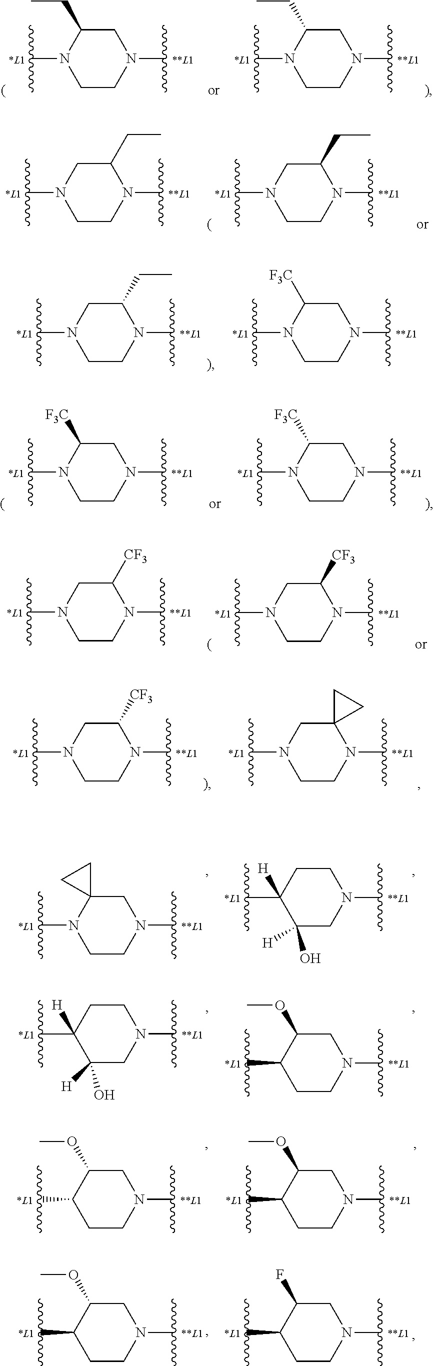

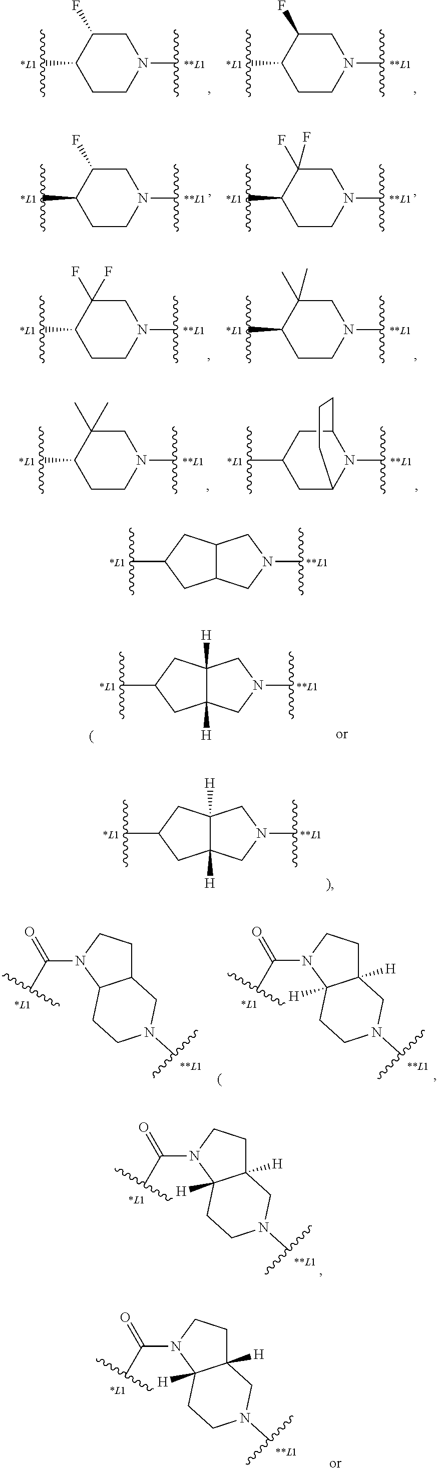

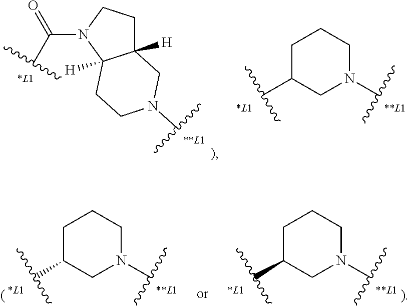

Aspect 11. The compound of any one of the preceding Aspects, wherein L² is selected from —O—, —C(O)—, —N(R^{a})—, *^{L2}—C(O)N(R^{a})—**^{L2}, *^{L2}—C(O)O—**^{L2}, *^{L2}—N(R^{a})C(O)—**^{L2}, *^{L2}-OC(O)**^{L2},

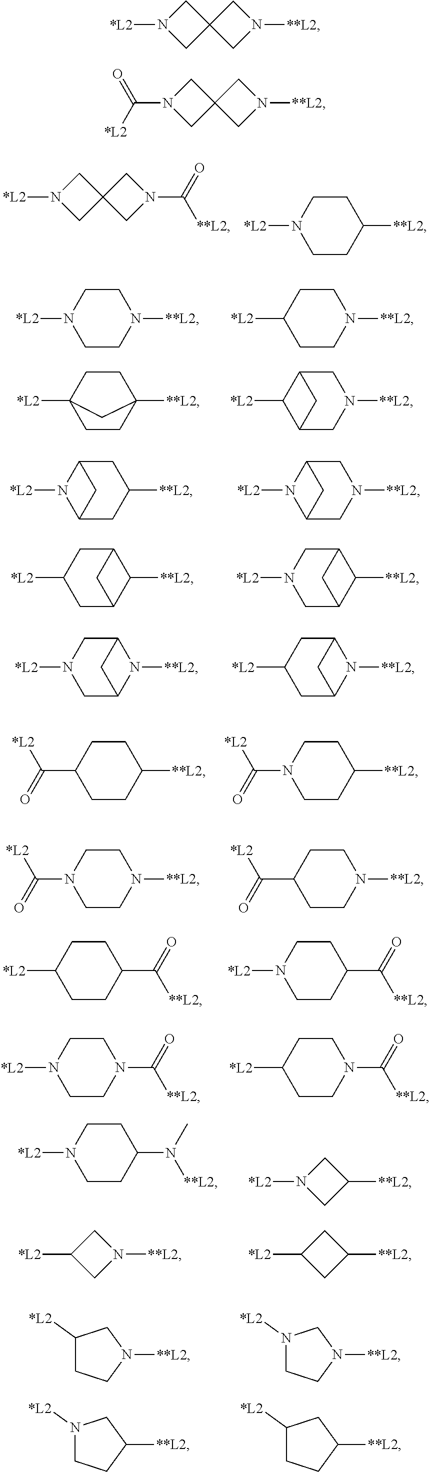

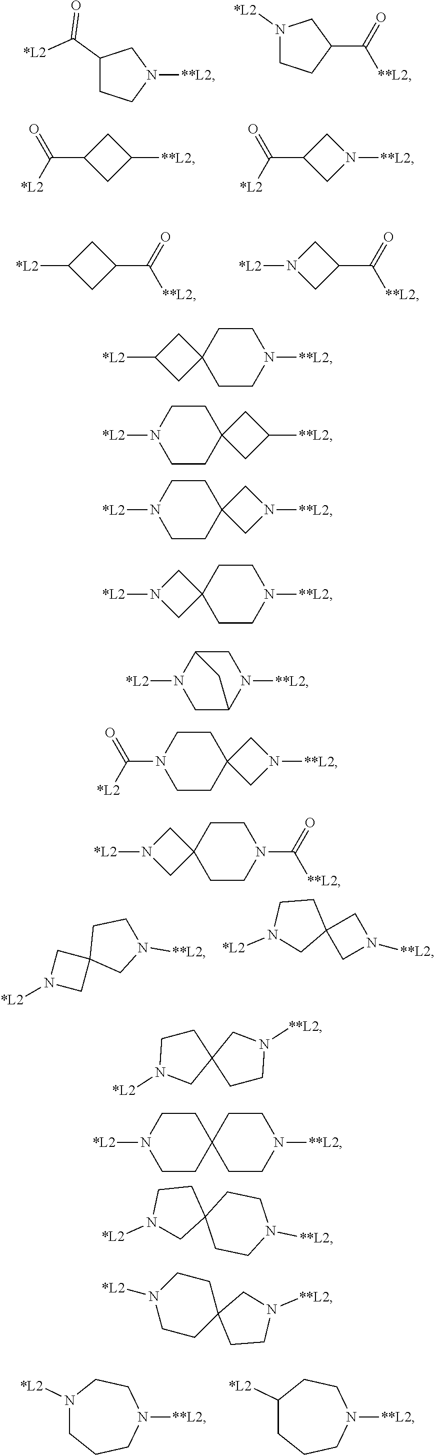

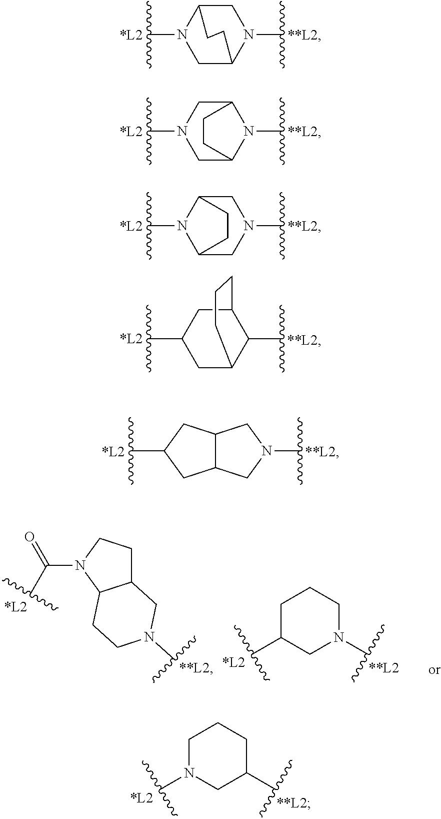

wherein each of said

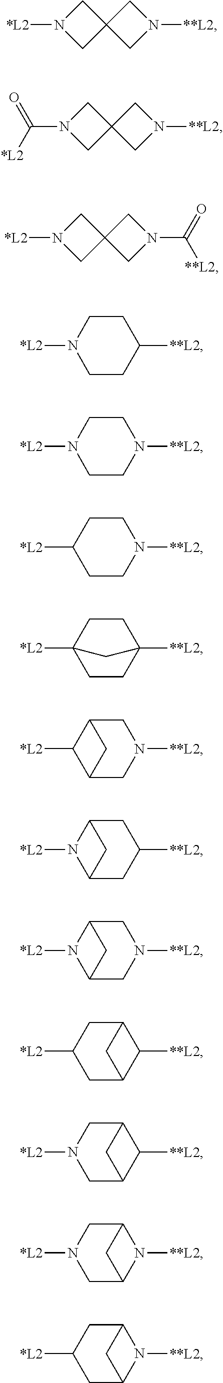

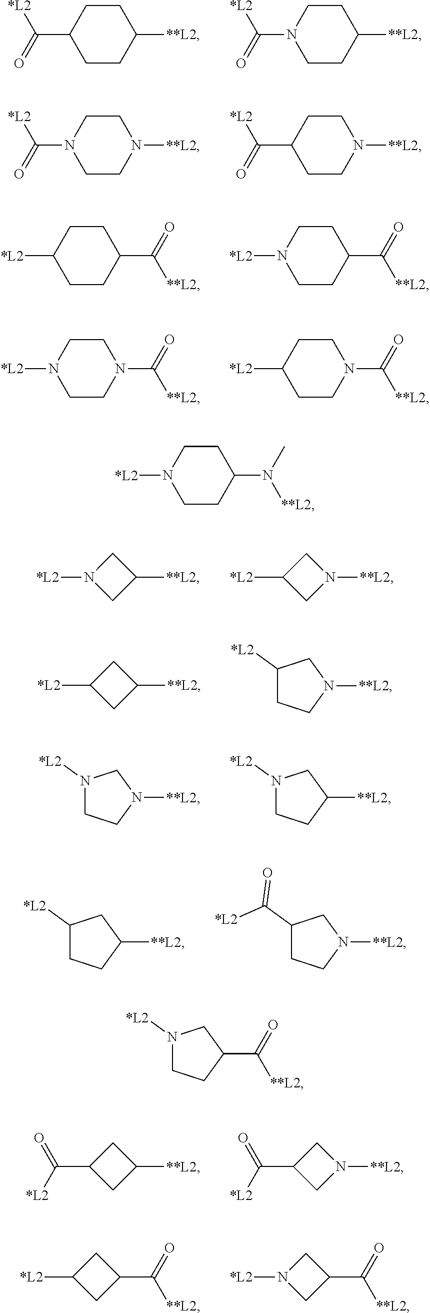

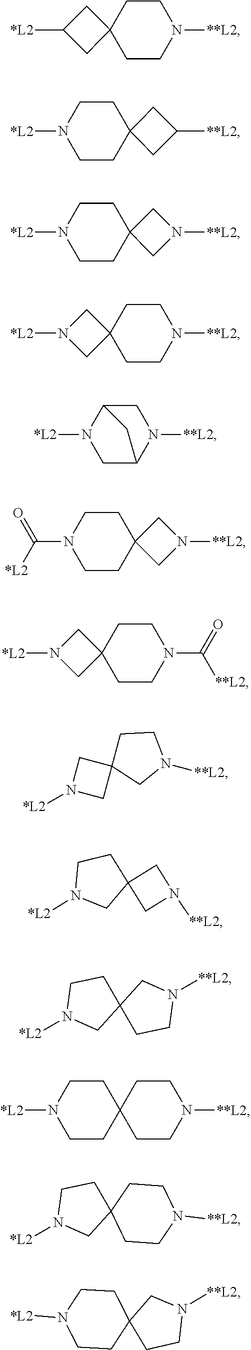

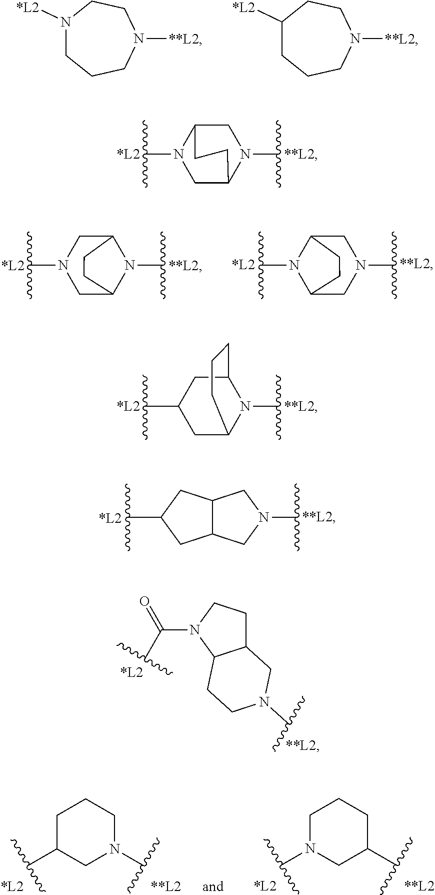

is optionally substituted with at least one R^{L2c};
each of said R^{L2c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca}
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L2c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 12. The compound of any one of the preceding Aspects, wherein L² is selected from —O—, —N(CH₃)—, —C(O)—, —NH—, *^{L2}—C(O)N(CH₃)—**^{L2}, *^{L2}—C(O)NH—**^{L2}, *^{L2}—C(O)O—**^{L2}, *^{L2}—C(O)N(C₂H₅)—**^{L2}, *^{L2}—C(O)N(C₃H₇)—**^{L2}, *^{L2}—N(CH₃)C(O)—**^{L2}, *^{L2}—NHC(O)—**^{L2}, *^{L2}—OC(O)—**^{L2}, *^{L2}—N(C₂H₅)C(O)—**^{L2}, *^{L2}—N(C₃H₇)C(O)—**^{L2},

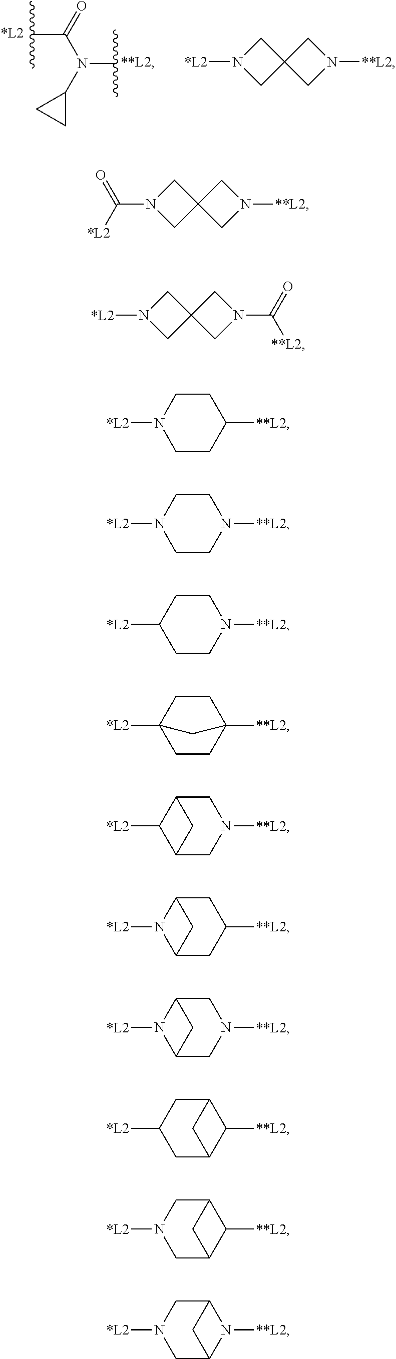

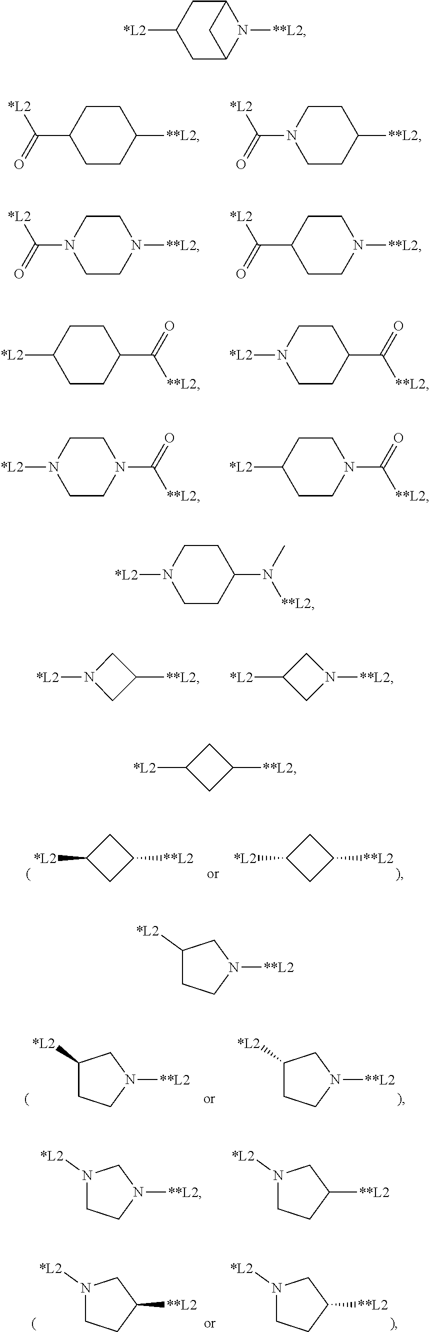

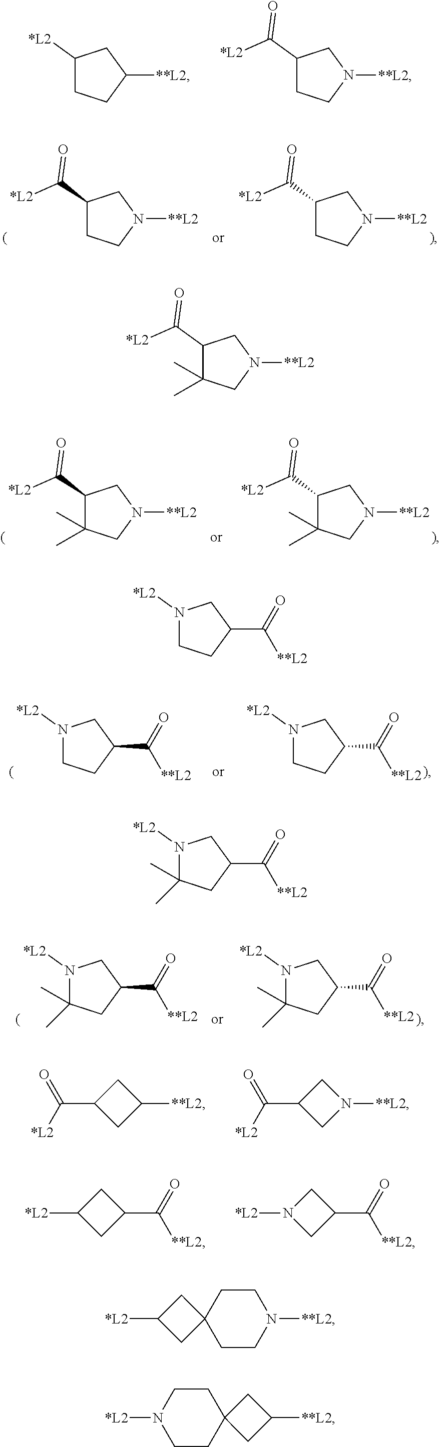

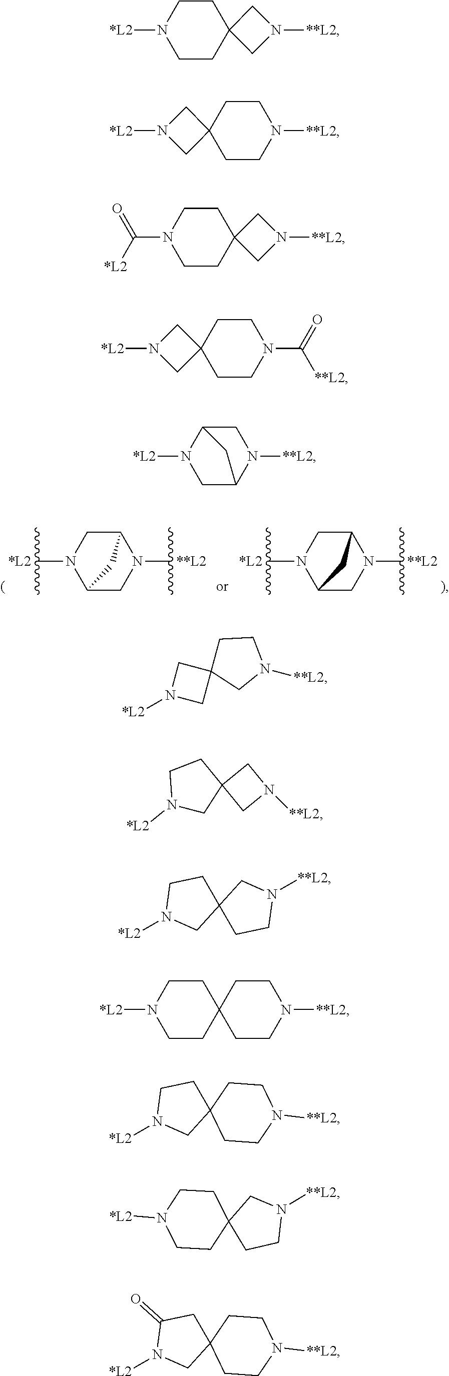

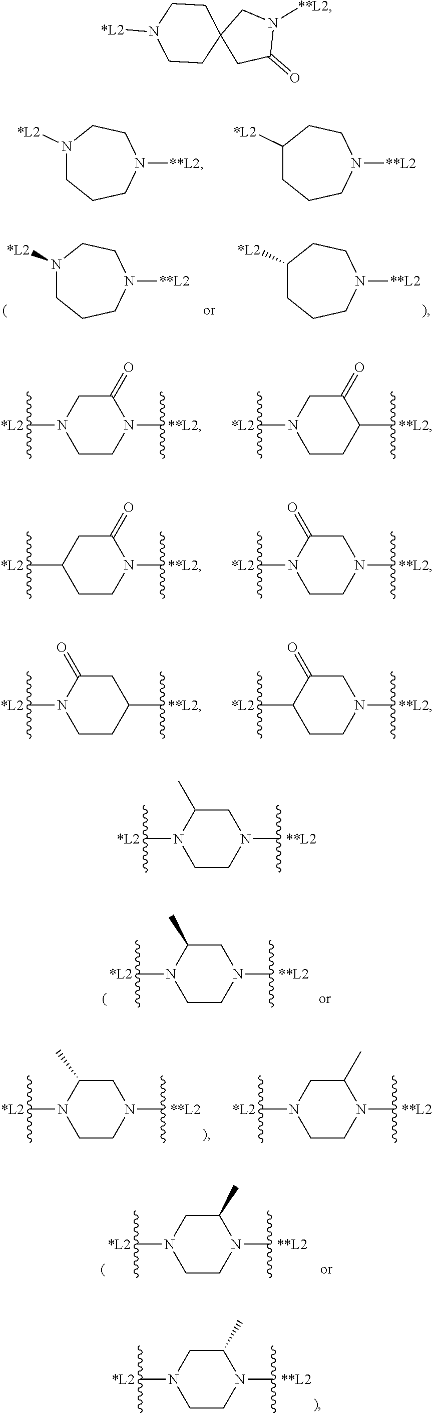

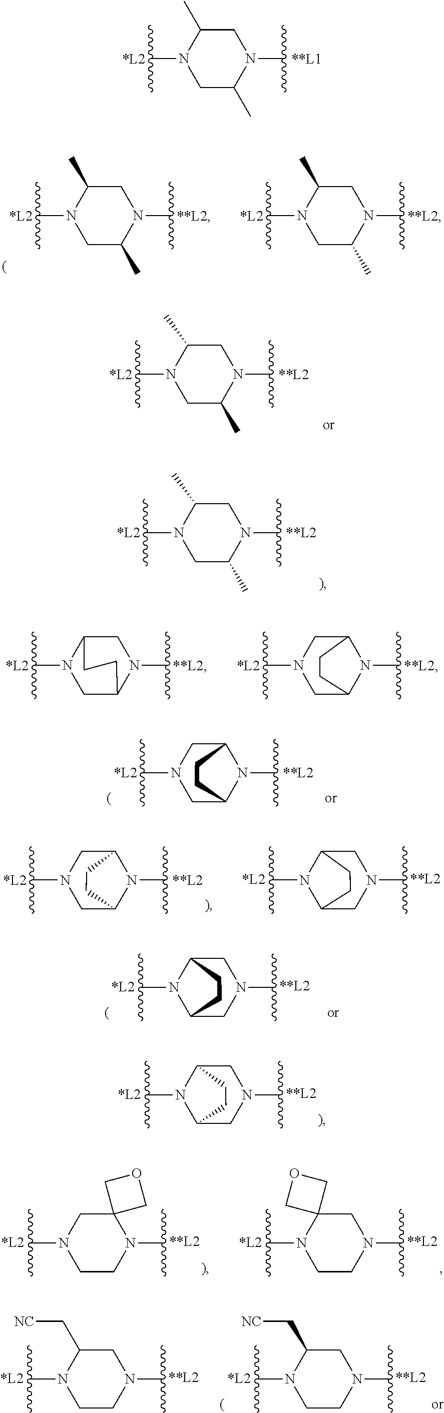

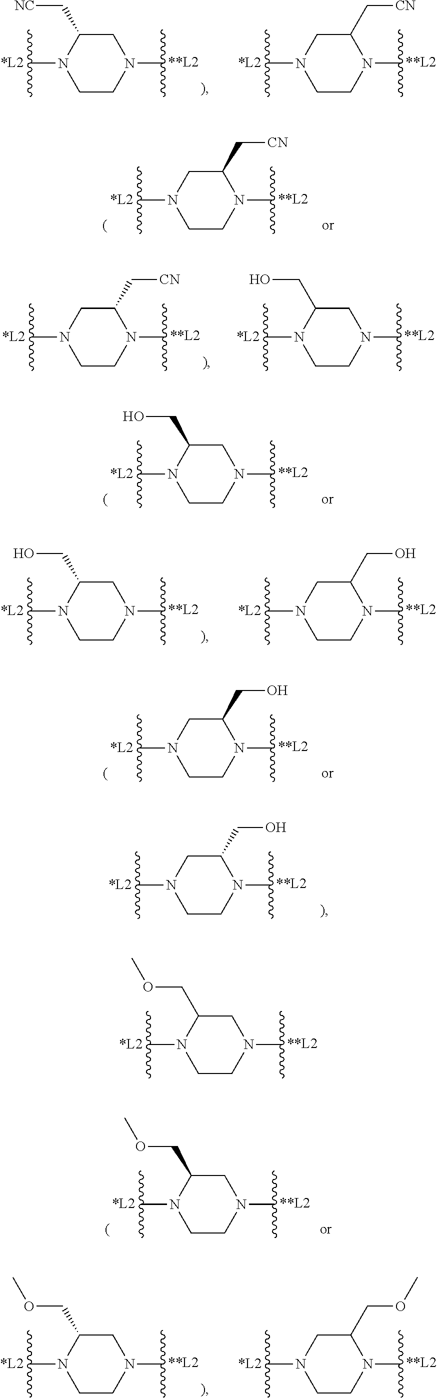

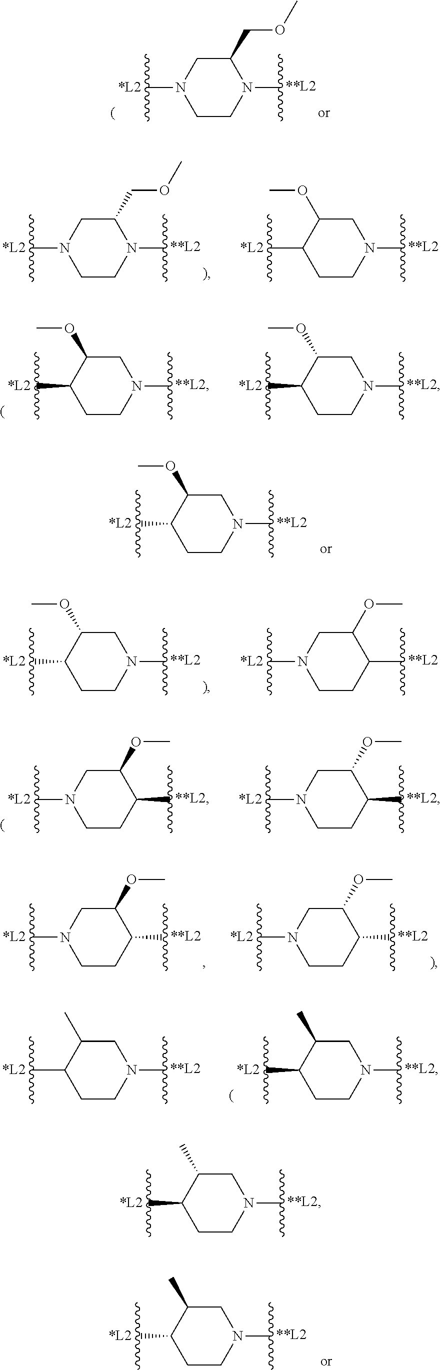

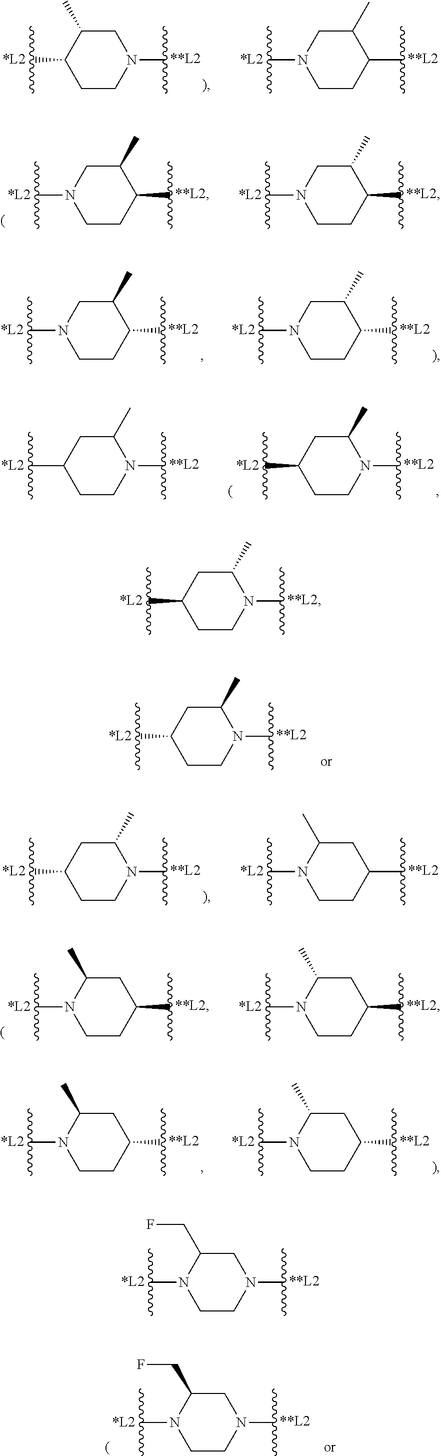

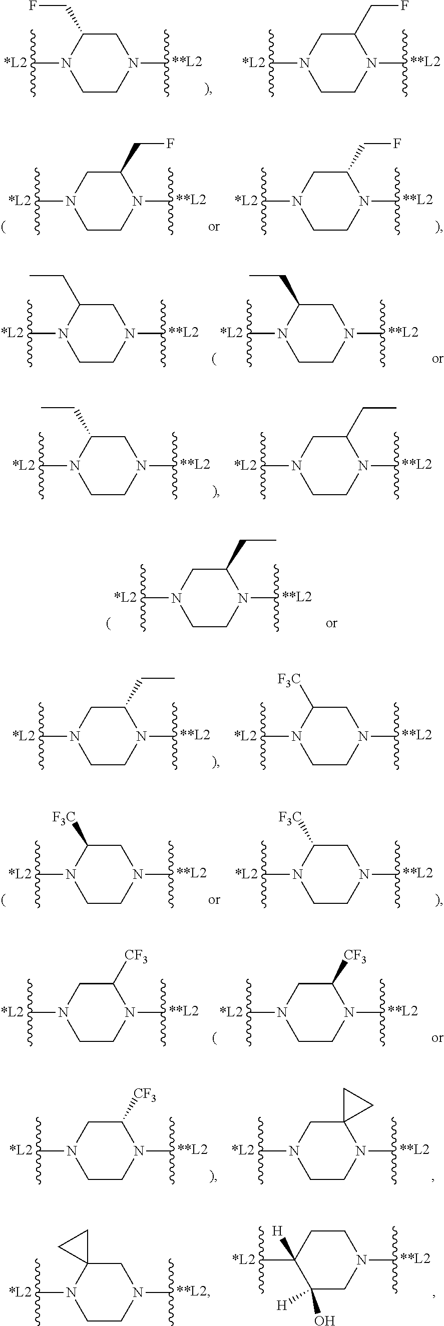

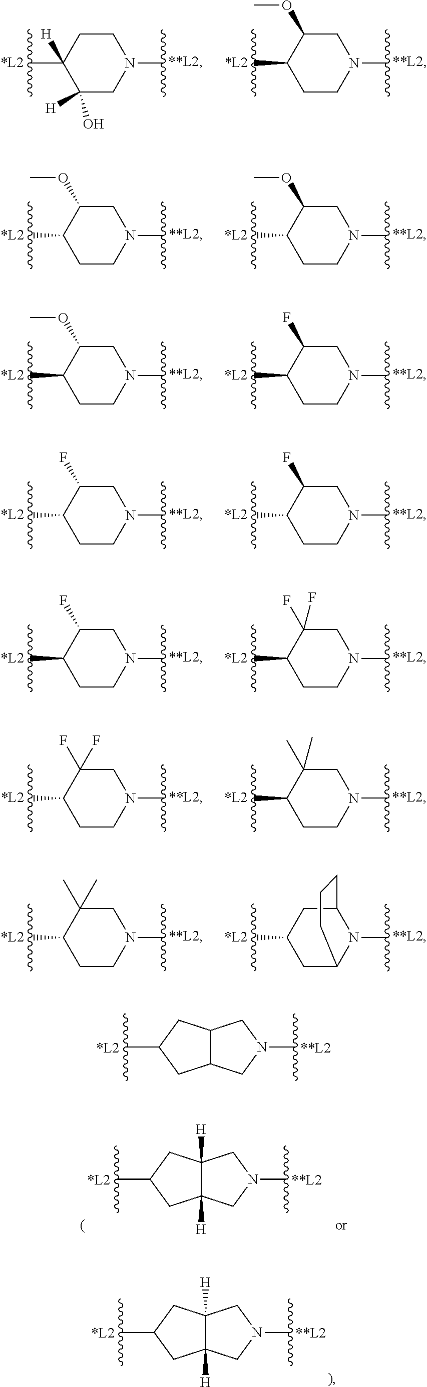

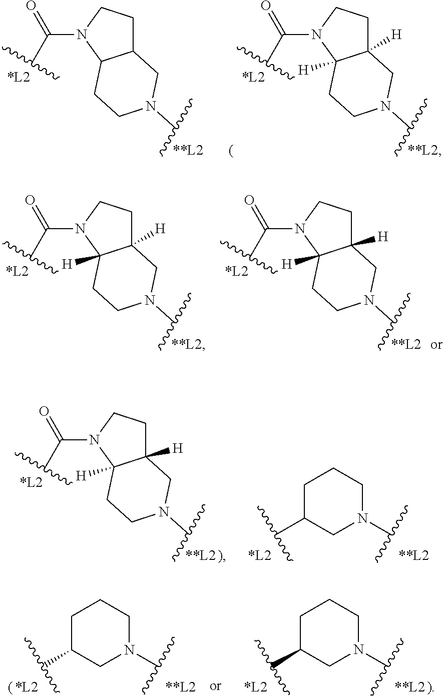

Aspect 13. The compound of any one of the preceding Aspects, wherein L³ is selected from —O—, —N(R^{a})—, —C(O)—, *^{L3}—C(O)N(R^{a})—**^{L3}, *^{L3}—C(O)O—**^{L3}, *^{L3}—N(R^{a})C(O)—**^{L3}, *^{L3}—OC(O)—**^{L3},

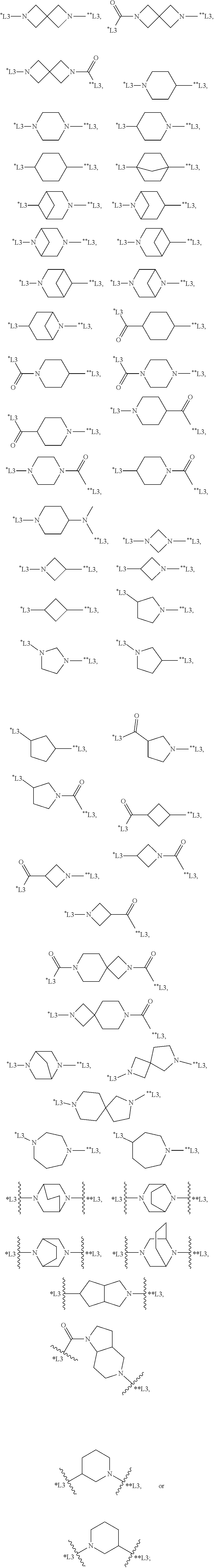

wherein each of said

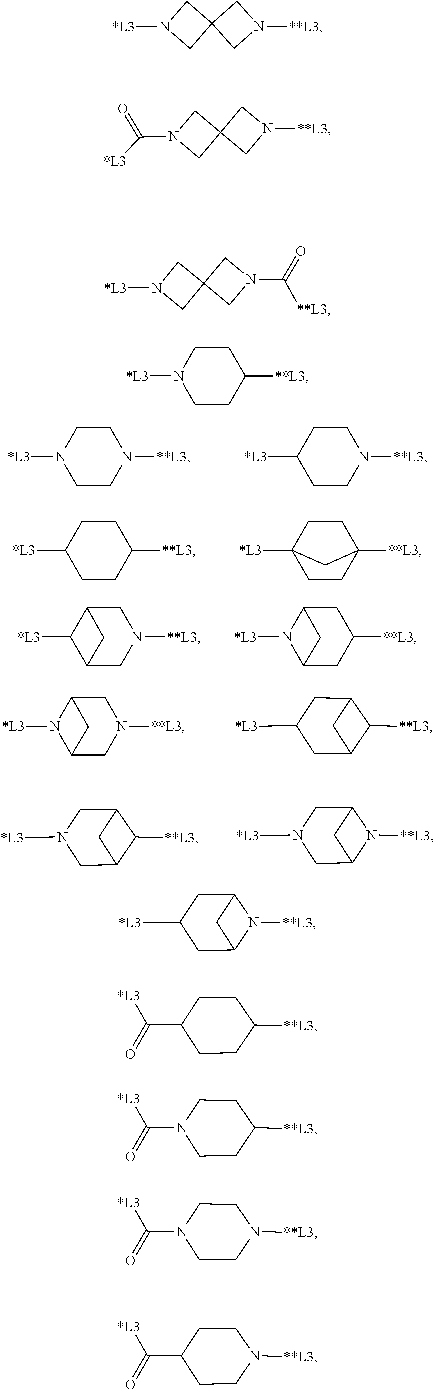

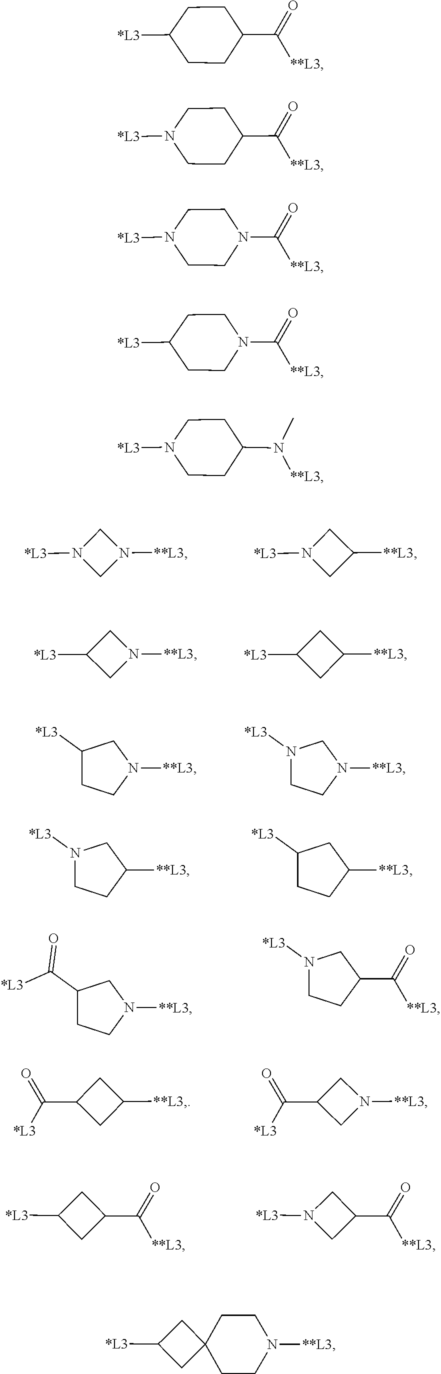

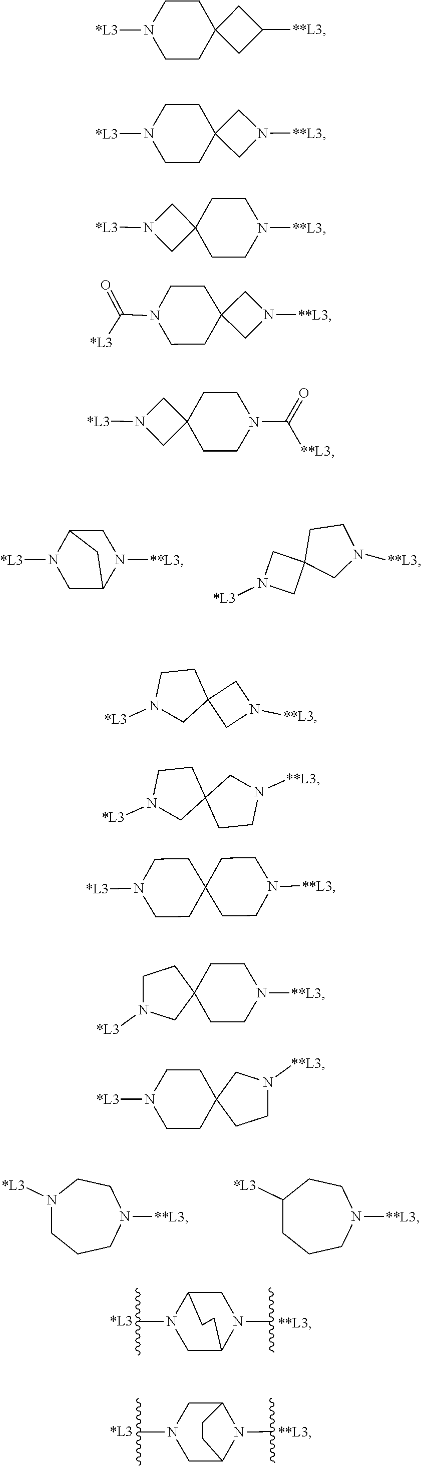

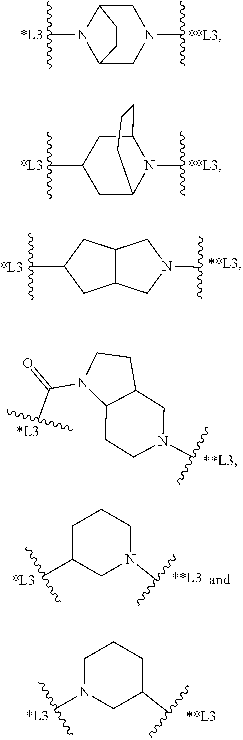

is optionally substituted with at least one R^{L3c};
each of said R^{L3c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca}
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or two R^{L3c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl; R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 14. The compound of any one of the preceding Aspects, wherein L³ is selected from —O—, —N(CH₃)—, —C(O)—, —NH—, *^{L3}—C(O)N(CH₃)—**^{L3}, *^{L3}—C(O)NH—**^{L3}, *^{L3}—C(O)O—**^{L3}, *^{L3}—C(O)N(C₂H₅)—**^{L3}, *^{L3}—C(O)N(C₃H₇)—**^{L3}, *^{L3}—N(CH₃)C(O)—**^{L3}, *^{L3}—NHC(O)—**^{L3}, *^{L3}—OC(O)—**^{L3}, *^{L3}—N(C₂H₅)C(O)—**^{L3}, *^{L3}—N(C₃H₇)C(O)—**^{L3},

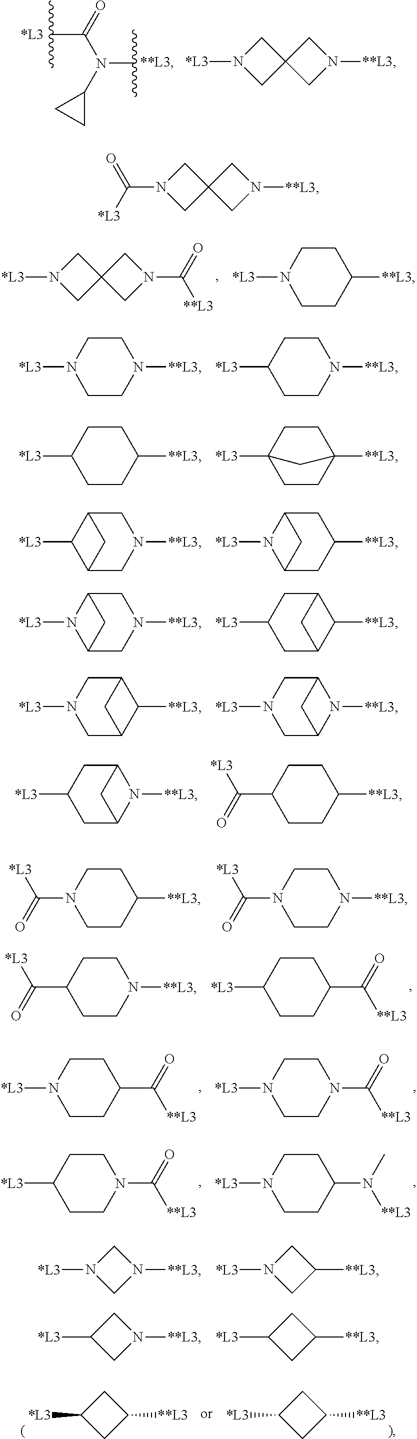

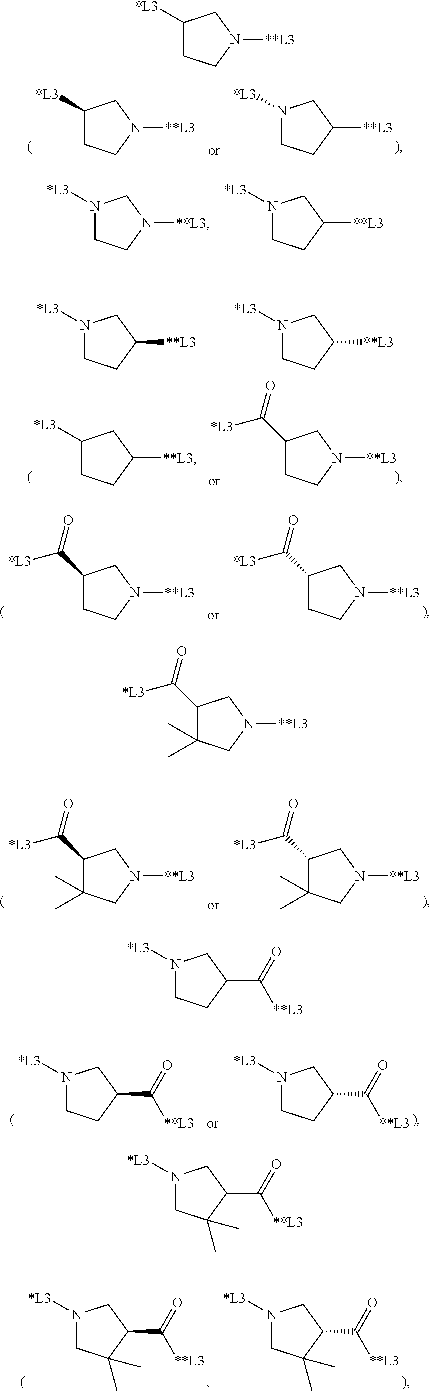

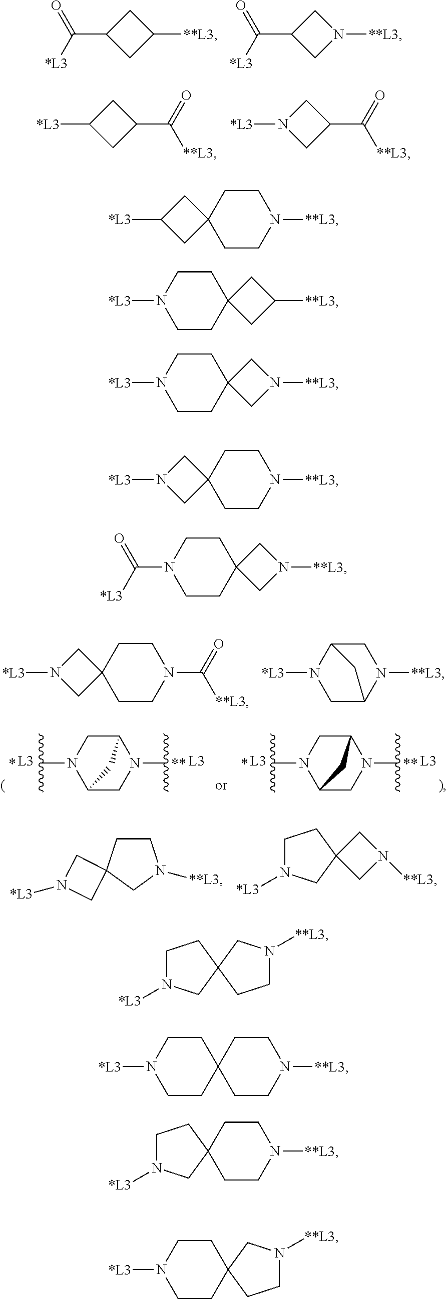

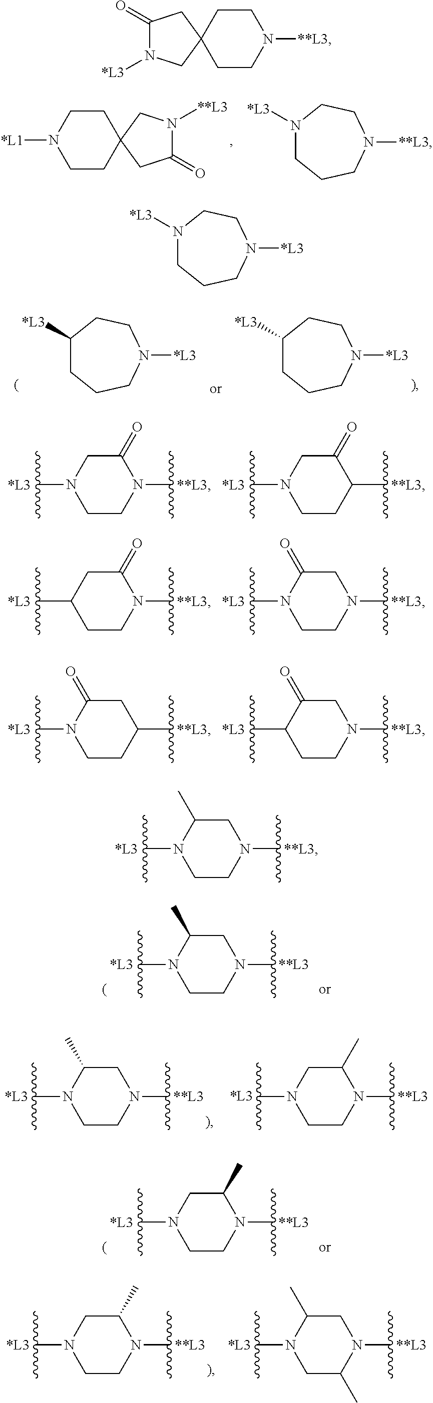

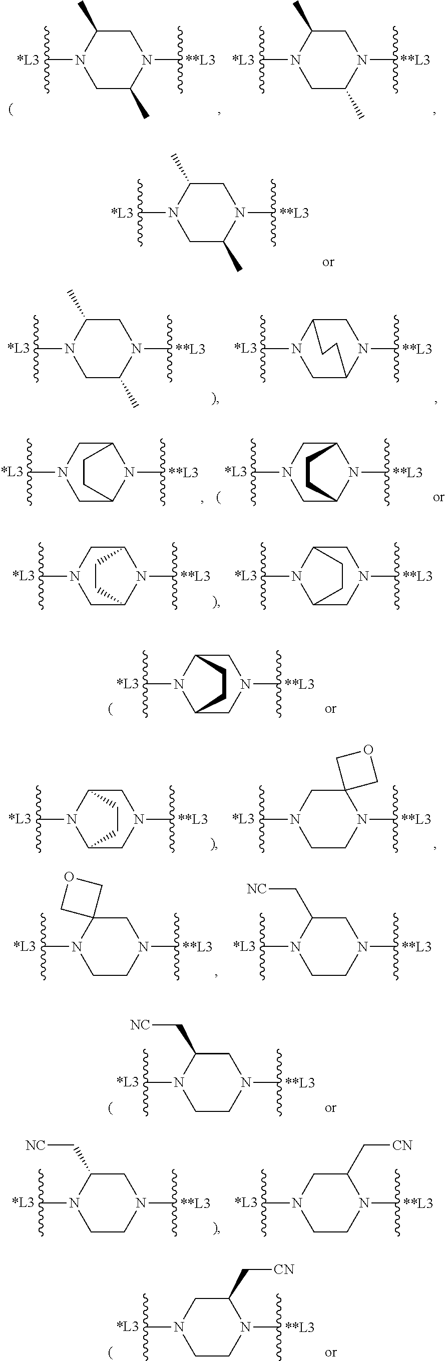

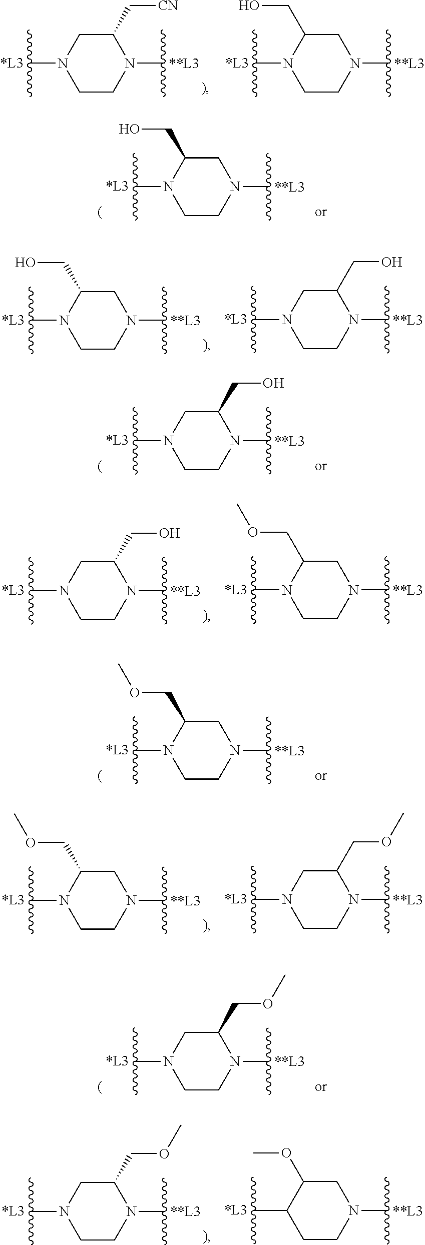

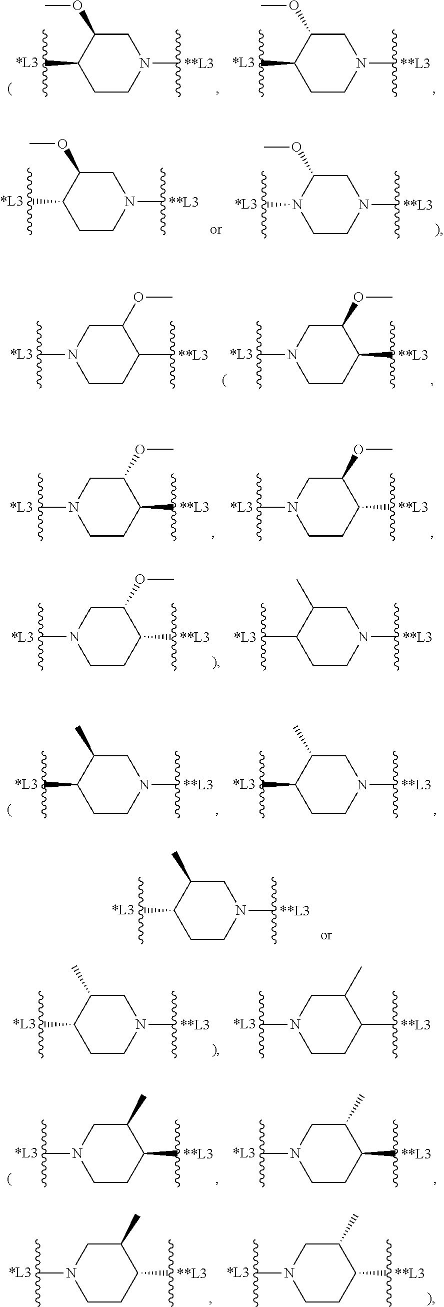

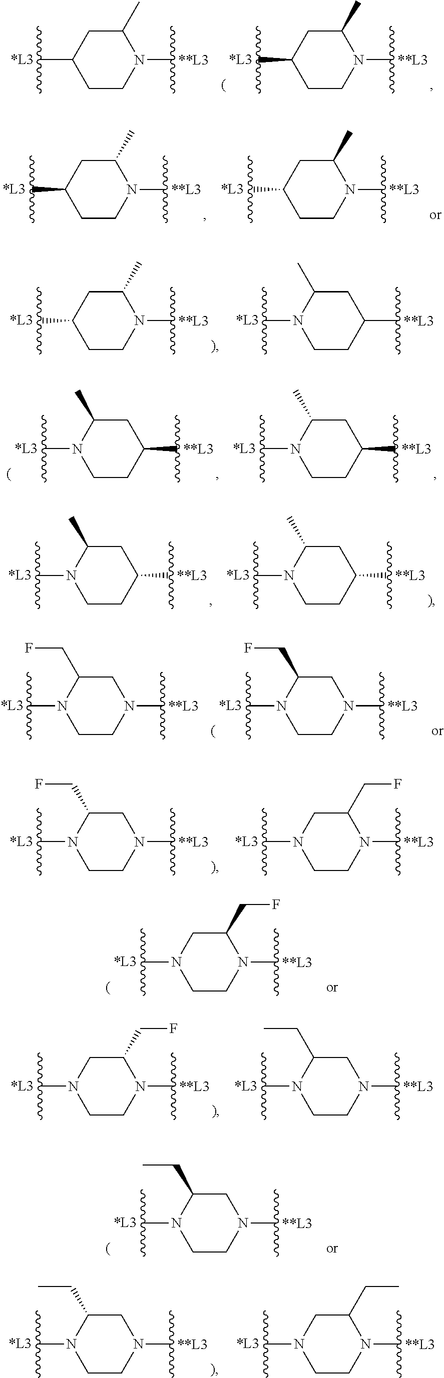

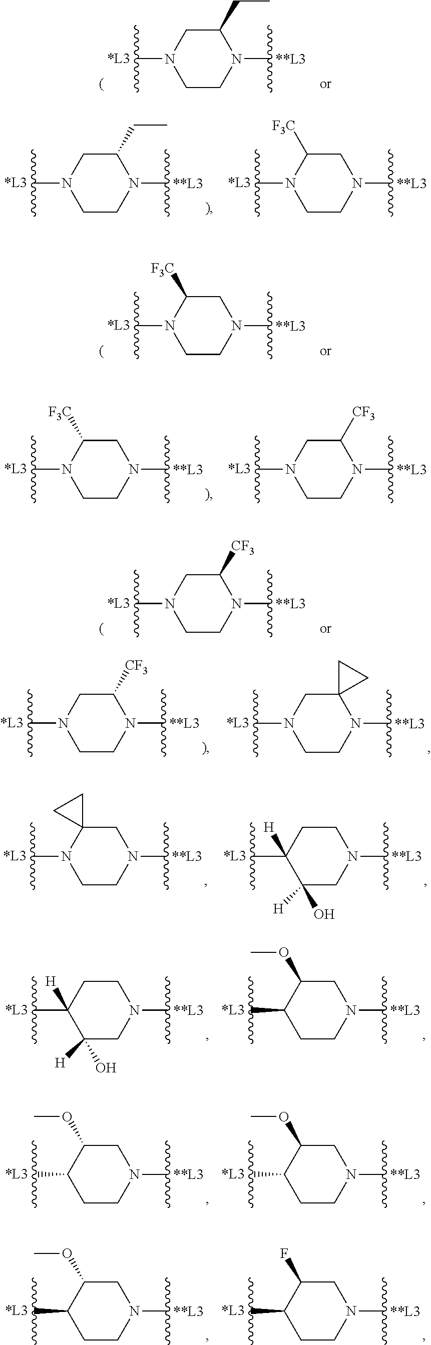

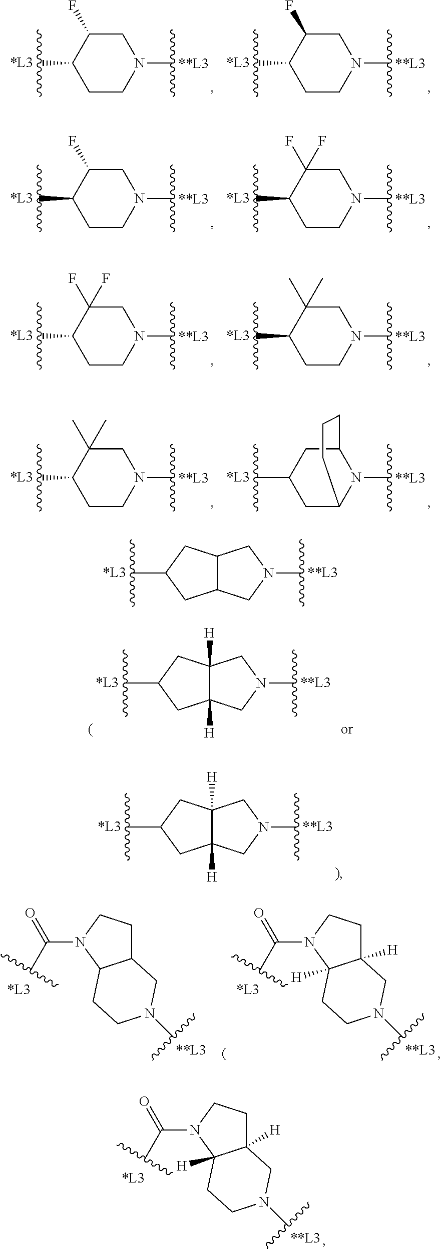

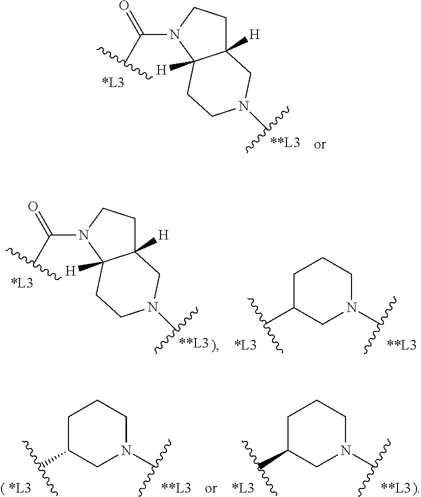 [SMILES: *C(=O)N1CC[C@@H]2CN(*)CC[C@@H]21 ; *C(=O)N1CC[C@@H]2CN(*)CC[C@@H]21 ; *C1CCCN(*)C1 ; *[C@@H]1CCCN(*)C1 ; *[C@H]1CCCN(*)C1]

Aspect 15. The compound of any one of the preceding Aspects, wherein

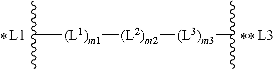

moiety is selected from,

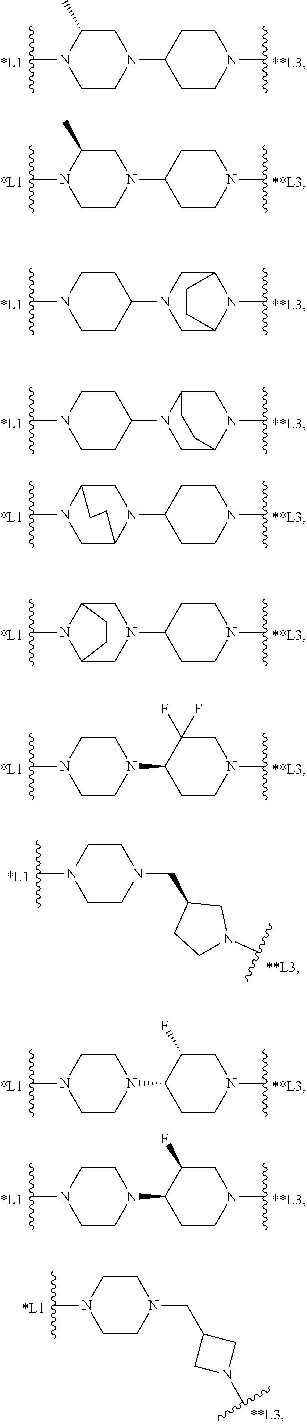

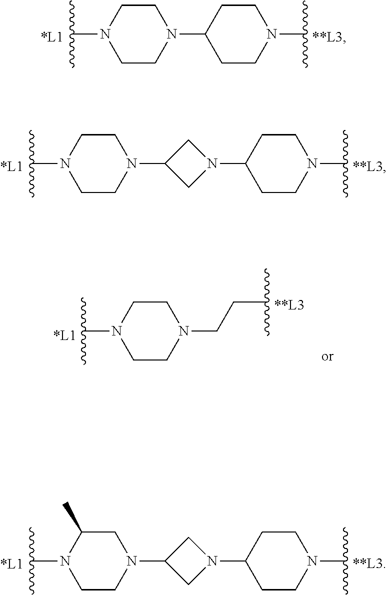 [SMILES: *N1CCC(N2CCN(*)CC2)CC1 ; *N1CCC(N2CC(N3CCN(*)CC3)C2)CC1 ; *CCN1CCN(*)CC1 ; *N1CCC(N2CC(N3CCN(*)[C@@H](C)C3)C2)CC1]

Aspect 16. The compound of any one of the preceding Aspects, wherein L⁴ is independently selected from a single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—; at each occurrence, R^{a} and R are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; preferably, L⁴ is independently selected from a single bond.

Aspect 17. The compound of any one of the preceding Aspects, wherein X⁷ is independently selected from —CR^{a}, or N;
R^{a} is independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
preferably, X⁷ is independently selected from —CH, —C(CH₃), or N; preferably X⁷ is independently selected from —CH.

Aspect 18. The compound of any one of the preceding Aspects, wherein X⁸ is independently selected from —NR^{a}—, —O—, —S— and —CR^{a}R^{b}—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
preferably, X⁸ is independently selected from —NH— and —CH₂—; preferably X⁸ is independently selected from —CH₂—.

Aspect 19. The compound of any one of the preceding Aspects, wherein

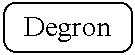

is selected from

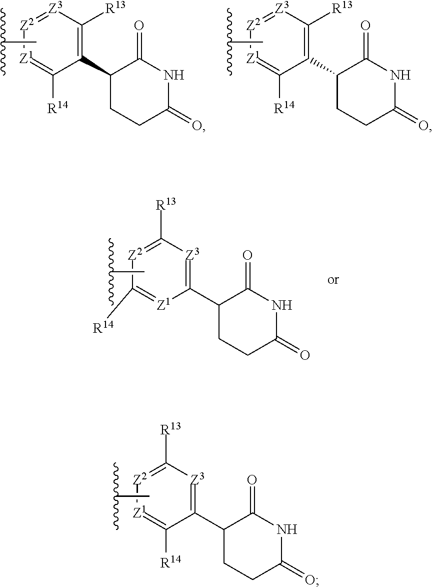

preferably,

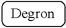

is selected from

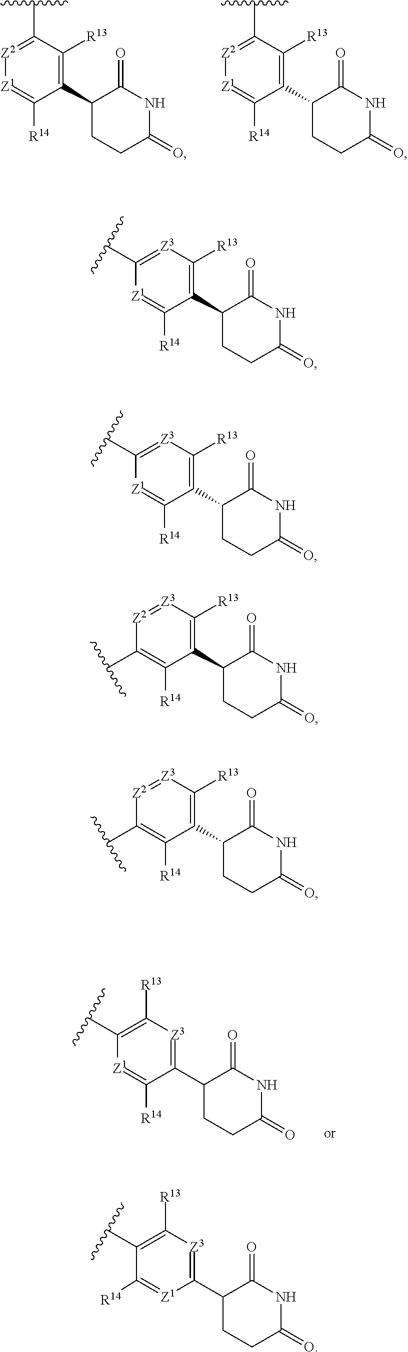

Aspect 20. The compound of any one of the preceding Aspects, wherein at most one of Z¹, Z² and Z³ is N;
preferably, Z¹, Z² and Z³ are each independently CR^{z}.

Aspect 21. The compound of any one of the preceding Aspects, wherein R^{z}, at each occurrence, is independently selected from hydrogen, —F, —Cl, —Br, —I, methyl, ethyl, propyl, butyl, pentyl, hexyl, heptyl, octyl, —NR^{Za}R^{Zb}, —OR^{Za}, —SR^{Za}, cyclopropyl, cyclobutyl, cyclopentyl, cyclohexyl, cycloheptyl, cyclooctyl, 3- to 8-membered heterocyclyl or CN; each of methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl is optionally substituted with at least one R^{Zc};
R^{za} and R^{Zb} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{Zd};
R^{Ze} and R^{Zd} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl, or 5- to 12-membered heteroaryl;
preferably, R^{z} is selected from H, —CH₃, —C₂H₅, F, —CH₂F, —CHF₂, —CF₃, —OCH₃, —OC₂H₅, —C₃H₇, —OCH₂F, —OCHF₂, —OCH₂CF₃, —OCF₃, —SCF₃, —CF₃ or —CH(OH)CH₃.

Aspect 22. The compound of any one of the preceding Aspects, wherein R¹³ and R¹⁴ are each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}—NR^{13a}CO₂R^{13b} or —NR^{13a}SO₂R^{13b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, —NR^{13c}COR^{13d}, —NR^{13c}CO₂R^{13d} or —NR^{13c}SO₂R^{13d};
R^{13a}, R^{13b}, R^{13c} and R^{13d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5- to 12-membered heteroaryl;
preferably, R¹³ and R¹⁴ are each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CN, —CH₂F, —CHF₂, —CF₃, —OCH₂F, —OCHF₂, —OCH₂CF₃, —OCF₃, —SCF₃, or phenyl.

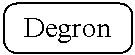

Aspect 23. The compound of any one of the preceding Aspects, wherein is

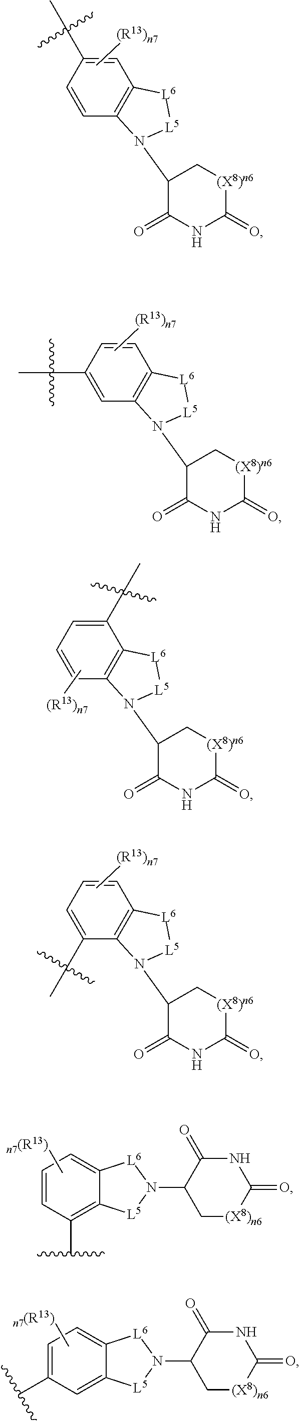

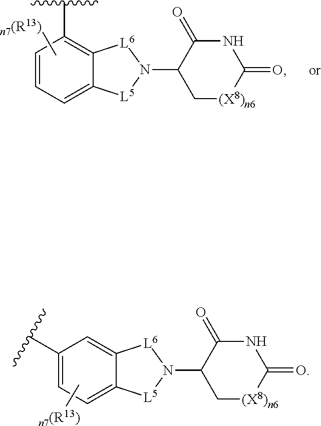

Aspect 24. The compound of any one of the preceding Aspects, wherein L⁵ and L⁶ are each independently selected from a single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
X⁸ is —CR^{a}R^{b}—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
preferably, L⁵ and L⁶ are each independently a single bond,

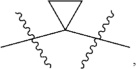 [SMILES: *C1(*)CC1]

—O—, —NH—, —NMe-, —N(CH₂CH₃)—, —CH₂—, —CHF—, —CF₂—, —C(CH₃)₂— or —CO—;
X⁸ is CH₂; and
n6 is 0 or 1.

Aspect 25. The compound of any one of the preceding Aspects, wherein R¹³ is independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}CO₂R^{13b} or —NR^{13a}SO₂R^{13b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, —NR^{13c}COR^{13d}, —NR^{13c}CO₂R^{13d} or —NR^{13c}SO₂R^{13d};
at each occurrence, R^{13a}, R^{13b}, R^{13c} and R^{13d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5- to 12-membered heteroaryl;
preferably, R¹³ is independently selected from hydrogen, F, Cl, Br, I, CN, —C₁-C₈alkyl, or —C₁-C₈alkoxy; preferably R¹³ is independently selected from hydrogen, F, Cl, Br, I, CN, -Me, -Et, —C₃H₇, —C₄H₉, —OMe, -OEt, —OC₃H₇ or —OC₄H₉;
n₇ is 0, 1 or 2.

Aspect 26. The compound of any one of the preceding Aspects, wherein

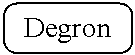

is

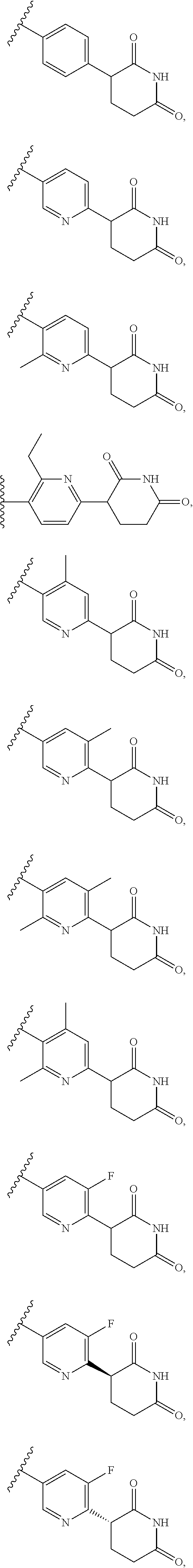

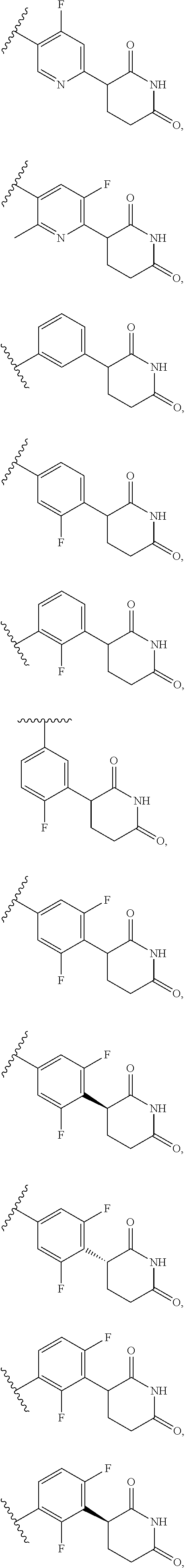

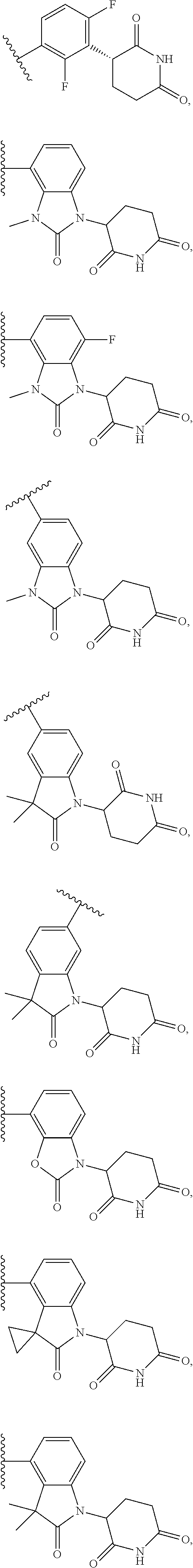

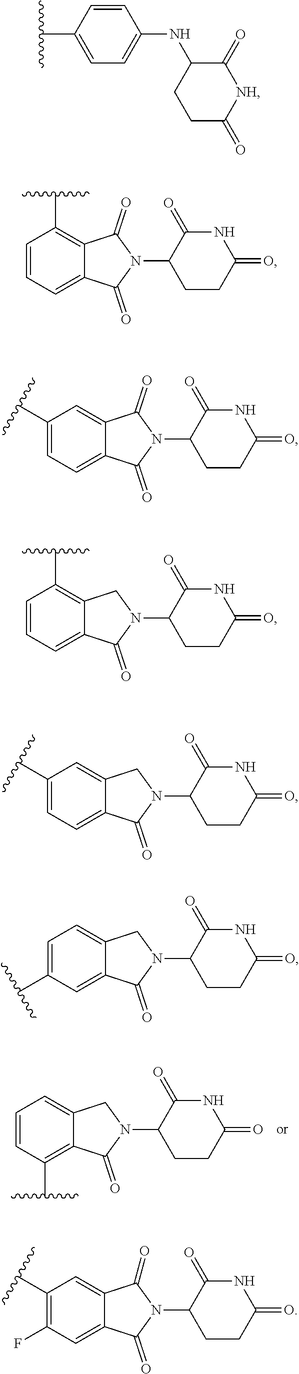

Aspect 27. The compound of any one of the preceding Aspects, wherein the compound is selected from

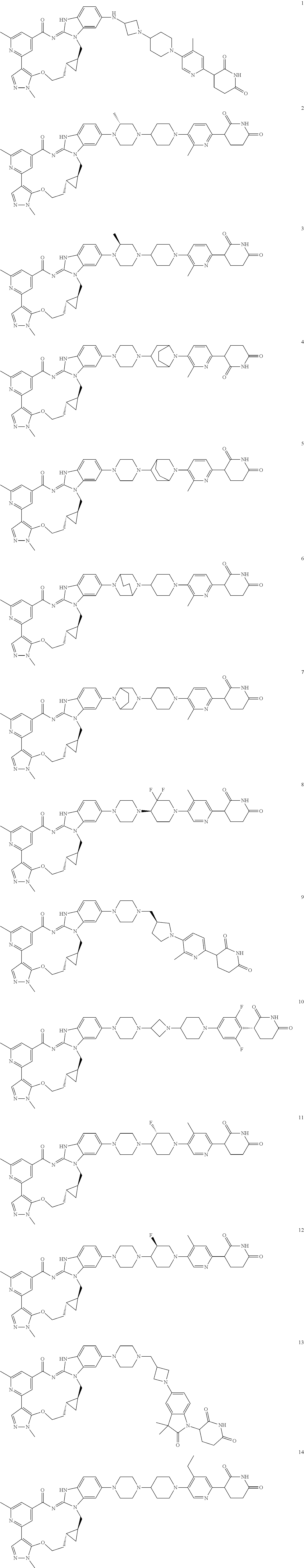

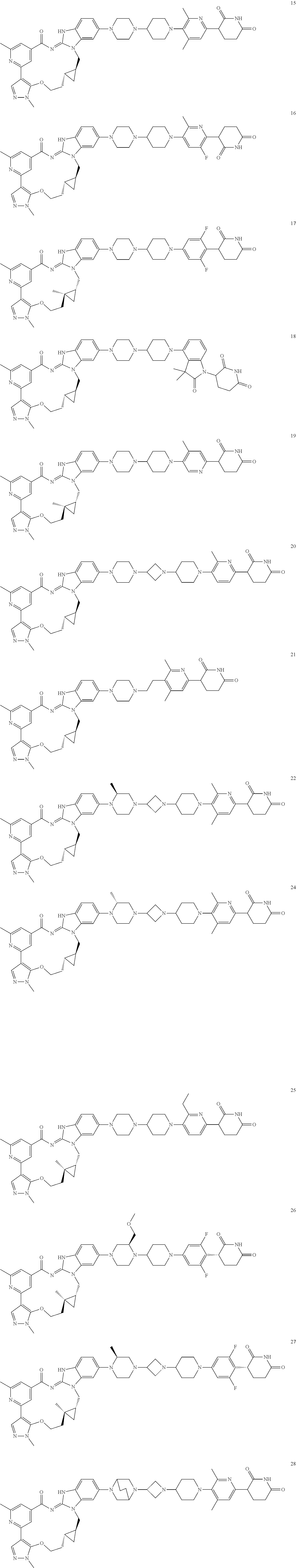

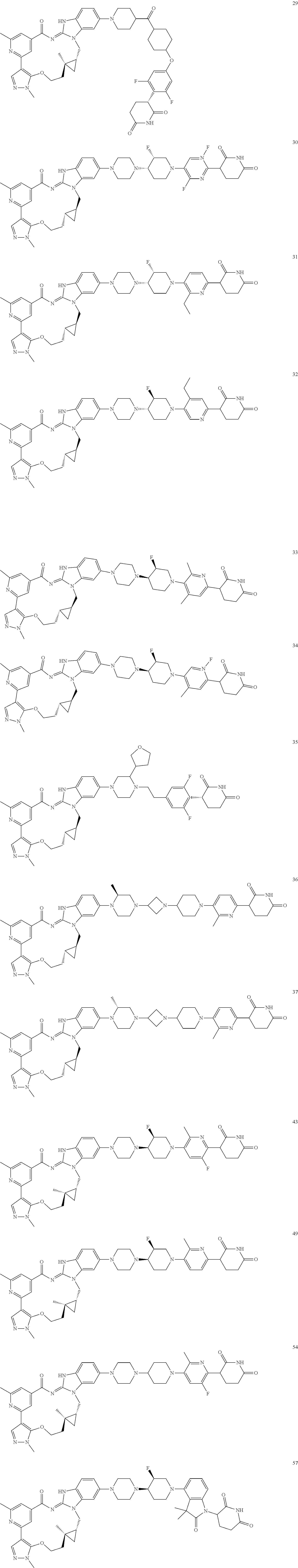

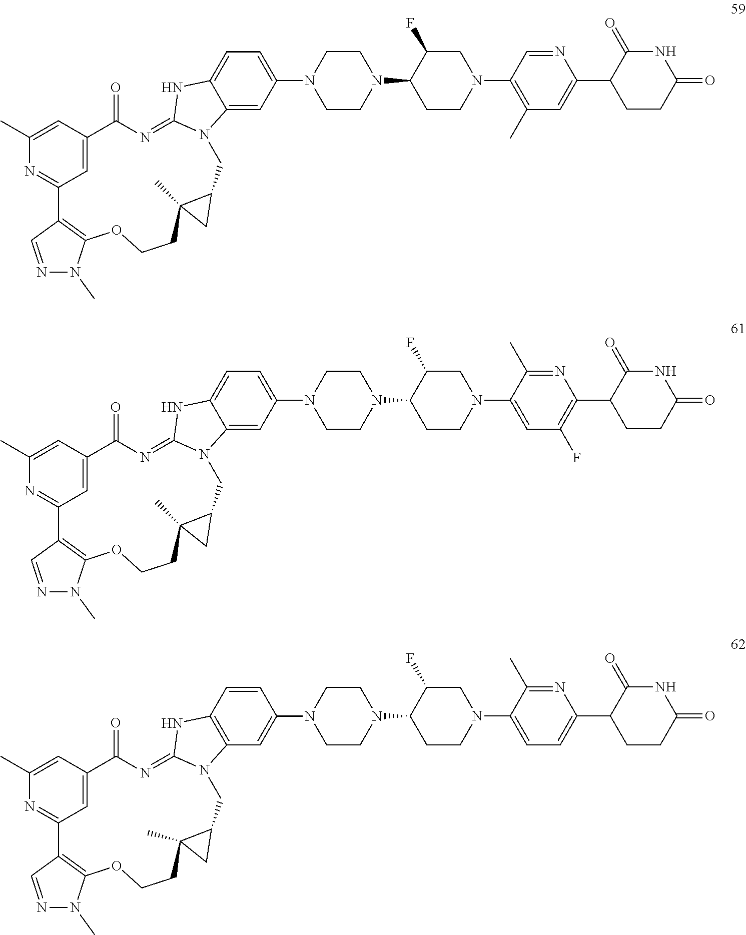 [SMILES: Cc1cc2cc(n1)-c1cnn(C)c1OCC[C@]1(C)C[C@H]1CN1/C(=N/C2=O)Nc2ccc(N3CCN([C@@H]4CCN(c5cnc(C6CCC(=O)NC6=O)cc5C)C[C@@H]4F)CC3)cc21 ; Cc1cc2cc(n1)-c1cnn(C)c1OCC[C@]1(C)C[C@H]1CN1/C(=N/C2=O)Nc2ccc(N3CCN([C@H]4CCN(c5cc(F)c(C6CCC(=O)NC6=O)nc5C)C[C@H]4F)CC3)cc21 ; Cc1cc2cc(n1)-c1cnn(C)c1OCC[C@]1(C)C[C@H]1CN1/C(=N/C2=O)Nc2ccc(N3CCN([C@H]4CCN(c5ccc(C6CCC(=O)NC6=O)nc5C)C[C@H]4F)CC3)cc21]

Aspect 28. A pharmaceutical composition comprising a compound of any one of Aspects 1-27 or a pharmaceutically acceptable salt, stereoisomer, tautomer or prodrug thereof, together with a pharmaceutically acceptable excipient.

Aspect 29. A method of treating a disease that can be affected by EGFR modulation, comprises administrating a subject in need thereof an effective amount of a compound of any one of Aspects 1-27 or a pharmaceutically acceptable salt, stereoisomer, tautomer or prodrug thereof.

Aspect 30. The method of Aspect 29, wherein the disease is selected from cancer, preferred pancreatic cancer, breast cancer, glioblastoma multiforme, head and neck cancer, or non-small cell lung cancer.

Aspect 31. Use of a compound of any one of Aspects 1-27 or a pharmaceutically acceptable salt, stereoisomer, tautomer or prodrug thereof in the preparation of a medicament for treating a disease that can be affected by EGFR modulation.

Aspect 32. The use of Aspect 31, wherein the disease is cancer, preferred pancreatic cancer, breast cancer, glioblastoma multiforme, head and neck cancer, or non-small cell lung cancer.

### DETAILED DESCRIPTION OF THE INVENTION

The following terms have the indicated meanings throughout the specification: Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane.

Alkylene includes but not limited to methylene, ethylene, propylene, and so on.

The term “halogen” includes fluoro (F), chloro (C₁), bromo (Br) and iodo (I).

The term “alkenyl” includes a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkynylene includes but not limited to ethynylene and so on.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “spiro cycloalkyl” includes a cyclic structure which contains carbon atoms and is formed by at least two rings sharing one atom.

The term “fused cycloalkyl” includes a bicyclic cycloalkyl group as defined herein which is saturated and is formed by two or more rings sharing two adjacent atoms.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆ cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “H” or “hydrogen” disclosed herein includes Hydrogen and the non-radioisotope deuterium.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group. the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H, et al. “Chromatographic resolution of enantiomers: Selective review. “J. Chromatogr., 113 (3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable. For another example, compounds including pyrazolyl may under go tautomerism to form a different ring like below:

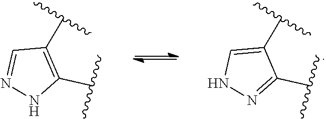 [SMILES: *c1cn[nH]c1* ; *c1c[nH]nc1*]

For another example, compounds including guanidinyl in the ring may under go tautomerism to form a different ring like below:

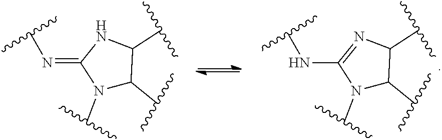 [SMILES: */N=C1\NC(*)C(*)N1* ; *NC1=NC(*)C(*)N1*]

“Prodrug” refers to a derivative of an active agent that requires a transformation within the body to release the active agent. In some embodiments, the transformation is an enzymatic transformation. Prodrugs are frequently, although not necessarily, pharmacologically inactive until converted to the active agent.

“deuterated analog” refers to a derivative of an active agent that an arbitrary hydrogen is substituted with deuterium. In some embodiments, the deuterated site is on the Warhead moiety. In some embodiments, the deuterated site is on the Linker moiety. In some embodiments, the deuterated site is on the Degron moiety.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “treated”, “treating” or “treatment” as used herein also generally refers to the acquisition of the desired pharmacological and/or physiological effect. The effect may be prophylactic according to the prevention of the disease or its symptoms in whole or in part; and/or may be therapeutic according to the partial or complete stabilization or cure of the disease and/or the side effect due to the disease. As used herein, “treated”, “treating” or “treatment” encompasses any treatment for the disease of a patient, including: (a) prevention of the disease or condition in the patient that may be predisposed to the disease or condition but has not yet been diagnosed; (b) inhibition of the symptoms of the disease, i.e., preventing its development; or (c) remission of the symptoms of the disease, i.e., causing regression of the disease or symptoms in whole or in part.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” or “Cₙ-Cₘ” indicates a range which includes the endpoints, wherein n and m are integers and indicate the number of carbons. Examples include C₁₋₈, C₁₋₆, C₁-C₈, C₁-C₆ and the like.

Unless otherwise specified, the percentages, proportions, ratios or parts used in the present application are by weight or volume. The amount used in the present application is a weight or volume amount. It can be determined easily by those skilled in the art.

Hereinafter, the present application will demonstrate the beneficial effects of the present application by way of examples. Those skilled in the art will recognize that these examples are illustrative and not restrictive. These examples will do not limit the scope of the present application in any way. The experimental methods described in the following examples, unless otherwise specified, are conventional methods; the reagents and materials, unless otherwise specified, are commercially available.

Unless specifically defined elsewhere in this document, all other technical and scientific terms used herein have the meaning commonly understood by one of ordinary skill in the art to which this invention belongs.

### Examples

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on an Agilent instrument operating at 400 MHz. ¹HNMR
spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 nm), Mass detector: 6120 SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS, LCMS-3: LC-MS spectrometer (Agilent 1260 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1290 Infinity II) Detector: MWD (190-400 nm), Mass detector: G6125C SQ Mobile phase: A: water with 0.1% Formic acid, B: acetonitrile with 0.100 Formic acid Column: Poroshell 120 EC-C18, 4.6×50 mm, 2.7 pm Gradient method: Flow: 1.2 mL/min Time (min) A (0) B (0)

| Time (min) | A(%) | B(%) |
|---|---|---|
| 0.00 | 90 | 10 |
| 1.5 | 5 | 95 |
| 2.0 | 5 | 95 |
| 2.1 | 90 | 10 |
| 3.0 | 90 | 10 |

Preparative HPLC was conducted on a column 150×21.2 mm ID, 5 pm, Gemini NXC 18) at a flow rate of 20 ml/min, injection volume 2 ml, at room temperature and UV Detection at 214 nm and 254 nm.

In the following examples, the abbreviations below are used:

| | |
|---|---|
| (BPin)₂ | 4,4,4′,4′,5,5,5′,5′-Octamethyl-2,2′-bi-1,3,2-dioxaborolane |
| Ac₂O | acetic anhydride |
| AcCl | Acetyl chloride |
| ACN or MeCN | Acetonitrile |
| AcOH or HOAc | Acetic acid |
| AcONa or NaOAc | Sodium acetate |
| BINAP | (±)-2,2′-Bis(diphenylphosphino)-1,1′-BINAPhthyl |
| Bn | benzyl |
| BnBr | Benzyl Bromide |
| Boc | t-Butyloxy carbonyl |
| Cbz | Benzyloxycarbonyl |
| DCM | Dichloromethane |
| DBU | 1,8-diazabicyclo[5.4.0]undec-7-ene |
| DCE | dichloroethane |
| DIBAL-H | Diisobutylaluminium hydride |
| DIEA or DIPEA | N,N-diisopropylethylamine |
| DMAP | 4-N,N-dimethylaminopyridine |
| DMP | Dess-Martin periodinane |
| DMF | N,N-Dimethylformamide |
| DMSO | Dimethyl sulfoxide |
| Dppf | 1,1″-bis(diphenylphosphino)ferrocene |
| EA or EtOAc | Ethyl acetate |
| EtOH | ethanol |
| FA | Formic acid |
| h, hr or hrs | Hour |
| HATU | 2-(7-Azabenzotriazol-1-yl)-N,N,N′,N′-tetramethyluronium |
| | hexafluorophosphate |
| Hex | Hexane |
| HPLC | High Performance Liquid Chromatography |
| IBX | 2-Iodoxybenzoic acid |
| IPA | 2-propanol |
| i-PrOH | Isopropyl alcohol |
| KHMDS | Potassium bis(trimethylsilyl)amide |
| LiHMDS | Lithium bis(trimethylsilyl)amide |
| KOAc or AcOK | Potassium Acetate |
| MeCN or ACN | Acetonitrile |
| MeOH | Methanol |
| Min | Minutes |
| ms or MS | Mass spectrum |
| MsCl | Methanesulfonyl chloride |
| MsOH | Methanesulfonic acid |
| MTBE | Methyl tert-butyl ether |
| o/n | overnight |
| Pd(dppf)Cl₂ | [1,1′- |
| | Bis(diphenylphosphino)ferrocene]dichloropalladium(II) |
| Pd₂(dba)₃ | Tris(dibenzylideneacetone)dipalladium |
| PE | Petroleum ether |
| PhMe | Toluene |
| PMB | 4-Methoxybenzyl |
| PPA | Polyphosphoric acid |
| R.T. or r.t. | Room temperature |
| Rt | Retention time |
| Selectfluor | 1-Chloromethyl-4-fluoro-1,4-diazoniabicyclo[2.2.2]octane |
| | bis(tetrafluoroborate) |
| SEMCl | 2-(Trimethylsilyl)ethoxymethyl chloride |
| STAB | Sodium Triacetoxyborohydride; Sodium triacetoborohydride |
| Sat. | saturated |
| SFC | Supercritical Fluid Chromatography |
| TBAF | Tetra-butyl ammonium fluoride |
| TBDPS | tert-Butyldiphenylsilyl |
| TBS | tert-Butyldimethylsilyl |
| TBSCl | tert-Butyldimethylsilyl chloride |
| Ti(OiPr)₄ | Titanium tetraisopropanolate |
| t-Bu | tert-butyl |
| t-BuOH | tert-Butanol |
| t-BuONa | Sodium tert-butoxide |
| t-BuOK | Potassium tert-butoxide |
| TEA | Triethylamine |
| Tf₂O | Triflic anhydride |
| TFA | Trifluoroacetic acid |
| THF | Tetrahydrofuran |
| TLC | Thin layer chromatography |
| TMSOK | Potassium trimethylsilanolate |
| Ts | para-Toluenesulfonyl |
| TsCl | 4-Toluenesulfonyl chloride |
| TsOH | p-toluenesulfonic acid |
| TsOH, Py | Pyridinium toluene-4-sulphonate |
| Xphos or X-phos | 2-Dicyclohexylphosphino-2′,4′,6′-triisopropylbiphenyl |
| XantPhos | 4,5-Bis(diphenylphosphino)-9,9-dimethylxanthene |
| Johnphos | (2-Biphenyl)di-tert-butylphosphine |
| TCFH | N,N,N′,N′-Tetramethylchloroformamidinium |
| | hexafluorophosphate |
| CDI | 1,1′-Carbonyldiimidazole |
| aq. | Aqueous |
| DCE | Dichloroethane |
| DEA | N,N-Diethylaniline |
| DIAD | Diisopropyl azodicarboxylate |
| DMA | N,N-Dimethylacetamide |
| DMC | Dimethyl carbonate |
| LCMS | Liquid Chromatography Mass Spectrometry |
| LDA | Lithium diisopropylamide |
| NCS | N-Chlorosuccinimide |
| NMR | Nuclear Magnetic Resonance |
| dtbpf | 1,1′-Bis(di-tert-butylphosphino)ferrocene |
| Pd(dtbpf)Cl₂ | [1,1′-Bis(di-tert- |
| | butylphosphino)ferrocene]dichloropalladium(II) |
| Pd-PEPPSI-IPentCl | [1,3-bis[2,6-bis(1-propylbutyl)phenyl]-4,5-dichloro- |
| | imidazol-2-ylidene]-dichloro-(3-chloropyridin-1-ium-1- |
| | yl)palladium |
| PPh₃ | triphenylphosphine |
| PPh₃O | Triphenylphosphine oxide |
| Prep-HPLC | Preparative-scale High-Performance Liquid |
| | Chromatography |
| RuPhos | 2-Dicyclohexylphosphino-2′,6′-diisopropoxy-1,1′-biphenyl |
| RuPhos Pd G3 | Methanesulfonato(2-dicyclohexylphosphino-2′,6′-di- |
| | isopropoxy-1,1′-biphenyl)(2′-amino-1,1′-biphenyl-2- |
| | yl)palladium(II) |
| T₃P | Propylphosphonic anhydride |

### Intermediate Synthesis

### Intermediate 1: 3-(3,3-dimethyl-2-oxo-4-(4-oxopiperidin-1-yl)indolin-1-yl)piperidine-2,6-dione

### Step 1: N-(2,6-bis(benzyloxy)pyridin-3-yl)-2-(2,6-dibromophenyl)acetamide

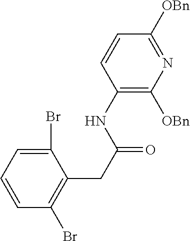 [SMILES: O=C(Cc1c(Br)cccc1Br)Nc1ccc(OCc2ccccc2)nc1OCc1ccccc1]

To a solution of 2-(2,6-dibromophenyl)acetic acid (10 g, 34.2 mmol), 2,6-bis(benzyloxy)pyridin-3-amine (11.5 g, 37.6 mml), DIEA (13.3 g, 102.6 mmol) in DMF (200 ml), were added HATU (19.5 g, 51.3 mmol) at 0° C. The mixture was stirred at RT overnight. The mixture was diluted with EA (500 mL) and then get solid filter. The mixture was concentrated under reduced pressure to afford crude product (15 g, 75.7%). [M+H]⁺=581.

### Step 2: 1-(2,6-bis(benzyloxy)pyridin-3-yl)-4-bromoindolin-2-one

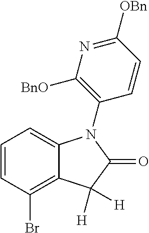 [SMILES: O=C1Cc2c(Br)cccc2N1c1ccc(OCc2ccccc2)nc1OCc1ccccc1]

A 500 mL round bottom flask equipped with a magnetic stirrer, were charged with N-(2,6-bis(benzyloxy)pyridin-3-yl)-2-(2,6-dibromophenyl)acetamide (15 g, 25.9 mmol), K₂CO₃ (17.8 g, 129.5 mmol), CuCl (2.56 g, 25.9 mmol), pentane-2,4-dione (5.17 g, 51.8 mmol) and NMP (200 mL). The mixture was degassed under vacuum and purged with N₂ for three times. The resulting mixture was stirred for 3 hours at 85° C. under N₂. After cooled to rt, the mixture was diluted with ethyl acetate (300 mL), then filtered through a pad of celite. The filtrate was washed with brine (300 mL), dried over anhydrous Na₂SO₄, concentrated under reduced pressure. The residue was purified by silica column chromatography (EA/PE, 15%) to afford product (8.00 g, 61.8%). [M+H]⁺=501.

### Step 3: 1-(2,6-bis(benzyloxy)pyridin-3-yl)-4-(1,4-dioxa-8-azaspiro[4.5]decan-8-yl)indolin-2-one

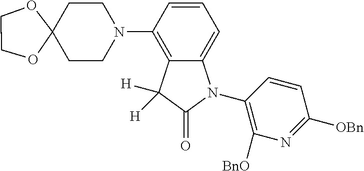 [SMILES: O=C1Cc2c(N3CCC4(CC3)OCCO4)cccc2N1c1ccc(OCc2ccccc2)nc1OCc1ccccc1]

The solution of 1-(2,6-bis(benzyloxy)pyridin-3-yl)-4-bromoindolin-2-one (8 g, 15.9 mmol), 1,4-dioxa-8-azaspiro[4.5]decane (3.43 g, 23.9 mmol), Cs₂CO₃ (15.62 g, 47.7 mmol), Pd-PEPPSI-IPentCl (0.67 g, 0.80 mmol) in dioxane (120 mL) was degassed under reduced pressure and purged with N₂ for five times, and stirred under N₂ at 100° C. overnight. After cooled to rt, the mixture was diluted with ethyl acetate (100 mL), then filtered through a pad of celite. The filtrate was washed with brine (150 mL), dried over anhydrous Na₂SO₄, concentrated under reduced pressure. The residue was purified by silica column chromatography (EA/PE, 40%) to afford product (2 g, 22.2%). [M+H]⁺=564.

### Step 4: 1-(2,6-bis(benzyloxy)pyridin-3-yl)-3,3-dimethyl-4-(1,4-dioxa-8-azaspiro[4.5]decan-8-yl)indolin-2-one

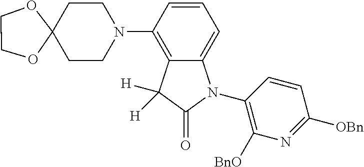 [SMILES: O=C1Cc2c(N3CCC4(CC3)OCCO4)cccc2N1c1ccc(OCc2ccccc2)nc1OCc1ccccc1]

The solution of 1-(2,6-bis(benzyloxy)pyridin-3-yl)-4-(1,4-dioxa-8-azaspiro[4.5]decan-8-yl)indolin-2-one (2 g, 3.55 mmol) and NaH (0.71 g, 17.75 mmol) in DMF (20 mL) was stirred at 0° C. for 30 min. Then CH₃I (2.5 g, 17.75 mmol) was added dropwise into the mixture while maintaining temperature at 0° C. The resulting mixture was stirred at RT for 4 hrs. The mixture was poured into EA (20 mL), and washed with brine (20 mL), water (3×20 mL), brine (20 mL) in turn, then dried over anhydrous Na₂SO₄, concentrated in vacuum. The residue was purified by silica column chromatography (EA/PE, 15%) to afford product (1.5 g, 71.4%). [M+H]⁺=592.

### Step 5: 3-(3,3-dimethyl-2-oxo-4-(1,4-dioxa-8-azaspiro[4.5]decan-8-yl)indolin-1-yl)piperidine-2,6-dione

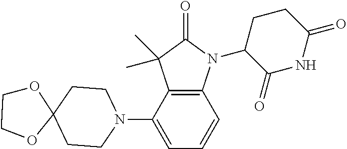 [SMILES: CC1(C)C(=O)N(C2CCC(=O)NC2=O)c2cccc(N3CCC4(CC3)OCCO4)c21]

A 100 mL round bottom flask equipped with a magnetic stirrer were charged with 1-(2,6-bis(benzyloxy)pyridin-3-yl)-3,3-dimethyl-4-(1,4-dioxa-8-azaspiro[4.5]decan-8-yl)indolin-2-one (1.5 g, 2.53 mmol), DCM/EtOH (10 mL/20 mL), and Pd/C (10 wt %, 1.5 g). The resulting mixture was degassed under reduced pressure and purged with H₂ for five times, then stirred at RT for overnight. The mixture was diluted with DCM/MeOH (50 mL/50 mL), then sonicated in an ultrasonic washer for 5 minutes, followed by filtration through a pad of celite. The filtrate was concentrated under vacuum. The residue was purified by silica column chromatography (PE/EA=1:1) to afford product (560.1 mg, 53.6%). [M+H]⁺=414. ¹H NMR (300 MHz, DMSO) δ 11.05 (s, 1H), 7.22 (t, J=7.4 Hz, 1H), 7.03 (d, J=8.2 Hz, 1H), 6.81 (d, J=7.7 Hz, 1H), 5.21 (s, 1H), 3.93 (s, 4H), 3.32 (s, 2H), 2.87 (t, J=5.5 Hz, 5H), 1.94 (s, 1H), 1.86-1.71 (m, 4H), 1.40 (s, 6H).

### Step 6: 3-(3,3-dimethyl-2-oxo-4-(4-oxopiperidin-1-yl)indolin-1-yl)piperidine-2,6-dione

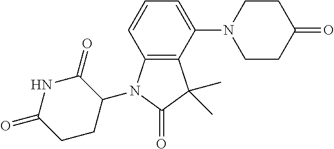 [SMILES: CC1(C)C(=O)N(C2CCC(=O)NC2=O)c2cccc(N3CCC(=O)CC3)c21]

The compound was prepared in a procedure similar to that in intermediate 3 step 4.

### Intermediate 2: 2,6-bis(benzyloxy)-3-(4-bromo-2,6-difluorophenyl)pyridine

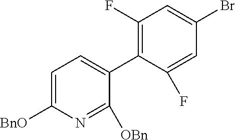 [SMILES: Fc1cc(Br)cc(F)c1-c1ccc(OCc2ccccc2)nc1OCc1ccccc1]

### Step 1: 2,6-bis(benzyloxy)-3-(4,4,5,5-tetramethyl-1,3,2-dioxaborolan-2-yl)pyridine

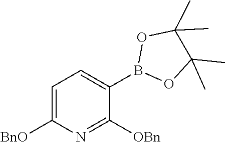 [SMILES: CC1(C)OB(c2ccc(OCc3ccccc3)nc2OCc2ccccc2)OC1(C)C]

4 reactions were carried out in parallel. To a solution of 2,6-dibenzyloxy-3-bromo-pyri dine (2.00 kg, 5.40 mol, 1.00 eq) in dioxane (12.0 L) was added 4,4,5,5-tetramethyl-1,3,2-dioxaborolane (760 g, 5.94 mol, 862 mL, 1.10 eq) and TEA (1.09 kg, 10.8 mol, 1.50 L, 2.00 eq), degassed and purged with N₂, then Pd(PPh₃)₂Cl₂ (189 g, 270 mmol, 0.05 eq) was added to the mixture. The mixture was stirred at 90° C. for 16 hrs. The reaction mixture was filtered and concentrated. 4 reactions were combined. The residue was purified by column chromatography (SiO₂, Petroleum ether/Ethyl acetate=1/0 to 0/1). 2,6-bis(benzyloxy)-3-(4,4,5,5-tetramethyl-1,3,2-dioxaborolan-2-yl)pyridine (5.80 kg, 13.9 mol, 64.3% yield) was obtained. [M+H]⁺=418.3

### Step 2: 5-bromo-1,3-difluoro-2-iodobenzene

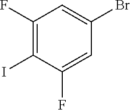

5 reactions were carried out in parallel. To a solution of 1-bromo-3,5-difluoro-benzene (1.00 kg, 5.18 mol, 595 mL) in THF (4.00 L) was added LDA (2 M, 2.59 L) at −70° C. The mixture was stirred at −70° C. for 1 hr. Then to the mixture was added solution of I₂ (1.33 kg, 5.23 mol, 1.05 L) in THF (1.00 L) at −70° C. The mixture was stirred at −70° C. for 1 hr. The reaction mixture was poured into H₂O (5.00 L), extracted with EtOAc (3.00 L×2), 5 reactions were combined, the combined organic layers were washed by brine (5.00 L), dried over Na₂SO₄, filtered and concentrated in vacuum at 40° C. to give a residue. The crude product was triturated with petroleum ether (6.00 L). Compound 5-bromo-1,3-difluoro-2-iodobenzene (4.50 kg, 14.1 mol, 54.4% yield) was obtained. [M+H]⁺=318.8

### Step 3: 2,6-bis(benzyloxy)-3-(4-bromo-2,6-difluorophenyl)pyridine

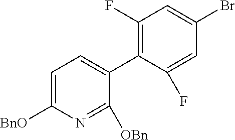 [SMILES: Fc1cc(Br)cc(F)c1-c1ccc(OCc2ccccc2)nc1OCc1ccccc1]

To a solution of 5-bromo-1,3-difluoro-2-iodobenzene (4.50 kg, 14.1 mol) and 2,6-bis(benzyloxy)-3-(4,4,5,5-tetramethyl-1,3,2-dioxaborolan-2-yl)pyridine (5.89 kg, 14.1 mol) in dioxane (22.5 L) and H₂O (4.50 L) was added Pd(PPh₃)₄ (1.63 kg, 1.41 mol), K₃PO₄ (8.99 kg, 42.3 mol). The mixture was stirred at 90° C. for 12 hrs. TLC (Petroleum ether:Ethyl acetate=10:1, R_{f} (starting material)=0.84, R_{f} (product)=0.66) showed the starting material was consumed completely. The mixture was filtered and filtrate was extracted with EtOAc (5.00 L), the combined organic was washed with brine (5.00 L), dried over Na₂SO₄, concentrated in vacuum. The residue was purified by column chromatography (SiO₂, Petroleum ether:Ethyl acetate=1:0 to 1:1). Compound 2,6-bis(benzyloxy)-3-(4-bromo-2,6-difluorophenyl)pyridine (3.00 kg, 6.01 mol, 42.5% yield, 96.5% purity) was obtained. ¹H NMR (400 MHz, CHLOROFORM-d) 7.47-7.40 (m, 1H), 7.39-7.34 (m, 2H), 7.34-7.29 (m, 2H), 7.29-7.25 (m, 4H), 7.25-7.17 (m, 2H), 7.12-7.05 (m, 2H), 6.42 (d, J=8.0 Hz, 1H), 5.32 (s, 2H), 5.28 (s, 2H); [M+H]⁺=482.1.

### Intermediate 3: (R)-3-(2,6-difluoro-4-(4-oxopiperidin-1-yl)phenyl)piperidine-2,6-dione

### Step 1: 8-(4-(2,6-bis(benzyloxy)pyridin-3-yl)-3,5-difluorophenyl)-1,4-dioxa-8-azaspiro[4.5]decane

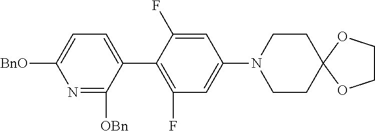 [SMILES: Fc1cc(N2CCC3(CC2)OCCO3)cc(F)c1-c1ccc(OCc2ccccc2)nc1OCc1ccccc1]

To the solution of intermediate 2 (30 g, 62.24 mmol), 1,4-dioxa-8-azaspiro[4.5]decane (10.68 g, 74.69 mmol) and Cs₂CO₃ (40.58 g, 124.48 mmol) in 500 mL dioxane, Pd₂(dba)₃ (2.85 g, 3.11 mmol) and Xantphos (3.6 g, 6.22 mmol) was added at N₂ atmosphere. The mixture was stirred at 80° C. for 16 hours under N₂ protected. The mixture was diluted with EtOAc and filtered. The filtrated was concentrated in vacuum and purified by silica column chromatography (EA:PE=0-80%) to afford the crude product. The crude was recrystallized with MeOH and filtered. The filter cake was dried to afford the title compound (26.8 g, 79% yield); [M+H]⁺=544.9.

### Step 2: 3-(2,6-difluoro-4-(1,4-dioxa-8-azaspiro[4.5]decan-8-yl)phenyl)piperidine-2,6-dione

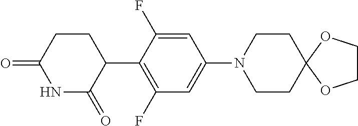 [SMILES: O=C1CCC(c2c(F)cc(N3CCC4(CC3)OCCO4)cc2F)C(=O)N1]

To the solution of 8-(4-(2,6-bis(benzyloxy)pyridin-3-yl)-3,5-difluorophenyl)-1,4-dioxa-8-azaspiro[4.5]decane (26.8 g, 49.26 mmol) in 400 mL DMF and 80 mL iPrOH, Pd/C (27 g, 10 wt. %, wet) was added. The mixture was stirred at 45° C. for 16 hours at H₂ atmosphere (4 bar). The mixture was filtered and the filter cake was washed with DMF. The combined liquid was concentrated in vacuum to afford the title compound (15 g, 83.2% yield); [M+H]⁺=367.1.

### Step 3: (R)-3-(2,6-difluoro-4-(1,4-dioxa-8-azaspiro[4.5]decan-8-yl)phenyl)piperidine-2,6-dione

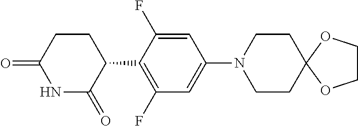 [SMILES: O=C1CC[C@H](c2c(F)cc(N3CCC4(CC3)OCCO4)cc2F)C(=O)N1]

The title compound was purified by chiral HPLC (CHIRALPAK IF (2*25 cm, 5 um), MtBE (0.1% DEA):(MeOH:DCM=1:1)=50:50, 100 bar, 20 ml/min) and the title compound corresponded to peak A @0.990 min/254 nm (4.47 g, 37% yield from 12 g racemate); [M+H]⁺=367.1.

### Step 4: (R)-3-(2,6-difluoro-4-(4-oxopiperidin-1-yl)phenyl)piperidine-2,6-dione

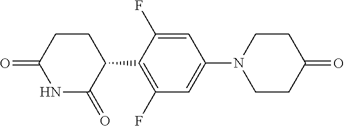 [SMILES: O=C1CCN(c2cc(F)c([C@H]3CCC(=O)NC3=O)c(F)c2)CC1]

(R)-3-(2,6-difluoro-4-(1,4-dioxa-8-azaspiro[4.5]decan-8-yl)phenyl)piperidine-2,6-dione (1 g, 2.73 mmol) was placed in 100 mL round bottom flask with a magnetic stir bar. Then, 10 mL 8N HCl aqueous was added. The mixture was stirred at room temperature for 30 minutes. The mixture was added dropwise to sat. aq. NaHCO₃ solution and finally pH=6-7. The liquid was extracted with DCM and separated. The organic phase was concentrated in vacuum and purified with silica gel column chromatography (MeOH:DCM=0-5%) to afford the title compound (850 mg, 96.7% yield); [M+H]⁺=323.1.

### Intermediate 4: 2′,6′-bis(benzyloxy)-3-fluoro-5-iodo-6-methyl-2,3′-bipyridine

### Step 1: 5-fluoro-2-methylpyridin-3-amine

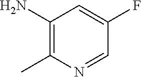

To a solution of 2-bromo-5-fluoropyridin-3-amine (20 g, 105.26 mmol) in dioxane/H₂O (200/40 mL) were added 2,4,6-trimethyl-1,3,5,2,4,6-trioxatriborinane (15.91 g, 126.32 mmol), Pd(dppf)Cl₂ (8.59 g, 10.53 mmol), and K₂CO₃ (43.57 g, 315.79 mmol). The resulting solution was stirred overnight at 120° C. under N₂ atmosphere. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with MeOH/DCM (0˜10%) to afford 5-fluoro-2-methylpyridin-3-amine (12 g, 90.1%). [M+H]⁺=127.1

### Step 2: 6-chloro-5-fluoro-2-methylpyridin-3-amine

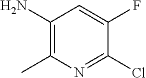

To a stirred mixture of 5-fluoro-2-methylpyridin-3-amine (6 g, 47.62 mmol) in DMF (120 mL) were added NCS (8.43 g, 47.62 mmol) at 0° C. The mixture was stirred overnight at 60° C. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with EA/PE (0˜50%) to afford 6-chloro-5-fluoro-2-methylpyridin-3-amine (3 g, 39.5%). [M+H]⁺=160.0

### Step 3: 2′,6′-bis(benzyloxy)-3-fluoro-6-methyl-[2,3′-bipyridin]-5-amine

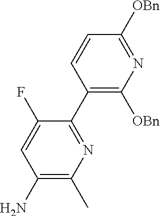 [SMILES: Cc1nc(-c2ccc(OCc3ccccc3)nc2OCc2ccccc2)c(F)cc1N]

To a solution of 6-chloro-5-fluoro-2-methylpyridin-3-amine (3 g, 18.63 mmol) in dioxane/H₂O (30/5 mL) were added 2,6-bis(benzyloxy)-3-(4,4,5,5-tetramethyl-1,3,2-dioxaborolan-2-yl)pyridine (9.32 g, 22.36 mmol), Pd(dppf)Cl₂ (1.52 g, 1.86 mmol), and K₂CO₃ (7.71 g, 55.89 mmol). The resulting solution was stirred overnight at 100° C. under N₂ atmosphere. After cooling to rt, diluted with H₂O and extracted with EtOAc. The combined organic layer was washed with brine, dried by Na₂SO₄ and concentrated. The filtrate was concentrated under reduced pressure. The crude product was purified by silica column chromatography (EA/PE=0-50%) to afford 2′,6′-bis(benzyloxy)-3-fluoro-6-methyl-[2,3′-bipyridin]-5-amine (5 g, 64.3%). [M+H]⁺=415.2.

### Step 4: 2′,6′-bis(benzyloxy)-3-fluoro-5-iodo-6-methyl-2,3′-bipyridine

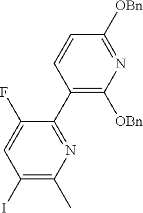 [SMILES: Cc1nc(-c2ccc(OCc3ccccc3)nc2OCc2ccccc2)c(F)cc1I]

To a solution of 2′,6′-bis(benzyloxy)-3-fluoro-6-methyl-[2,3′-bipyridin]-5-amine (3 g, 7.23 mmol) in ACN (30 mL) were added KI (6 g, 36.14 mmol), CuI (1.65 g, 8.67 mmol), and t-BuONO (3.72 g, 36.14 mmol). The resulting solution was stirred overnight at 80° C. under N₂ atmosphere. After cooling to rt, the mixture was diluted with H₂O and extracted with EtOAc. The combined organic layer was washed with brine, dried by Na₂SO₄ and concentrated. The filtrate was concentrated under reduced pressure. The crude product purified by silica column chromatography (EA/PE=0-15%) to afford 2′,6′-bis(benzyloxy)-3-fluoro-5-iodo-6-methyl-2,3′-bipyridine (2.5 g, 65.79%). [M+H]⁺=527.1.

### Intermediate 5: 3-(3-fluoro-6-methyl-5-(1,4-dioxa-8-azaspiro[4.5]decan-8-yl)pyridin-2-yl)piperidine-2,6-dione

### Step 1: 8-(2′,6′-bis(benzyloxy)-3-fluoro-6-methyl-[2,3′-bipyridin]-5-yl)-1,4-dioxa-8-azaspiro[4.5]decane

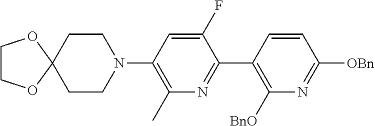 [SMILES: Cc1nc(-c2ccc(OCc3ccccc3)nc2OCc2ccccc2)c(F)cc1N1CCC2(CC1)OCCO2]

A mixture of intermediate 4 (5.2 g, 9.89 mmol), 1,4-dioxa-8-azaspiro[4.5]decane (2.11 g, 14.83 mmol), Ruphos (0.94 g, 0.20 mmol), Pd₂(dba)₃ (0.9 g, 0.99 mol), and Cs₂CO₃ (9.7 g, 29.66 mmol) in 1,4-dioxane (50 mL) was stirred for 8 h at 100° C. under N₂ atmosphere. After cooling to rt, diluted with H₂O and extracted with EtOAc, the combined organic layer was washed with brine, dried by Na₂SO₄ and concentrated. The filtrate was concentrated under reduced pressure. The crude product was purified by silica column chromatography (EA/PE=0-30%) to afford 8-(2′,6′-bis(benzyloxy)-3-fluoro-6-methyl-[2,3′-bipyridin]-5-yl)-1,4-dioxa-8-azaspiro[4.5]decane (4.5 g, 85.0%). [M+H]⁺=542.6.

### Step 2: 3-(3-fluoro-6-methyl-5-(1,4-dioxa-8-azaspiro[4.5]decan-8-yl)pyridin-2-yl)piperidine-2,6-dione

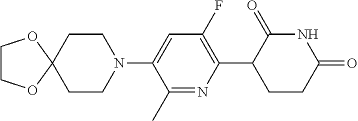 [SMILES: Cc1nc(C2CCC(=O)NC2=O)c(F)cc1N1CCC2(CC1)OCCO2]

To a stirred solution of 8-(2′,6′-bis(benzyloxy)-3-fluoro-6-methyl-[2,3′-bipyridin]-5-yl)-1,4-dioxa-8-azaspiro[4.5]decane (4.5 g, 8.31 mmol) in THF (100 mL) was added Pd/C (10 wt %, 4.5 g). The resulting mixture was degassed under reduced pressure and purged with H₂ for five times, then stirred overnight at rt. The resulting mixture was filtered, and the filter cake was washed with THF. The filtrate was concentrated under reduced pressure. And the residue was purified by column chromatography (EA/PE, 30-50%) to afford 3-(3-fluoro-6-methyl-5-(1,4-dioxa-8-azaspiro[4.5]decan-8-yl)pyridin-2-yl)piperidine-2,6-dione. (3.53 g, 9.8 mmol, 83.0%).

¹H NMR (300 MHz, DMSO) δ 10.86 (s, 1H), 7.35 (d, J=12 Hz, 1H), 4.13 (m, 1H), 3.32 (s, 1H), 2.94 (t, J=6 Hz, 4H), 2.71 (m, 1H), 2.62-2.50 (m, 1H), 2.37 (d, J=3 Hz, 3H), 2.26-2.15 (m, 1H), 2.03 (m, 1H), 1.78 (t, J=6 Hz, 4H), 1.36 (s, 1H), 1.22 (d, J=12 Hz, 1H), 0.99-0.80 (m, 1H).

### Step 3: 3-(3-fluoro-6-methyl-5-(4-oxopiperidin-1-yl)pyridin-2-yl)piperidine-2,6-dione

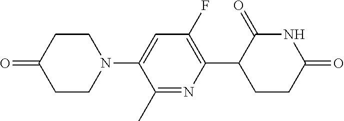 [SMILES: Cc1nc(C2CCC(=O)NC2=O)c(F)cc1N1CCC(=O)CC1]

The compound was prepared in a procedure similar to that in intermediate 3 step 4.

### Intermediate 6: 3-(4,6-dimethyl-5-(4-oxopiperidin-1-yl)pyridin-2-yl)piperidine-2,6-dione

### Step 1: 2′,6′-bis(benzyloxy)-5-bromo-4,6-dimethyl-2,3′-bipyridine

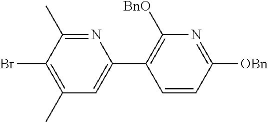 [SMILES: Cc1cc(-c2ccc(OCc3ccccc3)nc2OCc2ccccc2)nc(C)c1Br]

To a solution of 3-bromo-6-chloro-2,4-dimethylpyridine (9 g, 40.91 mmol) in dioxane/H₂O (100/20 mL) were added 2,6-bis(benzyloxy)-3-(4,4,5,5-tetramethyl-1,3,2-dioxaborolan-2-yl)pyridine (17.1 g, 40.91 mmol), Pd(PPh₃)₄ (4.64 g, 4.10 mmol), and K₂CO₃ (16.94 g, 122.73 mmol). The resulting solution was stirred for 5 h at 100° C. under N₂ atmosphere. After cooling to rt, diluted with H₂O and extracted with EtOAc, the combined organic layer was washed with brine, dried by Na₂SO₄ and concentrated. The filtrate was concentrated under reduced pressure. The crude product purified by silica column chromatography (EA/PE=0-50%) to afford 2′,6′-bis(benzyloxy)-5-bromo-4,6-dimethyl-2,3′-bipyridine (12 g, 62.2%). [M+H]⁺=475.1

### Step 2: 8-(2′,6′-bis(benzyloxy)-4,6-dimethyl-[2,3′-bipyridin]-5-yl)-1,4-dioxa-8-azaspiro[4.5]decane

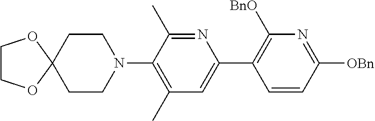 [SMILES: Cc1cc(-c2ccc(OCc3ccccc3)nc2OCc2ccccc2)nc(C)c1N1CCC2(CC1)OCCO2]

To a stirred mixture of 2′,6′-bis(benzyloxy)-5-bromo-4,6-dimethyl-2,3′-bipyridine (2.3 g, 4.84 mmol), 1,4-dioxa-8-azaspiro[4.5]decane (1.0 g, 7.26 mmol), and Cs₂CO₃ (4.7 g, 14.52 mmol) in DMF (20 mL) was added Pd-PEPPSI-IPentCl (406.7 mg, 0.48 mmol). The resulting mixture was stirred for 5 h at 100° C. under N₂ atmosphere. After cooling to rt, diluted with H₂O and extracted with EtOAc, the combined organic layer was washed with brine, dried by Na₂SO₄ and concentrated. The filtrate was concentrated under reduced pressure. The crude product purified by silica column chromatography (EA/PE=0-30%) to afford 8-(2′,6′-bis(benzyloxy)-4,6-dimethyl-[2,3′-bipyridin]-5-yl)-1,4-dioxa-8-azaspiro[4.5]decane (1.5 g, 57.7%). [M+H]⁺=538.3.

### Step 3:3-(4,6-dimethyl-5-(1,4-dioxa-8-azaspiro[4.5]decan-8-yl)pyridin-2-yl)piperidine-2,6-dione

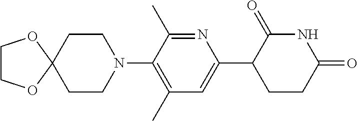 [SMILES: Cc1cc(C2CCC(=O)NC2=O)nc(C)c1N1CCC2(CC1)OCCO2]

To a stirred solution of 8-(2′,6′-bis(benzyloxy)-4,6-dimethyl-[2,3′-bipyridin]-5-yl)-1,4-dioxa-8-azaspiro[4.5]decane (3.4 g, 8.31 mmol) in THF (50 mL) was added Pd/C (10 wt %, 3.5 g). The resulting mixture was degassed under reduced pressure and purged with H₂ for five times, then stirred overnight at rt. The resulting mixture was filtered, the filter cake was washed with THF. The filtrate was concentrated under reduced pressure. And the residue was purified by column chromatography (EA/PE=30-50%) to afford 3-(4,6-dimethyl-5-(1,4-dioxa-8-azaspiro[4.5]decan-8-yl)pyridin-2-yl)piperidine-2,6-dione. (2.09 g, 83.0%). [M+H]⁺=359.4

¹H NMR (300 MHz, DMSO) δ 10.77 (s, 1H), 6.96 (s, 1H), 3.92 (s, 4H), 3.83 (m, 1H), 3.03 (t, J=6 Hz, 4H), 2.62-2.51 (m, 2H), 2.39 (s, 3H), 2.26 (s, 3H), 2.18 (m, 1H), 2.12-2.00 (m, 1H), 1.72 (t, J=6 Hz, 4H).

### Step 4: 3-(4,6-dimethyl-5-(4-oxopiperidin-1-yl)pyridin-2-yl)piperidine-2,6-dione

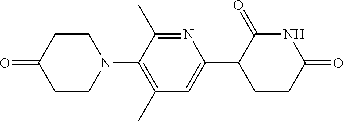 [SMILES: Cc1cc(C2CCC(=O)NC2=O)nc(C)c1N1CCC(=O)CC1]

The compound was prepared in a procedure similar to that in intermediate 3 step 4.

### Intermediate 7: 3-(4-ethyl-5-(4-oxopiperidin-1-yl)pyridin-2-yl)piperidine-2,6-dione

### Step 1: 5-bromo-2-chloro-4-vinylpyridine

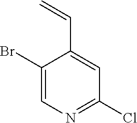

To a stirred mixture of 5-bromo-2-chloro-4-iodopyridine (16 g, 50.3 mmol), 4,4,5,5-tetramethyl-2-vinyl-1,3,2-dioxaborolane (7.75 g, 50.3 mmol), and Na₂CO₃ (16.00 g, 150.9 mmol) in 1,4-Dioxacyclohexane (200 mL) and H₂O (40 mL) was added Pd(dpppf)Cl₂ (3.65 g, 5.03 mmol). The resulting mixture was degassed under reduced pressure and purged with N₂ for three times. Then it was stirred at 100° C. for 5 hours. The reaction mixture was diluted with water and extracted with EA. The combined organic layers were washed with brine, dried, and concentrated. The residue was purified by column chromatography (EA/PE=20-30%) to afford 5-bromo-2-chloro-4-vinylpyridine (6.8 g, 62.0%). [M+H]⁺=218.

### Step 2: 8-(6-chloro-4-vinylpyridin-3-yl)-1,4-dioxa-8-azaspiro[4.5]decane

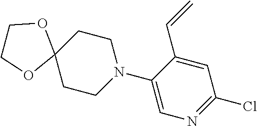 [SMILES: C=Cc1cc(Cl)ncc1N1CCC2(CC1)OCCO2]

To a stirred mixture of 5-bromo-2-chloro-4-vinylpyridine (6.8 g, 31.2 mmol), 1,4-dioxa-8-azaspiro[4.5]decane (4.46 g, 31.2 mmol), and Cs₂CO₃ (20.3 g, 62.4 mmol) in 1,4-dioxacyclohexane (140 mL) was added Pd-PEPPSI-IPentCl (1.3 g, 1.56 mmol). The resulting mixture was degassed under reduced pressure and purged with N₂ for three times. Then it was stirred overnight at 100° C. The reaction mixture was filtered through a celite pad, and the filtrate was concentrated. The residue was purified by column chromatography (EA/PE=26-28%) to afford 8-(6-chloro-4-vinylpyridin-3-yl)-1,4-dioxa-8-azaspiro[4.5]decane (4.2 g, 47.9%). [M+H]⁺=281.

### Step 3: 8-(2′,6′-bis(benzyloxy)-4-vinyl-[2,3′-bipyridin]-5-yl)-1,4-dioxa-8-azaspiro[4.5]decane

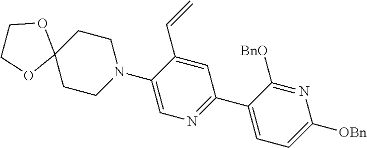 [SMILES: C=Cc1cc(-c2ccc(OCc3ccccc3)nc2OCc2ccccc2)ncc1N1CCC2(CC1)OCCO2]

To a stirred mixture of 8-(6-chloro-4-vinylpyridin-3-yl)-1,4-dioxa-8-azaspiro[4.5]decane (4.2 g, 14.95 mmol), 2,6-bis(benzyloxy)-3-(4,4,5,5-tetramethyl-1,3,2-dioxaborolan-2-yl)pyridine (6.23 g, 14.95 mmol), and K₂CO₃ (6.19 g, 44.85 mmol) in 1,4-Dioxacyclohexane (50 mL) and H₂O (10 mL) was added Pd(dpppf)Cl₂ (1.09 g, 1.5 mmol). The resulting mixture was degassed under reduced pressure and purged with N₂ for three times. Then it was stirred at 100° C. for 5 hours. The reaction mixture was diluted with water and extracted with EA. The combined organic layers were washed with brine, dried, and concentrated. The residue was purified by column chromatography (EA/PE=20-30%) to afford 8-(2′,6′-bis(benzyloxy)-4-vinyl-[2,3′-bipyridin]-5-yl)-1,4-dioxa-8-azaspiro[4.5]decane (6.3 g, 78.9%). [M+H]⁺=536

### Step 4:3-(4-ethyl-5-(1,4-dioxa-8-azaspiro[4.5]decan-8-yl)pyridin-2-yl)piperidine-2,6-dione

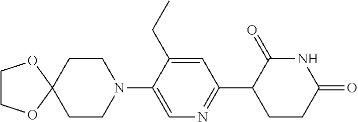 [SMILES: CCc1cc(C2CCC(=O)NC2=O)ncc1N1CCC2(CC1)OCCO2]

To a stirred solution of 8-(2′,6′-bis(benzyloxy)-4-vinyl-[2,3′-bipyridin]-5-yl)-1,4-dioxa-8-azaspiro[4.5]decane (6.3 g, 11.8 mmol) in THF (150 mL) was added Pd/C (10 wt %, 6.3 g). The resulting mixture was degassed under reduced pressure and purged with H₂ for five times, then stirred overnight at 50° C. The reaction mixture was diluted with THF/DCM(1:1), then filtered through a Celite pad. The filtrate was concentrated under vacuum. And the residue was purified by column chromatography (PE/EA, 30-50%) to afford 3-(4-ethyl-5-(1,4-dioxa-8-azaspiro[4.5]decan-8-yl)pyridin-2-yl)piperidine-2,6-dione (3.53 g, 9.8 mmol, 83.05%). [M+H]⁺=360. ¹H NMR (300 MHz, DMSO) δ 10.80 (s, 1H), 8.19 (s, 1H), 7.20 (s, 1H), 3.99-3.90 (m, 1H), 3.32 (s, 1H), 2.96 (t, J=3 Hz, 4H), 2.72-2.50 (m, 3H), 2.26-2.04 (m, 1H), 1.76 (t, J=6 Hz, 4H), 1.20 (t, J=9 Hz, 3H), 1.07 (s, 2H).

### Step 5: 3-(4-ethyl-5-(4-oxopiperidin-1-yl)pyridin-2-yl)piperidine-2,6-dione

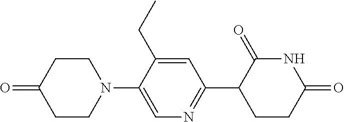 [SMILES: CCc1cc(C2CCC(=O)NC2=O)ncc1N1CCC(=O)CC1]

The compound was prepared in a procedure similar to that in intermediate 3 step 4.

### Intermediate 8: 2-(6-(2,6-dioxopiperidin-3-yl)-2,4-dimethylpyridin-3-yl)ethyl methanesulfonate

### Step 1: 2′,6′-bis(benzyloxy)-4,6-dimethyl-5-vinyl-2,3′-bipyridine

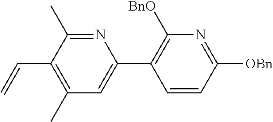 [SMILES: C=Cc1c(C)cc(-c2ccc(OCc3ccccc3)nc2OCc2ccccc2)nc1C]

To a stirred mixture of 2′,6′-bis(benzyloxy)-5-bromo-4,6-dimethyl-2,3′-bipyridine (3 g, 6.31 mmol), 4,4,5,5-tetramethyl-2-vinyl-1,3,2-dioxaborolane (1.17 g, 7.58 mmol), and K₂CO₃ (2.61 g, 18.93 mmol) in 1,4-dioxane (30 mL) and H₂O (6 mL) was added Pd(dpppf)Cl₂ (514.1 mg, 0.63 mmol). The resulting solution was stirred for 5 h at 100° C. under N₂ atmosphere. After cooling to rt, diluted with H₂O and extracted with EtOAc, the combined organic layer was washed with brine, dried by Na₂SO₄ and concentrated. The filtrate was concentrated under reduced pressure. The crude product purified by silica column chromatography (EA/PE=0-20%) to afford 2′,6′-bis(benzyloxy)-4,6-dimethyl-5-vinyl-2,3′-bipyridine (1.7 g, 63.9%). [M+H]⁺=423.2.

### Step 2: 2-(2′,6′-bis(benzyloxy)-4,6-dimethyl-[2,3′-bipyridin]-5-yl)ethan-1-ol

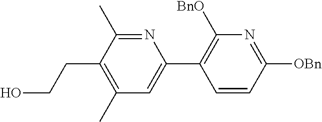 [SMILES: Cc1cc(-c2ccc(OCc3ccccc3)nc2OCc2ccccc2)nc(C)c1CCO]

To a stirred mixture of 2′,6′-bis(benzyloxy)-4,6-dimethyl-5-vinyl-2,3′-bipyridine (1.7 g, 4.01 mmol) in THF (20 mL) was added 9-BBN (0.5 M in THF, 40 mL, 20.0 mmol) dropwise at 0° C. The reaction mixture was stirred overnight at rt, then NaOH (2M in water, 4 mL, 8.03 mmol) and H₂O₂ (30%, 40.2 mL, 12.1 mmol) were added at 0° C. The reaction mixture was stirred at rt for 2 h, The mixture was diluted with H₂O and extracted with EA, and the residue was purified by column chromatography (EA/PE=0-35%) to afford 2-(2′,6′-bis(benzyloxy)-4,6-dimethyl-[2,3′-bipyridin]-5-yl)ethan-1-ol (1.4 g, 79.5%). [M+H]⁺=441.2.

### Step 3: 3-(5-(2-hydroxyethyl)-4,6-dimethylpyridin-2-yl)piperidine-2,6-dione

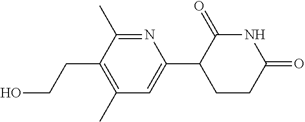 [SMILES: Cc1cc(C2CCC(=O)NC2=O)nc(C)c1CCO]

To a stirred solution 2-(2′,6′-bis(benzyloxy)-4,6-dimethyl-[2,3′-bipyridin]-5-yl)ethan-1-ol (1.4 g, 8.31 mmol) in THF (50 mL) was added Pd/C (10 wt %, 1.5 g). The resulting mixture was degassed under reduced pressure and purged with H₂ for five times, then stirred overnight at 50° C. The resulting mixture was filtered, the filter cake was washed with THF. The filtrate was concentrated under reduced pressure. And the residue was purified by column chromatography (EA/PE=30-50%) to afford 3-(5-(2-hydroxyethyl)-4,6-dimethylpyridin-2-yl)piperidine-2,6-dione (855.7 mg, 83.0%). [M+H]⁺=263.1.

¹H NMR (300 MHz, DMSO) δ 10.80 (s, 1H), 6.96 (s, 1H), 4.83-4.74 (m, 1H), 3.83 (m, 1H), 3.52 (m, 2H), 2.78 (t, J=9 Hz, 2H), 2.64-2.54 (m, 3H), 2.44 (s, 3H), 2.29 (s, 3H), 2.26-2.02 (m, 1H).

### Step 4: 2-(6-(2,6-dioxopiperidin-3-yl)-2,4-dimethylpyridin-3-yl)ethyl methanesulfonate

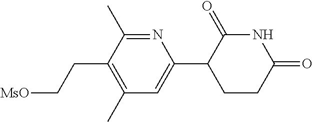 [SMILES: Cc1cc(C2CCC(=O)NC2=O)nc(C)c1CCOS(C)(=O)=O]

To a solution of 3-(5-(2-hydroxyethyl)-4,6-dimethylpyridin-2-yl)piperidine-2,6-dione (300 mg, 1.15 mmol) and Et₃N (347 mg, 3.44 mmol) in DCM (6 mL) and THF (6 mL), MsCl (172 mg, 1.49 mmol) was slowly added at 0° C. The mixture was stirred at 25° C. for 2 hours. The mixture was quenched with water (10 mL). The organic phase was separated and concentrated in vacuum. The residue was purified by prep-TLC (DCM:MeOH=20:1) to afford product (220 mg, 56.5% yield). [M+H]⁺=341.5.

### Intermediate 9: 3-(3-fluoro-5-((3R,4S)-3-fluoro-4-(piperazin-1-yl)piperidin-1-yl)-6-methylpyridin-2-yl)piperidine-2,6-dione

### Step 1: tert-butyl(3R,4S)-4-(4-benzylpiperazin-1-yl)-3-fluoropiperidine-1-carboxylate

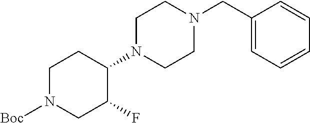 [SMILES: CC(C)(C)OC(=O)N1CC[C@H](N2CCN(Cc3ccccc3)CC2)[C@H](F)C1]

The solution of tert-butyl (3R,4S)-4-amino-3-fluoropiperidine-1-carboxylate (2.5 g, 11.46 mmol) N-benzyl-2-chloro-N-(2-chloroethyl)ethan-1-amine hydrochloride (3 g, 11.46 mmol) and NaHCO₃ (3.85 g, 45.84 mmol) in 50 mL EtOH was stirred at 80° C. for 16 hours. After LCMS shown the reaction was completed, the mixture was concentrated, diluted with water and extracted by DCM. The organic layer was separated and concentrated. The mixture was purified by silica column chromatography (MeOH:DCM=0-4%) to afford tert-butyl (3R,4S)-4-(4-benzylpiperazin-1-yl)-3-fluoropiperidine-1-carboxylate (2 g, 5.3 mmol, 46.3%). [M+H]⁺=378.6.

### Step 2: 1-benzyl-4-((3R,4S)-3-fluoropiperidin-4-yl)piperazine

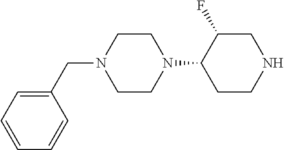 [SMILES: F[C@@H]1CNCC[C@@H]1N1CCN(Cc2ccccc2)CC1]

To a solution of tert-butyl (3R,4S)-4-(4-benzylpiperazin-1-yl)-3-fluoropiperidine-1-carboxylate (5 g, 13.2 mmol) in DCM (20 mL) was added TFA (10 mL). The reaction was stirred at room temperature for 2 hr and then concentrated in vacuo. The residue was dissolved in DCM (200 mL), washed with sat. NaHCO₃ solution (3×100 mL) and brine (50 mL), dried over Na₂SO₄, filtered and concentrated under reduced pressure to afford the product (3 g, 81.7%). [M+H]⁺=278.4.

### Step 3: 2′,6′-bis(benzyloxy)-5-((3R,4S)-4-(4-benzylpiperazin-1-yl)-3-fluoropiperidin-1-yl)-3-fluoro-6-methyl-2,3′-bipyridine

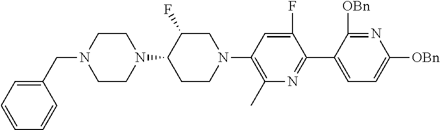 [SMILES: Cc1nc(-c2ccc(OCc3ccccc3)nc2OCc2ccccc2)c(F)cc1N1CC[C@H](N2CCN(Cc3ccccc3)CC2)[C@H](F)C1]

To a stirred solution of intermediate 4 (0.95 g, 1.8 mmol) in dioxane (20 mL) were added Cs₂CO₃ (1.2 g, 3.6 mmol), 1-benzyl-4-((3R,4S)-3-fluoropiperidin-4-yl)piperazine (0.5 g, 1.8 mmol) and Pd-Ruphos-G3 (0.3 g, 0.4 mmol) at room temperature under nitrogen atmosphere. The resulting mixture was stirred for 12 h at 100° C. under nitrogen atmosphere. The mixture was allowed to cool down to room temperature. The resulting mixture was concentrated under reduced pressure. The residue was diluted with EtOAc (100 mL), washed with water (3×50 mL) and brine (50 mL). The organic layer was dried over anhydrous Na₂SO₄, filtered and concentrated under reduced pressure.

The residue was purified by silica gel column chromatography, eluted with MeOH/DCM (0-5%) to afford the product (0.95 g, 78%). [M+H]⁺=676.7.

### Step 4: tert-butyl 4-((3R,4S)-1-(6-(2,6-dioxopiperidin-3-yl)-5-fluoro-2-methylpyridin-3-yl)-3-fluoropiperidin-4-yl)piperazine-1-carboxylate

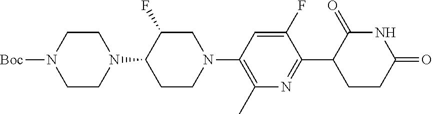 [SMILES: Cc1nc(C2CCC(=O)NC2=O)c(F)cc1N1CC[C@H](N2CCN(C(=O)OC(C)(C)C)CC2)[C@H](F)C1]

To a stirred mixture of 2′,6′-bis(benzyloxy)-5-((3R,4S)-4-(4-benzylpiperazin-1-yl)-3-fluoropiperidin-1-yl)-3-fluoro-6-methyl-2,3′-bipyridine (0.95 g, 1.4 mmol), di-tert-butyl dicarbonate (1.5 g, 7 mmol) in i-PrOH (40 mL) and DMF (40 mL) was added Pd on carbon (1 g, wet, 10 wt %). The resulting mixture was stirred under hydrogen atmosphere (1 atm) at room temperature for 24 hr. Then the mixture was filtered and the filtrate was concentrated. The residue was purified by silica gel column chromatography, eluted with MeOH/DCM (0-7%) to afford the product (0.46 g, 64.5%). [M+H]⁺=508.4.

### Step 5: 3-(3-fluoro-5-((3R,4S)-3-fluoro-4-(piperazin-1-yl)piperidin-1-yl)-6-methylpyridin-2-yl)piperidine-2,6-dione

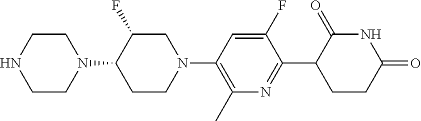 [SMILES: Cc1nc(C2CCC(=O)NC2=O)c(F)cc1N1CC[C@H](N2CCNCC2)[C@H](F)C1]

To a solution of tert-butyl 4-((3R,4S)-1-(6-(2,6-dioxopiperidin-3-yl)-5-fluoro-2-methylpyridin-3-yl)-3-fluoropiperidin-4-yl)piperazine-1-carboxylate (460 mg, 0.9 mmol) in DCM (10 mL) was added TFA (5 mL). The reaction was stirred at room temperature for 2 hr and then concentrated in vacuo. The residue was dissolved in DCM (50 mL), washed with sat. NaHCO₃ solution (3×20 mL) and brine (20 mL), dried over Na₂SO₄, filtered and concentrated under reduced pressure to afford the product (260 mg, 70.4%). [M+H]⁺=408.5.

### Intermediate 10: 3-(3-fluoro-5-((3S,4R)-3-fluoro-4-(piperazin-1-yl)piperidin-1-yl)-6-methylpyridin-2-yl)piperidine-2,6-dione

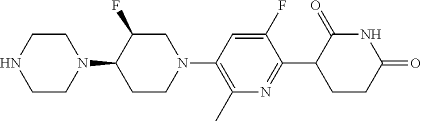 [SMILES: Cc1nc(C2CCC(=O)NC2=O)c(F)cc1N1CC[C@@H](N2CCNCC2)[C@@H](F)C1]

The title compound was prepared in a procedure similar to that in intermediate 9.

### Intermediate 11: 3-(5-((3S,4R)-3-fluoro-4-(piperazin-1-yl)piperidin-1-yl)-4,6-dimethylpyridin-yl)piperidine-2,6-dione

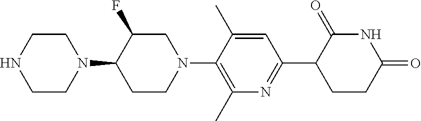 [SMILES: Cc1cc(C2CCC(=O)NC2=O)nc(C)c1N1CC[C@@H](N2CCNCC2)[C@@H](F)C1]

The title compound was prepared in a procedure similar to that in intermediate 9.

### Intermediate 12: 3-(4-methyl-5-(4-oxopiperidin-1-yl)pyridin-2-yl)piperidine-2,6-dione

### Step 1: 2′,6′-bis(benzyloxy)-5-bromo-4-methyl-2,3′-bipyridine

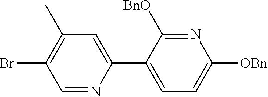 [SMILES: Cc1cc(-c2ccc(OCc3ccccc3)nc2OCc2ccccc2)ncc1Br]

To the solution of 5-bromo-2-iodo-4-methylpyridine (4.8 g, 16.1 mmol), 2,6-bis(benzyloxy)-3-(4,4,5,5-tetramethyl-1,3,2-dioxaborolan-2-yl)pyridine (6.7 g, 16.1 mmol) and K₂CO₃ (4.5 g, 32.2 mmol) in 100 mL 1,4-dioxane and 20 mL H₂O was added Pd(dppf)Cl₂ (1.2 g, 1.61 mmol). The mixture was stirred at 90° C. for 16 hours. The mixture was concentrated under reduced pressure to give the crude residue, which was purified by silica column chromatography (PE:EA=100:1-5:1) to afford the product (6.7 g, 90.5%). [M+H]⁺=461.5.

### Step 2: 8-(2′,6′-bis(benzyloxy)-4-methyl-[2,3′-bipyridin]-5-yl)-1,4-dioxa-8-azaspiro[4.5]decane

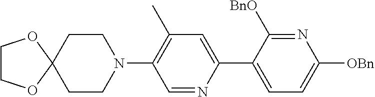 [SMILES: Cc1cc(-c2ccc(OCc3ccccc3)nc2OCc2ccccc2)ncc1N1CCC2(CC1)OCCO2]

To the solution of 2′,6′-bis(benzyloxy)-5-bromo-4-methyl-2,3′-bipyridine (6.7 g, 14.5 mmol), 1,4-dioxa-8-azaspiro[4.5]decane (5.2 g, 36.3 mmol) and Cs₂CO₃ (9.4 g, 29.0 mmol) in 80 mL DMA, were added Pd₂(dba)₃ (2.6 g, 2.9 mmol) and RuPhos (2.7 g, 5.8 mmol). The mixture was stirred at 100° C. for 16 hours under nitrogen atmosphere. The mixture was filtered through a celite pad and washed with DCM. The filtrate was concentrated under reduced pressure to give the crude residue, which was purified by silica column chromatography (PE:EA=50:1-2:1) to afford the product (5.3 g, 69.7%). [M+H]⁺=524.5.

### Step 3:3-(4-methyl-5-(1,4-dioxa-8-azaspiro[4.5]decan-8-yl)pyridin-2-yl)piperidine-2,6-dione

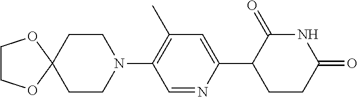 [SMILES: Cc1cc(C2CCC(=O)NC2=O)ncc1N1CCC2(CC1)OCCO2]

To the solution of 8-(2′,6′-bis(benzyloxy)-4-methyl-[2,3′-bipyridin]-5-yl)-1,4-dioxa-8-azaspiro[4.5]decane (5.3 g, 10.1 mmol) in 75 mL DMF and 75 mL iPrOH, Pd/C (2.0 g, 10 wt. %, wet) was added. The mixture was stirred at 50° C. for 20 hours under hydrogen atmosphere (balloon). The mixture was cooled to room temperature and filtered by celite directly. The filtrate was concentrated in vacuum to afford the desired product (2.7 g, 77.1%). [M+H]⁺=346.6.

### Step 4: 3-(4-methyl-5-(4-oxopiperidin-1-yl)pyridin-2-yl)piperidine-2,6-dione

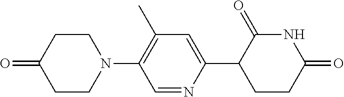 [SMILES: Cc1cc(C2CCC(=O)NC2=O)ncc1N1CCC(=O)CC1]

3-(4-methyl-5-(1,4-dioxa-8-azaspiro[4.5]decan-8-yl)pyridin-2-yl)piperidine-2,6-dione (2.7 g, 7.8 mmol) was placed in 250 mL round bottom flask with a magnetic stir bar. Then 45 mL 8N HCl aqueous was added. The mixture was stirred at room temperature for 2 hours. The mixture was added dropwise to sat. aq. NaHCO₃ and the pH was adjusted to 6-7. The liquid was extracted with DCM (40 mL×3) and separated. The combined organic phase was concentrated in vacuum and purified with combiflash (DCM:MeOH=25:1) to afford the title compound (2.2 g, 93.6% yield). [M+H]⁺=302.5.

### Intermediate 13: 3-(6-methyl-5-(4-oxopiperidin-1-yl)pyridin-2-yl)piperidine-2,6-dione

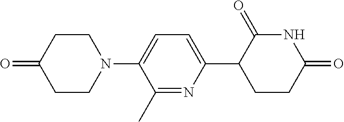 [SMILES: Cc1nc(C2CCC(=O)NC2=O)ccc1N1CCC(=O)CC1]

The title compound was prepared in a procedure similar to that in intermediate 12.

### Intermediate 14: (7¹R,7²S,E)-5⁶-bromo-1¹,2⁶,7²-trimethyl-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

### Step 1: tert-butyl((3-methylbut-3-en-1-yl)oxy)diphenylsilane

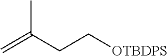

To a stirred solution of 3-methylbut-3-en-1-ol (20 g, 232.6 mmol) and imidazole (34.8 g, 511.2 mmol) in DMF (500 mL) being cooled to 0° C. was added TBDPSCl (76.7 g, 278.9 mmol) slowly. The resulting mixture was stirred at rt for overnight. The reaction mixture was diluted with H₂O (300 mL) and extracted with EA. The combined organic layers were washed with brine, dried and concentrated. The residue was purified by column chromatography (PE) to afford the crude product (65 g, 200 mmol, 86%).

### Step 2: ethyl 2-(2-((tert-butyldiphenylsilyl)oxy)ethyl)-2-methylcyclopropane-1-carboxylate

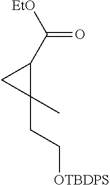

To a stirred solution of tert-butyl((3-methylbut-3-en-1-yl)oxy)diphenylsilane (60 g, 184.6 mmol) in DCM (600 mL) was added Rh₂(OAc)₄ (4.08 g, 9.2 mmol). The resulting mixture was degassed under reduced pressure and purged with N₂ for three times. To the above mixture being cooled to −10° C. was added ethyl 2-diazoacetate (84.2 g, 738.5 mmol) dropwise. The resulting mixture was stirred overnight at rt. The reaction mixture was filtered through a celite pad, and the filtrate was concentrated. The residue was purified by column chromatography (EA/PE=0-5%) to afford the crude product (60 g, 121.7 mmol, 65.9%). [M+H]⁺=411.

### Step 3: (2-(2-((tert-butyldiphenylsilyl)oxy)ethyl)-2-methylcyclopropyl)methanol

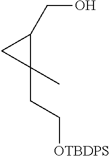

To a stirred solution of ethyl 2-(2-((tert-butyldiphenylsilyl)oxy)ethyl)-2-methylcyclopropane-1-carboxylate (36 g, 87.6 mmol) in THF (700 mL) being cooled to 0° C. was added LiAlH₄ (6.66 g, 175.2 mmol) portionwise. The resulting mixture was stirred at rt for 4 hours. The reaction mixture was quenched with sodium sulfate decahydrate at 0° C. for 30 minutes. Then it was diluted with DCM/MEOH, filtered, and concentrated. The residue was purified by column chromatography (EA/PE=18-20%) to afford the product (3.1 g, 8.4 mmol, 9.6%). [M+H]⁺=369.

### Step 4: 2-((2-(2-((tert-butyldiphenylsilyl)oxy)ethyl)-2-methylcyclopropyl)methyl)isoindoline-1,3-dione

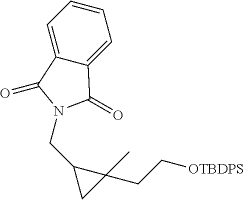

To a stirred solution of (2-(2-((tert-butyldiphenylsilyl)oxy)ethyl)-2-methylcyclopropyl)methanol (3.1 g, 8.4 mmol) and isoindoline-1,3-dione (1.23 g, 8.4 mmol) in THF (60 ml) was added PPh₃ (4.4 g, 16.8 mmol). The resulting mixture was degassed under reduced pressure and purged with N₂ for three times. To the above mixture being cooled to 0° C. was added DIAD (2.5 g, 12.6 mmol) dropwise. Then the resulting mixture was stirred at rt for 5 hours. The reaction mixture was diluted with H₂O and extracted with EA. The combined organic layers were washed with brine, dried and concentrated. The residue was purified by column chromatography (EA/PE, 7-9%) to afford product (2.8 g, 5.6 mmol, 66.7%). [M+H]⁺=498.

### Step 5: (2-(2-((tert-butyldiphenylsilyl)oxy)ethyl)-2-methylcyclopropyl)methanamine

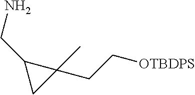

To a stirred solution of 2-((2-(2-((tert-butyldiphenylsilyl)oxy)ethyl)-2-methylcyclopropyl)methyl)isoindoline-1,3-dione (2.8 g, 5.6 mmol) in EtOH (60 mL) was added N₂H₄H₂O (1.12 g, 22.4 mmol). The resulting mixture was stirred overnight at rt. The reaction mixture was filtered and the filtrate was concentrated to afford the crude product (2.0 g, 5.4 mmol). The product was used directly in the next step without any purifications. [M+H]⁺=368.

### Step 6: 2-(2-(((5-bromo-2-nitrophenyl)amino)methyl)-1-methylcyclopropyl)ethan-1-ol

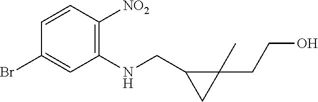 [SMILES: CC1(CCO)CC1CNc1cc(Br)ccc1[N+](=O)[O-]]

To a stirred solution of (2-(2-((tert-butyldiphenylsilyl)oxy)ethyl)-2-methylcyclopropyl)methanamine (2.0 g, 5.4 mmol) and 4-bromo-2-fluoro-1-nitrobenzene (1.3 g, 5.94 mmol) in DMSO (20 mL) was added DIEA (2.1 g, 16.3 mmol). The resulting mixture was degassed under reduced pressure and purged with N₂ for three times. Then it was stirred at 80° C. for 4 hours. The reaction mixture was diluted with H₂O and extracted with EA. The combined organic layers were washed with brine, dried, filtered and concentrated. The residue was purified by column chromatography (EA/PE=25-30%) to afford product (1.1 g, 3.3 mmol, 61.1%). [M+H]⁺=329.

### Step 7: 2-((1S,2R)-2-(((5-bromo-2-nitrophenyl)amino)methyl)-1-methylcyclopropyl)ethan-1-ol

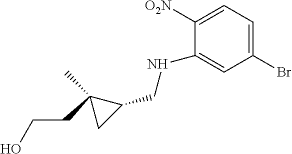 [SMILES: C[C@@]1(CCO)C[C@H]1CNc1cc(Br)ccc1[N+](=O)[O-]]

The crude product of step 6 (1.1 g) was purified by Prep-Chiral-HPLC with the following conditions (Column: CHIRALPAK IG-3 (4.6*50 mm, 3 um), Mobile Phase: Hex (0.1% DEA):EtOH=75:25, Flow rate: 1.0 mL/min, Temperature: 25° C., retention time: 2.759 min) to afford 2-((1S,2R)-2-(((5-bromo-2-nitrophenyl)amino)methyl)-1-methylcyclopropyl)ethan-1-ol (442.6 mg, 1.3 mmol, 40.2%, ee=100%). [M+H]⁺=329.

¹H NMR (400 MHz, CDCl₃) δ 8.16 (s, 1H), 8.03 (d, J=8 Hz, 1H), 6.98 (d, J=4.0 Hz, 1H), 6.74 (dd, J=9.1, 2.0 Hz, 1H), 3.94-3.84 (m, 1H), 3.88-3.79 (m, 1H), 3.53 (m, 1H), 2.97 (m, 1H), 1.83-1.72 (m, 1H), 1.37 (m, 1H), 1.16 (s, 3H), 1.20-1.08 (m, 1H), 0.68 (dd, J=8.7, 4.8 Hz, 1H), 0.27 (t, J=4 Hz, 1H).

### Step 8: methyl 2-(5-(2-((1S,2R)-2-(((5-bromo-2-nitrophenyl)amino)methyl)-1-methylcyclopropyl)ethoxy)-1-methyl-1H-pyrazol-4-yl)-6-methylisonicotinate

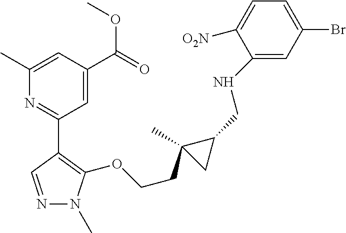 [SMILES: COC(=O)c1cc(C)nc(-c2cnn(C)c2OCC[C@]2(C)C[C@H]2CNc2cc(Br)ccc2[N+](=O)[O-])c1]

To a stirred solution of 2-((1S,2R)-2-(((5-bromo-2-nitrophenyl)amino)methyl)-1-methylcyclopropyl)ethan-1-ol (3.5 g, 10.6 mmol), methyl 2-(5-hydroxy-1-methyl-1H-pyrazol-4-yl)-6-methylisonicotinate (2.9 g, 11.7 mmol), and PPh₃ (3.4 g, 12.8 mmol) in THF (70 mL) was added DIAD (2.6 g, 12.8 mmol). Then the mixture was stirred for 2 h at room temperature. Then the mixture was concentrated and purified by silica gel column chromatography, eluted with EtOAc/PE (0-80%) to afford the product mixed with PPh₃O (6.2 g); [M+H]⁺=558.3.

### Step 9: methyl 2-(5-(2-((1S,2R)-2-(((2-amino-5-bromophenyl)amino)methyl)-1-methylcyclopropyl)ethoxy)-1-methyl-1H-pyrazol-4-yl)-6-methylisonicotinate

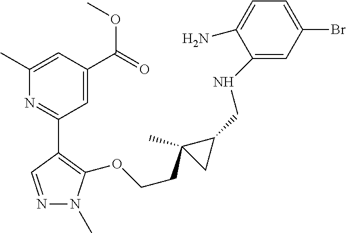 [SMILES: COC(=O)c1cc(C)nc(-c2cnn(C)c2OCC[C@]2(C)C[C@H]2CNc2cc(Br)ccc2N)c1]

To a stirred solution of methyl 2-(5-(2-((1S,2R)-2-(((5-bromo-2-nitrophenyl)amino)methyl)-1-methylcyclopropyl)ethoxy)-1-methyl-1H-pyrazol-4-yl)-6-methylisonicotinate (6.2 g, 11.1 mmol) in THF (70 mL) was added Raney nickel (3.0 g). The resulting mixture was stirred under hydrogen atmosphere (1 atm) at room temperature for 2 hr. Then the mixture was filtered and the filtrate was concentrated. The resulting mixture (5.5 g) was used for next step without purification; [M+H]⁺=528.3.

### Step 10: methyl 2-(5-(2-((1S,2R)-2-((6-bromo-2-imino-2,3-dihydro-1H-benzo[d]imidazol-1-yl)methyl)-1-methylcyclopropyl)ethoxy)-1-methyl-1H-pyrazol-4-yl)-6-methylisonicotinate

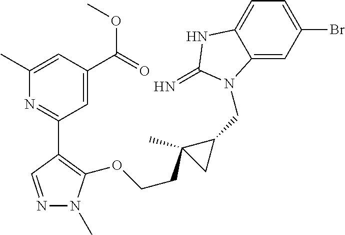 [SMILES: COC(=O)c1cc(C)nc(-c2cnn(C)c2OCC[C@]2(C)C[C@H]2Cn2c(=N)[nH]c3ccc(Br)cc32)c1]

A solution of methyl 2-(5-(2-((1S,2R)-2-(((2-amino-5-bromophenyl)amino)methyl)-1-methylcyclopropyl)ethoxy)-1-methyl-1H-pyrazol-4-yl)-6-methylisonicotinate (5.5 g, 10.4 mmol) and BrCN (1.7 g, 15.6 mmol) in MeOH (50 mL) was stirred for 4 h at room temperature. Then the mixture was concentrated and diluted with DCM (200 mL), and then washed with sat. aq. NaHCO₃ (3×100 mL) and brine (100 mL). The organic layer was dried over anhydrous Na₂SO₄, filtered and concentrated under reduced pressure. The residue was purified by silica gel column chromatography, eluted with MeOH/DCM (0-15%) to afford the product (4.5 g, 78.1%); [M+H]⁺=553.4.

### Step 11: (7¹R,7²S,E)-5⁶-bromo-1¹,2⁶,7²-trimethyl-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

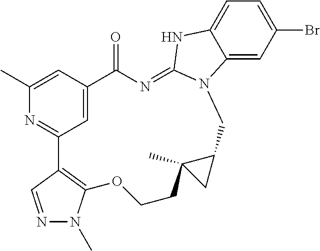 [SMILES: Cc1cc2cc(n1)-c1cnn(C)c1OCC[C@]1(C)C[C@H]1CN1/C(=N/C2=O)Nc2ccc(Br)cc21]

To a stirred solution of methyl 2-(5-(2-((1S,2R)-2-((6-bromo-2-imino-2,3-dihydro-1H-benzo[d]imidazol-1-yl)methyl)-1-methylcyclopropyl)ethoxy)-1-methyl-1H-pyrazol-4-yl)-6-methylisonicotinate (4.5 g, 8.1 mmol) in 80 mL THF was added LiHMDS (1N in THF, 20.3 mL) at room temperature for 15 min. The resulting mixture was stirred at room temperature for 1 hr. Then the mixture was diluted with EA (100 mL), washed with sat. aq. NH₄Cl (2×100 mL) and brine (100 mL). The organic layer was dried over anhydrous Na₂SO₄, filtered and concentrated under reduced pressure. The residue was suspended in 100 mL EA and stirred at room temperature for 1 h. Then the mixture was filtered to afford the product (3.2 g, 75.5%); [M+H]⁺=521.2.

### Intermediate 15: (7¹S,7²R,E)-5⁶-bromo-1¹,2⁶-dimethyl-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

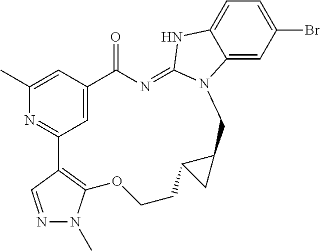 [SMILES: Cc1cc2cc(n1)-c1cnn(C)c1OCC[C@H]1C[C@@H]1CN1/C(=N/C2=O)Nc2ccc(Br)cc21]

### Step 1: (but-3-en-1-yloxy)(tert-butyl)diphenylsilane

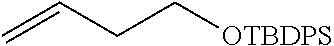

A 500 mL round bottom flask equipped with a magnetic stirrer, was charged with but-3-en-1-ol (5.0 g, 69.4 mmol), TBDPSCl (28.65 g, 104.6 mmol), imidazole (10.39 g, 152.7 mmol), DMF (80 mL). The resulting mixture was degassed under reduced pressure and purged with N₂ for three times, then stirred at rt for overnight. The mixture was diluted with EA (200 mL), washed with water (2×100 mL), brine (100 mL), dried and concentrated. The residue was purified by column chromatography (pure PE to PE/EA=10:1) to afford crude product (15 g, 48.2 mmol, 69.4%). ¹H NMR (300 MHz, DMSO-d6) δ ppm 7.68-7.58 (m, 4H), 7.53-7.37 (m, 6H), 5.82 (ddt, J=17.1, 10.2, 6.8 Hz, 1H), 5.13-4.97 (m, 2H), 3.69 (t, J=6.6 Hz, 2H), 2.28 (qt, J=6.6, 1.4 Hz, 2H), 0.99 (s, 9H).

### Step 2: ethyl 2-(2-((tert-butyldiphenylsilyl)oxy)ethyl)cyclopropane-1-carboxylate

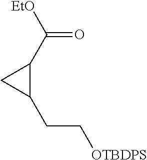

A three-neck round bottom flask equipped with a magnetic stirrer, was charged with (but-3-en-1-yloxy)(tert-butyl)diphenylsilane (10 g, 32.2 mmol), Rh₂(OAc)₄ (1.42 g, 3.22 mmol), DCM (200 ml). The mixture was degassed under reduced pressure and purged with N₂ for three times. Ethyl 2-diazoacetate (18.34 g, 160.7 mmol) in DCM (20 ml) was injected into the flask, the flask was degassed and purged with N₂ once more, then the resulting mixture was stirred at rt for overnight. The mixture was diluted with EA, washed with water and brine, dried, concentrated. The residue was purified by column chromatography (pure PE to PE/EA=20:1) to afford crude product (6 g, 15.1 mmol, 46.8%). [M+H]⁺=397.5

### Step 3: (2-(2-((tert-butyldiphenylsilyl)oxy)ethyl)cyclopropyl)methanol

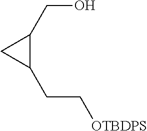

A round bottom flask equipped with a magnetic stirrer, was charged with ethyl 2-(2-((tert-butyldiphenylsilyl)oxy)ethyl)cyclopropane-1-carboxylate (4.0 g, 10.1 mmol), THF (70 mL). The temperature was lowered to 0° C. LiAlH₄ (1.15 g, 30.3 mmol) was added portionwise into the mixture while maintaining temperature at 0° C., The resulting mixture was stirred at rt for 2 hrs. The mixture was quenched with sodium sulfatedecahydrate at 0° C. for 30 minutes. Then diluted with DCM/MEOH (50 ml/50 ml), concentrated. The residue was purified by column chromatography (PE/EA=10:1 to 6:1 to afford product (730 mg, 2.05 mmol, 20.8%). [M+H]⁺=355.5.

### Step 4: 2-((2-(2-((tert-butyldiphenylsilyll)oxy)ethyll)cyclopropyl)methyll)isoindoline-1,3-dione

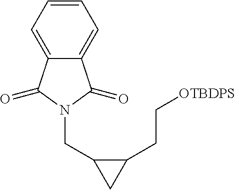

A round bottom flask equipped with a magnetic stirrer, was charged with (2-(2-((tert-butyldiphenylsilyl)oxy)ethyl)cyclopropyl)methanol (680 mg, 1.91 mmol), isoindoline-1,3-dione (564 mg, 3.82 mmol), PPh₃ (1.01 g, 3.82 mmol), and THF (10 ml). The mixture was degassed under reduced pressure and purged with N₂ for three times. DIAD (774.6 mg, 3.82 mmol) was injected into the flask, the flask was degassed and purged with N₂ once more, Then the resulting mixture was stirred at rt for 2 hrs. The mixture was diluted with EA, washed with water and brine, dried, concentrated. The residue was purified by column chromatography (PE/EA=10:1 to 7:1) to afford product (850 mg, 1.76 mmol, 85.4%). [M+H]⁺=484.5.

### Step 5: 2-(((1R,2S)-2-(2-((tert-butyldiphenylsilyl)oxy)ethyl)cyclopropyl)methyl)isoindoline-1,3-dione

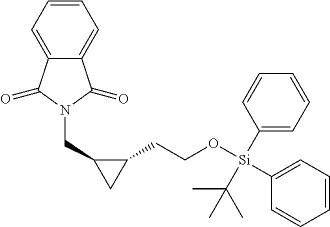 [SMILES: CC(C)(C)[Si](OCC[C@@H]1C[C@H]1CN1C(=O)c2ccccc2C1=O)(c1ccccc1)c1ccccc1]

The crude product (20 g) was purified by Prep-SFC with the following conditions (column: chiral ND (2) 3.0×100 mm, 3 μm; solvent A: CO₂, solvent B: IPA (0.1% DEA), gradient (B): 5% to 20% in 2 min, hold 1 min at 20%; retention time 1.704 min) to afford 2-(((1R,2S)-2-(2-((tert-butyldiphenylsilyl)oxy)ethyl)cyclopropyl)methyl)isoindoline-1,3-dione (8.12 g, 40.6%, ee=98.58%). ¹H NMR (300 MHz, DMSO-d6) δ ppm 7.84 (s, 4H), 7.52 (td, J=8.1, 1.6 Hz, 4H), 7.45-7.36 (m, 6H), 3.61-3.47 (m, 3H), 3.35 (dd, J=14.2, 7.5 Hz, 1H), 1.52 (dq, J=12.6, 6.4 Hz, 1H), 1.32-1.19 (m, 1H), 0.98 (d, J=7.1 Hz, 1H), 0.92 (s, 9H), 0.84 (s, 2H), 0.49 (dt, J=8.8, 4.6 Hz, 1H), 0.30 (dt, J=9.6, 5.0 Hz, 1H). [M+H]⁺=484.5.

### Step 6: 2-(((1S,2R)-2-(2-((tert-butyldiphenylsilyl)oxy)ethyl)cyclopropyl)methyl)isoindoline-1,3-dione

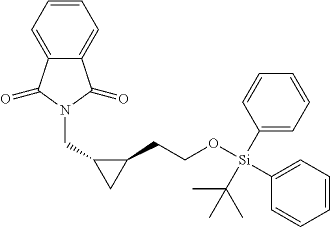 [SMILES: CC(C)(C)[Si](OCC[C@H]1C[C@@H]1CN1C(=O)c2ccccc2C1=O)(c1ccccc1)c1ccccc1]

The crude product (20 g) was purified by Prep-SFC with the following conditions (column: chiral ND (2) 3.0×100 mm, 3 μm; solvent A: CO₂, solvent B: IPA (0.1% DEA), gradient (B): 5% to 20% in 2 min, hold 1 min at 20%; retention time 2.041 min) to afford 2-(((1S,2R)-2-(2-((tert-butyldiphenylsilyl)oxy)ethyl)cyclopropyl)methyl)isoindoline-1,3-dione (9.98 g, 49.9%, ee=99.56%). ¹H NMR (300 MHz, DMSO-d6) δ ppm 7.84 (s, 4H), 7.52 (td, J=8.1, 1.6 Hz, 4H), 7.45-7.36 (m, 6H), 3.61-3.47 (m, 3H), 3.35 (dd, J=14.2, 7.5 Hz, 1H), 1.52 (dq, J=12.6, 6.4 Hz, 1H), 1.32-1.19 (m, 1H), 0.98 (d, J=7.1 Hz, 1H), 0.92 (s, 9H), 0.84 (s, 2H), 0.49 (dt, J=8.8, 4.6 Hz, 1H), 0.30 (dt, J=9.6, 5.0 Hz, 1H). [M+H]⁺=484.5.

### Step 7: ((1S,2R)-2-(2-((tert-butyldiphenylsilyl)oxy)ethyl)cyclopropyl)methanamine

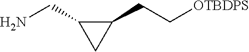

To the solution of 2-(((1S,2R)-2-(2-((tert-butyldiphenylsilyl)oxy)ethyl)cyclopropyl)methyl)isoindoline-1,3-dione (3.5 g, 7.2 mmol) in EtOH (100 mL) was added hydrazine hydrate (6 mL). The resulting mixture was stirred at 70° C. for 2 hours. After LCMS showed the reaction was completed, the mixture was filtered to remove the solids. The filtrate was concentrated under reduced pressure to give the product (2.4 g, 93.7%). [M+H]⁺=354.5.

### Step 8: 5-bromo-N-(((1S,2R)-2-(2-((tert-butyldiphenylsilyl)oxy)ethyl)cyclopropyl)methyl)-2-nitroaniline

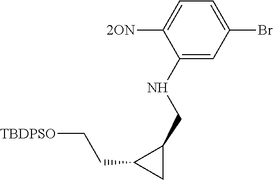

To a solution of ((1S,2R)-2-(2-((tert-butyldiphenylsilyl)oxy)ethyl)cyclopropyl)methanamine (1.0 g, 2.8 mmol) and 4-bromo-2-fluoro-1-nitrobenzene (684 mg, 3.1 mmol) in MeCN (30 mL) was added DIEA (728 mg, 5.6 mol) at 25° C. The mixture was stirred at 60° C. for 12 hours. The reaction concentrated under reduced pressure to give a residue, which was purified by column chromatography (PE:EA=10:1) to give product (1.4 g, 89.7% yield). [M+H]⁺=553.5

### Step 9: 2-((1R,2S)-2-(((5-bromo-2-nitrophenyl)amino)methyl)cyclopropyl)ethan-1-ol

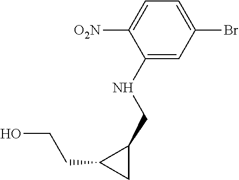 [SMILES: O=[N+]([O-])c1ccc(Br)cc1NC[C@H]1C[C@@H]1CCO]

To the solution of 5-bromo-N-(((1S,2R)-2-(2-((tert-butyldiphenylsilyl)oxy)ethyl)cyclopropyl)methyl)-2-nitroaniline (1.4 g, 2.5 mmol) in THF (10 mL) was added TBAF (1.0 mol/L in THF, 4.0 mL). The resulting mixture was stirred at RT for 3 hours. After LCMS showed the reaction was completed, the reaction was concentrated under reduced pressure to give the crude residue, which was purified by silica column chromatography (PE:EA=2:1) to afford the product (670 mg, 84.1%). [M+H]⁺=315.5.

### Step 10: methyl 2-(5-(2-((1R,2S)-2-(((5-bromo-2-nitrophenyl)amino)methyl)cyclopropyl)ethoxy)-1-methyl-1H-pyrazol-4-yl)-6-methylisonicotinate

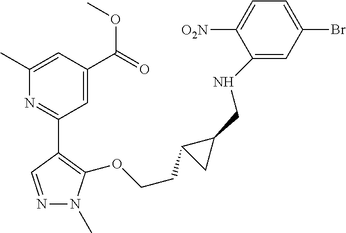 [SMILES: COC(=O)c1cc(C)nc(-c2cnn(C)c2OCC[C@H]2C[C@@H]2CNc2cc(Br)ccc2[N+](=O)[O-])c1]

To a solution of 2-((1R,2S)-2-(((5-bromo-2-nitrophenyl)amino)methyl)cyclopropyl)ethan-1-ol (665 mg, 2.1 mmol), methyl 2-(5-hydroxy-1-methyl-1H-pyrazol-4-yl)-6-methylisonicotinate (576 mg, 2.3 mmol) and PPh₃ (719 mg, 2.7 mmol) in THF (30 mL) was added DIAD (554 mg, 2.7 mmol) dropwise at 15° C. The mixture was stirred at RT for 30 minutes. The reaction mixture was concentrated under reduced pressure to give the crude residue, which was purified by column chromatography (DCM:EA=3:1) to give product (1.0 g, 86.9% yield). [M+H]⁺=544.5.

### Step 11: methyl 2-(5-(2-((1R,2S)-2-(((2-amino-5-bromophenyl)amino)methyl)cyclopropyl)ethoxy)-1-methyl-1H-pyrazol-4-yl)-6-methylisonicotinate

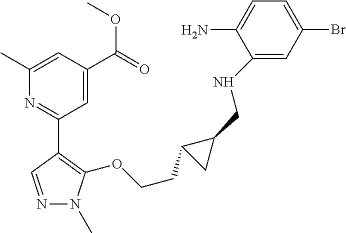 [SMILES: COC(=O)c1cc(C)nc(-c2cnn(C)c2OCC[C@H]2C[C@@H]2CNc2cc(Br)ccc2N)c1]

The title compound was prepared in a procedure similar to that in Example 9 step 4. [M+H]⁺=514.5.

### Step 12: methyl 2-(5-(2-((1R,2S)-2-((6-bromo-2-imino-2,3-dihydro-1H-benzo[d]imidazol-1-yl)methyl)cyclopropyl)ethoxy)-1-methyl-1H-pyrazol-4-yl)-6-methylisonicotinate

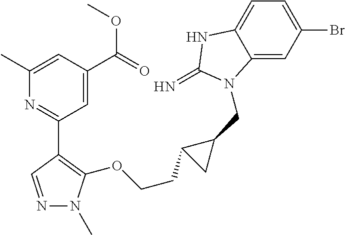 [SMILES: COC(=O)c1cc(C)nc(-c2cnn(C)c2OCC[C@H]2C[C@@H]2Cn2c(=N)[nH]c3ccc(Br)cc32)c1]

The title compound was prepared in a procedure similar to that in Example 9 step 5. [M+H]⁺=539.5.

### Step 13: (7¹S,7²R,E)-5⁶-bromo-1¹,2⁶-dimethyl-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

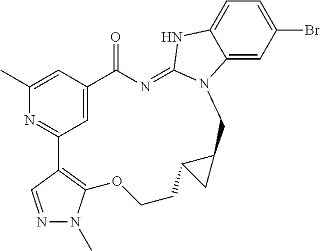 [SMILES: Cc1cc2cc(n1)-c1cnn(C)c1OCC[C@H]1C[C@@H]1CN1/C(=N/C2=O)Nc2ccc(Br)cc21]

To a solution of methyl 2-(5-(2-((1R,2S)-2-((6-bromo-2-imino-2,3-dihydro-1H-benzo[d]imidazol-1-yl)methyl)cyclopropyl)ethoxy)-1-methyl-1H-pyrazol-4-yl)-6-methylisonicotinate (900 mg, 1.7 mmol) in DMF (30 mL) was added LiHMDS (1.0 mol/L in THF, 4.0 mL) dropwise at 0° C. The mixture was stirred at RT for 10 minutes. the mixture was quenched with NH₄Cl/water and extracted by DCM. The organic layer was dried over anhydrous Na₂SO₄, filtered and concentrated under reduced pressure to give the crude residue, which was purified by silica column chromatography (DCM:MeOH=20:1) to afford the product (420 mg, 49.6%). [M+H]⁺=507.5.

### Intermediate 16: (7¹S,7²R,E)-1¹,2⁶-dimethyl-5⁶-(3-(tetrahydrofuran-3-yl)piperazin-1-yl)-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

### Step 1: 2-(tetrahydrofuran-3-yl)pyrazine

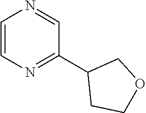 [SMILES: c1cnc(C2CCOC2)cn1]

To the solution of 2-bromopyrazine (5.0 g, 31.4 mmol) and 3-iodotetrahydrofuran (9.3 g, 47.2 mmol) in 100 mL DMA, were added NiI₂ (2.94 g, 9.42 mmol), picolinimidamide (HCl salt, 2.47 g, 15.7 mmol), Mn (5.2 g, 94.2 mmol) and TFA (1.07 g, 9.42 mmol). The mixture was stirred at 100° C. for 16 hours under N₂. After LCMS showed the reaction was completed, the mixture was filtered and concentrated under reduced pressure. The resulting mixture was purified by silica column chromatography (DCM:MeOH=100:1-95:5) to afford the product (820 mg, 18.2%). [M+H]⁺=151.4.

### Step 2: 2-(tetrahydrofuran-3-yl)piperazine(AcOH salt)

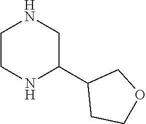 [SMILES: C1CNC(C2CCOC2)CN1]

To a solution of 2-(tetrahydrofuran-3-yl)pyrazine (310 mg, 2.06 mmol) in AcOH/MeOH (6 mL/6 mL) was added PtO₂ (150 mg) at room temperature. And then the mixture was exchanged with H₂ three times and stirred under H₂ atmosphere at 40° C. for 20 hours. Reaction was monitored by LC-MS. The mixture was filtered through a pad of Celite and washed with MeOH (20 mL). The filtrate was concentrated under vacuum to obtain the title crude product without purification. [M+H]⁺=157.5.

### Step 3: (7¹S,7²R,E)-1¹,2⁶-dimethyl-5⁶-(3-(tetrahydrofuran-3-yl)piperazin-1-yl)-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

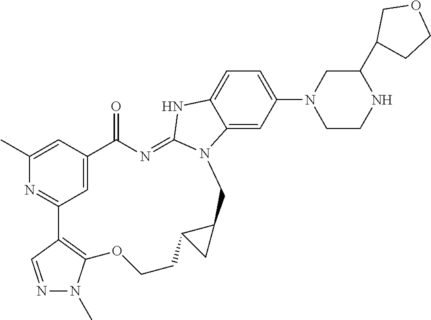 [SMILES: Cc1cc2cc(n1)-c1cnn(C)c1OCC[C@H]1C[C@@H]1CN1/C(=N/C2=O)Nc2ccc(N3CCNC(C4CCOC4)C3)cc21]

To the solution of intermediate 15 (200.0 mg, 0.394 mmol), 2-(tetrahydrofuran-3-yl)piperazine(AcOH salt) (crude, 200 mg) and t-BuONa (189 mg, 1.97 mmol) in 10 mL DMA, were added Pd₂(dba)₃ (72 mg, 0.079 mmol) and RuPhos (73 mg, 0.158 mmol). The mixture was stirred at 90° C. for 1.5 hours under N₂. After LCMS showed the reaction was completed, the mixture was diluted with aqueous NH₄Cl (20 mL) and extracted by DCM (25 mL×2). The organic layer was washed with brine, dried over anhydrous Na₂SO₄, filtered and concentrated under reduced pressure. The resulting mixture was purified by silica column chromatography (2% NH₄OH, DCM:MeOH=100:1-80:20) to afford the product (150 mg, 65.5%). [M+H]⁺=583.4.

### Intermediate 17: (7¹S,7²R,E)-5⁶-((S)-4-(azetidin-3-yl)-2-methylpiperazin-1-yl)-1¹,2⁶-dimethyl-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

### Step 1: Tert-butyl (S)-4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-3-methylpiperazine-1-carboxylate

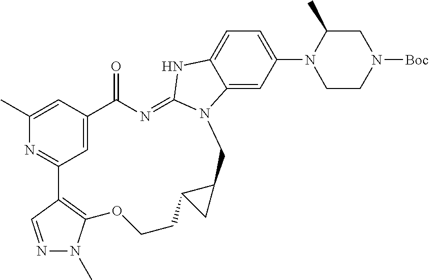 [SMILES: Cc1cc2cc(n1)-c1cnn(C)c1OCC[C@H]1C[C@@H]1CN1/C(=N/C2=O)Nc2ccc(N3CCN(C(=O)OC(C)(C)C)C[C@@H]3C)cc21]

To a solution of (7¹S,7²R,E)-5⁶-bromo-1¹,2⁶-dimethyl-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one (1.5 g, 2.96 mmol), tert-butyl (S)-3-methylpiperazine-1-carboxylate (890 mg, 4.45 mmol), Pd₂(dba)₃ (275 mg, 0.3 mmol), Ruphos (286 mg, 0.6 mmol) and NaO^{t}Bu (568 mg, 5.92 mmol) in DMA (30 mL). The resulting mixture was stirred at 100° C. for 6 hrs. The reaction was concentrated in vacuo, the residue was purified by silica gel column (DCM:CH₃OH=15:1) to afford tert-butyl (S)-4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-3-methylpiperazine-1-carboxylate (1.3 g, 70.1%). [M+H]⁺=627.

### Step 2: (7¹S,7²R,E)-1¹,2⁶-dimethyl-5⁶-((S)-2-methylpiperazin-1-yl)-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

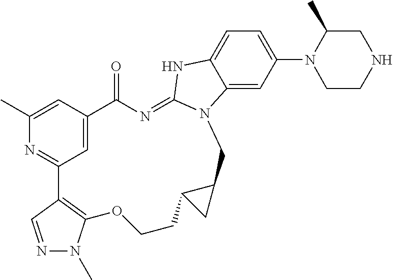 [SMILES: Cc1cc2cc(n1)-c1cnn(C)c1OCC[C@H]1C[C@@H]1CN1/C(=N/C2=O)Nc2ccc(N3CCNC[C@@H]3C)cc21]

To a mixture of tert-butyl (S)-4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-3-methylpiperazine-1-carboxylate (1.3 g, 2.07 mmol) in DCM (20 mL) was added TFA (7 mL). The reaction mixture was stirred at r.t for 2 hrs, the resulting mixture was concentrated in vacuum. The residue was treated with sat NaHCO₃ solution to PH=7-8, then extracted with DCM:MeOH(10:1) (100 mL×3). The combined organic phase was washed with brine (80 mL), dried over Na₂SO₄, filtered and concentrated in vacuum to afford (7¹S,7²R,E)-1¹,2⁶-dimethyl-5⁶-((S)-2-methylpiperazin-1-yl)-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one (800 mg, 73.4%). [M+H]⁺=527.

### Step 3: tert-butyl 3-((S)-4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-3-methylpiperazin-1-yl)azetidine-1-carboxylate

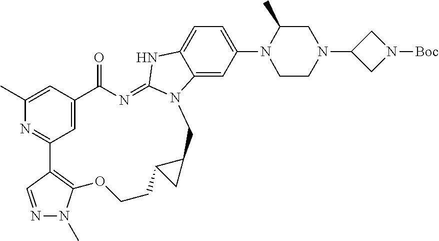 [SMILES: Cc1cc2cc(n1)-c1cnn(C)c1OCC[C@H]1C[C@@H]1CN1/C(=N/C2=O)Nc2ccc(N3CCN(C4CN(C(=O)OC(C)(C)C)C4)C[C@@H]3C)cc21]

To a mixture of (7¹S,7²R,E)-1¹,2⁶-dimethyl-5⁶-((S)-2-methylpiperazin-1-yl)-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one (600 mg, 1.14 mmol), tert-butyl 3-oxoazetidine-1-carboxylate (390 mg, 2.28 mmol) in DCE (25 mL) was added STAB (483 mg, 2.28 mmol). The reaction mixture was stirred at 50° C. for 3 hrs, the resulting mixture was quenched with sat NaHCO₃ solution (20 mL), then extracted with DCM (30 mL×3). The combined organic phase was washed with brine (20 mL), dried over Na₂SO₄, filtered and concentrated in vacuum, the residue was purified by silica gel column (DCM:CH3OH=15:1) to afford tert-butyl 3-((S)-4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-3-methylpiperazin-1-yl)azetidine-1-carboxylate (500 mg, 64.4%). [M+H]⁺=682.

### Step 4: (7¹S,7²R,E)-5⁶-((S)-4-(azetidin-3-yl)-2-methylpiperazin-1-yl)-1¹,2⁶-dimethyl-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

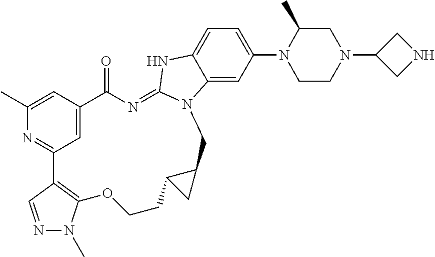 [SMILES: Cc1cc2cc(n1)-c1cnn(C)c1OCC[C@H]1C[C@@H]1CN1/C(=N/C2=O)Nc2ccc(N3CCN(C4CNC4)C[C@@H]3C)cc21]

To a mixture of tert-butyl 3-((S)-4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-3-methylpiperazin-1-yl)azetidine-1-carboxylate (500 mg, 0.73 mmol) in DCM (30 mL) was added TFA (6 mL). The reaction mixture was stirred at r.t for 1 hrs, the resulting mixture was concentrated in vacuum. The residue was treated with sat NaHCO₃ solution to PH=7-8, then extracted with DCM:MeOH (10:1) (40 mL×3). The combined organic phase was washed with brine (40 mL), dried over Na₂SO₄, filtered and concentrated in vacuum to afford (7¹S,7²R,E)-5⁶-((S)-4-(azetidin-3-yl)-2-methylpiperazin-1-yl)-1¹,2⁶-dimethyl-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one (400 mg, 93.9%). [M+H]⁺=582.

### Intermediate 18: (7¹S,7²R,E)-5⁶-(5-(azetidin-3-yl)-2,5-diazabicyclo[2.2.2]octan-2-yl)-1¹,2⁶-dimethyl-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

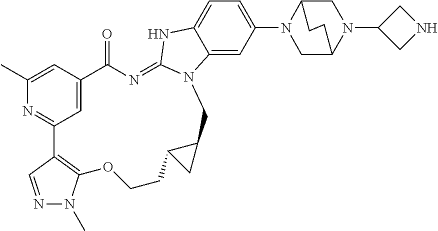 [SMILES: Cc1cc2cc(n1)-c1cnn(C)c1OCC[C@H]1C[C@@H]1CN1/C(=N/C2=O)Nc2ccc(N3CC4CCC3CN4C3CNC3)cc21]

Intermediate 18 was prepared in a manner similar to that in intermediate 17. [M+H]⁺=594.5 Intermediate 19: (7¹S,7²R,E)-1¹,2⁶-dimethyl-5⁶-((S)-2-methylpiperazin-1-yl)-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

### Step 1: tert-butyl (S)-4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-3-methylpiperazine-1-carboxylate

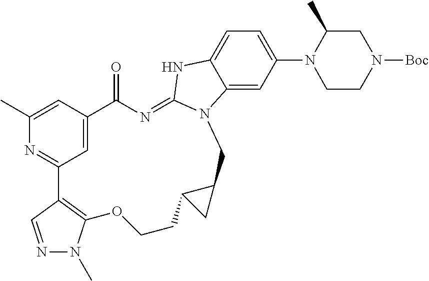 [SMILES: Cc1cc2cc(n1)-c1cnn(C)c1OCC[C@H]1C[C@@H]1CN1/C(=N/C2=O)Nc2ccc(N3CCN(C(=O)OC(C)(C)C)C[C@@H]3C)cc21]

To the solution of intermediate 15 (280 mg, 0.55 mmol), tert-butyl (S)-3-methylpiperazine-1-carboxylate (320 mg, 1.38 mmol) and t-BuONa (180 mg, 1.65 mmol) in 10 mL DMA was added Pd-PEPPSI-IPentCl (105 mg, 0.11 mmol). The resulting mixture was stirred at 100° C. for 2 hours under N₂. After LCMS showed the reaction was completed, the mixture was concentrated under reduced pressure to give the crude residue, which was purified by silica column chromatography (DCM:MeOH=100:1-20:1) to afford the product (330 mg, 95.3%). [M+H]⁺=627.5.

### Step 2: (7¹S,7²R,E)-1¹,2⁶-dimethyl-5⁶-((S)-2-methylpiperazin-1-yl)-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

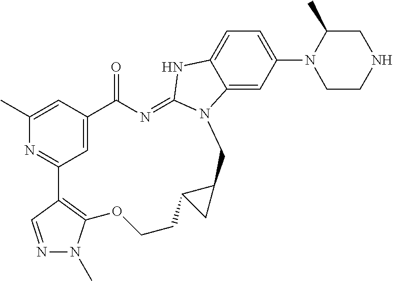 [SMILES: Cc1cc2cc(n1)-c1cnn(C)c1OCC[C@H]1C[C@@H]1CN1/C(=N/C2=O)Nc2ccc(N3CCNC[C@@H]3C)cc21]

To the solution of tert-butyl (S)-4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-3-methylpiperazine-1-carboxylate (330 mg, 0.53 mmol) in 4 mL DCM was added TFA (1 mL). After stirring at room temperature for 1 hour, the reaction mixture was concentrated, basified with NaHCO₃ aqueous, extracted with DCM (3×20 mL). The combined organic phase was washed with brine (1×15 mL), dried over Na₂SO₄, filtered and concentrated in vacuum to afford the desired product (260 mg, 93.8%), which was used without further purification. [M+H]⁺=527.5.

### Intermediate 20: ((3R)-1-(6-(2,6-dioxopiperidin-3-yl)-2-methylpyridin-3-yl)pyrrolidin-3-yl)methyl methanesulfonate

### Step 1: 3-bromo-6-iodo-2-methylpyridine

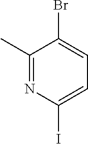

To a mixture of 3,6-dibromo-2-methylpyridine (50 g, 199.2 mmol) and NaI (59.76 g, 398.4 mmol) in ACN (500 ml) was added TMSI (7.97 g, 39.85 mmol). The resulting mixture was degassed under reduced pressure and purged with N₂ for three times. Then it was stirred overnight at 95° C. The reaction mixture was cooled to room temperature, diluted with a 1:1 mixture of EA and H₂O. The mixture was stirred for 15 min and the aqueous and organic phases were then separated. The organic layer was washed sequentially with equal volume of saturated aqueous sodium bicarbonate solution, sodium thiosulfate (5% aqueous solution) and brine. The organic layer was dried, filtered and concentrated under reduced pressure to afford 3-bromo-6-iodo-2-methylpyridine (50 g, 84.51%) which was used directly in the next step without any purifications. [M+H]⁺=298.

### Step 2: 2′,6′-bis(benzyloxy)-5-bromo-6-methyl-2,3′-bipyridine

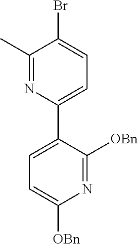 [SMILES: Cc1nc(-c2ccc(OCc3ccccc3)nc2OCc2ccccc2)ccc1Br]

Pd(PPh₃)₄ (1.05 g, 0.91 mmol) was added to 3-bromo-6-iodo-2-methylpyridine (5 g, 16.84 mmol), 2,6-bis(benzyloxy)-3-(4,4,5,5-tetramethyl-1,3,2-dioxaborolan-2-yl)pyridine (7.37 g, 17.67 mmol), and aq K₂CO₃ (2M, 25 ml) in dioxane (125 ml). The resulting mixture was stirred at 100° C. for 3 hours. The reaction mixture was diluted with water and extracted with EA. The combined organic layers were washed with water and then saturated brine, dried over anhydrous Na₂SO₄, filtered and concentrated. The residue was purified by column chromatography, eluted with PE/EA (19:1) to afford 2′,6′-bis(benzyloxy)-5-bromo-6-methyl-2,3′-bipyridine (4.6050 g, 59.57%). [M+H]⁺=461.1.

### Step 3: (R)-(1-(2′,6′-bis(benzyloxy)-6-methyl-[2,3′-bipyridin]-5-yl)pyrrolidin-3-yl)methanol

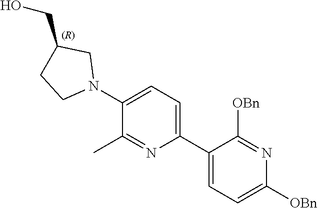

To a stirred mixture of (R)-pyrrolidin-3-ylmethanol (3 g, 29.7 mmol), 2′,6′-bis(benzyloxy)-5-bromo-6-methyl-2,3′-bipyridine (16.4 g, 35.6 mmol), and K₃PO₄ (18.9 g, 89.1 mmol) in 1,4-dioxacyclohexane (160 mL) were added Pd₂(dba)₃ (2.7 g, 3.0 mmol) and Johnphos (1.8 g, 5.9 mmol). The resulting mixture was degassed under reduced pressure and purged with N₂ for three times. Then it was stirred overnight at 100° C. The reaction mixture was filtered through a Celite pad, and the filtrate was concentrated. The residue was purified by column chromatography, eluted with PE/EA (2:3) to afford (R)-(1-(2′,6′-bis(benzyloxy)-6-methyl-[2,3′-bipyridin]-5-yl)pyrrolidin-3-yl)methanol (2.8 g, 19.55%). [M+H]⁺=482.

### Step 4: 3-(5-((R)-3-(hydroxymethyl)pyrrolidin-1-yl)-6-methylpyridin-2-yl)piperidine-2,6-dione

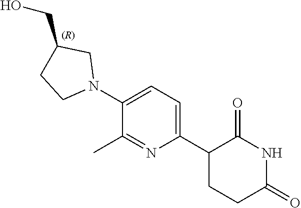

To a stirred solution of (R)-(1-(2′,6′-bis(benzyloxy)-6-methyl-[2,3′-bipyridin]-5-yl)pyrrolidin-3-yl)methanol (2.8 g, 5.81 mmol) in THF (60 mL) was added Pd/C (10 wt %, 6.3 g). The resulting mixture was degassed under reduced pressure and purged with H₂ for five times, then stirred overnight at 50° C. The reaction mixture was diluted with THF/DCM (1:1), then filtered through a celite pad. The filtrate was concentrated under vacuum. The residue was purified by column chromatography, eluted with DCM/MeOH (19:1) to afford 3-(5-((R)-3-(hydroxymethyl)pyrrolidin-1-yl)-6-methylpyridin-2-yl)piperidine-2,6-dione (613.4 mg, 34.85%). [M+H]⁺=304.

### Step 5: ((3R)-1-(6-(2,6-dioxopiperidin-3-yl)-2-methylpyridin-3-yl)pyrrolidin-3-yl)methyl methanesulfonate

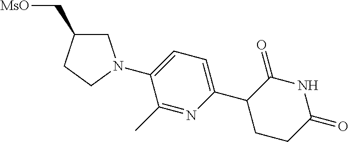 [SMILES: Cc1nc(C2CCC(=O)NC2=O)ccc1N1CC[C@@H](COS(C)(=O)=O)C1]

To the solution of 3-(5-((R)-3-(hydroxymethyl)pyrrolidin-1-yl)-6-methylpyridin-2-yl)piperidine-2,6-dione (303 mg, 1.0 mmol) and TEA (303 mg, 3.0 mmol) in 10 mL DCM was added methanesulfonic anhydride (348 mg, 2.0 mmol). The resulting mixture was stirred at RT for 1 hour. The mixture was concentrated under reduced pressure to give the crude residue, which was purified by silica column chromatography (DCM:MeOH=100:1-20:1) to afford the desired product (340 mg, 89.0%). [M+H]⁺=382.5.

### Intermediate 21: (7¹S,7²R,E)-1¹,2⁶-dimethyl-5⁶-(4-oxopiperidin-1-yl)-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

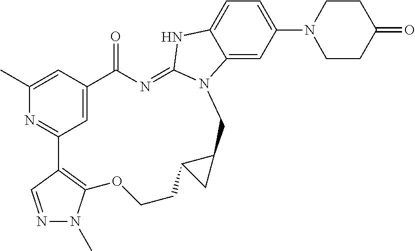 [SMILES: Cc1cc2cc(n1)-c1cnn(C)c1OCC[C@H]1C[C@@H]1CN1/C(=N/C2=O)Nc2ccc(N3CCC(=O)CC3)cc21]

### Step 1: (7¹S,7²R,E)-1¹,2⁶-dimethyl-5⁶-(1,4-dioxa-8-azaspiro[4.5]decan-8-yl)-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

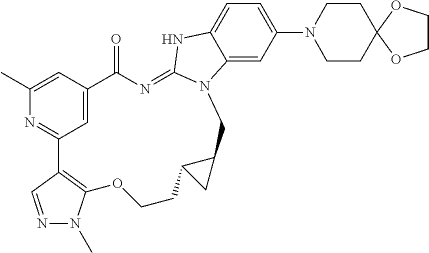 [SMILES: Cc1cc2cc(n1)-c1cnn(C)c1OCC[C@H]1C[C@@H]1CN1/C(=N/C2=O)Nc2ccc(N3CCC4(CC3)OCCO4)cc21]

To the solution of (7¹S,7²R,E)-5⁶-bromo-1¹,2⁶-dimethyl-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one (3.0 g, 5.9 mmol), 1,4-dioxa-8-azaspiro[4.5]decane (1.7 g, 11.8 mmol) and t-BuONa (1.2 g, 11.8 mmol) in 80 mL DMA, were added Pd₂(dba)₃ (540 mg, 0.59 mmol) and RuPhos (550 mg, 1.18 mmol). The mixture was stirred at 100° C. for 3 hours under N₂. The mixture was diluted with water and extracted by EtOAc. The organic layer was washed with brine, dried over anhydrous Na₂SO₄, filtered and concentrated under reduced pressure. The resulting mixture was purified by silica column chromatography (DCM:MeOH=100:1-15:1) to afford the product (2.9 g, 85.8%). [M+H]⁺=570.5.

### Step 2: (7¹S,7²R,E)-1¹,2⁶-dimethyl-5⁶-(4-oxopiperidin-1-yl)-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

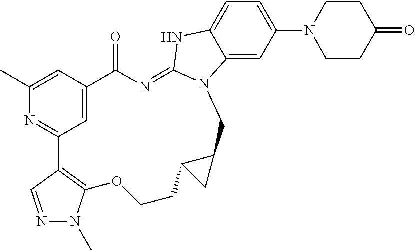 [SMILES: Cc1cc2cc(n1)-c1cnn(C)c1OCC[C@H]1C[C@@H]1CN1/C(=N/C2=O)Nc2ccc(N3CCC(=O)CC3)cc21]

(7¹S,7²R,E)-1¹,2⁶-dimethyl-5⁶-(1,4-dioxa-8-azaspiro[4.5]decan-8-yl)-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one (2.9 g, 5.1 mmol) was placed in 250 mL round bottom flask with a magnetic stir bar. Then, 45 mL 8N HCl aqueous was added. The mixture was stirred at room temperature for 2 hours. The mixture was added dropwise to sat. aq. NaHCO₃ solution and finally pH=6-7. The liquid was extracted with DCM and separated. The organic phase was concentrated in vacuum and purified with combiflash (DCM:MeOH=20:1) to afford the title compound (2.5 g, 93.6% yield). [M+H]⁺=526.5.

### Intermediate 22: 3-(5-(3,8-diazabicyclo[3.2.1]octan-8-yl)-6-methylpyridin-2-yl)piperidine-2,6-dione

### Step 1: tert-butyl 8-(2′,6′-bis(benzyloxy)-6-methyl-[2,3′-bipyridin]-5-yl)-3,8-diazabicyclo[3.2.1]octane-3-carboxylate

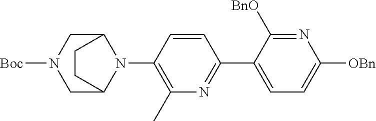 [SMILES: Cc1nc(-c2ccc(OCc3ccccc3)nc2OCc2ccccc2)ccc1N1C2CCC1CN(C(=O)OC(C)(C)C)C2]

To a solution of 2′,6′-bis(benzyloxy)-5-bromo-6-methyl-2,3′-bipyridine (1 g, 2.17 mmol) in dioxane (30 mL) were added tert-butyl 3,8-diazabicyclo[3.2.1]octane-3-carboxylate (691 mg, 3.26 mmol), Pd₂(dba)₃ (393 mg, 0.43 mmol), Ruphos (400 mg, 0.86 mmol), Cs₂CO₃ (1.4 g, 4.3 mmol). The resulting solution was stirred for 16 h at 100° C. under N₂ atmosphere. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with DCM/MeOH (20:1) to afford tert-butyl 8-(2′,6′-bis(benzyloxy)-6-methyl-[2,3′-bipyridin]-5-yl)-3,8-diazabicyclo[3.2.1]octane-3-carboxylate (800 mg, 62.3%). [M+H]⁺=593.4

### Step 2: tert-butyl 8-(6-(2,6-dioxopiperidin-3-yl)-2-methylpyridin-3-yl)-3,8-diazabicyclo[3.2.1]octane-3-carboxylate

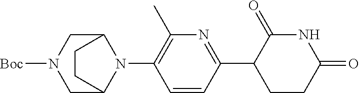 [SMILES: Cc1nc(C2CCC(=O)NC2=O)ccc1N1C2CCC1CN(C(=O)OC(C)(C)C)C2]

To a solution of tert-butyl 8-(2′,6′-bis(benzyloxy)-6-methyl-[2,3′-bipyridin]-5-yl)-3,8-diazabicyclo[3.2.1]octane-3-carboxylate (800 mg, 1.35 mmol) in i-PrOH (15 mL) and DMF (15 mL), was added Pd/C (20 wt %, 160 mg). The resulting mixture was stirred for 16 h at 50° C. under H₂ atmosphere. The mixture was filtrated, and the filtrate was concentrated under vacuum. The residue was purified by silica column chromatography (DCM:MeOH=10:1) to afford tert-butyl 8-(6-(2,6-dioxopiperidin-3-yl)-2-methylpyridin-3-yl)-3,8-diazabicyclo[3.2.1]octane-3-carboxylate (300 mg, 54%). [M+H]⁺=415.3.

### Step 3: 3-(5-(3,8-diazabicyclo[3.2.1]octan-8-yl)-6-methylpyridin-2-yl)piperidine-2,6-dione

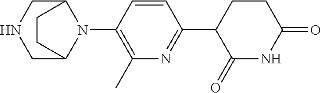 [SMILES: Cc1nc(C2CCC(=O)NC2=O)ccc1N1C2CCC1CNC2]

To the solution of tert-butyl 8-(6-(2,6-dioxopiperidin-3-yl)-2-methylpyridin-3-yl)-3,8-diazabicyclo[3.2.1]octane-3-carboxylate (300 mg, 0.72 mmol) in DCM (10 mL) was added HCl (4M in dioxane, 5 mL). The mixture was stirred in a flask at room temperature for 2 h. The mixture was evaporated in vacuum to afford the crude product (200 mg, 88.5%), which was used for next step without further purification.

### Intermediate 23: 3-(5-((R)-3,3-difluoro-4-(piperazin-1-yl)piperidin-1-yl)-6-methylpyridin-2-yl)piperidine-2,6-dione

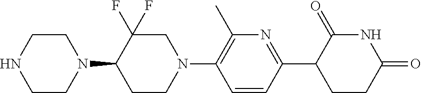 [SMILES: Cc1nc(C2CCC(=O)NC2=O)ccc1N1CC[C@@H](N2CCNCC2)C(F)(F)C1]

The title compound was prepared in a procedure similar to that in intermediate 9.

### Intermediate 24: 3-(5-((3R,4S)-3-fluoro-4-(piperazin-1-yl)piperidin-1-yl)-4-methylpyridin-2-yl)piperidine-2,6-dione

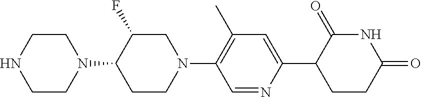 [SMILES: Cc1cc(C2CCC(=O)NC2=O)ncc1N1CC[C@H](N2CCNCC2)[C@H](F)C1]

The title compound was prepared in a procedure similar to that in intermediate 9.

### Intermediate 25: (1-(1-(2,6-dioxopiperidin-3-yl)-3,3-dimethyl-2-oxoindolin-5-yl)azetidin-3-yl)methyl 4-methylbenzenesulfonate

### Step 1: methyl 2-(2,5-dibromophenyl)-2-methylpropanoate

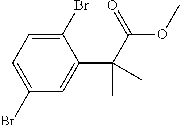

To a stirred solution of 2-(2,5-dibromophenyl)acetic acid (15.0 g, 51.0 mmol) in dry DMF (300 mL), was added NaH (60 wt %, 10.2 g, 255 mmol) portionwise at 0° C. The mixture was degassed under reduced pressure and purged with N₂ for three times, then stirred at 0° C. for 30 minutes. Mel (43.4 g, 306 mmol) was injected into the mixture while maintaining vigorous stirring at 0° C. The resulting mixture was stirred at RT for overnight. The reaction was quenched by addition of 1N HCl while maintaining vigorous stirring at 0° C. The mixture was extracted with EA, washed with water and brine, dried and concentrated. The residue was purified by silica column chromatography PE/EA (9:1) to afford methyl 2-(2,5-dibromophenyl)-2-methylpropanoate (12.0 g, 70%). [M+H]⁺=335.0.

### Step 2: 2-(2,5-dibromophenyl)-2-methylpropanoic acid

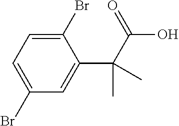

A 250 mL round bottom flask equipped with a magnetic stirrer, were charged with methyl 2-(2,5-dibromophenyl)-2-methylpropanoate (12.0 g, 35.7 mmol), MeOH (150 mL), and a solution of NaOH (14.3 g, 357 mmol) in water (15 mL). The resulting mixture was stirred at 70° C. overnight. After cooled to RT, the mixture was acidified by 1N HCl, then the MeOH was removed by evaporation. The residue was diluted with EA, washed with water and brine, dried and concentrated. The residue was triturated with PE to afford product (11.0 g, 96%). [M+H]⁺=321.0.

### Step 3: N-(2,6-bis(benzyloxy)pyridin-3-yl)-2-(2,5-dibromophenyl)-2-methylpropanamide

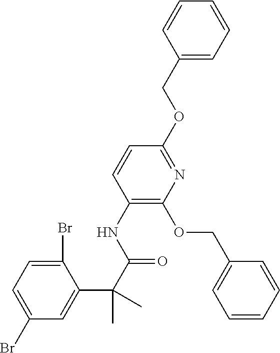 [SMILES: CC(C)(C(=O)Nc1ccc(OCc2ccccc2)nc1OCc1ccccc1)c1cc(Br)ccc1Br]

Into a stirred mixture of 2,6-bis(benzyloxy)pyridin-3-aminium chloride (12.6 g, 41.2 mmol) and 2-(2,5-dibromophenyl)-2-methylpropanoic acid (11 g, 34.3 mmol) in dry MeCN (200 mL) under N₂, was added 1-methyl-1H-imidazole (14.2 g, 171.5 mmol) by a syringe. The mixture was stirred at room temperature for 10 minutes, then a solution of N,N,N,N-Tetramethylchloroformamidinium Hexafluorophosphate (11.5 g, 41.2 mmol) in dry MeCN (50 mL) was added into the mixture by a syringe. The mixture was stirred at 50° C. under N₂ for 4 hours. After cooled to room temperature, the reaction was quenched by addition of water, then the mixture was concentrated in vacuum by an evaporator. The residue was purified by column chromatography PE/EA (19:1) to afford N-(2,6-bis(benzyloxy)pyridin-3-yl)-2-(2,5-dibromophenyl)-2-methylpropanamide (18 g, 86.1%). [M+H]⁺=609.1.

### Step 4: 1-(2,6-bis(benzyloxy)pyridin-3-yl)-5-bromo-3,3-dimethylindolin-2-one

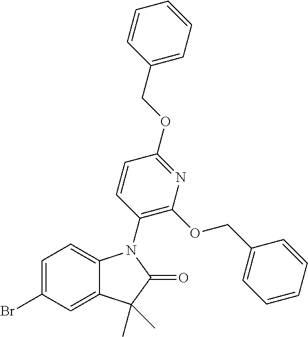 [SMILES: CC1(C)C(=O)N(c2ccc(OCc3ccccc3)nc2OCc2ccccc2)c2ccc(Br)cc21]

To a stirred mixture of N-(2,6-bis(benzyloxy)pyridin-3-yl)-2-(2,5-dibromophenyl)-2-methylpropanamide (10.0 g, 16.4 mmol) and K₂CO₃ (11.3 g, 81.9 mmol) in NMP (150 mL), was added CuCl (1.62 g, 16.4 mmol). The suspension was degassed under vacuum and purged with N₂ three times. Pentane-2,4-dione (3.28 g, 32.8 mmol) was added into the mixture by a syringe. The suspension was stirred at 85° C. under N₂ for 2 hrs. After cooled to room temperature, the mixture was poured into EA, and washed with brine, water, brine in turn, then dried over anhydrous Na₂SO₄, concentrated in vacuum. The residue was purified by column chromatography PE/EA (9:1) to afford 1-(2,6-bis(benzyloxy)pyridin-3-yl)-5-bromo-3,3-dimethylindolin-2-one (6.0 g, 69.2%). [M+H]⁺=529.1.

### Step 5

### 5-(3-((benzyloxy)methyl)azetidin-1-yl)-1-(2,6-bis(benzyloxy)pyridin-3-yl)-3,3-dimethylindolin-2-one

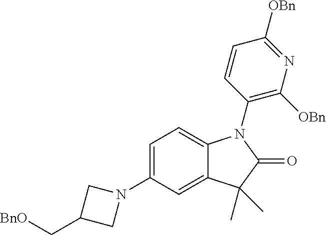 [SMILES: CC1(C)C(=O)N(c2ccc(OCc3ccccc3)nc2OCc2ccccc2)c2ccc(N3CC(COCc4ccccc4)C3)cc21]

The solution of 1-(2,6-bis(benzyloxy)pyridin-3-yl)-5-bromo-3,3-dimethylindolin-2-one (6.0 g, 11.3 mmol), 3-((benzyloxy)methyl)azetidine (2.02 g, 11.3 mmol), Cs₂CO₃ (11.1 g, 34.0 mmol), Pd₂(dba)₃ (1.04 g, 1.13 mmol), RuPhos (1.06 g, 2.26 mmol) in dioxane (80 mL) was degassed under reduced pressure and purged with N₂ for five times, and stirred under N₂ at 100° C. overnight. After cooled to rt, the mixture was diluted with ethyl acetate, then filtered through a pad of celite. The filtrate was washed with brine, dried over anhydrous Na₂SO₄, concentrated under reduced pressure. The residue was purified by silica column chromatography PE/EA (3:2) to afford 5-(3-((benzyloxy)methyl)azetidin-1-yl)-1-(2,6-bis(benzyloxy)pyridin-3-yl)-3,3-dimethylindolin-2-one (4 g, 56.3%). [M+H]⁺=626.3.

### Step 6: 3-(5-(3-(hydroxymethyl)azetidin-1-yl)-3,3-dimethyl-2-oxoindolin-1-yl)piperidine-2,6-dione

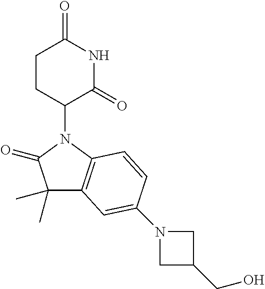 [SMILES: CC1(C)C(=O)N(C2CCC(=O)NC2=O)c2ccc(N3CC(CO)C3)cc21]

A 100 mL round bottom flask equipped with a magnetic stirrer, were charged with 5-(3-((benzyloxy)methyl)azetidin-1-yl)-1-(2,6-bis(benzyloxy)pyridin-3-yl)-3,3-dimethylindolin-2-one (4, 4.79 mmol), i-PrOH/DMF (50 mL/50 mL), and dry Pd/C (4.0 g). The resulting mixture was degassed under reduced pressure and purged with H₂ for five times, then stirred overnight at 50° C. The mixture was diluted with i-PrOH/DMF (1:1), then sonicated in an ultrasonic washer for 5 minutes, followed by filtration through a pad of celite. The filtrate was concentrated under vacuum. The residue was purified by C18 column chromatography (mobile phase A: water, mobile phase B: MeCN) to afford 3-(5-(3-(hydroxymethyl)azetidin-1-yl)-3,3-dimethyl-2-oxoindolin-1-yl)piperidine-2,6-dione (1.63 g, 73.1%). [M+H]⁺=358.1.

### Step 7: (1-(1-(2,6-dioxopiperidin-3-yl)-3,3-dimethyl-2-oxoindolin-5-yl)azetidin-3-yl)methyl 4-methylbenzenesulfonate

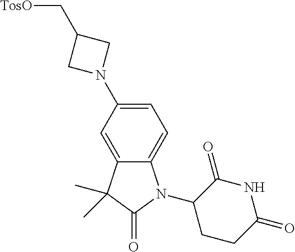

The solution of 3-(5-(3-(hydroxymethyl)azetidin-1-yl)-3,3-dimethyl-2-oxoindolin-1-yl)piperidine-2,6-dione (100 mg, 0.28 mmol) and TosCl (80 mg, 0.42 mmol) in 5 mL pyridine was stirred for 16 h at room temperature. The mixture was concentrated and the residue was purified by silica gel column chromatography, eluted with DCM/MeOH (20:1) to afford the product (120 mg, 83.8%); [M+H]⁺=512.4.

### Intermediate 26: 3-(6-ethyl-5-(4-oxopiperidin-1-yl)pyridin-2-yl)piperidine-2,6-dione

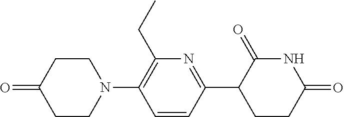 [SMILES: CCc1nc(C2CCC(=O)NC2=O)ccc1N1CCC(=O)CC1]

The title compound was prepared in a procedure similar to that in intermediate 7.

### Intermediate 27: (R)-2-(4-(2,6-dioxopiperidin-3-yl)-3,5-difluorophenyl)acetaldehyde

### Step 1: ethyl 4-(4-bromo-2,6-difluorophenyl)-4-cyanobutanoate

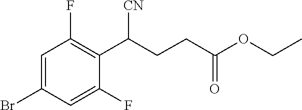 [SMILES: CCOC(=O)CCC(C#N)c1c(F)cc(Br)cc1F]

To a solution of 2-(4-bromo-2,6-difluorophenyl)acetonitrile (10 g, 43.1 mmol) in THF (150 mL) was added LDA (2 M in THF, 24 mL, 48 mmol) dropwise in 20 min at −65° C., the reaction solution was stirred for 1 hour at this temperature, then to the solution was added ethyl 3-bromopropanoate (9.4 g, 51.7 mmol) in THF (30 mL) dropwise in 10 min. The resulting solution was stirred for 30 min at −65° C., and then allowed to warm to room temperature naturally. The reaction was quenched by the addition of sat. aq. NH₄Cl (50 mL), and extracted with EtOAc (100 mL×3). The combined organic layers were washed with brine, dried over anhydrous Na₂SO₄. After filtration, the filtrate was concentrated under reduced pressure to afford the product (13.8 g, 96.5%). [M+H]⁺=332.0.

### Step 2: 4-(4-bromo-2,6-difluorophenyl)-4-cyanobutanoic acid

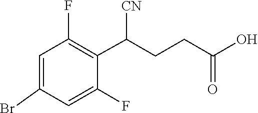 [SMILES: N#CC(CCC(=O)O)c1c(F)cc(Br)cc1F]

To a solution of ethyl 4-(4-bromo-2,6-difluorophenyl)-4-cyanobutanoate (13.5 g, 40.7 mmol) in THF/H₂O (90 mL/30 mL) was added LiOH (2.9 g, 0.122 mol). The reaction mixture was stirred for 12 h at room temperature. The resulting mixture was diluted with water, and extracted with EtOAc (50 mL×2). The pH value of water phase was adjusted to 4-5 with 1 N HCl (10 mL), and extracted with EtOAc (50 mL×3). The combined organic layers were washed with brine (50 mL×3), and dried over anhydrous Na₂SO₄. After filtration, the filtrate was concentrated under reduced pressure to afford the product (10.2 g, 82.5%). [M+H]⁺=304.2.

### Step 3: 3-(4-bromo-2,6-difluorophenyl)piperidine-2,6-dione

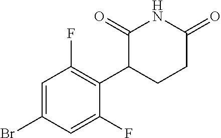 [SMILES: O=C1CCC(c2c(F)cc(Br)cc2F)C(=O)N1]

To a stirred solution of 4-(4-bromo-2,6-difluorophenyl)-4-cyanobutanoic acid (10.2 g, 33.5 mmol) in toluene (100 mL) was added conc. H₂SO₄ (2 mL, 36.9 mmol). The resulting solution was stirred at 100° C. for 3 h. The reaction mixture was concentrated under vacuum, then the mixture was poured into water. The pH value was adjusted to 7-8 with sat. aq. NaHCO₃ (40 mL), and resulting solution was extracted with EtOAc (50 mL×3). The combined organic layers were washed with water and brine, dried over anhydrous Na₂SO₄, filtered and concentrated to afford the product (8.2 g, 80.4%). [M+H]⁺=304.3.

### Step 4: (R,E)-3-(4-(2-ethoxyvinyl)-2,6-difluorophenyl)piperidine-2,6-dione

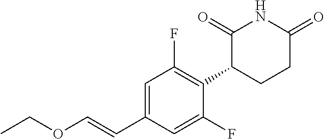 [SMILES: CCO/C=C/c1cc(F)c([C@H]2CCC(=O)NC2=O)c(F)c1]

To a stirred solution of 3-(4-bromo-2,6-difluorophenyl)piperidine-2,6-dione (8.2 g, 27.0 mmol) and (E)-2-(2-ethoxyvinyl)-4,4,5,5-tetramethyl-1,3,2-dioxaborolane (6.4 g, 32.4 mmol) in DMF/H₂O (100 mL/20 mL) were added Pd(dtbpf)Cl₂ (883 mg, 1.35 mmol) and CsF (8.2 g, 54.0 mmol). The resulting mixture was stirred for 2 h at 80° C. under nitrogen atmosphere. The reaction solution was diluted with water, extracted with EtOAc (100 mL×2). The combined organic layers were washed with water and brine, dried over anhydrous Na₂SO₄, filtered and concentrated under reduced pressure. The residue was purified by SFC (1H (3*25 cm, 5 um), 13% EtOH/87% CO₂, 100 bar, 100 ml/min) and the title compound corresponded to peak A@1.679 min/254 nm. (3.1 g, 39.0%). [M+H]⁺=296.1.

### Step 5: (R)-2-(4-(2,6-dioxopiperidin-3-yl)-3,5-difluorophenyl)acetaldehyde

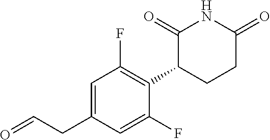 [SMILES: O=CCc1cc(F)c([C@H]2CCC(=O)NC2=O)c(F)c1]

(R,E)-3-(4-(2-ethoxyvinyl)-2,6-difluorophenyl)piperidine-2,6-dione (3.1 g, 10.4 mmol) was dissolved in FA (50 mL). The resulting solution was stirred for 2 h at room temperature. The reaction solution was evaporated to dryness to afford the product (2.6 g, 91.8%). [M+H]⁺=268.1.

### Intermediate 28: (7¹S,7²R,E)-1¹,2⁶-dimethyl-5⁶-(piperazin-1-yl)-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

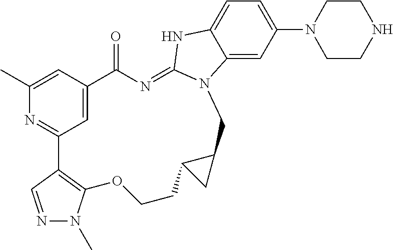 [SMILES: Cc1cc2cc(n1)-c1cnn(C)c1OCC[C@H]1C[C@@H]1CN1/C(=N/C2=O)Nc2ccc(N3CCNCC3)cc21]

The title compound was prepared in a procedure similar to that in intermediate 19. [M+H]⁺=513.5.

### Intermediate 29: 3-(5-((3S,4R)-3-fluoro-4-(piperazin-1-yl)piperidin-1-yl)-6-methylpyridin-2-yl)piperidine-2,6-dione

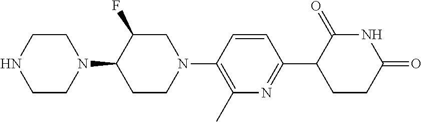 [SMILES: Cc1nc(C2CCC(=O)NC2=O)ccc1N1CC[C@@H](N2CCNCC2)[C@@H](F)C1]

The title compound was prepared in a procedure similar to that in intermediate 9.

### Intermediate 30: (7¹R,7²S,E)-1¹,2⁶,7²-trimethyl-5⁶-(piperazin-1-yl)-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphan-3-one

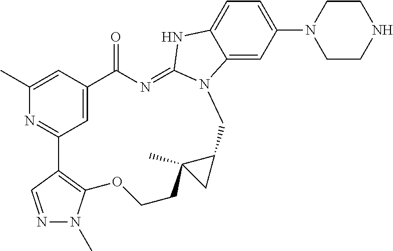 [SMILES: Cc1cc2cc(n1)-c1cnn(C)c1OCC[C@]1(C)C[C@H]1CN1/C(=N/C2=O)Nc2ccc(N3CCNCC3)cc21]

The title compound was prepared in a procedure similar to that in intermediate 19. [M+H]⁺=527.5

### Intermediate 31: 3-(5-((3R,4S)-3-fluoro-4-(piperazin-1-yl)piperidin-1-yl)-6-methylpyridin-2-yl)piperidine-2,6-dione

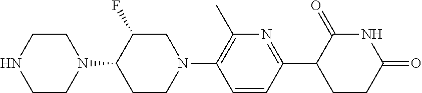 [SMILES: Cc1nc(C2CCC(=O)NC2=O)ccc1N1CC[C@H](N2CCNCC2)[C@H](F)C1]

The title compound was prepared in a procedure similar to that in intermediate 9.

### Intermediate 33: 3-(4-((3S,4R)-3-fluoro-4-(piperazin-1-yl)piperidin-1-yl)-3,3-dimethyl-2-oxoindolin-1-yl)piperidine-2,6-dione

### Step 1: N-(2,6-bis(benzyloxy)pyridin-3-yl)-2-(2,6-dibromophenyl)acetamide

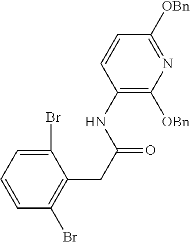 [SMILES: O=C(Cc1c(Br)cccc1Br)Nc1ccc(OCc2ccccc2)nc1OCc1ccccc1]

To a solution of 2-(2,6-dibromophenyl)acetic acid (10 g, 34.2 mmol), 2,6-bis(benzyloxy)pyridin-3-amine (11.5 g, 37.6 mml), DIEA (13.3 g, 102.6 mmol) in DMF (200 ml), were added HATU (19.5 g, 51.3 mmol) at 0° C. The mixture was stirred at RT overnight. The mixture was diluted with EA (500 mL) and then get solid filter. The mixture was concentrated under reduced pressure to afford crude product (15 g, 75.7%). [M+H]⁺=581.

### Step 2: 1-(2,6-bis(benzyloxy)pyridin-3-yl)-4-bromoindolin-2-one

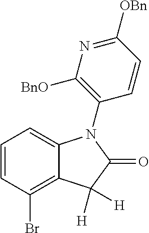 [SMILES: O=C1Cc2c(Br)cccc2N1c1ccc(OCc2ccccc2)nc1OCc1ccccc1]

A 500 mL round bottom flask equipped with a magnetic stirrer, were charged with N-(2,6-bis(benzyloxy)pyridin-3-yl)-2-(2,6-dibromophenyl)acetamide (15 g, 25.9 mmol), ((2-bromoethoxy)methyl)benzene (15.4 g, 71.5 mmol), K₂CO₃ (17.8 g, 129.5 mmol), CuCl (2.56 g, 25.9 mmol), pentane-2,4-dione (5.17 g, 51.8 mmol) and NMP (200 mL). The mixture was degassed under vacuum and purged with N₂ for three times. The resulting mixture was stirred for 3 hours at 85° C. under N₂. After cooled to rt, the mixture was diluted with ethyl acetate (300 mL), then filtered through a pad of celite. The filtrate was washed with brine (300 mL), dried over anhydrous Na₂SO₄, concentrated under reduced pressure. The residue was purified by silica column chromatography (EA/PE, 15%) to afford product (8.00 g, 61.8%). [M+H]⁺=501.

### Step 3: 4-((3S,4R)-4-(4-benzylpiperazin-1-yl)-3-fluoropiperidin-1-yl)-1-(2,6-bis(benzyloxy)pyridin-3-yl)indolin-2-one

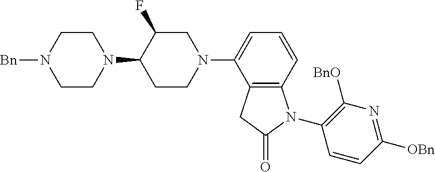 [SMILES: O=C1Cc2c(N3CC[C@@H](N4CCN(Cc5ccccc5)CC4)[C@@H](F)C3)cccc2N1c1ccc(OCc2ccccc2)nc1OCc1ccccc1]

The solution of 1-(2,6-bis(benzyloxy)pyridin-3-yl)-4-bromoindolin-2-one (1 g, 2 mmol, see step 2 in intermediate 10 synthesis), 1-benzyl-4-((3S,4R)-3-fluoropiperidin-4-yl)piperazine (831 mg, 3 mmol, prepared in a manner similar to that in intermediate 20), ^{r-}BuONa (576 mg, 6 mmol), Pd-PEPPSI-IPentCl (195 mg, 0.2 mmol) in DMA (15 mL) was degassed under reduced pressure and purged with N₂ for five times, and stirred under N₂ at 90° C. for 1 hour. After cooled to rt, the mixture was diluted with DCM (100 mL), then filtered through a pad of celite. The filtrate was washed with brine (150 mL), dried over anhydrous Na₂SO₄, concentrated under reduced pressure. The residue was purified by silica column chromatography (EA/PE, 40%) to afford product (810 mg, 60%). [M+H]⁺=698.3.

### Step 4: 4-((3S,4R)-4-(4-benzylpiperazin-1-yl)-3-fluoropiperidin-1-yl)-1-(2,6-bis(benzyloxy)pyridin-3-yl)-3,3-dimethylindolin-2-one

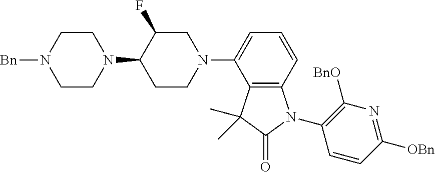 [SMILES: CC1(C)C(=O)N(c2ccc(OCc3ccccc3)nc2OCc2ccccc2)c2cccc(N3CC[C@@H](N4CCN(Cc5ccccc5)CC4)[C@@H](F)C3)c21]

4-((3S,4R)-4-(4-benzylpiperazin-1-yl)-3-fluoropiperidin-1-yl)-1-(2,6-bis(benzyloxy)pyridin-3-yl)indolin-2-one (697 mg, 1 mmol) in DMF (10 mL) and stirred under N₂ at 0° C. NaH (100 mg, 2.5 mmol, 60% in oil) added at this temperature. After 30 min CH₃I (355 mg, 2.5 mmol) was added and stirred under N₂ at room temperature for 1 hour. To this reaction solution was quenched with 20 mL water, it was extracted with DCM. The organic layer was washed with brine, saturated aqueous sodium thiosulfate and water, dried over anhydrous sodium sulfate. It was filtered and the resulting residue was concentrated, it was purified by using column chromatography (EA/PE, 40%) to afford product (500 mg, 69%). [M+H]⁺=726.5.

### Step 5: tert-butyl 4-((3S,4R)-1-(1-(2,6-dioxopiperidin-3-yl)-3,3-dimethyl-2-oxoindolin-4-yl)-3-fluoropiperidin-4-yl)piperazine-1-carboxylate

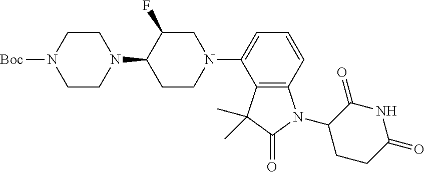 [SMILES: CC(C)(C)OC(=O)N1CCN([C@@H]2CCN(c3cccc4c3C(C)(C)C(=O)N4C3CCC(=O)NC3=O)C[C@@H]2F)CC1]

Under N₂, to a solution of 4-((3S,4R)-4-(4-benzylpiperazin-1-yl)-3-fluoropiperidin-1-yl)-1-(2,6-bis(benzyloxy)pyridin-3-yl)-3,3-dimethylindolin-2-one (726 mg, 1 mmol) in DMF/ⁱ⁻PrOH (5 mL/5 mL) was added 10% Pd/C (100 mg) and (Boc)₂O at room temperature. And then the mixture was exchanged with H₂ two times and stirred under H₂ atmosphere at 50° C. for 15 hours. The mixture was filtered through a pad of Celite and washed with MeOH (50 mL). The filtrate was concentrated under vacuum to obtain the product (500 mg, 90%); [M+H]⁺=558.5.

### Step 6: 3-(4-((3S,4R)-3-fluoro-4-(piperazin-1-yl)piperidin-1-yl)-3,3-dimethyl-2-oxoindolin-1-yl)piperidine-2,6-dione

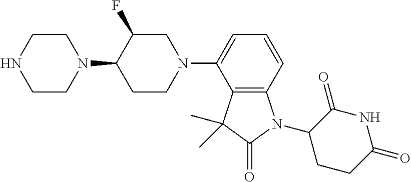 [SMILES: CC1(C)C(=O)N(C2CCC(=O)NC2=O)c2cccc(N3CC[C@@H](N4CCNCC4)[C@@H](F)C3)c21]

To a solution of tert-butyl 4-((3S,4R)-1-(1-(2,6-dioxopiperidin-3-yl)-3,3-dimethyl-2-oxoindolin-4-yl)-3-fluoropiperidin-4-yl)piperazine-1-carboxylate (279 mg, 0.5 mmol) in DCM (3 mL) was added TFA (1 mL). The reaction was stirred at room temperature for 2 hours and then concentrated in vacuo. The residue was dissolved in DCM (20 mL), washed with sat. NaHCO₃ solution (3×20 mL) and brine (50 mL), dried over Na₂SO₄, filtered and concentrated under reduced pressure to afford the product (180 mg, 78.6%); [M+H]⁺=458.5.

### Intermediate 34: 3-(5-((3S,4R)-3-fluoro-4-(piperazin-1-yl)piperidin-1-yl)-4-methylpyridin-2-yl)piperidine-2,6-dione

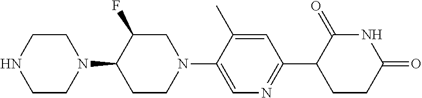 [SMILES: Cc1cc(C2CCC(=O)NC2=O)ncc1N1CC[C@@H](N2CCNCC2)[C@@H](F)C1]

The title compound was prepared in a procedure similar to that in intermediate 9.

### Compound Synthesis

### Example 1: 3-(5-(4-(3-(((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)amino)azetidin-1-yl)piperidin-1-yl)-4-methylpyridin-2-yl)piperidine-2,6-dione

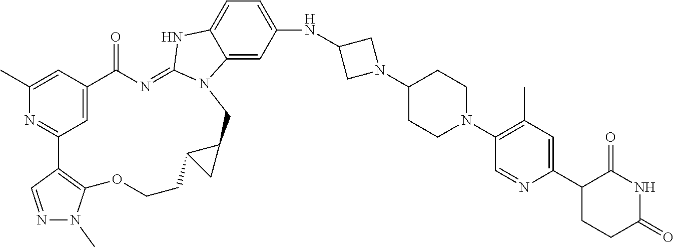 [SMILES: Cc1cc2cc(n1)-c1cnn(C)c1OCC[C@H]1C[C@@H]1CN1/C(=N/C2=O)Nc2ccc(NC3CN(C4CCN(c5cnc(C6CCC(=O)NC6=O)cc5C)CC4)C3)cc21]

The title compound was prepared in a manner similar to that in Example 3.

¹H NMR (500 MHz, DMSO) δ 12.27 (s, 1H), 10.73 (s, 1H), 8.64 (s, 1H), 8.07 (s, 2H), 7.81 (s, 1H), 7.42 (s, 1H), 7.18 (d, J=8.6 Hz, 1H), 7.07 (s, 1H), 6.51 (s, 1H), 6.45 (d, J=8.5 Hz, 1H), 6.06 (s, 1H), 4.24-4.10 (m, 3H), 4.08-3.97 (m, 2H), 3.80-3.75 (m, 3H), 3.68 (s, 3H), 3.06-3.01 (m, 3H), 2.67-2.59 (m, 3H), 2.21-2.03 (m, 11H), 1.81-1.69 (m, 3H), 1.39-1.28 (m, 3H), 1.25-1.22 (m, 1H), 1.10-1.02 (m, 1H), 0.50-0.40 (m, 1H), 0.36-0.28 (m, 1H); [M+H]⁺=784.5.

### Example 2: 3-(5-(4-((R)-4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-3-methylpiperazin-1-yl)piperidin-1-yl)-6-methylpyridin-2-yl)piperidine-2,6-dione

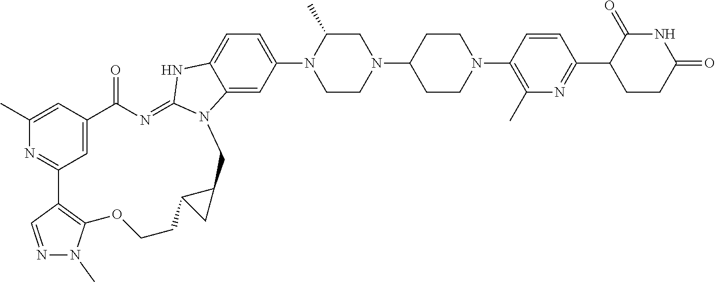 [SMILES: Cc1cc2cc(n1)-c1cnn(C)c1OCC[C@H]1C[C@@H]1CN1/C(=N/C2=O)Nc2ccc(N3CCN(C4CCN(c5ccc(C6CCC(=O)NC6=O)nc5C)CC4)C[C@H]3C)cc21]

The title compound was prepared in a manner similar to that in Example 3.

¹H NMR (500 MHz, DMSO) δ 12.44 (s, 1H), 10.78 (s, 1H), 8.71 (s, 1H), 7.88 (s, 1H), 7.51 (s, 1H), 7.36 (dd, J=8.3, 6.0 Hz, 2H), 7.10 (d, J=8.1 Hz, 1H), 7.04 (s, 1H), 6.88 (d, J=7.8 Hz, 1H), 4.35-4.21 (m, 3H), 4.11 (s, 1H), 3.89 (dd, J=9.5, 5.3 Hz, 2H), 3.75 (s, 3H), 3.14 (d, J=9.6 Hz, 4H), 3.01 (s, 1H), 2.91 (d, J=9.8 Hz, 1H), 2.74-2.55 (m, 8H), 2.43-2.35 (m, 5H), 2.25-2.18 (m, 1H), 2.14-2.03 (m, 2H), 1.91 (s, 2H), 1.71-1.56 (m, 2H), 1.50-1.38 (m, 1H), 1.32 (d, J=4.4 Hz, 1H), 1.19-1.11 (m, 1H), 0.98 (d, J=6.3 Hz, 3H), 0.58-0.46 (m, 1H), 0.38 (dd, J=8.3, 4.7 Hz, 1H). [M+H]⁺=812.6.

### Example 3: 3-(5-(4-((S)-4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5-yl)-3-methylpiperazin-1-yl)piperidin-1-yl)-6-methylpyridin-2-yl)piperidine-2,6-dione

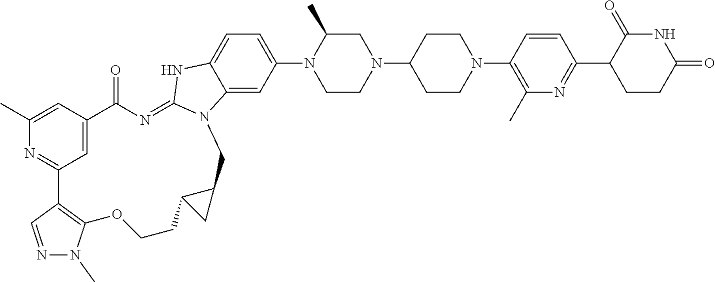 [SMILES: Cc1cc2cc(n1)-c1cnn(C)c1OCC[C@H]1C[C@@H]1CN1/C(=N/C2=O)Nc2ccc(N3CCN(C4CCN(c5ccc(C6CCC(=O)NC6=O)nc5C)CC4)C[C@@H]3C)cc21]

To a solution of intermediate 19 (80 mg, 0.15 mmol) and intermediate 13 (65 mg, 0.21 mmol) in DCE (8 mL) was added STAB (95 mg, 0.45 mmol). Then the mixture was stirred at 50° C. overnight. The reaction was quenched with NaHCO₃ aqueous and extracted with DCM (3×15 mL).

The combined organic phase was washed with brine (1×10 mL), dried over Na₂SO₄, filtered and concentrated in vacuum. The residue was purified by silica gel column (DCM: CH₃OH=10:1), followed by Prep-HPLC chromatography to afford the title product (32 mg, 26.1%). ¹H NMR (500 MHz, DMSO) δ 12.44 (s, 1H), 10.78 (s, 1H), 8.71 (s, 1H), 7.88 (s, 1H), 7.50 (s, 1H), 7.36 (d, J=8.2 Hz, 2H), 7.11 (d, J=8.1 Hz, 1H), 7.04 (s, 1H), 6.89 (d, J=8.5 Hz, 1H), 4.35-4.21 (m, 3H), 4.11 (t, J=7.0 Hz, 1H), 3.94-3.84 (m, 2H), 3.75 (s, 3H), 3.14 (d, J=9.6 Hz, 3H), 3.01 (t, J=9.1 Hz, 1H), 2.90 (s, 1H), 2.74-2.53 (m, 9H), 2.43-2.35 (m, 5H), 2.26-2.17 (m, 1H), 2.08 (d, J=6.7 Hz, 2H), 1.91 (s, 2H), 1.71-1.55 (m, 2H), 1.50-1.39 (m, 1H), 1.33 (d, J=4.5 Hz, 1H), 1.19-1.09 (m, 1H), 0.98 (d, J=6.2 Hz, 3H), 0.52 (s, 1H), 0.41-0.31 (m, 1H). [M+H]⁺=812.6.

### Example 4: 3-(5-(3-(1-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperidin-4-yl)-3,8-diazabicyclo[3.2.1]octan-8-yl)-6-methylpyridin-2-yl)piperidine-2,6-dione

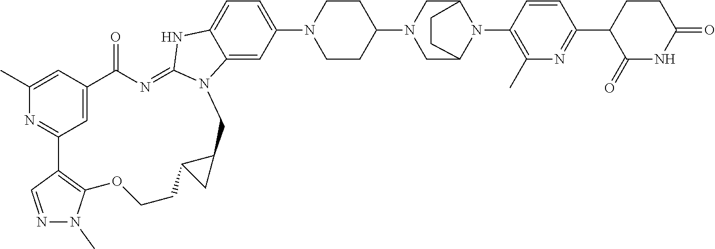 [SMILES: Cc1cc2cc(n1)-c1cnn(C)c1OCC[C@H]1C[C@@H]1CN1/C(=N/C2=O)Nc2ccc(N3CCC(N4CC5CCC(C4)N5c4ccc(C5CCC(=O)NC5=O)nc4C)CC3)cc21]

The title compound was prepared in a manner similar to that in Example 3 with intermediates 21 and 22.

¹H NMR (500 MHz, DMSO) δ 12.43 (s, 1H), 10.76 (s, 1H), 8.71 (s, 1H), 7.88 (s, 1H), 7.50 (s, 1H), 7.34 (d, J=8.7 Hz, 1H), 7.21 (d, J=8.0 Hz, 1H), 7.08-6.98 (m, 2H), 6.90 (d, J=9.4 Hz, 1H), 4.34-4.23 (m, 1H), 4.11 (s, 1H), 3.85 (d, J=8.9 Hz, 1H), 3.75 (s, 3H), 3.69-3.66 (m, 2H), 3.31-3.28 (m, 3H), 2.80-2.78 (m, 4H), 2.54-2.51 (m, 6H), 2.46-2.44 (m, 4H), 2.36 (s, 2H), 2.25-2.15 (m, 1H), 2.09 (d, J=5.2 Hz, 2H), 1.88 (d, J=11.9 Hz, 2H), 1.81 (d, J=7.3 Hz, 2H), 1.75 (s, 2H), 1.61 (d, J=10.7 Hz, 2H), 1.44 (s, 1H), 1.33 (s, 1H), 1.15 (s, 1H), 0.53 (s, 1H), 0.38 (s, 1H). [M+H]⁺=824.0

### Example 5: 3-(5-(5-(1-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperidin-4-yl)-2,5-diazabicyclo[2.2.2]octan-2-yl)-6-methylpyridin-2-yl)piperidine-2,6-dione

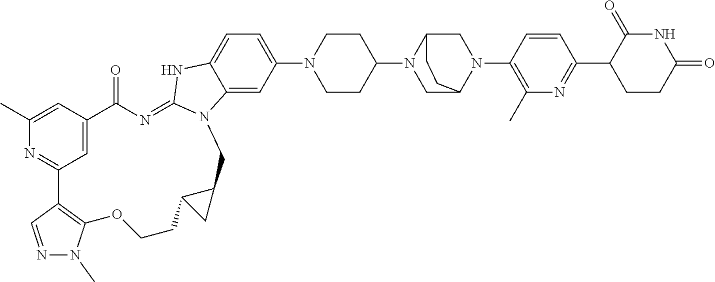 [SMILES: Cc1cc2cc(n1)-c1cnn(C)c1OCC[C@H]1C[C@@H]1CN1/C(=N/C2=O)Nc2ccc(N3CCC(N4CC5CCC4CN5c4ccc(C5CCC(=O)NC5=O)nc4C)CC3)cc21]

The title compound was prepared in a manner similar to that in Example 3.

1H NMR (500 MHz, DMSO) δ 12.43 (s, 1H), 10.76 (s, 1H), 8.71 (s, 1H), 7.88 (s, 1H), 7.50 (s, 1H), 7.34 (d, J=8.6 Hz, 2H), 7.04 (d, J=8.4 Hz, 2H), 6.90 (d, J=8.9 Hz, 1H), 4.35-4.20 (m, 3H), 4.11 (d, J=6.9 Hz, 1H), 3.85 (dd, J=8.7, 5.3 Hz, 1H), 3.75 (s, 3H), 3.64 (d, J=8.4 Hz, 2H), 3.56 (t, J=11.0 Hz, 1H), 3.17 (d, J=9.8 Hz, 2H), 3.09 (s, 1H), 2.89 (d, J=8.8 Hz, 1H), 2.79 (t, J=11.1 Hz, 2H), 2.65-2.54 (m, 7H), 2.38 (s, 3H), 2.19 (dt, J=14.8, 7.5 Hz, 1H), 2.15-1.88 (m, 6H), 1.71 (d, J=10.0 Hz, 2H), 1.58-1.40 (m, 3H), 1.34 (d, J=4.5 Hz, 1H), 1.19-1.09 (m, 1H), 0.53 (d, J=3.8 Hz, 1H), 0.44-0.33 (m, 1H). [M+H]⁺=824.5

### Example 6: 3-(5-(4-(5-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-2,5-diazabicyclo[2.2.2]octan-2-yl)piperidin-1-yl)-6-methylpyridin-2-yl)piperidine-2,6-dione

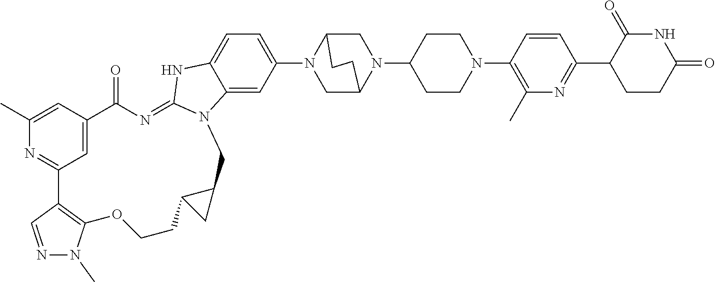 [SMILES: Cc1cc2cc(n1)-c1cnn(C)c1OCC[C@H]1C[C@@H]1CN1/C(=N/C2=O)Nc2ccc(N3CC4CCC3CN4C3CCN(c4ccc(C5CCC(=O)NC5=O)nc4C)CC3)cc21]

The title compound was prepared in a manner similar to that in Example 3.

¹H NMR (500 MHz, DMSO) δ 12.34 (s, 1H), 10.77 (s, 1H), 8.71 (s, 1H), 7.88 (s, 1H), 7.50 (s, 1H), 7.35 (d, J=8.0 Hz, 1H), 7.31 (d, J=8.7 Hz, 1H), 7.09 (d, J=8.1 Hz, 1H), 6.72 (s, 1H), 6.61 (d, J=8.7 Hz, 1H), 4.35-4.19 (m, 3H), 4.16-4.08 (m, 1H), 4.06-3.98 (m, 1H), 3.88 (dd, J=9.3, 5.4 Hz, 1H), 3.75 (s, 3H), 3.62 (d, J=8.7 Hz, 1H), 3.24 (d, J=9.3 Hz, 2H), 3.13-3.09 (m, 2H), 3.04-2.96 (m, 2H), 2.63 (t, J=11.1 Hz, 2H), 2.59-2.54 (m, 6H), 2.39-2.35 (m, 3H), 2.25-2.16 (m, 1H), 2.06-2.01 (m, 5H), 1.91-1.84 (m, 1H), 1.84-1.75 (m, 1H), 1.64-1.61 (m, 1H), 1.56-1.41 (m, 3H), 1.36-1.32 (m, 1H), 1.20-1.10 (m, 1H), 0.57-0.50 (m, 1H), 0.41-0.33 (m, 1H); [M+H]⁺=824.5.

### Example 7: 3-(5-(4-(8-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-3,8-diazabicyclo[3.2.1]octan-3-yl)piperidin-1-yl)-6-methylpyridin-2-yl)piperidine-2,6-dione

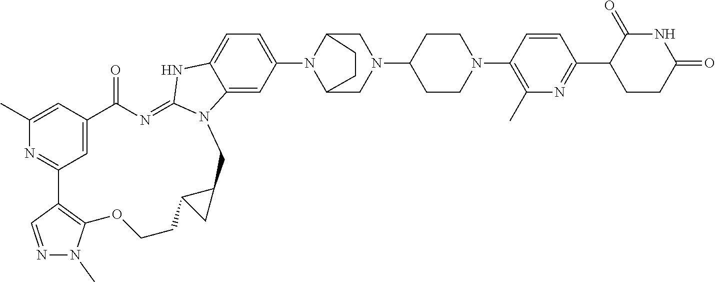 [SMILES: Cc1cc2cc(n1)-c1cnn(C)c1OCC[C@H]1C[C@@H]1CN1/C(=N/C2=O)Nc2ccc(N3C4CCC3CN(C3CCN(c5ccc(C6CCC(=O)NC6=O)nc5C)CC3)C4)cc21]

The title compound was prepared in a manner similar to that in Example 3.

¹H NMR (500 MHz, DMSO) δ 12.36 (s, 1H), 10.77 (s, 1H), 8.71 (s, 1H), 7.88 (s, 1H), 7.50 (s, 1H), 7.32 (t, J=9.3 Hz, 2H), 7.08 (d, J=8.1 Hz, 1H), 6.95 (s, 1H), 6.79 (d, J=8.4 Hz, 1H), 4.36-4.21 (m, 5H), 4.16-4.07 (m, 1H), 3.87 (dd, J=9.1, 5.3 Hz, 1H), 3.75 (s, 3H), 3.06 (d, J=10.9 Hz, 2H), 2.64-2.54 (m, 11H), 2.36 (s, 3H), 2.25-2.15 (m, 2H), 2.10-2.07 (m, 2H), 1.95-1.74 (m, 6H), 1.61-1.50 (m, 2H), 1.48-1.45 (m, 1H), 1.37-1.28 (m, 1H), 1.15 (s, 1H), 0.53 (s, 1H), 0.37 (d, J=3.8 Hz, 1H); [M+H]⁺=824.5.

### Example 8: 3-(5-((R)-4-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)-3,3-difluoropiperidin-1-yl)-6-methylpyridin-2-yl)piperidine-2,6-dione

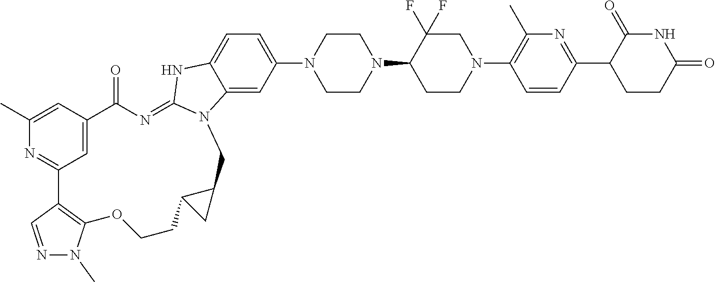 [SMILES: Cc1cc2cc(n1)-c1cnn(C)c1OCC[C@H]1C[C@@H]1CN1/C(=N/C2=O)Nc2ccc(N3CCN([C@@H]4CCN(c5ccc(C6CCC(=O)NC6=O)nc5C)CC4(F)F)CC3)cc21]

The title compound was prepared in a manner similar to that in Example 30 with intermediates 23 and intermediate 15.

¹H NMR (500 MHz, DMSO) δ 12.57 (s, 1H), 10.87 (s, 1H), 8.86 (s, 1H), 8.04 (s, 1H), 7.69 (s, 1H), 7.56 (s, 1H), 7.40 (d, J=8.6 Hz, 1H), 7.26 (s, 1H), 7.17 (d, J=15.6 Hz, 1H), 6.97 (d, J=9.3 Hz, 1H), 4.38-4.25 (m, 4H), 4.18 (s, 1H), 3.99 (s, 2H), 3.78 (s, 3H), 3.28-3.05 (m, 6H), 2.89 (s, 1H), 2.68-2.53 (m, 7H), 2.46-2.41 (m, 5H), 2.25 (s, 1H), 2.13-2.02 (m, 4H), 1.48 (s, 1H), 1.33 (s, 1H), 1.17 (s, 1H), 0.53 (s, 1H), 0.42-0.35 (m, 1H). [M+H]⁺=834.7.

### Example 9: 3-(5-((S)-3-((4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)methyl)pyrrolidin-1-yl)-6-methylpyridin-2-yl)piperidine-2,6-dione

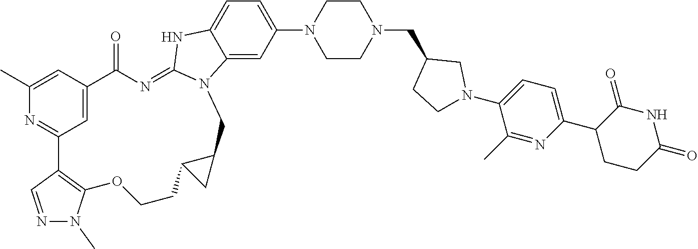 [SMILES: Cc1cc2cc(n1)-c1cnn(C)c1OCC[C@H]1C[C@@H]1CN1/C(=N/C2=O)Nc2ccc(N3CCN(C[C@@H]4CCN(c5ccc(C6CCC(=O)NC6=O)nc5C)C4)CC3)cc21]

To the solution of intermediate 28 (90 mg, 0.17 mmol), intermediate 20 (80 mg, 0.21 mmol) and KI (65 mg, 0.35 mmol) in MeCN (5 mL) and DMSO (2 mL) was added DIEA (67 mg, 0.52 mmol). The resulting mixture was heated at 80° C. overnight under N₂. After LCMS showed the reaction was completed, the mixture was quenched with water and extracted with DCM (3×20 mL). The combined organic phase was washed with brine (1×15 mL), dried over Na₂SO₄, filtered and concentrated under reduced pressure to give the crude residue, which was purified by silica column chromatography (DCM:MeOH=100:1-10:1), followed by Prep-HPLC chromatography to afford the title product (21 mg, 15%).

¹H NMR (500 MHz, DMSO) δ 12.43 (s, 1H), 10.75 (s, 1H), 8.71 (s, 1H), 7.88 (s, 1H), 7.50 (s, 1H), 7.34 (s, 1H), 7.15 (d, J=8.3 Hz, 1H), 7.06 (s, 1H), 7.02 (d, J=8.2 Hz, 1H), 6.90 (d, J=7.6 Hz, 1H), 4.35-4.20 (m, 3H), 4.11 (t, J=6.7 Hz, 1H), 3.84 (dd, J=8.9, 5.3 Hz, 1H), 3.75 (s, 3H), 3.25-3.14 (m, 7H), 3.04 (d, J=9.6 Hz, 1H), 2.66-2.54 (m, 1OH), 2.47-2.36 (m, 5H), 2.20 (dd, J=10.8, 6.4 Hz, 1H), 2.09 (d, J=3.9 Hz, 3H), 1.66 (d, J=6.7 Hz, 1H), 1.49-1.40 (m, 1H), 1.34 (d, J=4.2 Hz, 1H), 1.19-1.10 (m, 1H), 0.56-0.50 (m, 1H), 0.41-0.34 (m, 1H). [M+H]⁺=798.6.

### Example 10: (R)-3-(4-(4-(3-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)azetidin-1-yl)piperidin-1-yl)-2,6-difluorophenyl)piperidine-2,6-dione

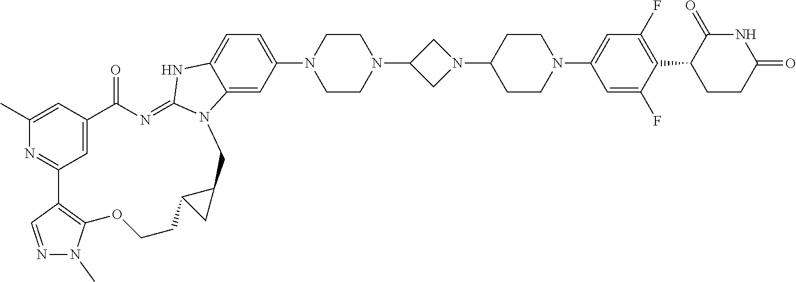 [SMILES: Cc1cc2cc(n1)-c1cnn(C)c1OCC[C@H]1C[C@@H]1CN1/C(=N/C2=O)Nc2ccc(N3CCN(C4CN(C5CCN(c6cc(F)c([C@H]7CCC(=O)NC7=O)c(F)c6)CC5)C4)CC3)cc21]

The title compound was prepared in a manner similar to that in Example 27.

¹H NMR (500 MHz, DMSO) δ 12.44 (s, 1H), 10.87 (s, 1H), 8.70 (s, 1H), 7.88 (s, 1H), 7.50 (s, 1H), 7.35 (d, J=8.7 Hz, 1H), 7.05 (s, 1H), 6.89 (d, J=8.0 Hz, 1H), 6.62 (d, J=13.1 Hz, 2H), 4.35-4.21 (m, 3H), 4.11 (t, J=6.7 Hz, 1H), 4.04 (dd, J=12.4, 4.7 Hz, 1H), 3.75 (s, 3H), 3.63 (s, 2H), 3.45 (s, 1H), 3.21-3.13 (m, 4H), 2.98-2.73 (m, 6H), 2.64-2.53 (m, 4H), 2.47-2.39 (m, 4H), 2.09 (d, J=12.0 Hz, 2H), 2.00-1.92 (m, 1H), 1.71 (s, 3H), 1.45 (d, J=5.8 Hz, 1H), 1.34 (d, J=4.6 Hz, 1H), 1.27-1.13 (m, 4H), 0.53 (s, 1H), 0.42-0.35 (m, 1H). [M+H]⁺=874.6.

### Example 11: 3-(5-((3R,4S)-4-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)-3-fluoropiperidin-1-yl)-4-methylpyridin-2-yl)piperidine-2,6-dione

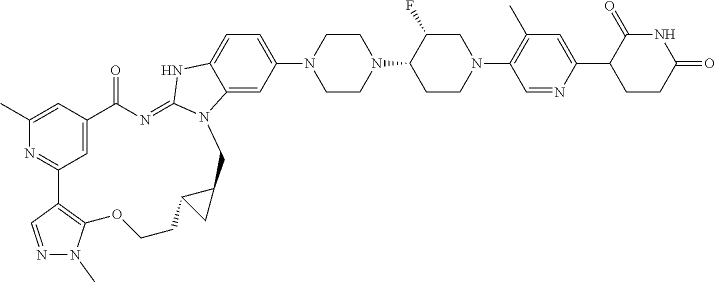 [SMILES: Cc1cc2cc(n1)-c1cnn(C)c1OCC[C@H]1C[C@@H]1CN1/C(=N/C2=O)Nc2ccc(N3CCN([C@H]4CCN(c5cnc(C6CCC(=O)NC6=O)cc5C)C[C@H]4F)CC3)cc21]

The title compound was prepared in a manner similar to that in Example 30 with intermediates 15 and 24.

¹H NMR (500 MHz, DMSO) δ 12.43 (s, 1H), 10.81 (s, 1H), 8.71 (s, 1H), 8.16 (s, 1H), 7.88 (s, 1H), 7.51 (s, 1H), 7.35 (d, J=8.7 Hz, 1H), 7.15 (s, 1H), 7.07 (s, 1H), 6.91 (d, J=8.6 Hz, 1H), 5.17-5.03 (m, 1H), 4.36-4.22 (m, 3H), 4.11 (t, J=6.8 Hz, 1H), 3.90 (dd, J=8.8, 5.4 Hz, 1H), 3.75 (s, 3H), 3.27-3.15 (m, 6H), 3.00-2.79 (m, 6H), 2.64-2.53 (m, 6H), 2.28 (s, 3H), 2.20 (d, J=7.3 Hz, 1H), 2.15-1.98 (m, 3H), 1.83 (d, J=10.2 Hz, 1H), 1.51-1.40 (m, 1H), 1.34 (d, J=4.8 Hz, 1H), 1.19-1.12 (m, 1H), 0.59-0.51 (m, 1H), 0.39 (dd, J=8.5, 4.8 Hz, 1H). [M+H]⁺=816.6.

### Example 12: 3-(5-((3S,4R)-4-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)-3-fluoropiperidin-1-yl)-4-methylpyridin-2-yl)piperidine-2,6-dione

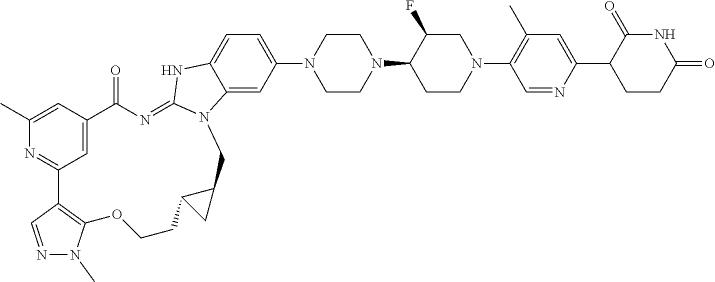 [SMILES: Cc1cc2cc(n1)-c1cnn(C)c1OCC[C@H]1C[C@@H]1CN1/C(=N/C2=O)Nc2ccc(N3CCN([C@@H]4CCN(c5cnc(C6CCC(=O)NC6=O)cc5C)C[C@@H]4F)CC3)cc21]

The title compound was prepared in a manner similar to that in Example 30.

¹H NMR (500 MHz, DMSO) δ 12.43 (s, 1H), 10.81 (s, 1H), 8.71 (s, 1H), 8.16 (s, 1H), 7.88 (s, 1H), 7.51 (s, 1H), 7.35 (d, J=8.7 Hz, 1H), 7.15 (s, 1H), 7.07 (s, 1H), 6.91 (d, J=8.6 Hz, 1H), 5.17-5.03 (m, 1H), 4.36-4.22 (m, 3H), 4.11 (t, J=6.8 Hz, 1H), 3.90 (dd, J=8.8, 5.4 Hz, 1H), 3.75 (s, 3H), 3.27-3.15 (m, 6H), 3.00-2.79 (m, 6H), 2.64-2.53 (m, 6H), 2.28 (s, 3H), 2.20 (d, J=7.3 Hz, 1H), 2.15-2.01 (m, 3H), 1.83 (d, J=10.2 Hz, 1H), 1.51-1.40 (m, 1H), 1.34 (d, J=4.8 Hz, 1H), 1.19-1.12 (m, 1H), 0.59-0.51 (m, 1H), 0.39 (dd, J=8.5, 4.8 Hz, 1H). [M+H]⁺=816.6.

### Example 13: 3-(5-(3-((4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)methyl)azetidin-1-yl)-3,3-dimethyl-2-oxoindolin-1-yl)piperidine-2,6-dione

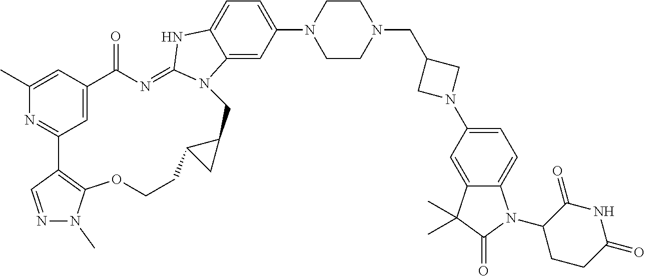 [SMILES: Cc1cc2cc(n1)-c1cnn(C)c1OCC[C@H]1C[C@@H]1CN1/C(=N/C2=O)Nc2ccc(N3CCN(CC4CN(c5ccc6c(c5)C(C)(C)C(=O)N6C5CCC(=O)NC5=O)C4)CC3)cc21]

The title compound was prepared in a manner similar to that in Example 9 with intermediates 28 and 25.

¹H NMR (500 MHz, DMSO) δ 12.37 (s, 1H), 10.96 (s, 1H), 8.64 (s, 1H), 7.81 (s, 1H), 7.44 (s, 1H), 7.28 (d, J=8.6 Hz, 1H), 6.98 (s, 1H), 6.82 (d, J=8.5 Hz, 1H), 6.69 (d, J=7.6 Hz, 1H), 6.48 (s, 1H), 6.19 (d, J=8.0 Hz, 1H), 5.07 (s, 1H), 4.30-4.12 (m, 3H), 4.09-3.96 (m, 1H), 3.92-3.72 (m, 2H), 3.68 (s, 3H), 3.41-3.19 (m, 6H), 3.09 (s, 3H), 2.91-2.75 (m, 2H), 2.59-2.50 (m, 5H), 2.43 (s, 3H), 2.13-1.96 (m, 1H), 1.91-1.77 (m, 1H), 1.38-1.06 (m, 9H), 0.53-0.38 (m, 1H), 0.37-0.22 (m, 1H). [M+H]⁺=852.7.

### Example 14: 3-(5-(4-(4-4((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)piperidin-1-yl)-6-ethylpyridin-2-yl)piperidine-2,6-dione

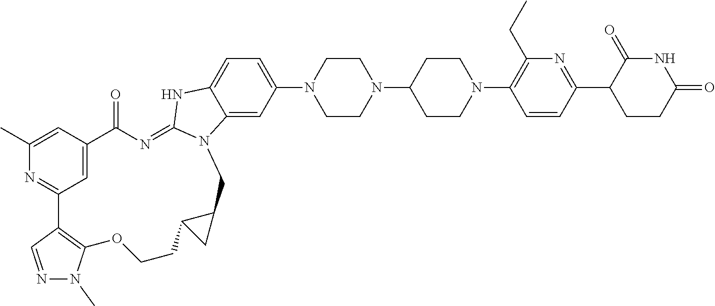 [SMILES: CCc1nc(C2CCC(=O)NC2=O)ccc1N1CCC(N2CCN(c3ccc4c(c3)N3C[C@H]5C[C@@H]5CCOc5c(cnn5C)-c5cc(cc(C)n5)C(=O)/N=C/3N4)CC2)CC1]

The title compound was prepared in a manner similar to that in Example 3 with intermediates 28 and 26.

¹H NMR (500 MHz, DMSO) δ 12.43 (s, 1H), 10.79 (d, J=4.8 Hz, 1H), 8.71 (s, 1H), 7.91-7.84 (m, 1H), 7.50 (d, J=4.4 Hz, 1H), 7.44-7.30 (m, 2H), 7.12 (dd, J=7.7, 3.9 Hz, 1H), 7.05 (s, 1H), 6.90 (s, 1H), 4.35-4.21 (m, 3H), 4.11 (s, 1H), 3.91 (s, 1H), 3.76-3.73 (m, 2H), 3.21-3.05 (m, 6H), 2.84-2.54 (m, 15H), 2.39 (s, 1H), 2.20-2.08 (m, 3H), 1.97-1.86 (m, 2H), 1.63 (s, 2H), 1.44 (s, 1H), 1.33 (s, 1H), 1.21-1.13 (m, 3H), 0.56-0.50 (m, 1H), 0.41-0.34 (m, 1H). [M+H]⁺=812.6.

### Example 15: 3-(5-(4-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)piperidin-1-yl)-4,6-dimethylpyridin-2-yl)piperidine-2,6-dione

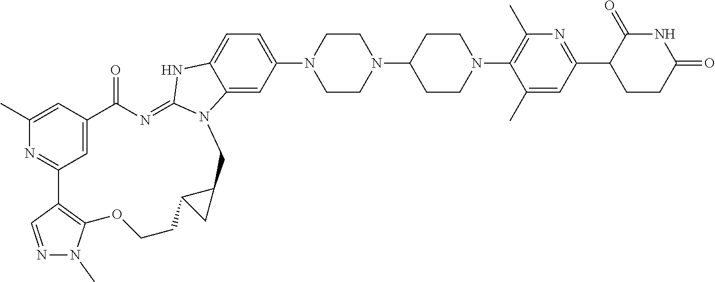 [SMILES: Cc1cc2cc(n1)-c1cnn(C)c1OCC[C@H]1C[C@@H]1CN1/C(=N/C2=O)Nc2ccc(N3CCN(C4CCN(c5c(C)cc(C6CCC(=O)NC6=O)nc5C)CC4)CC3)cc21]

The title compound was prepared in a manner similar to that in Example 3 with intermediates 28 and 6.

¹H NMR (500 MHz, DMSO) δ 12.44 (s, 1H), 10.78 (s, 1H), 8.71 (s, 1H), 7.88 (s, 1H), 7.50 (s, 1H), 7.35 (d, J=8.7 Hz, 1H), 7.05 (s, 1H), 6.90 (d, J=8.7 Hz, 2H), 4.28 (dd, J=11.7, 4.7 Hz, 3H), 4.11 (t, J=6.6 Hz, 1H), 3.84 (dd, J=9.2, 5.3 Hz, 1H), 3.75 (s, 3H), 3.20-3.07 (m, 6H), 2.95 (d, J=13.2 Hz, 2H), 2.78-2.68 (m, 4H), 2.60-2.54 (m, 5H), 2.41 (d, J=7.6 Hz, 4H), 2.28 (s, 3H), 2.22-2.03 (m, 3H), 1.86 (d, J=10.4 Hz, 2H), 1.59 (d, J=8.2 Hz, 2H), 1.44 (dd, J=14.3, 6.3 Hz, 1H), 1.34 (d, J=4.6 Hz, 1H), 1.18-1.11 (m, 1H), 0.56-0.50 (m, 1H), 0.42-0.34 (m, 1H). [M+H]⁺=812.6.

### Example 16: 3-(5-(4-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)piperidin-1-yl)-3-fluoro-6-methylpyridin-2-yl)piperidine-2,6-dione

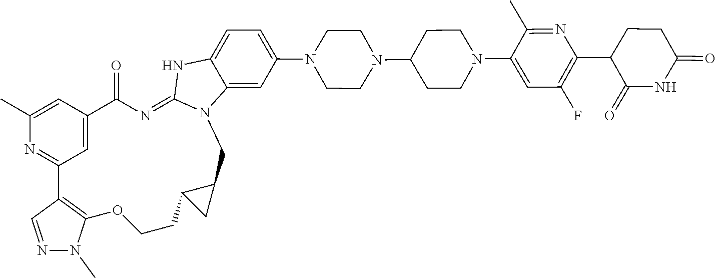 [SMILES: Cc1cc2cc(n1)-c1cnn(C)c1OCC[C@H]1C[C@@H]1CN1/C(=N/C2=O)Nc2ccc(N3CCN(C4CCN(c5cc(F)c(C6CCC(=O)NC6=O)nc5C)CC4)CC3)cc21]

The title compound was prepared in a manner similar to that in Example 3 with intermediates 28 and 5.

¹H NMR (500 MHz, DMSO) δ 12.44 (s, 1H), 10.87 (s, 1H), 8.71 (s, 1H), 7.88 (s, 1H), 7.50 (s, 1H), 7.37-7.31 (m, 2H), 7.05 (d, J=1.2 Hz, 1H), 6.90 (dd, J=8.8, 1.7 Hz, 1H), 4.36-4.22 (m, 3H), 4.18-4.08 (m, 2H), 3.75 (s, 3H), 3.18 (s, 6H), 2.74-2.63 (m, 7H), 2.58-2.52 (m, 4H), 2.42-2.37 (m, 4H), 2.30-2.20 (m, 1H), 2.15-2.01 (m, 2H), 1.98-1.91 (m, 2H), 1.68-1.58 (m, 2H), 1.49-1.41 (m, 1H), 1.38-1.31 (m, 1H), 1.19-1.11 (m, 1H), 0.58-0.52 (m, 1H), 0.41-0.35 (m, 1H). [M+H]⁺=816.70.

### Example 17: (R)-3-(2,6-difluoro-4-(4-(4-((7¹R,7²S,E)-1¹,2⁶,7²-trimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)piperidin-1-yl)phenyl)piperidine-2,6-dione

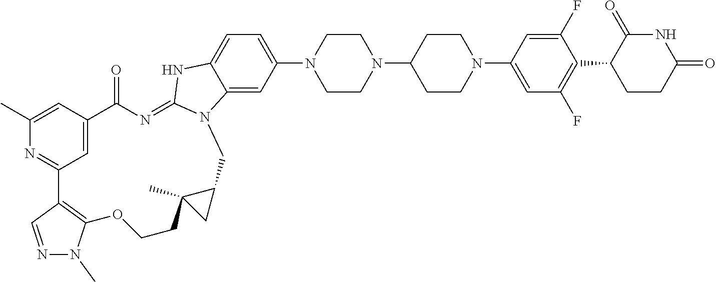 [SMILES: Cc1cc2cc(n1)-c1cnn(C)c1OCC[C@]1(C)C[C@H]1CN1/C(=N/C2=O)Nc2ccc(N3CCN(C4CCN(c5cc(F)c([C@H]6CCC(=O)NC6=O)c(F)c5)CC4)CC3)cc21]

The title compound was prepared in a manner similar to that in Example 3 with intermediates 30 and intermediate 3.

¹H NMR (500 MHz, DMSO) δ 12.46 (s, 1H), 10.87 (s, 1H), 8.52 (s, 1H), 7.87 (s, 1H), 7.53 (s, 1H), 7.36 (d, J=8.8 Hz, 1H), 7.17 (s, 1H), 6.88 (d, J=8.8 Hz, 1H), 6.64 (d, J=12.8 Hz, 2H), 4.57 (d, J=13.4 Hz, 2H), 4.14-4.02 (m, 2H), 3.90 (dd, J=13.7, 9.2 Hz, 1H), 3.81 (d, J=12.7 Hz, 2H), 3.74 (s, 3H), 3.16 (d, J=4.6 Hz, 4H), 2.82-2.75 (m, 4H), 2.69 (s, 3H), 2.65-2.53 (m, 6H), 2.12-2.05 (m, 1H), 2.00-1.93 (m, 1H), 1.88 (d, J=11.1 Hz, 2H), 1.52-1.41 (m, 7.7 Hz, 3H), 1.36 (s, 3H), 1.05 (d, J=14.7 Hz, 1H), 0.75 (s, 1H), 0.60 (s, 1H). [M+H]⁺=833.6

### Example 18: 3-(4-(4-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)piperidin-1-yl)-3,3-dimethyl-2-oxoindolin-1-yl)piperidine-2,6-dione

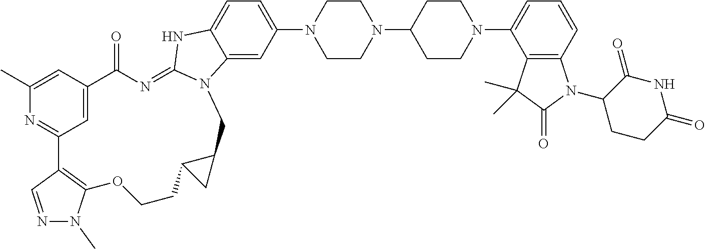 [SMILES: Cc1cc2cc(n1)-c1cnn(C)c1OCC[C@H]1C[C@@H]1CN1/C(=N/C2=O)Nc2ccc(N3CCN(C4CCN(c5cccc6c5C(C)(C)C(=O)N6C5CCC(=O)NC5=O)CC4)CC3)cc21]

The title compound was prepared in a manner similar to that in Example 3 with intermediates 28 and 1.

¹H NMR (500 MHz, DMSO) δ 12.44 (s, 1H), 11.06 (s, 1H), 8.71 (s, 1H), 7.88 (s, 1H), 7.50 (s, 1H), 7.36 (d, J=8.7 Hz, 1H), 7.24 (t, J=7.9 Hz, 1H), 7.08-7.01 (m, 2H), 6.91 (d, J=8.9 Hz, 1H), 6.80 (s, 1H), 5.21 (s, 1H), 4.38-4.21 (m, 3H), 4.11 (s, 1H), 3.75 (s, 3H), 3.19 (s, 3H), 2.93-2.87 (m, 3H), 2.80-2.73 (m, 2H), 2.73 (s, 3H), 2.64-2.60 (m, 2H), 2.58-2.54 (m, 5H), 2.44-2.39 (m, 1H), 2.13-2.09 (m, 1H), 1.95-1.93 (m, 3H), 1.65-1.58 (m, 2H), 1.47-1.42 (m, 7H), 1.36-1.34 (m, 1H), 1.19-1.15 (m, 1H), 0.56-0.52 (m, 1H), 0.43-0.35 (m, 1H). [M+H]⁺=866.8.

### Example 19: 3-(6-methyl-5-(4-(4-((7¹R,7²S,E)-1¹,2⁶,7²-trimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)piperidin-1-yl)pyridin-2-yl)piperidine-2,6-dione

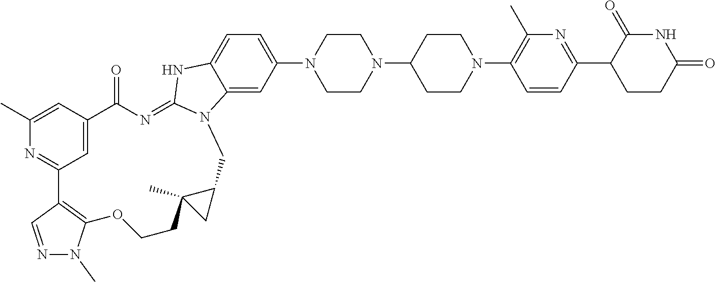 [SMILES: Cc1cc2cc(n1)-c1cnn(C)c1OCC[C@]1(C)C[C@H]1CN1/C(=N/C2=O)Nc2ccc(N3CCN(C4CCN(c5ccc(C6CCC(=O)NC6=O)nc5C)CC4)CC3)cc21]

The title compound was prepared in a manner similar to that in Example 3.

¹H NMR (500 MHz, DMSO) δ 12.62 (s, 1H), 10.88 (s, 1H), 8.65 (s, 1H), 7.99 (s, 1H), 7.68 (s, 1H), 7.56 (s, 1H), 7.45 (d, J=8.7 Hz, 1H), 7.31 (s, 1H), 7.27 (s, 1H), 7.01 (d, J=8.7 Hz, 1H), 4.62 (d, J=13.7 Hz, 1H), 4.57 (dd, J=9.8, 4.2 Hz, 1H), 4.13 (s, 2H), 4.01-3.89 (m, 4H), 3.77 (s, 3H), 3.72 (s, 2H), 3.48 (s, 1H), 3.29 (d, J=10.6 Hz, 4H), 3.10-3.03 (m, 2H), 2.74 (t, J=11.5 Hz, 2H), 2.64 (s, 3H), 2.61-2.53 (m, 2H), 2.48 (s, 3H), 2.25 (d, J=9.5 Hz, 3H), 2.09 (dd, J=13.2, 5.2 Hz, 1H), 1.91-1.85 (m, 2H), 1.46 (dd, J=14.2, 8.4 Hz, 1H), 1.39 (s, 3H), 1.08 (d, J=15.1 Hz, 1H), 0.75 (t, J=4.4 Hz, 1H), 0.65-0.56 (m, 1H). [M+H]⁺=812.5

### Example 20: 3-(5-(4-(3-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)azetidin-1-yl)piperidin-1-yl)-6-methylpyridin-2-yl)piperidine-2,6-dione

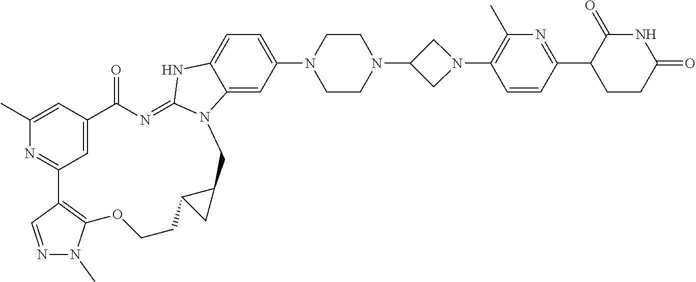 [SMILES: Cc1cc2cc(n1)-c1cnn(C)c1OCC[C@H]1C[C@@H]1CN1/C(=N/C2=O)Nc2ccc(N3CCN(C4CN(c5ccc(C6CCC(=O)NC6=O)nc5C)C4)CC3)cc21]

The title compound was prepared in a manner similar to that in Example 27.

¹H NMR (500 MHz, DMSO) δ 12.44 (s, 1H), 10.78 (s, 1H), 8.71 (s, 1H), 7.88 (s, 1H), 7.50 (s, 1H), 7.36 (d, J=4.5 Hz, 1H), 7.34 (d, J=4.9 Hz, 1H), 7.09 (d, J=8.2 Hz, 1H), 7.05 (s, 1H), 6.90 (dd, J=8.9, 1.7 Hz, 1H), 4.30 (d, J=6.9 Hz, 3H), 4.11 (t, J=6.8 Hz, 1H), 3.88 (dd, J=9.4, 5.3 Hz, 1H), 3.75 (s, 3H), 3.43 (t, J=5.8 Hz, 2H), 3.17 (s, 4H), 3.02 (s, 2H), 2.92 (dd, J=12.3, 6.1 Hz, 1H), 2.87 (d, J=6.2 Hz, 2H), 2.65-2.53 (m, 7H), 2.44 (s, 4H), 2.37 (s, 3H), 2.24-2.14 (m, 2H), 2.13-2.03 (m, 2H), 1.77 (d, J=10.0 Hz, 2H), 1.50-1.41 (m, 1H), 1.35 (d, J=8.9 Hz, 3H), 1.15 (dt, J=8.4, 4.4 Hz, 1H), 0.58-0.50 (m, 1H), 0.38 (dt, J=9.2, 4.6 Hz, 1H). [M+H]⁺=853.6.

### Example 21: 3-(5-(2-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)ethyl)-4,6-dimethylpyridin-2-yl)piperidine-2,6-dione

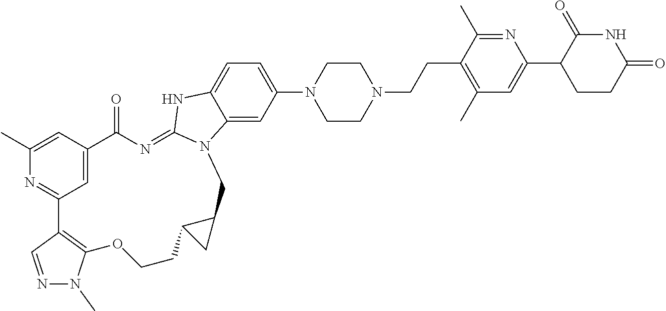 [SMILES: Cc1cc2cc(n1)-c1cnn(C)c1OCC[C@H]1C[C@@H]1CN1/C(=N/C2=O)Nc2ccc(N3CCN(CCc4c(C)cc(C5CCC(=O)NC5=O)nc4C)CC3)cc21]

The titled compound was prepared in a manner similar to that in Example 9 with intermediates 8 and 28.

¹H NMR (500 MHz, DMSO) δ 12.44 (s, 1H), 10.79 (s, 1H), 8.71 (s, 1H), 7.88 (s, 1H), 7.50 (s, 1H), 7.35 (d, J=8.7 Hz, 1H), 7.07 (s, 1H), 6.98 (s, 1H), 6.91 (d, J=8.8 Hz, 1H), 4.29 (d, J=9.0 Hz, 3H), 4.14-4.10 (m, 1H), 3.86-3.83 (m, 1H), 3.75 (s, 3H), 3.21 (s, 3H), 2.84-2.79 (m, 2H), 2.69-2.66 (m, 4H), 2.65-2.62 (m, 1H), 2.59-2.56 (m, 3H), 2.55 (s, 4H), 2.47 (s, 3H), 2.39-2.34 (m, 1H), 2.33 (s, 2H), 2.25-2.19 (m, 1H), 2.12-2.05 (m, 2H), 1.45 (s, 1H), 1.35 (s, 1H), 1.15 (d, J=3.5 Hz, 1H), 0.58-0.52 (m, 1H), 0.41-0.36 (m, 1H); [M+H]⁺=757.4

### Example 22: 3-(5-(4-(3-((S)-4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-3-methylpiperazin-1-yl)azetidin-1-yl)piperidin-1-yl)-4,6-dimethylpyridin-2-yl)piperidine-2,6-dione

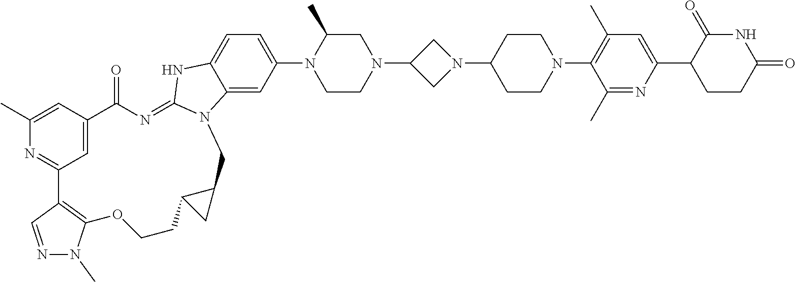 [SMILES: Cc1cc2cc(n1)-c1cnn(C)c1OCC[C@H]1C[C@@H]1CN1/C(=N/C2=O)Nc2ccc(N3CCN(C4CN(C5CCN(c6c(C)cc(C7CCC(=O)NC7=O)nc6C)CC5)C4)C[C@@H]3C)cc21]

The title compound was prepared in a manner similar to that in Example 27.

¹H NMR (500 MHz, DMSO) δ 12.45 (s, 1H), 10.78 (s, 1H), 8.71 (s, 1H), 7.88 (s, 1H), 7.50 (s, 1H), 7.36 (d, J=8.6 Hz, 1H), 7.05 (s, 1H), 6.96 (d, J=32.4 Hz, 1H), 6.94-6.85 (m, 1H), 4.36-4.20 (m, 3H), 4.16-4.07 (m, 1H), 3.95-3.86 (m, 1H), 3.85-3.81 (m, 1H), 3.78-3.72 (m, 3H), 3.53-3.49 (m, 2H), 3.21 (s, 1H), 3.07-2.88 (m, 7H), 2.71-2.63 (m, 1H), 2.60-2.56 (m, 6H), 2.48-2.44 (m, 2H), 2.40-2.37 (m, 4H), 2.30-2.26 (m, 3H), 2.23-2.15 (m, 2H), 2.13-2.02 (m, 2H), 1.83-1.70 (m, 2H), 1.50-1.41 (m, 1H), 1.40-1.27 (m, 3H), 1.18-1.16 (m, 1H), 0.99-0.97 (m, 3H), 0.56-0.48 (m, 1H), 0.41-0.34 (m, 1H); [M+H]⁺=881.5.

### Example 24: 3-(5-(4-(3-((R)-4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-3-methylpiperazin-1-yl)azetidin-1-yl)piperidin-1-yl)-4,6-dimethylpyridin-2-yl)piperidine-2,6-dione

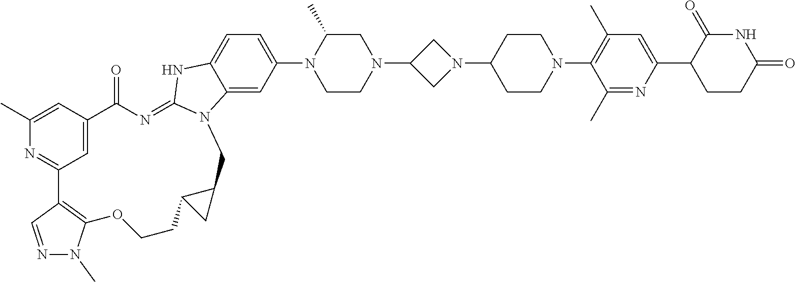 [SMILES: Cc1cc2cc(n1)-c1cnn(C)c1OCC[C@H]1C[C@@H]1CN1/C(=N/C2=O)Nc2ccc(N3CCN(C4CN(C5CCN(c6c(C)cc(C7CCC(=O)NC7=O)nc6C)CC5)C4)C[C@H]3C)cc21]

The title compound was prepared in a manner similar to that in Example 27.

¹H NMR (500 MHz, DMSO) δ 12.45 (s, 1H), 10.78 (s, 1H), 8.71 (s, 1H), 7.88 (s, 1H), 7.50 (s, 1H), 7.36 (d, J=8.7 Hz, 1H), 7.05 (s, 1H), 6.97-6.92 (m, 1H), 6.88 (d, J=8.0 Hz, 1H), 4.35-4.21 (m, 3H), 4.16-4.06 (m, 1H), 3.96-3.88 (m, 1H), 3.83 (dd, J=9.2, 5.3 Hz, 1H), 3.75 (s, 3H), 3.55-3.39 (m, 2H), 3.25-3.17 (m, 1H), 3.07-2.75 (m, 8H), 2.69-2.64 (m, 1H), 2.61-2.54 (m, 5H), 2.47-2.43 (m, 1H), 2.39-2.35 (m, 4H), 2.26-2.23 (m, 3H), 2.22-2.14 (m, 2H), 2.14-2.02 (m, 3H), 1.83-1.65 (m, 2H), 1.50-1.39 (m, 1H), 1.37-1.25 (m, 3H), 1.19-1.11 (m, 1H), 0.98 (d, J=6.1 Hz, 3H), 0.58-0.49 (m, 1H), 0.42-0.33 (m, 1H); [M+H]⁺=881.5.

### Example 25: 3-(6-ethyl-5-(4-(4-((7¹R,7²S,E)-1¹,2⁶,7²-trimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)piperidin-1-yl)pyridin-2-yl)piperidine-2,6-dione

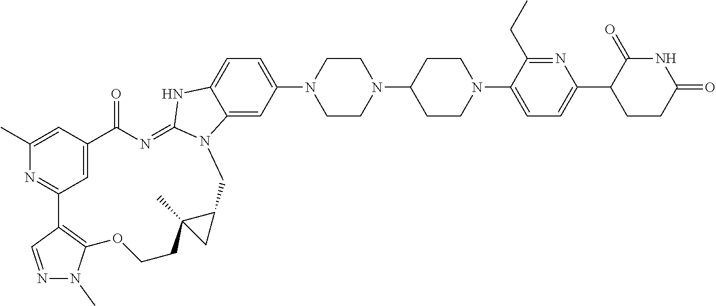 [SMILES: CCc1nc(C2CCC(=O)NC2=O)ccc1N1CCC(N2CCN(c3ccc4c(c3)N3C[C@@H]5C[C@@]5(C)CCOc5c(cnn5C)-c5cc(cc(C)n5)C(=O)/N=C/3N4)CC2)CC1]

The title compound was prepared in a manner similar to that in Example 3.

¹H NMR (500 MHz, DMSO) δ 12.59-12.26 (m, 1H), 10.79 (s, 1H), 8.59-8.48 (m, 1H), 7.95-7.82 (m, 1H), 7.53 (s, 1H), 7.47-7.39 (m, 1H), 7.39-7.32 (m, 1H), 7.19 (s, 1H), 7.12 (d, J=8.1 Hz, 1H), 6.90 (d, J=8.8 Hz, 1H), 4.58 (d, J=13.7 Hz, 2H), 4.09 (t, J=7.0 Hz, 1H), 3.97-3.86 (m, 2H), 3.74 (s, 3H), 3.18 (d, J=4.5 Hz, 4H), 3.11-3.03 (m, 2H), 2.84-2.76 (m, 1H), 2.75-2.60 (m, 7H), 2.59-2.53 (m, 6H), 2.39 (t, J=11.7 Hz, 1H), 2.23-2.08 (m, 2H), 1.92 (d, J=10.3 Hz, 2H), 1.63 (d, J=11.3 Hz, 2H), 1.44 (dd, J=14.0, 7.5 Hz, 1H), 1.37 (s, 3H), 1.18 (t, J=7.4 Hz, 3H), 1.06 (d, J=14.3 Hz, 1H), 0.76 (t, J=4.3 Hz, 1H), 0.61 (dd, J=7.7, 3.3 Hz, 1H). [M+H]⁺=826.6.

### Example 26: (R)-3-(2,6-difluoro-4-(4-((S)-2-(methoxymethyl)-4-((7¹R,7²S,E)-1¹,2⁶,7²-trimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)piperidin-1-yl)phenyl)piperidine-2,6-dione

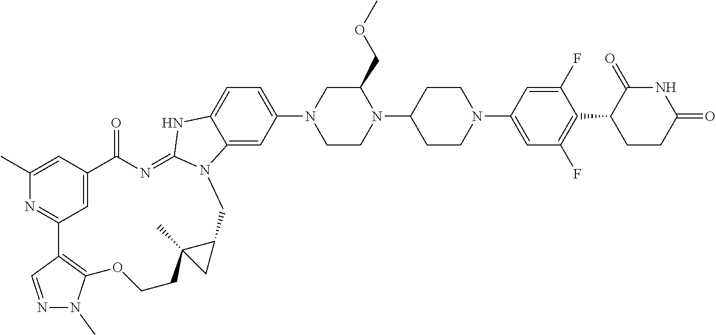 [SMILES: COC[C@@H]1CN(c2ccc3c(c2)N2C[C@@H]4C[C@@]4(C)CCOc4c(cnn4C)-c4cc(cc(C)n4)C(=O)/N=C/2N3)CCN1C1CCN(c2cc(F)c([C@H]3CCC(=O)NC3=O)c(F)c2)CC1]

The title compound was prepared in a manner similar to that in Example 3.

¹H NMR (500 MHz, DMSO) δ 12.44 (s, 1H), 10.87 (s, 1H), 8.52 (s, 1H), 7.87 (s, 1H), 7.53 (s, 1H), 7.37 (d, J=8.7 Hz, 1H), 7.13 (s, 1H), 6.86 (d, J=8.6 Hz, 1H), 6.65 (s, 1H), 6.63 (s, 1H), 4.63-4.51 (m, 2H), 4.13-4.00 (m, 2H), 3.90 (dd, J=13.7, 9.2 Hz, 1H), 3.83 (d, J=11.8 Hz, 2H), 3.74 (s, 3H), 3.57 (dd, J=9.4, 4.2 Hz, 1H), 3.50 (dd, J=9.5, 5.7 Hz, 1H), 3.29 (s, 3H), 3.27 (s, 1H), 3.22-3.14 (m, 1H), 3.11-3.01 (m, 2H), 2.98 (dd, J=11.0, 6.8 Hz, 1H), 2.90 (t, J=11.0 Hz, 1H), 2.85-2.70 (m, 4H), 2.70-2.62 (m, 1H), 2.59-2.51 (m, 5H), 2.09 (qd, J=12.9, 3.6 Hz, 1H), 1.99-1.92 (m, 1H), 1.88 (d, J=10.9 Hz, 1H), 1.74 (d, J=11.1 Hz, 1H), 1.62 (ddd, J=12.4, 9.3, 4.7 Hz, 1H), 1.49-1.39 (m, 2H), 1.37 (s, 3H), 1.05 (d, J=14.5 Hz, 1H), 0.77-0.68 (m, 1H), 0.61 (dd, J=7.7, 3.2 Hz, 1H). [M+H]⁺=877.7.

### Example 27: (R)-3-(2,6-difluoro-4-(4-(3-((S)-3-methyl-4-((7¹R,7²S,E)-1¹,2⁶,7²-trimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)azetidin-1-yl)piperidin-1-yl)phenyl)piperidine-2,6-dione

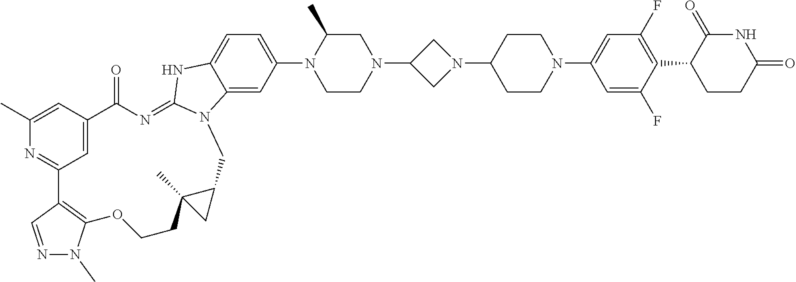 [SMILES: Cc1cc2cc(n1)-c1cnn(C)c1OCC[C@]1(C)C[C@H]1CN1/C(=N/C2=O)Nc2ccc(N3CCN(C4CN(C5CCN(c6cc(F)c([C@H]7CCC(=O)NC7=O)c(F)c6)CC5)C4)C[C@@H]3C)cc21]

The title compound was prepared in a manner similar to that in Example 3 with intermediates 17 and intermediate 3.

¹H NMR (500 MHz, DMSO) δ 12.47 (s, 1H), 10.86 (s, 1H), 8.52 (s, 1H), 7.87 (s, 1H), 7.53 (s, 1H), 7.37 (d, J=8.7 Hz, 1H), 7.21 (s, 1H), 6.86 (d, J=7.8 Hz, 1H), 6.63 (s, 1H), 6.60 (s, 1H), 4.57 (dd, J=12.8, 7.3 Hz, 2H), 4.09 (t, J=6.9 Hz, 1H), 4.04 (dd, J=12.5, 4.8 Hz, 1H), 3.89 (dd, J=13.1, 8.8 Hz, 2H), 3.74 (s, 3H), 3.59 (d, J=12.5 Hz, 2H), 3.44-3.38 (m, 2H), 3.19 (d, J=11.4 Hz, 1H), 2.99 (t, J=8.7 Hz, 1H), 2.87 (dd, J=13.1, 7.7 Hz, 4H), 2.81-2.74 (m, 2H), 2.62 (d, J=10.7 Hz, 1H), 2.56 (s, 3H), 2.54 (d, J=2.2 Hz, 2H), 2.38 (dd, J=25.2, 7.7 Hz, 2H), 2.20 (d, J=10.2 Hz, 2H), 2.08 (qd, J=13.7, 4.4 Hz, 1H), 2.00-1.92 (m, 1H), 1.69 (d, J=10.8 Hz, 2H), 1.44 (dd, J=14.7, 8.4 Hz, 1H), 1.36 (s, 3H), 1.21 (d, J=9.6 Hz, 2H), 1.06 (d, J=13.6 Hz, 1H), 0.94 (d, J=6.2 Hz, 3H), 0.78-0.71 (m, 1H), 0.61 (dd, J=7.8, 3.3 Hz, 1H). [M+H]⁺=902.7.

### Example 28: 3-(5-(4-(3-(5-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-2,5-diazabicyclo[2.2.2]octan-2-yl)azetidin-1-yl)piperidin-1-yl)-4,6-dimethylpyridin-2-yl)piperidine-2,6-dione

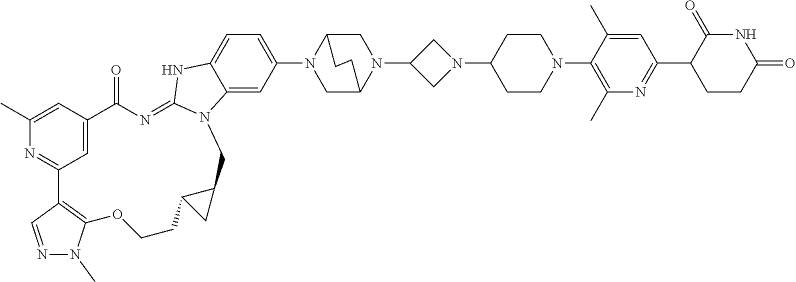 [SMILES: Cc1cc2cc(n1)-c1cnn(C)c1OCC[C@H]1C[C@@H]1CN1/C(=N/C2=O)Nc2ccc(N3CC4CCC3CN4C3CN(C4CCN(c5c(C)cc(C6CCC(=O)NC6=O)nc5C)CC4)C3)cc21]

The title compound was prepared in a manner similar to that in Example 3 with intermediates 18 and intermediate 6.

¹H NMR (500 MHz, DMSO) δ 12.34 (s, 1H), 10.77 (s, 1H), 8.71 (s, 1H), 7.88 (s, 1H), 7.50 (s, 1H), 7.31 (d, J=8.7 Hz, 1H), 6.98-6.91 (m, 1H), 6.72 (s, 1H), 6.60 (d, J=7.8 Hz, 1H), 4.34-4.21 (m, 3H), 4.15-4.09 (m, 1H), 4.02 (s, 1H), 3.87-3.80 (m, 1H), 3.75 (s, 3H), 3.60-3.58 (m, 1H), 3.24-3.22 (m, 1H), 3.11-2.70 (m, 9H), 2.59-2.53 (m, 5H), 2.37 (s, 3H), 2.29-2.22 (m, 3H), 2.21-2.01 (m, 4H), 1.99-1.84 (m, 3H), 1.82-1.57 (m, 3H), 1.50-1.46 (m, 2H), 1.38-1.29 (m, 2H), 1.23 (s, 3H), 1.18-1.11 (m, 1H), 0.57-0.49 (m, 1H), 0.41-0.34 (m, 1H). [M+H]⁺=893.7.

### Example 29: (R)-3-(2,6-difluoro-4-((1-(1-((7¹R,7²S,E)-1¹,2⁶,7²-trimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperidine-4-carbonyl)piperidin-4-yl)oxy)phenyl)piperidine-2,6-dione

### Step 1: 2,6-bis(benzyloxy)-3-(2,6-difluoro-4-(4,4,5,5-tetramethyl-1,3,2-dioxaborolan-2-yl)phenyl)pyridine

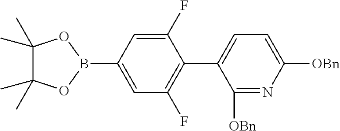 [SMILES: CC1(C)OB(c2cc(F)c(-c3ccc(OCc4ccccc4)nc3OCc3ccccc3)c(F)c2)OC1(C)C]

To a solution of 2,6-bis(benzyloxy)-3-(4-bromo-2,6-difluorophenyl)pyridine (35 g, 72.8 mmol) in dioxane (300 mL) were added B(pin)₂ (37.0 g, 145.5 mmol), Pd(dppf)Cl₂ (5.3 g, 7.3 mmol), and K₂CO₃ (30.1 g, 218.3 mmol). The resulting solution was stirred overnight at 100° C. under N₂ atmosphere. After cooled to room temperature and filtered, the filter cake was washed with EA. The filtrate was concentrated under reduced pressure. The crude product purified by silica column chromatography (EA:PE=0-10%) to afford 2,6-bis(benzyloxy)-3-(2,6-difluoro-4-(4,4,5,5-tetramethyl-1,3,2-dioxaborolan-2-yl)phenyl)pyridine (30 g, 77.94%). [M+H]⁺=530.39

### Step 2: 4-(2,6-bis(benzyloxy)pyridin-3-yl)-3,5-difluorophenol

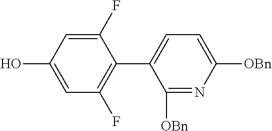 [SMILES: Oc1cc(F)c(-c2ccc(OCc3ccccc3)nc2OCc2ccccc2)c(F)c1]

To a stirred mixture of 2,6-bis(benzyloxy)-3-(2,6-difluoro-4-(4,4,5,5-tetramethyl-1,3,2-dioxaborolan-2-yl)phenyl)pyridine (30 g, 56.7 mmol) in AcOH (100 mL) and THF (100 mL) was added H₂O₂ (100 mL) in portions at 0° C. The mixture was stirred overnight at rt. Then saturated Na₂S₂O₃ aqueous solution was added and then the mixture was extracted with EA. The organic layer was washed with brine, dried over anhydrous Na₂SO₄. After filtration, the filtrate was concentrated under reduced pressure and purified by silica column chromatography (EA:PE=0-30%) to afford 4-(2,6-bis(benzyloxy)pyridin-3-yl)-3,5-difluorophenol (21 g, 88.4%). [M+H]⁺=420.4.

### Step 3: tert-butyl 4-(4-(2,6-bis(benzyloxy)pyridin-3-yl)-3,5-difluorophenoxy)piperidine-1-carboxylate

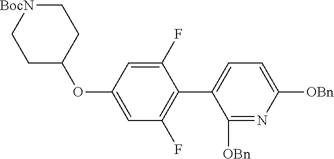 [SMILES: CC(C)(C)OC(=O)N1CCC(Oc2cc(F)c(-c3ccc(OCc4ccccc4)nc3OCc3ccccc3)c(F)c2)CC1]

A 500 mL round bottom flask equipped with a magnetic stirrer were charged with 4-(2,6-bis(benzyloxy)pyridin-3-yl)-3,5-difluorophenol (20.5 g, 48.9 mmol), tert-butyl 4-((methylsulfonyl)oxy)piperidine-1-carboxylate (20.5 g, 73.4 mmol), Cs₂CO₃ (47.8 g, 146.6 mmol), and DMF (210 mL). The resulting mixture was degassed under reduced pressure and purged with N₂ for three times, then stirred at 110° C. for 2 hours. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by column chromatography (EA/PE, 12-16%) to afford tert-butyl 4-(4-(2,6-bis(benzyloxy)pyridin-3-yl)-3,5-difluorophenoxy)piperidine-1-carboxylate (14 g, 23.3 mmol, 47.6%). [M+H]⁺=603.0.

### Step 4: tert-butyl 4-(4-(2,6-dioxopiperidin-3-yl)-3,5-difluorophenoxy)piperidine-1-carboxylate

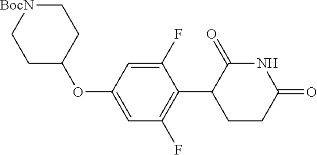 [SMILES: CC(C)(C)OC(=O)N1CCC(Oc2cc(F)c(C3CCC(=O)NC3=O)c(F)c2)CC1]

A 500 mL round bottom flask equipped with a magnetic stirrer, were charged with tert-butyl 4-(4-(2,6-bis(benzyloxy)pyridin-3-yl)-3,5-difluorophenoxy)piperidine-1-carboxylate (14 g, 23.3 mmol), dry THF (240 ml), and Pd/C (10 wt %, 27 g). The resulting mixture was degassed under reduced pressure and purged with H₂ for five times, then stirred overnight at 50° C. The mixture was diluted with THF/DCM/MeOH (200 mL/200 mL/200 mL), then sonicated in an ultrasonic washer for 5 minutes, followed by filtration through a pad of celite. The filtrate was concentrated under vacuum. The residue was purified by column chromatography (EA/PE, 40-50%) to afford tert-butyl 4-(4-(2,6-dioxopiperidin-3-yl)-3,5-difluorophenoxy)piperidine-1-carboxylate (6.9 g, 70.0%). [M+H]⁺=425.

### Step 5: tert-butyl (R)-4-(4-(2,6-dioxopiperidin-3-yl)-3,5-difluorophenoxy)piperidine-1-carboxylate

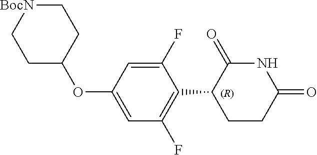

The crude product (6.7 g) was purified by chiral-SFC with the following conditions (column: chiralpak AD-3 3×100 mm, 3 mm, 10% to 50% in 2.0 min, hold 1.0 min at 50%, MeOH (0.1% DEA), retention time 1.612 min) to afford tert-butyl (R)-4-(4-(2,6-dioxopiperidin-3-yl)-3,5-difluorophenoxy)piperidine-1-carboxylate. [M+H]⁺=425.

### Step 6: (R)-3-(2,6-difluoro-4-(piperidin-4-yloxy)phenyl)piperidine-2,6-dione

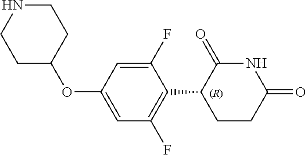

A solution of tert-butyl (R)-4-(4-(2,6-dioxopiperidin-3-yl)-3,5-difluorophenoxy)piperidine-1-carboxylate (500 mg, 1.18 mmol) in DCM (10 mL) was added HCl (4M in dioxane) (3 mL). The mixture was stirred in a flask at room temperature for 2 h. The mixture was evaporated in vacuum to afford the crude product (350 mg, 91.6%), which was used for next step without further purification. [M+H]⁺=325.

### Step 7: tert-butyl (R)-4-(4-(4-(2,6-dioxopiperidin-3-yl)-3,5-difluorophenoxy)piperidine-1-carbonyl)piperidine-1-carboxylate

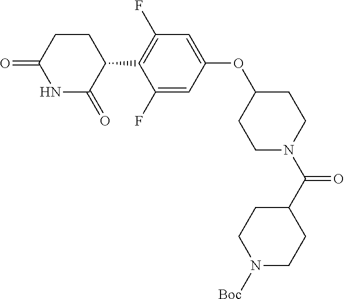 [SMILES: CC(C)(C)OC(=O)N1CCC(C(=O)N2CCC(Oc3cc(F)c([C@H]4CCC(=O)NC4=O)c(F)c3)CC2)CC1]

To a solution of (R)-3-(2,6-difluoro-4-(piperidin-4-yloxy)phenyl)piperidine-2,6-dione (350 mg, 1.08 mmol) in DCM (5 mL) were added 1-(tert-butoxycarbonyl)piperidine-4-carboxylic acid (370 mg, 1.62 mmol), DIEA (278 mg, 2.16 mmol) and T3P (50 wt % in EA) (1.37 g, 682.2 mmol). The resulting solution was stirred for 1 h at rt. The reaction was quenched with water and extracted with DCM. The combined organic layers were washed with brine, dried over anhydrous Na₂SO₄. After filtration, the filtrate was concentrated under reduced pressure. The residue was purified by silica gel column chromatography, eluted with DCM/MeOH (10:1) to afford tert-butyl (R)-4-(4-(4-(2,6-dioxopiperidin-3-yl)-3,5-difluorophenoxy)piperidine-1-carbonyl)piperidine-1-carboxylate (300 mg, 52%). [M+H]⁺=536.2.

### Step 8: (R)-3-(2,6-difluoro-4-((1-(piperidine-4-carbonyl)piperidin-4-yl)oxy)phenyl)piperidine-2,6-dione

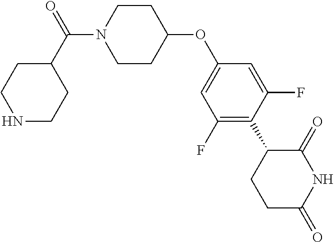 [SMILES: O=C1CC[C@H](c2c(F)cc(OC3CCN(C(=O)C4CCNCC4)CC3)cc2F)C(=O)N1]

To the solution of tert-butyl (R)-4-(4-(4-(2,6-dioxopiperidin-3-yl)-3,5-difluorophenoxy)piperidine-1-carbonyl)piperidine-1-carboxylate (300 mg, 0.56 mmol) in DCM (10 mL) was added HCl (4M in dioxane, 3 mL). The mixture was stirred in a flask at room temperature for 2 h. The mixture was evaporated in vacuum to afford the crude product (200 mg, 81.9%), which was used for next step without further purification. [M+H]⁺=436.2.

### Step 9: (R)-3-(2,6-difluoro-4-((1-(1-((7¹R,7²S,E)-1¹,2⁶,7²-trimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperidine-4-carbonyl)piperidin-4-yl)oxy)phenyl)piperidine-2,6-dione

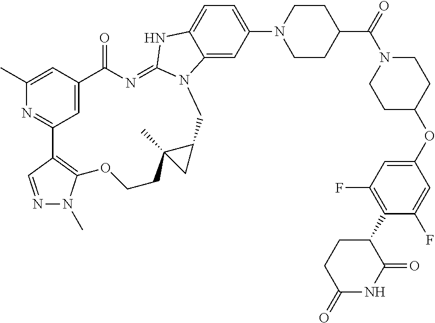 [SMILES: Cc1cc2cc(n1)-c1cnn(C)c1OCC[C@]1(C)C[C@H]1CN1/C(=N/C2=O)Nc2ccc(N3CCC(C(=O)N4CCC(Oc5cc(F)c([C@H]6CCC(=O)NC6=O)c(F)c5)CC4)CC3)cc21]

The titled compound was prepared in a manner similar to that in Example 30.

¹H NMR (500 MHz, DMSO) δ 12.45 (s, 1H), 10.92 (s, 1H), 8.52 (s, 1H), 7.87 (s, 1H), 7.53 (s, 1H), 7.36 (d, J=8.7 Hz, 1H), 7.18 (s, 1H), 6.91 (d, J=8.5 Hz, 1H), 6.84 (d, J=10.8 Hz, 2H), 4.70 (s, 1H), 4.58 (d, J=13.0 Hz, 2H), 4.18-4.08 (m, 2H), 3.89-3.86 (m, 5H), 3.74 (s, 3H), 2.86-2.73 (m, 6H), 2.56 (s, 3H), 2.17-2.07 (m, 2H), 1.97-1.95 (m, 4H), 1.75 (s, 5H), 1.62 (s, 1H), 1.43 (d, J=6.2 Hz, 1H), 1.36 (s, 3H), 1.06 (d, J=12.4 Hz, 1H), 0.75 (s, 1H), 0.61 (d, J=4.9 Hz, 1H). [M+H]⁺=876.0

### Example 30: 3-(5-((3R,4S)-4-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)-3-fluoropiperidin-1-yl)-3-fluoro-6-methylpyridin-2-yl)piperidine-2,6-dione

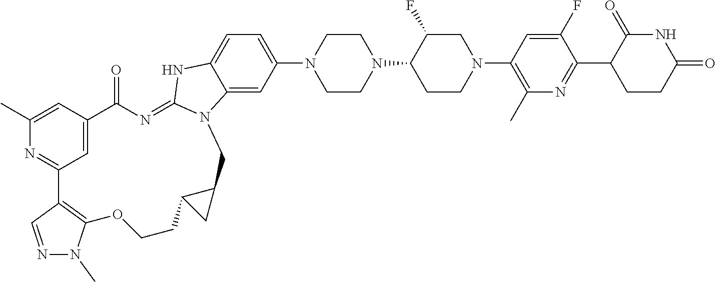 [SMILES: Cc1cc2cc(n1)-c1cnn(C)c1OCC[C@H]1C[C@@H]1CN1/C(=N/C2=O)Nc2ccc(N3CCN([C@H]4CCN(c5cc(F)c(C6CCC(=O)NC6=O)nc5C)C[C@H]4F)CC3)cc21]

To a stirred solution of intermediate 15 (100 mg, 0.2 mmol) in DMA (5 mL) were added t-BuONa (38 mg, 0.4 mmol), Pd₂(dba)₃ (36 mg, 0.04 mmol), Ruphos (37 mg, 0.08 mmol) and intermediate 9 (80 mg, 0.2 mmol) at room temperature under nitrogen atmosphere. The resulting mixture was stirred for 10 min at 100° C. under nitrogen atmosphere. The mixture was allowed to cool down to room temperature. The resulting mixture was concentrated under reduced pressure. The residue was diluted with EtOAc (100 mL), washed with water (3×50 mL) and brine (50 mL). The organic layer was dried over anhydrous Na₂SO₄, filtered and concentrated under reduced pressure. The residue was purified by silica gel column chromatography, eluted with MeOH/DCM (0-5%) to afford the product (23.3 mg, 14.2%).

¹H NMR (500 MHz, DMSO) δ 12.58 (s, 1H), 10.89 (s, 1H), 8.81 (s, 1H), 7.99 (s, 1H), 7.63 (s, 1H), 7.45 (d, J=11.3 Hz, 1H), 7.42 (d, J=8.7 Hz, 1H), 7.23 (s, 1H), 7.00 (d, J=8.6 Hz, 1H), 5.52-5.42 (m, 1H), 4.39-4.24 (m, 5H), 4.19-4.15 (m, 3H), 3.91 (s, 3H), 3.64 (s, 2H), 3.34 (s, 3H), 3.14 (s, 2H), 3.03-2.93 (m, 1H), 2.88-2.83 (m, 1H), 2.76-2.70 (m, 1H), 2.63 (s, 3H), 2.60-2.52 (m, 2H), 2.43 (s, 3H), 2.35-2.22 (m, 2H), 2.13-2.03 (m, 3H), 1.54-1.44 (m, 1H), 1.38-1.33 (m, 1H), 1.19-1.16 (m, 1H), 0.57-0.50 (m, 1H), 0.41-0.38 (m, 1H). [M+H]⁺=834.6.

### Example 31: 3-(5-((3R,4S)-4-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)-3-fluoropiperidin-1-yl)-6-ethylpyridin-2-yl)piperidine-2,6-dione

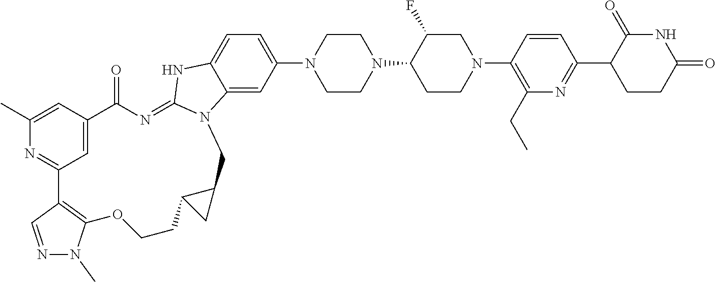 [SMILES: CCc1nc(C2CCC(=O)NC2=O)ccc1N1CC[C@H](N2CCN(c3ccc4c(c3)N3C[C@H]5C[C@@H]5CCOc5c(cnn5C)-c5cc(cc(C)n5)C(=O)/N=C/3N4)CC2)[C@H](F)C1]

The title compound was prepared in a manner similar to that in Example 30.

¹H NMR (500 MHz, DMSO) δ 12.44 (s, 1H), 10.80 (s, 1H), 8.71 (s, 1H), 7.88 (s, 1H), 7.51 (s, 1H), 7.43 (d, J=8.1 Hz, 1H), 7.35 (t, J=7.0 Hz, 1H), 7.14 (d, J=8.1 Hz, 1H), 7.06 (d, J=11.0 Hz, 1H), 6.94-6.89 (m, 1H), 5.17-5.03 (m, 1H), 4.37-4.21 (m, 3H), 4.11 (t, J=6.6 Hz, 1H), 3.92 (dd, J=7.9, 5.6 Hz, 1H), 3.75 (s, 3H), 3.22-3.12 (m, 5H), 2.91-2.71 (m, 8H), 2.65-2.54 (m, 8H), 2.24-2.02 (m, 4H), 1.82 (d, J=12.4 Hz, 1H), 1.49-1.41 (m, 1H), 1.34 (d, J=4.5 Hz, 1H), 1.20-1.15 (m, 3H), 0.57-0.51 (m, 1H), 0.42-0.35 (m, 1H). [M+H]⁺=830.6.

### Example 32: 3-(5-((3S,4R)-4-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)-3-fluoropiperidin-1-yl)-6-ethylpyridin-2-yl)piperidine-2,6-dione

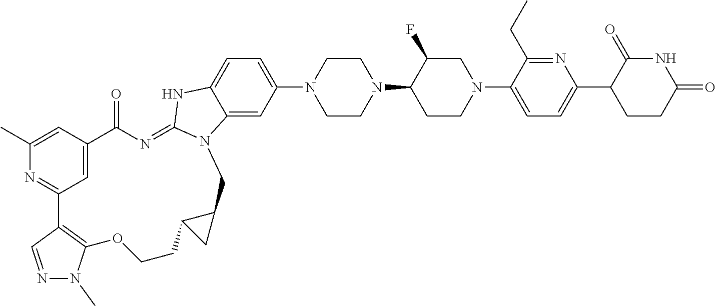 [SMILES: CCc1nc(C2CCC(=O)NC2=O)ccc1N1CC[C@@H](N2CCN(c3ccc4c(c3)N3C[C@H]5C[C@@H]5CCOc5c(cnn5C)-c5cc(cc(C)n5)C(=O)/N=C/3N4)CC2)[C@@H](F)C1]

The title compound was prepared in a manner similar to that in Example 30.

¹H NMR (500 MHz, DMSO) δ 12.60 (s, 1H), 10.84 (s, 1H), 8.85 (s, 1H), 8.04 (s, 1H), 7.68 (s, 1H), 7.54 (d, J=8.2 Hz, 1H), 7.43 (d, J=8.7 Hz, 1H), 7.23 (s, 1H), 7.21 (s, 1H), 7.01 (dd, J=8.8, 1.7 Hz, 1H), 5.51-5.42 (m, 1H), 4.40-4.26 (m, 3H), 4.18 (t, J=7.1 Hz, 1H), 3.97 (dd, J=8.3, 5.4 Hz, 2H), 3.78 (s, 3H), 3.50-3.31 (m, 7H), 3.24 (d, J=10.9 Hz, 1H), 3.14 (s, 1H), 3.07-2.72 (m, 5H), 2.65 (s, 3H), 2.59 (t, J=6.6 Hz, 2H), 2.30 (d, J=10.8 Hz, 1H), 2.26-2.03 (m, 4H), 1.56-1.45 (m, 1H), 1.35 (qd, J=9.4, 4.9 Hz, 1H), 1.25-1.14 (m, 4H), 0.53 (dt, J=8.5, 4.3 Hz, 1H), 0.39 (dt, J=9.1, 4.7 Hz, 1H). [M+H]⁺=830.6.

### Example 33: 3-(5-((3S,4R)-4-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)-3-fluoropiperidin-1-yl)-4,6-dimethylpyridin-2-yl)piperidine-2,6-dione

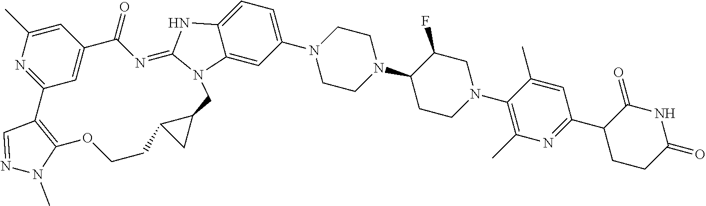 [SMILES: Cc1cc2cc(n1)-c1cnn(C)c1OCC[C@H]1C[C@@H]1CN1/C(=N/C2=O)Nc2ccc(N3CCN([C@@H]4CCN(c5c(C)cc(C6CCC(=O)NC6=O)nc5C)C[C@@H]4F)CC3)cc21]

The title compound was prepared in a manner similar to that in Example 30.

¹H NMR (500 MHz, DMSO) δ 10.72 (s, 1H), 8.64 (s, 1H), 7.81 (s, 1H), 7.44 (s, 1H), 7.29 (d, J=8.8 Hz, 1H), 7.00-6.96 (m, 2H), 6.86-6.84 (m, 2H), 4.37-4.12 (m, 4H), 4.06-4.04 (m, 1H), 3.81-3.76 (m, 2H), 3.68 (s, 3H), 3.14-2.96 (m, 6H), 2.94-2.89 (m, 2H), 2.77 (s, 5H), 2.58-2.52 (m, 3H), 2.30 (d, J=5.3 Hz, 2H), 2.22 (d, J=13.9 Hz, 4H), 2.12 (s, 1H), 2.08-1.90 (m, 3H), 1.67 (s, 1H), 1.40 (s, 1H), 1.28 (s, 1H), 1.11-1.06 (m, 2H), 0.48 (s, 1H), 0.32 (s, 1H). [M+H]⁺=830.6

### Example 34: 3-(5-((3S,4R)-4-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)-3-fluoropiperidin-1-yl)-3-fluoro-6-methylpyridin-2-yl)piperidine-2,6-dione

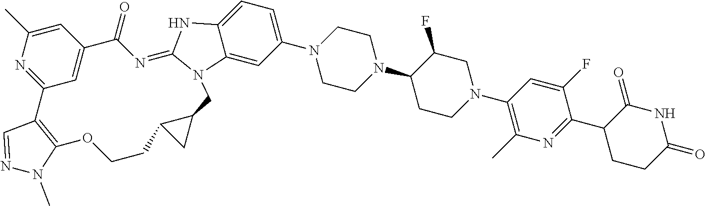 [SMILES: Cc1cc2cc(n1)-c1cnn(C)c1OCC[C@H]1C[C@@H]1CN1/C(=N/C2=O)Nc2ccc(N3CCN([C@@H]4CCN(c5cc(F)c(C6CCC(=O)NC6=O)nc5C)C[C@@H]4F)CC3)cc21]

The title compound was prepared in a procedure similar to that in example 30.

¹H NMR (500 MHz, DMSO) δ 12.45 (s, 1H), 10.87 (s, 1H), 8.71 (s, 1H), 7.88 (s, 1H), 7.51 (s, 1H), 7.36-7.32 (m, 2H), 7.06 (s, 1H), 6.91 (d, J=8.6 Hz, 1H), 5.16-5.06 (m, 1H), 4.29-4.27 (m, 3H), 4.21-4.00 (m, 2H), 3.75 (s, 3H), 3.43 (s, 2H), 3.23-3.18 (m, 4H), 2.96-2.77 (m, 6H), 2.75-2.68 (m, 1H), 2.57 (d, J=12.5 Hz, 5H), 2.40 (s, 2H), 2.26 (d, J=9.6 Hz, 1H), 2.12-2.06 (m, 3H), 1.84 (s, 1H), 1.46-1.35 (m, 2H), 1.23-1.08 (m, 2H), 0.54 (s, 1H), 0.38 (s, 1H). [M+H]⁺=834.6

### Example 35: (3R)-3-(4-(2-(4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-2-(tetrahydrofuran-3-yl)piperazin-1-yl)ethyl)-2,6-difluorophenyl)piperidine-2,6-dione

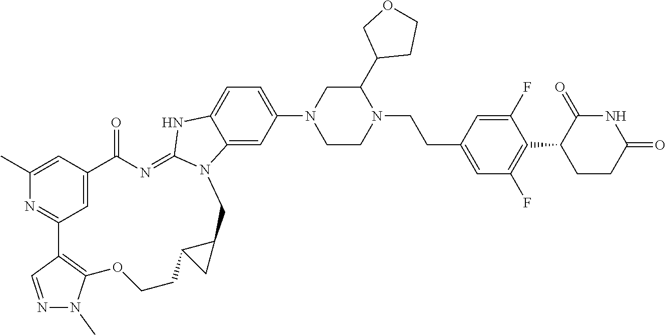 [SMILES: Cc1cc2cc(n1)-c1cnn(C)c1OCC[C@H]1C[C@@H]1CN1/C(=N/C2=O)Nc2ccc(N3CCN(CCc4cc(F)c([C@H]5CCC(=O)NC5=O)c(F)c4)C(C4CCOC4)C3)cc21]

The titled compound was prepared in a manner similar to that in Example 3 with intermediates 16 and intermediate 27.

¹H NMR (500 MHz, DMSO) δ 10.96 (s, 1H), 8.71 (s, 1H), 8.31 (s, 1H), 7.88 (s, 1H), 7.50 (s, 1H), 7.36 (d, J=6.4 Hz, 1H), 7.07 (d, J=3.5 Hz, 1H), 7.05 (d, J=3.3 Hz, 1H), 6.90 (d, J=8.6 Hz, 1H), 4.35-4.27 (m, 2H), 4.22-4.18 (m, 2H), 3.75 (s, 4H), 3.65-3.57 (m, 1H), 3.51-3.47 (m, 3H), 3.23 (s, 2H), 3.20-3.18 (m, 2H), 2.99-2.93 (m, 2H), 2.85-2.78 (m, 5H), 2.64 (s, 2H), 2.59-2.51 (m, 5H), 2.15-2.09 (m, 2H), 2.02-1.97 (m, 1H), 1.92 (s, 1H), 1.75-1.71 (m, 1H), 1.55-1.38 (m, 1H), 1.21-1.05 (m, 2H), 0.39 (s, 1H); [M+H]⁺=834.6

### Example 36: 3-(5-(4-(3-((S)-4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-3-methylpiperazin-1-yl)azetidin-1-yl)piperidin-1-yl)-6-methylpyridin-2-yl)piperidine-2,6-dione

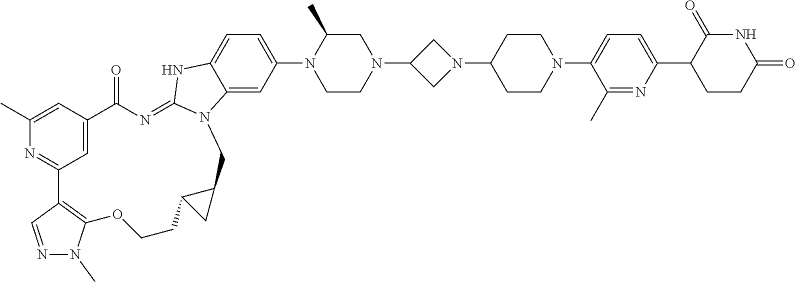 [SMILES: Cc1cc2cc(n1)-c1cnn(C)c1OCC[C@H]1C[C@@H]1CN1/C(=N/C2=O)Nc2ccc(N3CCN(C4CN(C5CCN(c6ccc(C7CCC(=O)NC7=O)nc6C)CC5)C4)C[C@@H]3C)cc21]

The titled compound was prepared in a manner similar to that in Example 27.

¹H NMR (500 MHz, DMSO) δ 12.44 (s, 1H), 10.78 (s, 1H), 8.71 (s, 1H), 7.88 (s, 1H), 7.50 (s, 1H), 7.37-7.35 (m, 2H), 7.16-6.98 (m, 2H), 6.88 (d, J=8.6 Hz, 1H), 4.33-4.24 (m, 3H), 4.13-4.10 (m, 1H), 3.90-3.87 (m, 2H), 3.75 (s, 3H), 3.43 (s, 2H), 3.21 (d, J=11.7 Hz, 1H), 3.04-2.98 (m, 3H), 2.90-2.80 (m, 3H), 2.69-2.59 (m, 3H), 2.55 (s, 3H), 2.54 (s, 2H), 2.45-2.43 (m, 1H), 2.37 (s, 3H), 2.33 (d, J=8.4 Hz, 1H), 2.24-2.14 (m, 3H), 2.12-2.04 (m, 2H), 1.76 (s, 2H), 1.51-1.40 (m, 1H), 1.35 (d, J=8.7 Hz, 3H), 1.20-1.06 (m, 1H), 0.97 (d, J=6.2 Hz, 3H), 0.56-0.47 (m, 1H), 0.43-0.29 (m, 1H). [M+H]⁺=867.7

### Example 37: 3-(5-(4-(3-((R)-4-((7¹S,7²R,E)-1¹,2⁶-dimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)-3-methylpiperazin-1-yl)azetidin-1-yl)piperidin-1-yl)-6-methylpyridin-2-yl)piperidine-2,6-dione

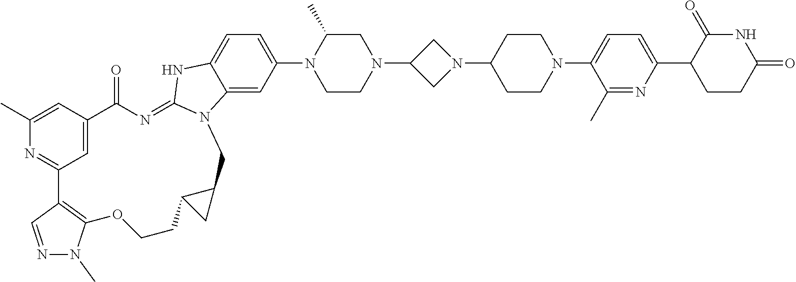 [SMILES: Cc1cc2cc(n1)-c1cnn(C)c1OCC[C@H]1C[C@@H]1CN1/C(=N/C2=O)Nc2ccc(N3CCN(C4CN(C5CCN(c6ccc(C7CCC(=O)NC7=O)nc6C)CC5)C4)C[C@H]3C)cc21]

The titled compound was prepared in a manner similar to that in Example 27.

¹H NMR (500 MHz, DMSO) δ 12.44 (s, 1H), 10.78 (s, 1H), 8.71 (s, 1H), 7.88 (s, 1H), 7.50 (s, 1H), 7.37-7.35 (m, 2H), 7.16-6.98 (m, 2H), 6.88 (d, J=8.6 Hz, 1H), 4.33-4.24 (m, 3H), 4.13-4.10 (m, 1H), 3.90-3.87 (m, 2H), 3.75 (s, 3H), 3.43 (s, 2H), 3.21 (d, J=11.7 Hz, 1H), 3.04-2.98 (m, 3H), 2.90-2.80 (m, 3H), 2.69-2.59 (m, 3H), 2.55 (s, 3H), 2.54 (s, 2H), 2.45-2.43 (m, 1H), 2.37 (s, 3H), 2.33 (d, J=8.4 Hz, 1H), 2.24-2.14 (m, 3H), 2.12-2.04 (m, 2H), 1.76 (s, 2H), 1.51-1.40 (m, 1H), 1.35 (d, J=8.7 Hz, 3H), 1.20-1.06 (m, 1H), 0.97 (d, J=6.2 Hz, 3H), 0.56-0.47 (m, 1H), 0.43-0.29 (m, 1H). [M+H]⁺=867.7

### Example 43: 3-(3-fluoro-5-((3S,4R)-3-fluoro-4-(4-((7¹R,7²S,E)-1¹,2⁶,7²-trimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)piperidin-1-yl)-6-methylpyridin-2-yl)piperidine-2,6-dione

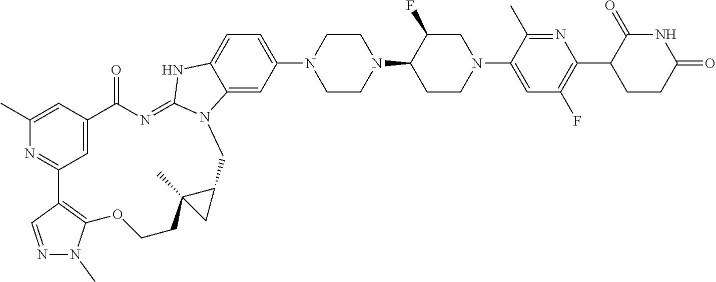 [SMILES: Cc1cc2cc(n1)-c1cnn(C)c1OCC[C@]1(C)C[C@H]1CN1/C(=N/C2=O)Nc2ccc(N3CCN([C@@H]4CCN(c5cc(F)c(C6CCC(=O)NC6=O)nc5C)C[C@@H]4F)CC3)cc21]

The titled compound was prepared in a manner similar to that in Example 30 with intermediates 14 and 10.

¹H NMR (500 MHz, DMSO) δ 12.54 (s, 1H), 10.82 (s, 1H), 8.53 (s, 1H), 7.91 (s, 1H), 7.54 (s, 1H), 7.42 (s, 2H), 7.29 (s, 1H), 6.97 (s, 1H), 5.57-5.47 (m, 1H), 4.68-4.52 (m, 2H), 4.16 (dd, J=11.2, 5.1 Hz, 1H), 4.11 (d, J=6.5 Hz, 1H), 3.97-3.84 (m, 2H), 3.81-3.69 (m, 5H), 3.62-3.58 (m, 1H), 3.37 (s, 2H), 3.21 (s, 2H), 3.02-2.82 (m, 3H), 2.76-2.68 (m, 1H), 2.62-2.53 (m, 6H), 2.42 (s, 3H), 2.34-2.14 (m, 2H), 2.10-2.00 (m, 1H), 1.46 (d, J=5.7 Hz, 1H), 1.37 (s, 3H), 1.15 (d, J=5.8 Hz, 1H), 1.10-1.04 (m, 1H), 0.95 (dd, J=15.1, 5.9 Hz, 1H), 0.76 (s, 1H), 0.61 (d, J=4.6 Hz, 1H). [M+H]⁺=848.7

### Example 49: 3-(5-((3S,4R)-3-fluoro-4-(4-((7¹R,7²S,E)-1¹,2⁶,7²-trimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)piperidin-1-yl)-6-methylpyridin-2-yl)piperidine-2,6-dione

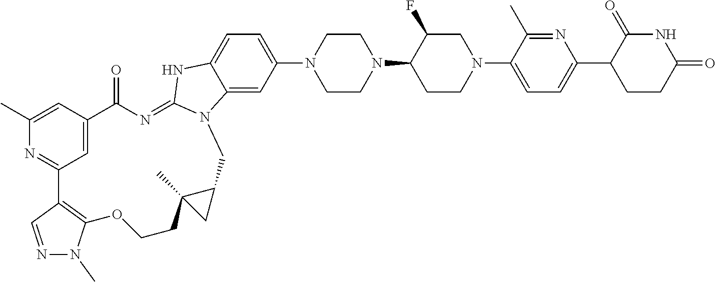 [SMILES: Cc1cc2cc(n1)-c1cnn(C)c1OCC[C@]1(C)C[C@H]1CN1/C(=N/C2=O)Nc2ccc(N3CCN([C@@H]4CCN(c5ccc(C6CCC(=O)NC6=O)nc5C)C[C@@H]4F)CC3)cc21]

The titled compound was prepared in a manner similar to that in Example 30 with intermediates 14 and 29.

¹H NMR (500 MHz, DMSO) δ 12.46 (s, 1H), 10.79 (s, 1H), 8.52 (s, 1H), 7.87 (s, 1H), 7.53 (s, 1H), 7.39 (d, J=6.9 Hz, 1H), 7.37 (s, 1H), 7.20 (d, J=1.7 Hz, 1H), 7.12 (d, J=8.2 Hz, 1H), 6.91 (dd, J=8.8, 1.9 Hz, 1H), 5.16-5.06 (m, 1H), 4.58 (d, J=13.5 Hz, 2H), 4.10 (t, J=6.8 Hz, 1H), 3.94-3.84 (m, 2H), 3.74 (s, 3H), 3.25-3.08 (m, 6H), 2.92-2.75 (m, 6H), 2.60-2.53 (m, 7H), 2.42 (s, 3H), 2.26-2.16 (m, 1H), 2.14-2.01 (m, 2H), 1.84 (d, J=9.7 Hz, 1H), 1.44 (dd, J=14.6, 8.5 Hz, 1H), 1.37 (s, 3H), 1.06 (d, J=15.3 Hz, 1H), 0.78-0.71 (m, 1H), 0.61 (dd, J=8.0, 3.6 Hz, 1H). [M+H]⁺=830.7.

### Example 54: 3-(3-fluoro-6-methyl-5-(4-(4-((7¹R,7²S,E)-1¹,2⁶,7²-trimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)piperidin-1-yl)pyridin-2-yl)piperidine-2,6-dione

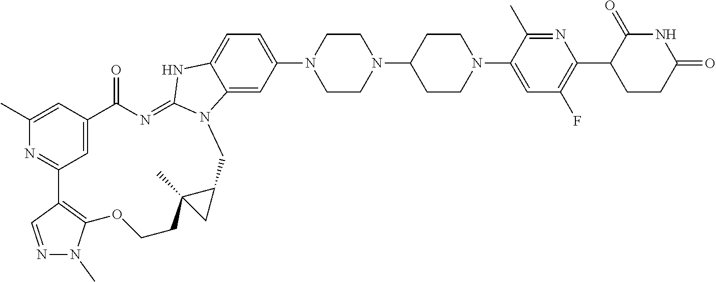 [SMILES: Cc1cc2cc(n1)-c1cnn(C)c1OCC[C@]1(C)C[C@H]1CN1/C(=N/C2=O)Nc2ccc(N3CCN(C4CCN(c5cc(F)c(C6CCC(=O)NC6=O)nc5C)CC4)CC3)cc21]

The titled compound was prepared in a manner similar to that in Example 3 with intermediates 30 and 5.

¹H NMR (500 MHz, DMSO) δ 12.47 (s, 1H), 10.87 (s, 1H), 8.52 (s, 1H), 7.87 (s, 1H), 7.53 (s, 1H), 7.37 (d, J=8.7 Hz, 1H), 7.32 (d, J=11.5 Hz, 1H), 7.19 (s, 1H), 6.90 (dd, J=8.8, 1.7 Hz, 1H), 4.58 (d, J=13.5 Hz, 2H), 4.14 (dd, J=11.1, 5.2 Hz, 1H), 4.10 (t, J=6.8 Hz, 1H), 3.91 (dd, J=13.8, 9.0 Hz, 1H), 3.74 (s, 3H), 3.18 (d, J=4.3 Hz, 6H), 2.79-2.61 (m, 7H), 2.59-2.53 (m, 5H), 2.43-2.34 (m, 4H), 2.30-2.18 (m, 1H), 2.08-2.00 (m, 1H), 1.94 (d, J=10.0 Hz, 2H), 1.64 (d, J=9.9 Hz, 2H), 1.44 (dd, J=14.5, 7.9 Hz, 1H), 1.37 (s, 3H), 1.11-1.01 (m, 1H), 0.78-0.73 (m, 1H), 0.61 (dd, J=8.1, 3.6 Hz, 1H). [M+H]⁺=830.7.

### Example 57: 3-(4-((3S,4R)-3-fluoro-4-(4-((7¹R,7²S,E)-1¹,2⁶,7²-trimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)piperidin-1-yl)-3,3-dimethyl-2-oxoindolin-1-yl)piperidine-2,6-dione

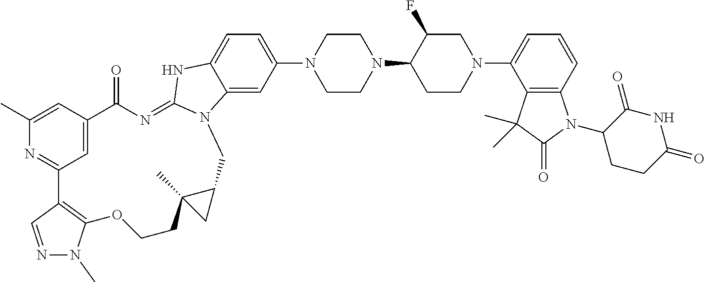 [SMILES: Cc1cc2cc(n1)-c1cnn(C)c1OCC[C@]1(C)C[C@H]1CN1/C(=N/C2=O)Nc2ccc(N3CCN([C@@H]4CCN(c5cccc6c5C(C)(C)C(=O)N6C5CCC(=O)NC5=O)C[C@@H]4F)CC3)cc21]

The title compound was prepared in a manner similar to that in Example 30 with intermediates 14 and 33.

¹H NMR (500 MHz, DMSO) δ 12.44 (s, 1H), 11.06 (s, 1H), 8.52 (s, 1H), 7.87 (s, 1H), 7.53 (s, 1H), 7.38 (d, J=8.7 Hz, 1H), 7.25 (t, J=7.8 Hz, 1H), 7.20 (s, 1H), 7.07 (d, J=8.0 Hz, 1H), 6.91 (d, J=9.2 Hz, 1H), 6.83 (s, 1H), 5.30-5.18 (m, 1H), 5.15-5.05 (m, 1H), 4.59-4.52 (m, 2H), 4.10 (t, J=7.4 Hz, 1H), 3.92 (d, J=7.3 Hz, 1H), 3.74 (s, 3H), 3.25-3.04 (m, 6H), 3.02-2.76 (m, 8H), 2.65-2.60 (m, 2H), 2.56 (s, 4H), 2.06-2.02 (m, 1H), 1.97-1.94 (m, 1H), 1.88-1.78 (m, 1H), 1.43 (dd, J=8.2, 3.8 Hz, 7H), 1.37 (s, 3H), 1.12-1.01 (m, 1H), 0.76 (t, J=4.2 Hz, 1H), 0.65-0.57 (m, 1H); [M+H]⁺=898.7.

### Example 59: 3-(5-((3S,4R)-3-fluoro-4-(4-((7¹R,7²S,E)-1¹,2⁶,7²-trimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)piperidin-1-yl)-4-methylpyridin-2-yl)piperidine-2,6-dione

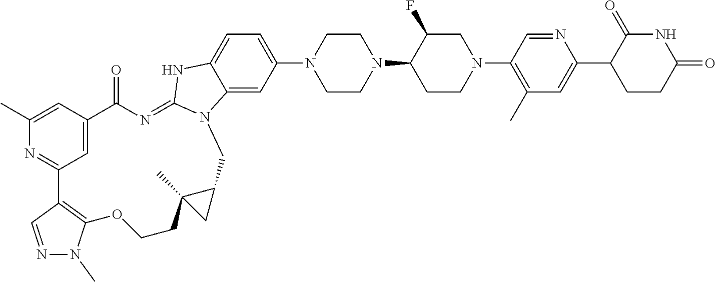 [SMILES: Cc1cc2cc(n1)-c1cnn(C)c1OCC[C@]1(C)C[C@H]1CN1/C(=N/C2=O)Nc2ccc(N3CCN([C@@H]4CCN(c5cnc(C6CCC(=O)NC6=O)cc5C)C[C@@H]4F)CC3)cc21]

The title compound was prepared in a manner similar to that in Example 30 with intermediates 14 and 34.

¹H NMR (500 MHz, DMSO) δ 12.60 (s, 1H), 10.88 (s, 1H), 8.63 (s, 1H), 8.25 (s, 1H), 7.98 (s, 1H), 7.66 (s, 1H), 7.45 (d, J=8.7 Hz, 1H), 7.31 (s, 1H), 7.28 (s, 1H), 7.01 (d, J=8.3 Hz, 1H), 5.57-5.37 (m, 1H), 4.68-4.53 (m, 2H), 4.13 (t, J=6.0 Hz, 1H), 3.98 (dd, J=9.2, 5.4 Hz, 1H), 3.95-3.90 (m, 1H), 3.77 (s, 3H), 3.73-3.70 (m, 1H), 3.67-3.61 (m, 2H), 3.33-3.21 (m, 6H), 3.10 (dd, J=14.1, 5.6 Hz, 1H), 3.05-3.00 (m, 1H), 2.99-2.91 (m, 1H), 2.67-2.53 (m, 7H), 2.38-2.28 (m, 4H), 2.27-2.19 (m, 1H), 2.18-2.07 (m, 2H), 1.46 (dd, J=13.9, 8.0 Hz, 1H), 1.38 (s, 3H), 1.08 (d, J=14.0 Hz, 1H), 0.75 (t, J=3.9 Hz, 1H), 0.62 (dd, J=7.4, 3.1 Hz, 1H). [M+H]⁺=830.7.

### Example 61: 3-(3-fluoro-5-((3R,4S)-3-fluoro-4-(4-((7¹R,7²S,E)-1¹,2⁶,7²-trimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-56-yl)piperazin-1-yl)piperidin-1-yl)-6-methylpyridin-2-yl)piperidine-2,6-dione

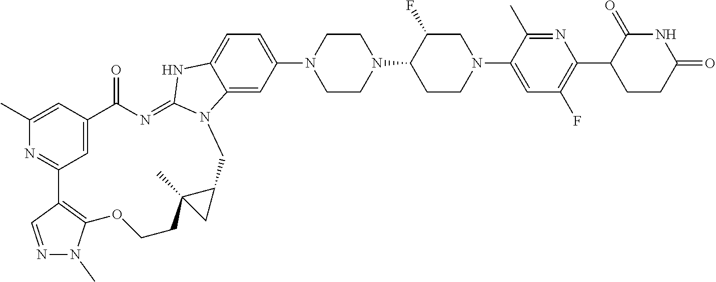 [SMILES: Cc1cc2cc(n1)-c1cnn(C)c1OCC[C@]1(C)C[C@H]1CN1/C(=N/C2=O)Nc2ccc(N3CCN([C@H]4CCN(c5cc(F)c(C6CCC(=O)NC6=O)nc5C)C[C@H]4F)CC3)cc21]

The title compound was prepared in a manner similar to that in Example 30 with intermediates 14 and 9.

¹H NMR (500 MHz, DMSO) δ 12.46 (s, 1H), 10.87 (s, 1H), 8.52 (s, 1H), 7.87 (s, 1H), 7.53 (s, 1H), 7.36 (dd, J=14.3, 10.1 Hz, 2H), 7.20 (s, 1H), 6.91 (d, J=8.9 Hz, 1H), 5.18-5.04 (m, 1H), 4.58 (d, J=13.6 Hz, 2H), 4.18-4.06 (m, 2H), 3.91 (dd, J=13.6, 8.9 Hz, 1H), 3.74 (s, 3H), 3.25-3.16 (m, 5H), 2.94-2.79 (m, 6H), 2.75-2.54 (m, 8H), 2.40 (s, 3H), 2.26 (dd, J=20.4, 11.8 Hz, 1H), 2.05 (dd, J=11.6, 7.1 Hz, 2H), 1.84 (d, J=11.0 Hz, 1H), 1.48-1.41 (m, 1H), 1.37 (s, 3H), 1.06 (d, J=14.4 Hz, 1H), 0.76 (t, J=4.4 Hz, 1H), 0.64-0.57 (m, 1H). [M+H]⁺=848.6.

### Example 62: 3-(5-((3R,4S)-3-fluoro-4-(4-((7¹R,7²S,E)-1¹,2⁶,7²-trimethyl-3-oxo-5²,5³-dihydro-1¹H,5¹H-10-oxa-4-aza-5 (2,1)-benzo[d]imidazola-2 (2,4)-pyridina-1 (4,5)-pyrazola-7 (1,2)-cyclopropanacyclodecaphane-5⁶-yl)piperazin-1-yl)piperidin-1-yl)-6-methylpyridin-2-yl)piperidine-2,6-dione

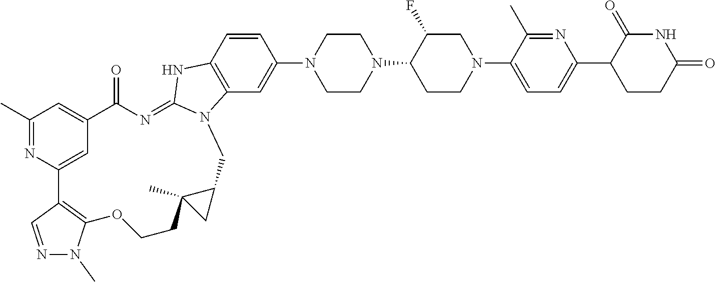 [SMILES: Cc1cc2cc(n1)-c1cnn(C)c1OCC[C@]1(C)C[C@H]1CN1/C(=N/C2=O)Nc2ccc(N3CCN([C@H]4CCN(c5ccc(C6CCC(=O)NC6=O)nc5C)C[C@H]4F)CC3)cc21]

The title compound was prepared in a manner similar to that in Example 30 with intermediates 14 and 31.

¹H NMR (500 MHz, DMSO) δ 12.45 (s, 1H), 10.79 (s, 1H), 8.52 (s, 1H), 7.87 (s, 1H), 7.53 (s, 1H), 7.38 (dd, J=8.4, 4.8 Hz, 2H), 7.20 (s, 1H), 7.12 (d, J=8.1 Hz, 1H), 6.91 (d, J=8.7 Hz, 1H), 5.16-5.05 (m, 1H), 4.61-4.56 (m, 2H), 4.10 (t, J=6.9 Hz, 1H), 3.93-3.87 (m, 2H), 3.74 (s, 3H), 3.39-3.36 (m, 1H), 3.19 (s, 5H), 2.88-2.76 (m, 6H), 2.63-2.57 (m, 2H), 2.56-2.55 (m, 4H), 2.54-2.51 (m, 1H), 2.42 (s, 3H), 2.25-2.17 (m, 1H), 2.11-2.01 (m, 2H), 1.85-1.83 (m, 1H), 1.44 (d, J=6.7 Hz, 1H), 1.37 (s, 3H), 1.06 (d, J=14.8 Hz, 1H), 0.77 (d, J=4.6 Hz, 1H), 0.63-0.58 (m, 1H); [M+H]⁺=830.5

### Cell Line

H1975-clone #8 (L858R/C797S): EGFR-L858R/C797S were stably expressed in H1975 cell lines by lentivirus-mediated over-expression, respectively. The EGFR over-expressed cells then underwent knockout, in which the EGFR targeting sgRNA was designed to only target the endogenous EGFR copies and preserve the exogenous EGFR copies. Followed by the knockout, the edited H1975 cells were seeded in 96 well plates at the concentration of 1 cell/cell, cultured for about 2 weeks to allow single clones formation. The formed clones were screened by DNA sequencing and whole exon sequencing analysis for the desired edition. H1975-clone #8 were finally confirmed as homozygous L858R/C₇₉₇S EGFR clones.

BaF3-L858R (abbv. L858R) cell were purchased from Kangyuan Bochuang Biotechnology (Beijing) Co., Ltd.

### Cell Degradation

### Cell Treatment

On day 1, H1975-clone #8 (L858R/C797S) cells are seeded at 5000 cells/well in cell culture medium [RPMI1640 (Gibco, Cat #72400-047), 10% heat-inactive FBS, 1% PS(Gibco, Cat #10378)] in Corning 96 well plate (Cat #3599).

On day 2, BaF3-L858R cells are seeded at 50000 cells/well at a volume of 54 μl/well in cell culture medium [RPMI1640 (Gibco, phenol red free, Cat #11835-030), 10% heat-inactive FBS, 1% PS(Gibco, Cat #10378)] in Corning 96 well plate (Cat #3799).

H1975-#8 and BaF3-L858R cells are treated with compounds diluted in 0.1% DMSO cell culture medium on day 2, incubate for 16 h, 37° C., 5% CO₂. the final concentration of compounds in all assay is start with 10 uM, 5-fold dilution, total 8 doses were included.

### HTRF Assay

After 16 h treatment, for H1975-#8 cells, add 100 ul HTRF 1× lysis buffer to each well; for BaF3-L858R cells, add 20 μl 4× lysis buffer to each well; seal the plate and incubate 1 hour at room temperature on a plate shaker; Once the cells are lysed, 16 μL of cell lysate are transferred to a PE 384-well HTRF detection plate; 4 μL of pre-mixed HTRF antibodies are added to each well; Cover the plate with a plate sealer, spin 1000 rpm for 1 min, Incubate overnight at room temperature; Read on BMG PheraStar with HTRF protocol (337 nm-665 nm-620 nm).

The inhibition (degradation) percentage of the compound was calculated by the following equation:

$$Inhibition percentage of Compound = 100 - 100 \times ( Signal - low control ) / ( High control - low control ) , wherein signal = each test compound group$$

Low control=only lysis buffer without cells, indicating that EGFR is completely degraded;

High control=Cell group with added DMSO and without compound, indicating microplate readings without EGFR degradation;

Dmax is the maximum percentage of inhibition (degradation).

The IC₅₀(DC₅₀) value of a compound can be obtained by fitting the following equation

$$Y = Bottom + ( TOP - Bottom ) / ( 1 + ( ( {IC}_{50} / X ) \hat{} hillslope ) )$$

Wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the ability the inhibitory ability of the compound is; Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 1
Degradation (BaF3-L858R and H1975-clone#8 (L858R/C797S))
result for Examples

| | BaF3-L858R | | H1975-clone#8 (L858R/C797S) | |
|---|---|---|---|---|
| Example No | DC50 (nM) | Dmax (%) | DC50 (nM) | Dmax (%) |
| 1 | 15.69 | 68.38 | | |
| 3 | 11.57 | 85.48 | 3.1 | 65.24 |
| 4 | 52.48 | 63.63 | | |
| 5 | 72.16 | 70.33 | | |
| 6 | 17.08 | 82.18 | | |
| 9 | 85.45 | 74.92 | | |
| 10 | 6.97 | 91.3 | >10000 | 31.17 |
| 11 | 7.62 | 71.22 | 1.44 | 51.82 |
| 12 | 18.44 | 73.61 | 16.05 | 54.37 |
| 13 | 8.09 | 82.22 | | |
| 14 | 9.37 | 84.41 | 5.8 | 51.39 |
| 15 | 4.48 | 82.49 | 4.96 | 46.15 |
| 16 | 17.98 | 76.37 | 7.82 | 73.26 |
| 17 | 2.32 | 84.14 | 1.6 | 68.72 |
| 18 | 6.69 | 84.61 | 5.12 | 66.32 |
| 19 | 1.93 | 94.52 | 3 | 74.98 |
| 20 | 12.44 | 86.85 | 0.603 | 59.1 |
| 21 | 1.65 | 82.8 | 19.25 | 40.24 |
| 22 | 3.97 | 83.23 | 2.83 | 67.87 |
| 24 | 3.66 | 80 | 2.08 | 49.84 |
| 25 | 2.42 | 91.52 | 1.65 | 57.01 |
| 26 | 4.02 | 85.67 | 3.41 | 58.9 |
| 27 | 1.25 | 79.06 | 1.31 | 63.65 |
| 28 | 6.31 | 78.24 | 2.71 | 48.95 |
| 29 | 3.9 | 67.78 | 0.986 | 49.17 |
| 30 | 20.99 | 89.28 | 9.57 | 57.53 |
| 31 | 4.61 | 89.53 | 9.56 | 69.27 |
| 32 | 6.44 | 84.45 | 10.51 | 60.87 |
| 33 | 8.38 | 89.08 | 4.74 | 44.3 |
| 34 | 7.47 | 83.76 | 11.87 | 57.03 |
| 35 | 21.96 | 59.02 | 8.44 | 41.41 |
| 36 | 13.36 | 68.46 | 2.61 | 41.74 |
| 37 | 7.92 | 64.55 | 8.34 | 55.94 |
| 43 | 1.74 | 86.24 | 2.81 | 88.8 |
| 49 | 0.418 | 86.69 | 0.942 | 86.9 |
| 54 | 3.35 | 91.93 | 2.11 | 77.7 |
| 57 | 3.2 | 91.28 | 4.21 | 61.69 |
| 59 | 3.54 | 95.47 | 0.635 | 72.71 |
| 61 | 0.99 | 91.81 | 1.29 | 80.81 |
| 62 | 0.69 | 93.85 | 0.612 | 76.75 |

1-32. (canceled)

33. A compound of Formula (I): 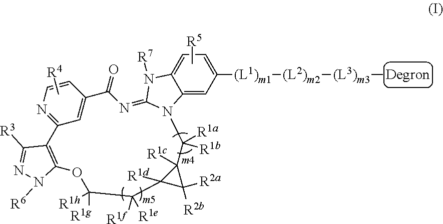
or a N-oxide thereof, or a pharmaceutically acceptable salt thereof, or a stereoisomer thereof, or a deuterated analog thereof, or a prodrug thereof,
wherein:
R^{1a}, R^{1b}, R^{1c}, R^{1d}, R^{1e}, R^{1f}, R^{1g}, R^{1h}, R^{2a}, R², R³, R⁴ and R⁵ are each independently hydrogen, halogen, —C₁₋₄alkyl, —C₁₋₄alkoxy, —C₃₋₆cycloalkyl or —CN; each said —C₁₋₄alkyl, —C₁₋₄alkoxy, or —C₃₋₆cycloalkyl is optionally substituted with at least one substituent selected from hydrogen, halogen, —C₁₋₈alkoxy, —C₃₋₈cycloalkyl or —CN;
R⁶ and R⁷ are each independently hydrogen, —C₁₋₃alkyl or —C₃₋₆cycloalkyl; each said —C₁₋₃alkyl —C₃₋₆cycloalkyl is optionally substituted with at least one substituent selected from hydrogen, halogen, —C₁₋₈alkoxy, —C₃₋₈cycloalkyl or —CN;
m1, m2 and m3 are each independently 0, 1 or 2, provided that m1+m2+m3≤4 and m1+m2+m3≥1;
m4 and m5 are each independently 0, 1 or 2, provided that m4+m5=2;
L¹ is independently selected from —C₁₋₃alkylene-, —O—, —NR^{a}—, —C(O)—, *^{L1}—C(O)NR^{a}—**^{L1}, *^{L1}—C(O)O—**^{L1}, *^{L1}—NR^{a}C(O)—**^{L1}, *^{L1}—OC(O)—**^{L1}, 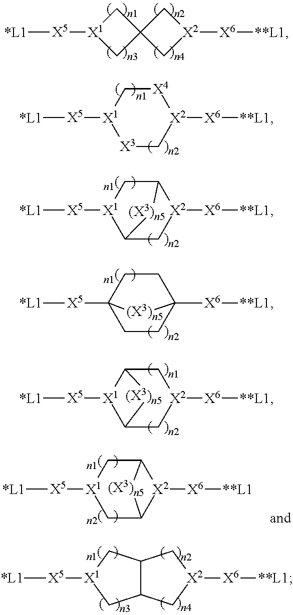
wherein each of said —C₁₋₃alkylene-, 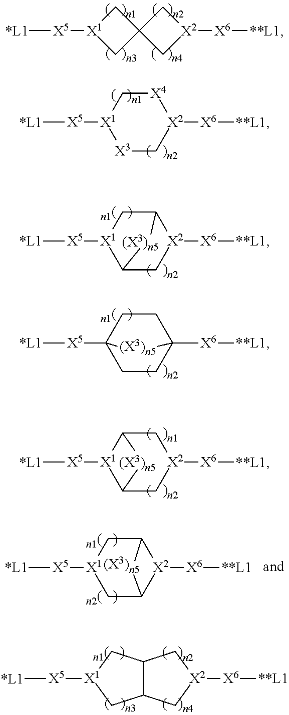
is optionally substituted with at least one R^{L1c};
wherein *^{L1} refers to the position attached to the 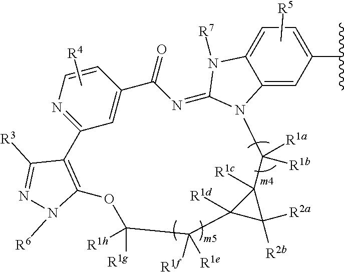
moiety, and **^{L1} refers to the position attached to the 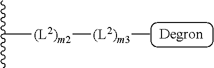
moiety;
L² is independently selected from —C₁₋₃alkylene-, —O—, —NR^{a}—, —C(O)—, *^{L2}—C(O)NR^{a}**^{L2}, *^{L2}—C(O)O—**^{L2}, *^{L2}—NR^{a}C(O)—**^{L2}, *^{L2}—OC(O)—**^{L2}, 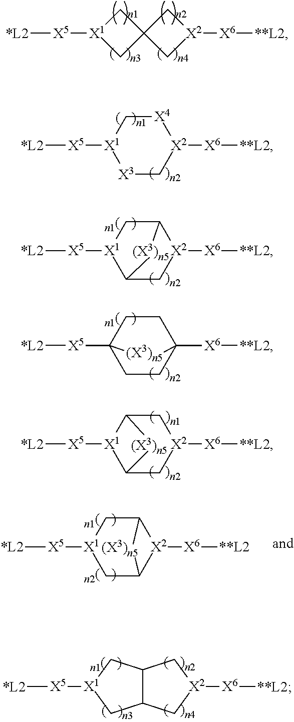
wherein each of said —C₁₋₃alkylene-, 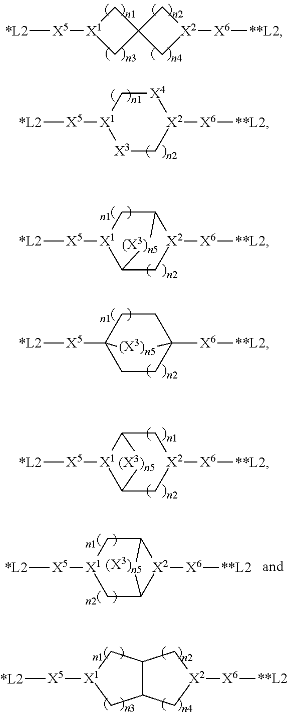
is optionally substituted with at least one R^{L2c};
wherein *^{L2} refers to the position attached to the 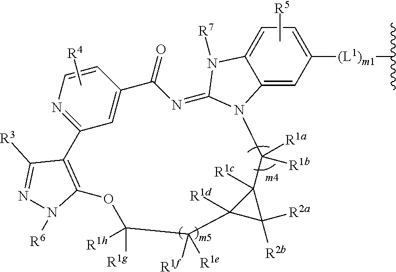
moiety, and **^{L2} refers to the position attached to the 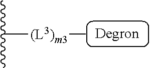
moiety;
L³ is independently selected from —C₁₋₃alkylene-, —O—, —NR^{a}—, —C(O)—, *^{L3}—C(O)NR^{a}**^{L3}, *^{L3}—C(O)O—**^{L3}, *^{L3}—NR^{a}C(O)—**^{L3}, *^{L3}—OC(O)—**^{L3}, 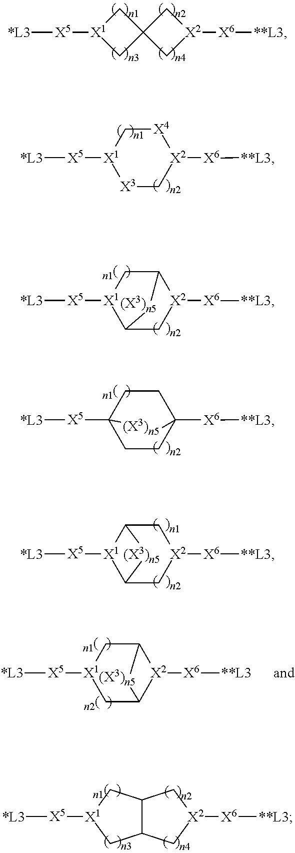
wherein each of said —C₁₋₃alkylene-, 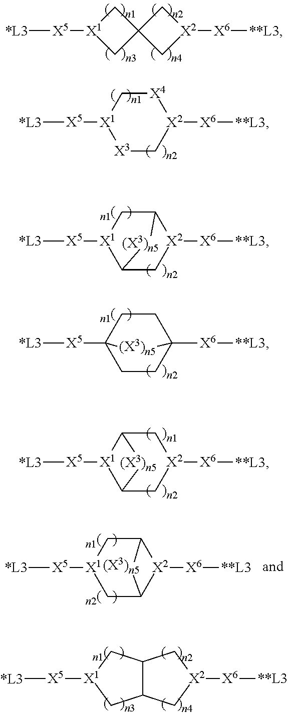
is optionally substituted with at least one R^{L3c};
wherein *^{L3} refers to the position attached to 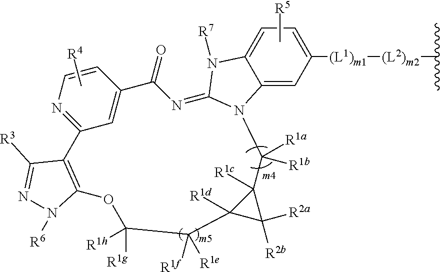
moiety, and **^{L3} refers to the position attached to the 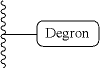
moiety;
each of said R^{L1c}, R^{L2c} and R^{L3c} are independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; each of said —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl and 5- to 12-membered heteroaryl is optionally substituted with at least one R^{Lca},
R^{Lca} is independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L2c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L3c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl; 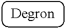
is selected from 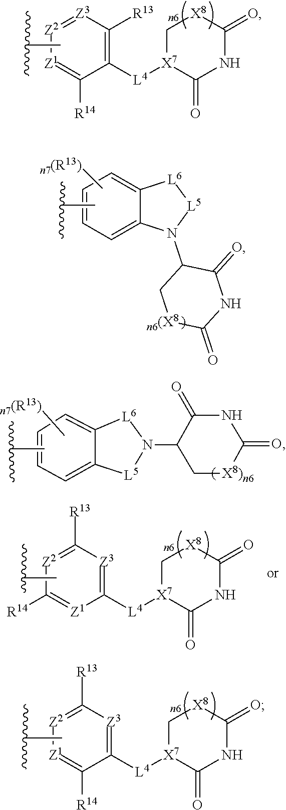
Z¹, Z² and Z³ are each independently N or CR^{Z}, provided that Z¹, Z² and Z³ are not N at the same time;
R^{z}, at each occurrence, is independently selected from absence, hydrogen, halogen, —C₁₋₈alkyl, —NR^{Za}R^{Zb}, —OR^{Za}, —SR^{Za}, C₃-C₈cycloalkyl, 3- to 8-membered heterocyclyl or CN; each of —C₁₋₈alkyl, C₃-C₈cycloalkyl, 3- to 8-membered heterocyclyl is optionally substituted with at least one R^{Zc}; 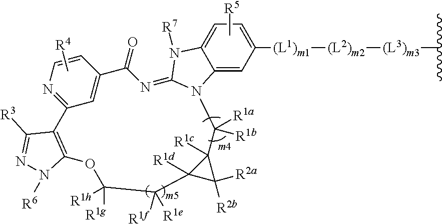
moiety is linked to the 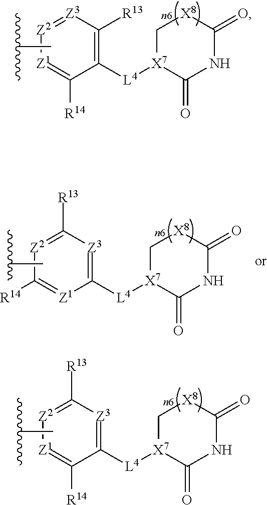
moiety via any one of Z¹, Z² or Z³ which is CR^{z} and R^{z} is absence;
R^{Za} and R^{Zb} are each independently selected from absence, hydrogen, —C₁-C₈alkyl, C₃-C₈cycloalkyl, 3- to 8-membered heterocyclyl, C₆-C₁₂aryl, or 5- to 12-membered heteroaryl, each of said —C₁₋₈alkyl, C₃-C₈cycloalkyl, 3- to 8-membered heterocyclyl, C₆-C₁₂aryl, or 5- to 12-membered heteroaryl is optionally substituted with at least one substituent R^{Zd};
R^{Zc} and R^{Zd} are each independently halogen, hydroxy, —C₁-C₈alkyl, —C₁-C₈alkoxy, C₃-C₈cycloalkyl, 3- to 8-membered heterocyclyl, C₆-C₁₂aryl, or 5- to 12-membered heteroaryl;
R¹³ and R¹⁴ are each independently selected from absence, hydrogen, halogen, —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, CO₂R^{13a}, CONR^{13a}R^{13b}, —NR^{13a}R^{13b}, —NR^{13a}COR^{13b}, —NR^{13a}CO₂R^{13b}, or —NR^{13a}SO₂R^{13b}; each of —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, —NR^{13c}COR^{13d}, —NR^{13c}CO₂R^{13d}, or —NR^{13c}SO₂R^{13d};
at each occurrence, R^{13a}, R^{13b}, R^{13c} and R^{13d} are each independently absence, hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl;
L⁴, L⁵ and L⁶ are each independently selected from a absence, single bond, —O—, —NR^{a}—, —(CR^{a}R^{b})ₙ₈—, —O(CR^{a}R^{b})ₙ₈—, —NR^{a}(CR^{a}R^{b})ₙ₈ or —C(O)—;
at each occurrence, X¹, X² and X⁷ are each independently selected from —CR^{a}, or N;
at each occurrence, X³, X⁴ and X⁸ are each independently selected from —NR^{a}—, —O—, —S— and —CR^{a}R^{b}—;
at each occurrence, X⁵ and X⁶ are each independently selected from absence, single bond, —C(O)—, —NR^{a}— and —O—;
at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;
n1, n2, n3, n4 and n5 are each independently 0, 1, 2 or 3;
n6, n7 and n8 are each independently 0, 1, 2, 3 or 4;
provided that:
for any one of L¹, L² or L³, when X¹ is N, X⁵ is single bond, absence, —C(O)—; and/or when X² is N, X⁶ is single bond, absence, —C(O)—;
when L¹ is 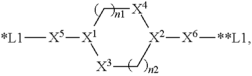
when X¹ is N, X⁵ is single bond, absence, —C(O)—; and/or X³ is —CR^{a}R^{b}—; when X² is N X⁶ is single bond, absence, —C(O)—, and/or X⁴ is —CR^{a}R^{b}—;
when L² is 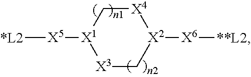
when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—;
when L³ is 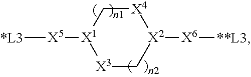
when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—.

34. The compound of claim 33, wherein the compound is selected from formula (IIa), (IIb), (IIc), (IId), (Ilia), (IIIb), (IIIc), (IIId), (Va), (Vb), (Vc), (Vd), (Ve), (Vf): 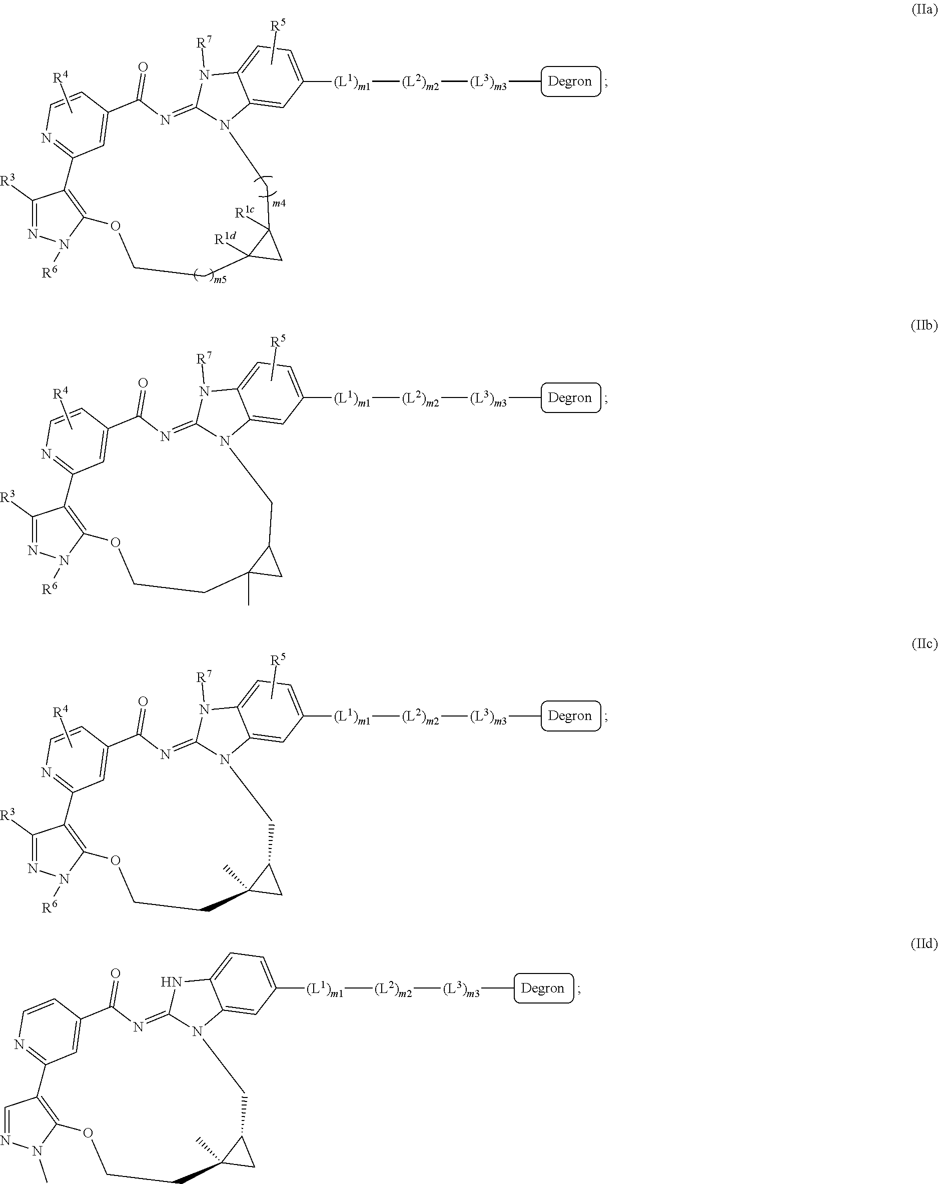 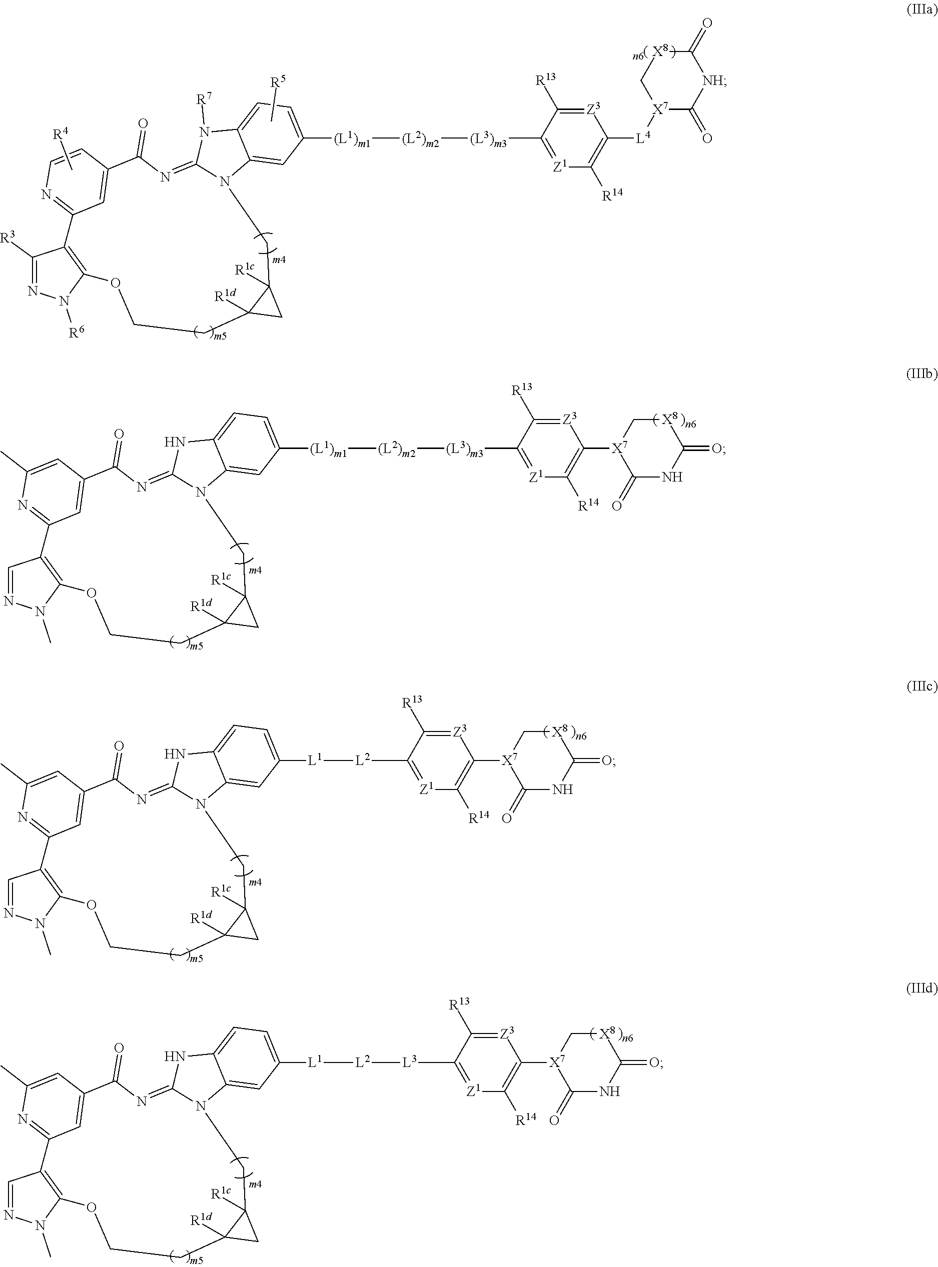 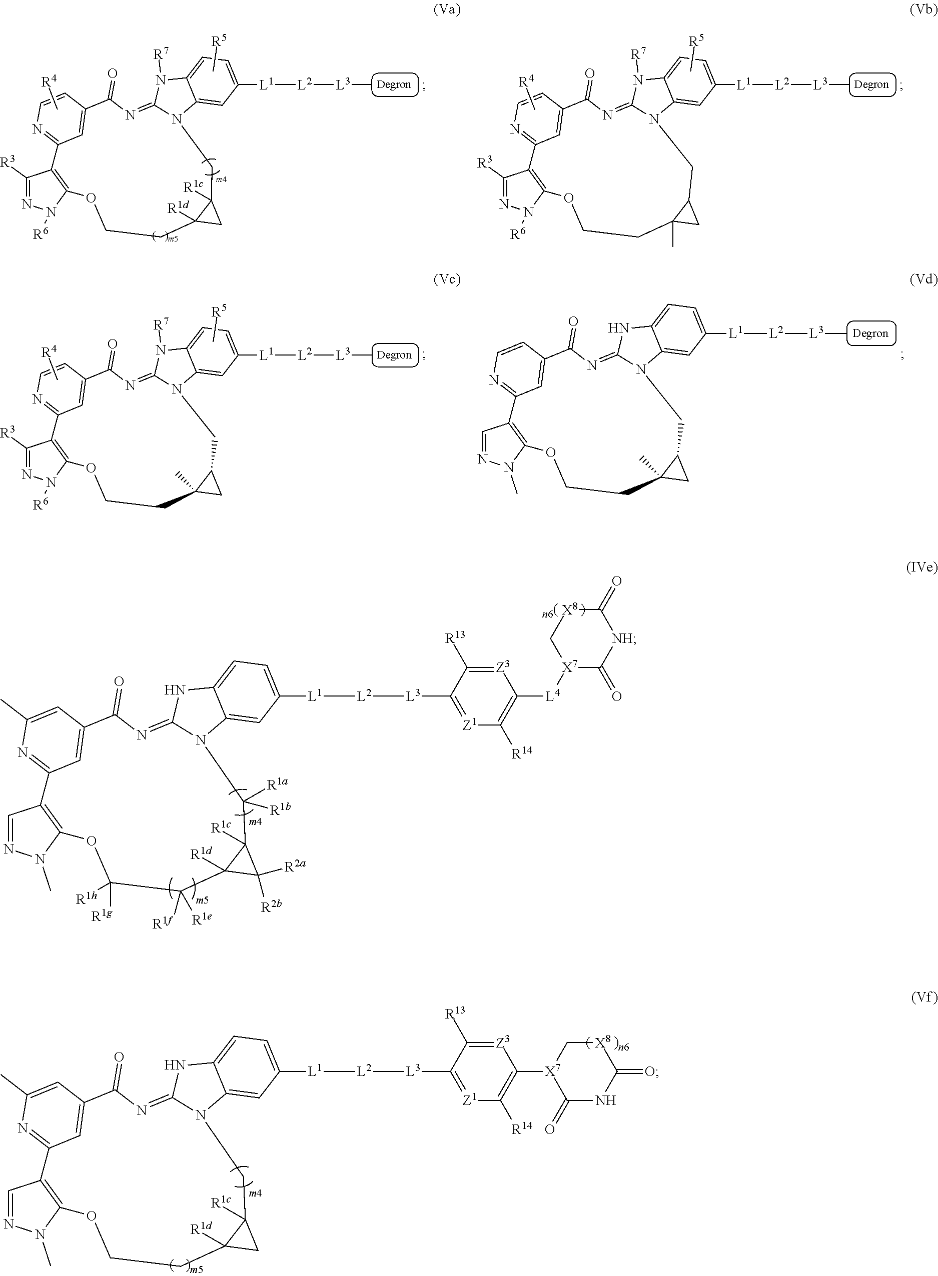
wherein R^{1c} and R^{1d} are each independently H, methyl or ethyl, provided that at most one of R^{1c} and R^{1d} is H.

35. The compound of claim 33, wherein the compound is selected from formula (IVa) 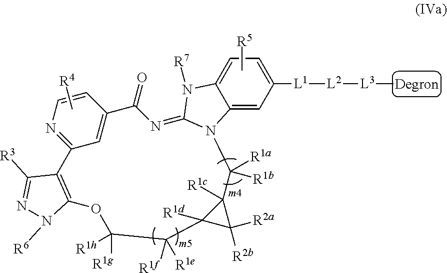
or a N-oxide thereof, or a pharmaceutically acceptable salt thereof, or a stereoisomer thereof, or a deuterated analog thereof, or a prodrug thereof,
wherein:
R^{1a}, R^{1b}, R^{1c}, R^{1d}, R^{1e}, R^{1f}, R^{1g}, R^{1h}, R^{2a}, R², R³, R⁴ and R⁵ are each independently hydrogen, halogen, —C₁₋₄alkyl, —C₁₋₄alkoxy, —C₃₋₆cycloalkyl or —CN; each said —C₁₋₄alkyl, —C₁₋₄alkoxy, or —C₃₋₆cycloalkyl is optionally substituted with at least one substituent selected from hydrogen, halogen, —C₁₋₈alkoxy, —C₃₋₈cycloalkyl or —CN;
R⁶ and R⁷ are each independently hydrogen, —C₁₋₃alkyl or —C₃₋₆cycloalkyl; each said —C₁₋₃alkyl —C₃-6cycloalkyl is optionally substituted with at least one substituent selected from hydrogen, halogen, —C₁₋₈alkoxy, —C₃₋₈cycloalkyl or —CN;
m4 and m5 are each independently 0, 1 or 2, provided that m4+m5=2;
wherein
L¹ is independently selected from 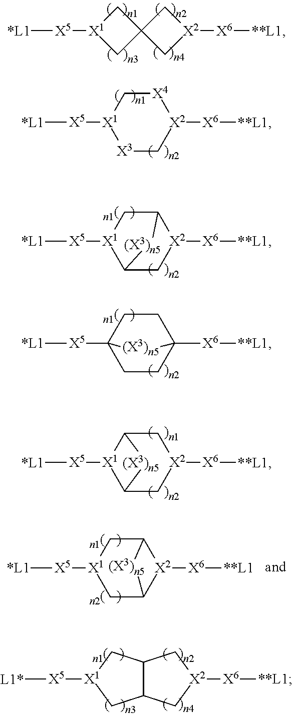
wherein each of 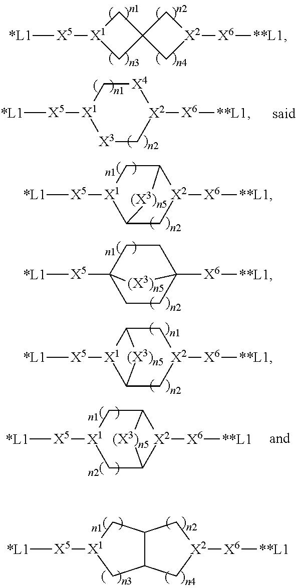
is optionally substituted with at least one R^{L1c};
wherein *^{L1} refers to the position attached to the 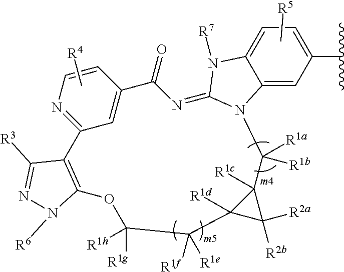
moiety, and **^{L1} refers to the position attached to the 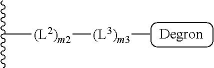
moiety;
L² is independently selected from 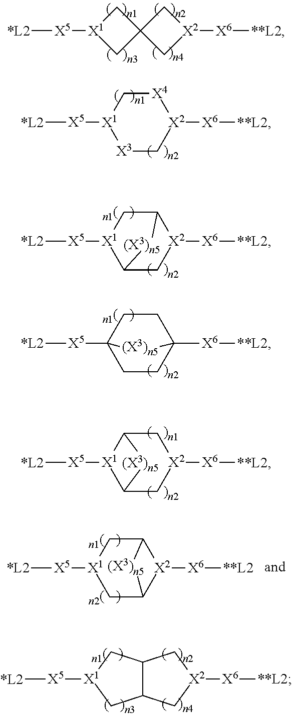
wherein each of said 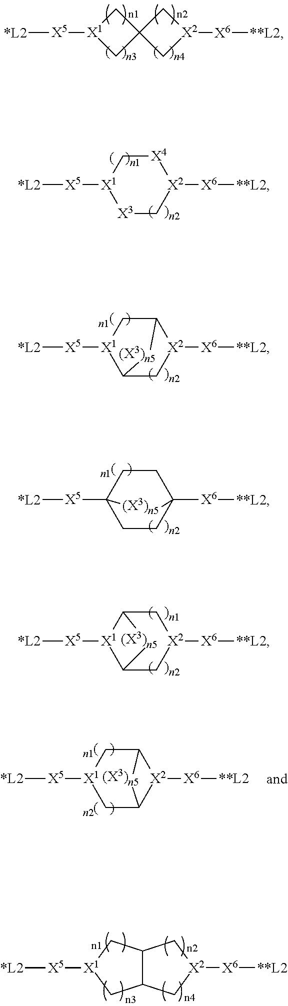
is optionally substituted with at least one R^{L2c};
wherein *^{L2} refers to the position attached to the 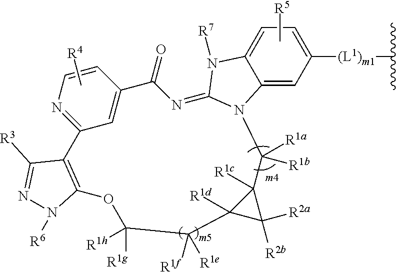
moiety, and **^{L2} refers to the position attached to the 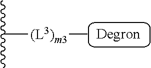
moiety;
L³ is independently selected from 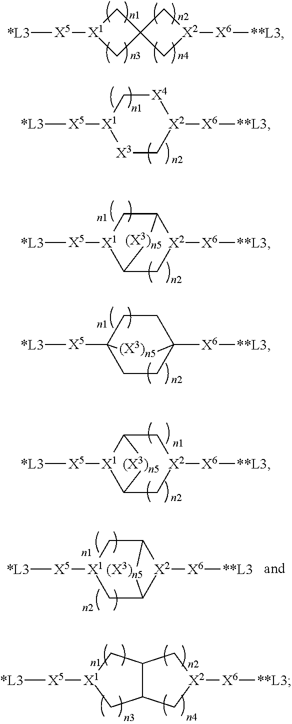
wherein each of said 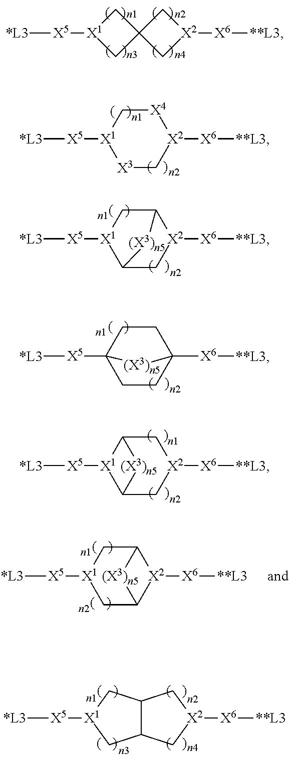
is optionally substituted with at least one R^{L3c};
wherein *^{L3} refers to the position attached to the 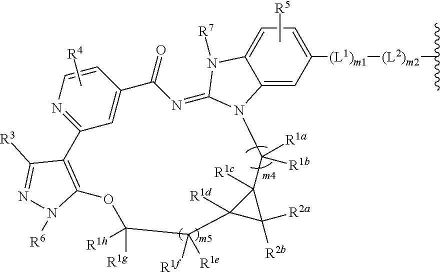
moiety, and **^{L3} refers to the position attached to the 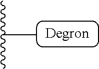
moiety;
each of said R^{L1c}, R^{L2c} and R^{L3c} are independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; each of said —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl and 5- to 12-membered heteroaryl is optionally substituted with at least one R^{Lca},
R^{Lca} is independently absence, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L2c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
two R^{L3c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl; 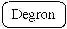
is selected from 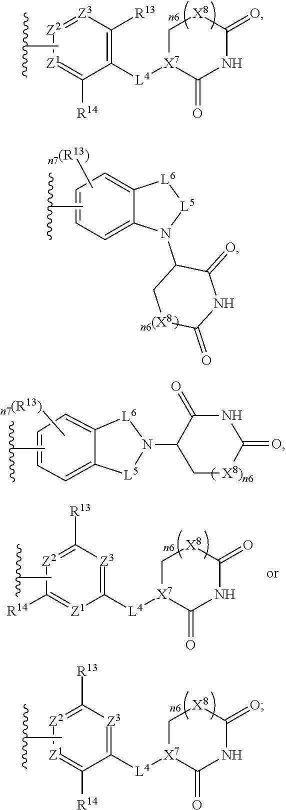
Z¹, Z² and Z³ are each independently N or CR^{Z}, provided that Z¹, Z² and Z³ are not N at the same time;
R^{Z}, at each occurrence, is independently selected from absence, hydrogen, halogen, —C₁₋₈alkyl, —NR^{Za}R^{Zb}, OR^{Za}, —SR^{Za} C₃-C₈cycloalkyl, 3- to 8-membered heterocyclyl or CN; each of —C₁₋₈alkyl, C₃-C₈cycloalkyl, 3- to 8-membered heterocyclyl is optionally substituted with at least one R^{Zc};
the 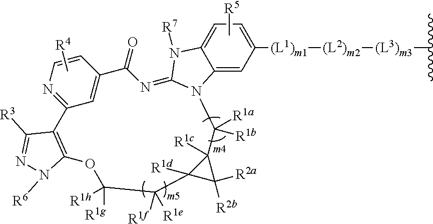
moiety is linked to the 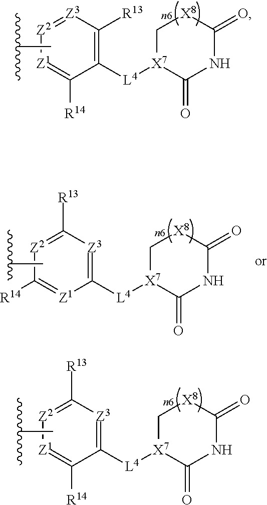
moiety via any one of Z¹, Z² or Z³ which is CR^{z} and R^{z} is absence;
R^{Za} and R^{Zb} are each independently selected from absence, hydrogen, —C₁-C₈alkyl, C₃-C₈cycloalkyl, 3- to 8-membered heterocyclyl, C₆-C₁₂aryl, or 5- to 12-membered heteroaryl, each of said —C₁₋₈alkyl, C₃-C₈cycloalkyl, 3- to 8-membered heterocyclyl, C₆-C₁₂aryl, or 5- to 12-membered heteroaryl is optionally substituted with at least one substituent R^{Za};
R^{Zc} and R^{Za} are each independently halogen, hydroxy, —C₁-C₈alkyl, —C₁₋₈alkoxy, C₃-C₈cycloalkyl, 3- to 8-membered heterocyclyl, C₆-C₁₂aryl, or 5- to 12-membered heteroaryl;
R¹³ and R¹⁴ are each independently selected from absence, hydrogen, halogen, —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}CO₂R^{13b}, or —NR^{13a}SO₂R^{13b}; each of —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{13c}, —SO₂R^{13c}, —SO₂NR^{13c}R^{13a}, —COR^{13c}, —CO₂R^{13c}, —CONR^{13c}R^{13a}, —NR^{13c}R^{13a}, —NR^{13c}COR^{13a}, —NR^{13c}CO₂R^{13d}, or —NR^{13c}SO₂R^{13a};
at each occurrence, R^{13a}, R^{13b}, R^{13c} and R^{13a} are each independently absence, hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl;
L⁴, L⁵ and L⁶ are each independently selected from a absence, single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
at each occurrence, X¹, X² and X⁷ are each independently selected from —CR^{a}, or N;
at each occurrence, X³, X⁴ and X⁸ are each independently selected from —NR^{a}—, —O—, —S— and —CR^{a}R^{b}—;
at each occurrence, X⁵ and X⁶ are each independently selected from absence, single bond, —C(O)—, —NR^{a}— and —O—;
at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;
n1, n2, n3, n4 and n5 are each independently 0, 1, 2 or 3;
n6, n7 and n8 are each independently 0, 1, 2, 3 or 4;
provided that:
for any one of L1, L² or L³, when X¹ is N, X⁵ is single bond, absence, —C(O)—; and/or when X² is N, X⁶ is single bond, absence, —C(O)—;
when L¹ is 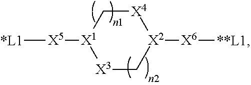
when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—;
when L² is 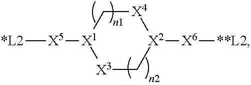
when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—;
when L³ is 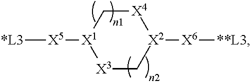
when X¹ is N, X⁵ is single bond, absence, —C(O)—; and/or X³ is —CR^{a}R^{b}—; when X² is N, X⁶ is single bond, absence, —C(O)—, and/or X⁴ is —CR^{a}R^{b}—.

36. The compound of claim 33, wherein R⁶ and R⁷ are each independently hydrogen, methyl, ethyl, propyl, cyclopropyl, cyclobutyl, cyclopentyl or cyclohexyl.

37. The compound of claim 33, wherein R^{1a}, R^{1b}, R^{1c}, R^{1d}, R^{1e}, R^{1f}, R^{1g}, R^{1h}, R^{2a}, R^{2b}, R³, R⁴ and R⁵ are each independently hydrogen, F, Cl, Br, I, methyl, ethyl, propyl, butyl, methoxy, ethoxy, propoxy, butoxy, cyclopropyl, cyclobutyl, cyclopentyl, cyclohexyl or —CN; wherein each said methyl, ethyl, propyl, butyl, methoxy, ethoxy, propoxy, butoxy, cyclopropyl, cyclobutyl, cyclopentyl or cyclohexyl is optionally substituted with at least one substituent selected from hydrogen, F, Cl, Br, I, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN.

38. The compound of claim 33, wherein the 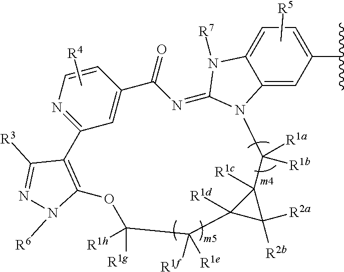
moiety is selected from

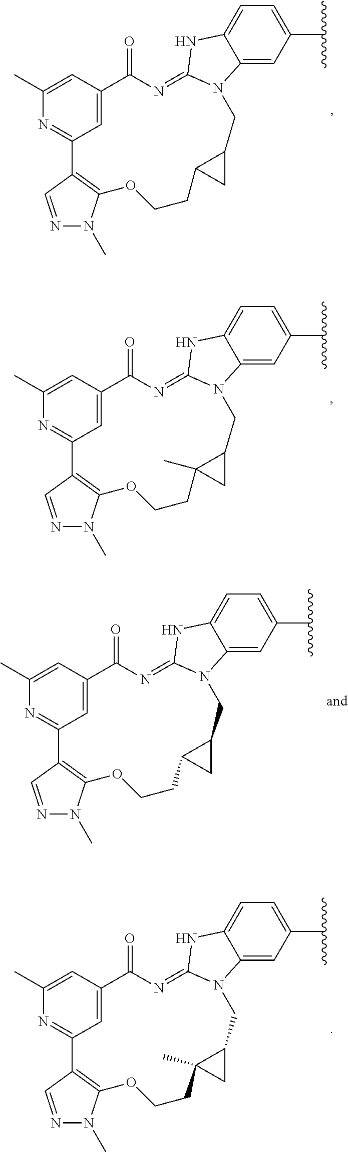 [SMILES: *c1ccc2c(c1)N1CC3CC3CCOc3c(cnn3C)-c3cc(cc(C)n3)C(=O)/N=C/1N2 ; *c1ccc2c(c1)N1CC3CC3(C)CCOc3c(cnn3C)-c3cc(cc(C)n3)C(=O)/N=C/1N2 ; *c1ccc2c(c1)N1C[C@H]3C[C@@H]3CCOc3c(cnn3C)-c3cc(cc(C)n3)C(=O)/N=C/1N2 ; *c1ccc2c(c1)N1C[C@@H]3C[C@@]3(C)CCOc3c(cnn3C)-c3cc(cc(C)n3)C(=O)/N=C/1N2]

39. The compound of claim 33, wherein L¹ is selected from —O—, —C(O)—, —N(R^{a}), *^{L1}—C(O)N(R^{a})—**^{L1}, *^{L1}—C(O)O—**^{L1}, *^{L1}—N(R^{a})C(O)—**^{L1}, *^{L1}—OC(O)—**^{L1}, 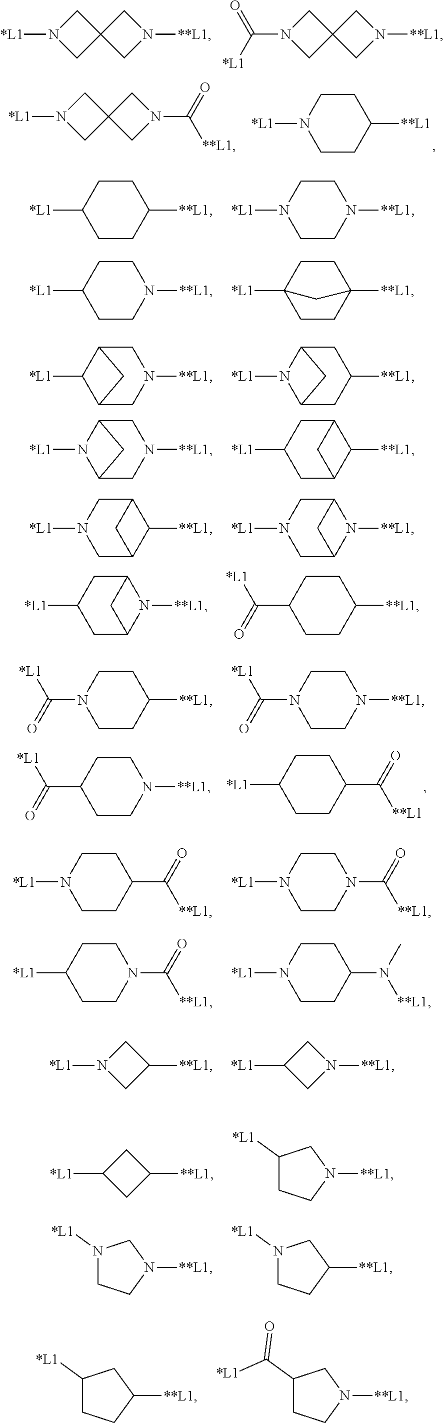 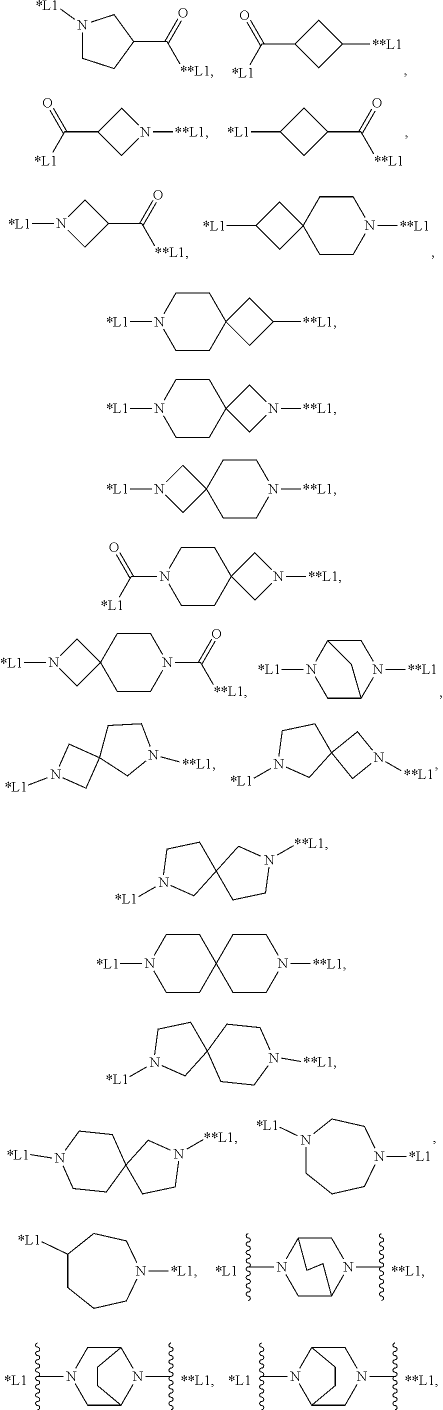 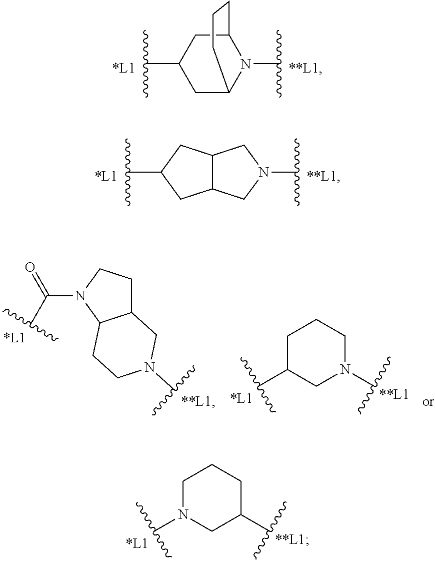 [SMILES: *C1CC2CCCC(C1)N2* ; *C1CC2CN(*)CC2C1 ; *C(=O)N1CCC2CN(*)CCC21 ; *C1CCCN(*)C1 ; *C1CCCN(*)C1]
wherein each of said 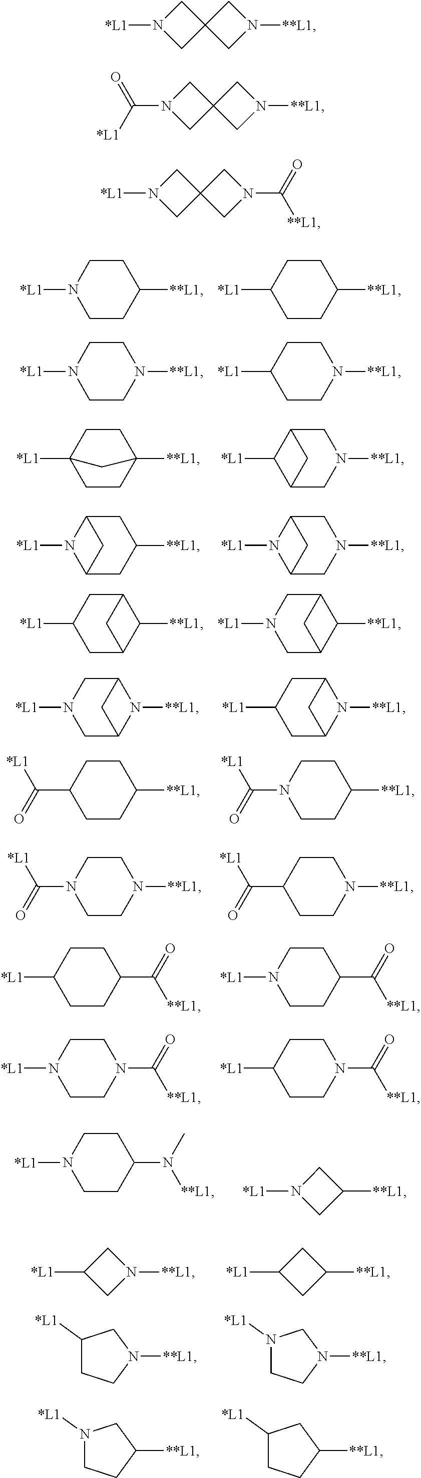 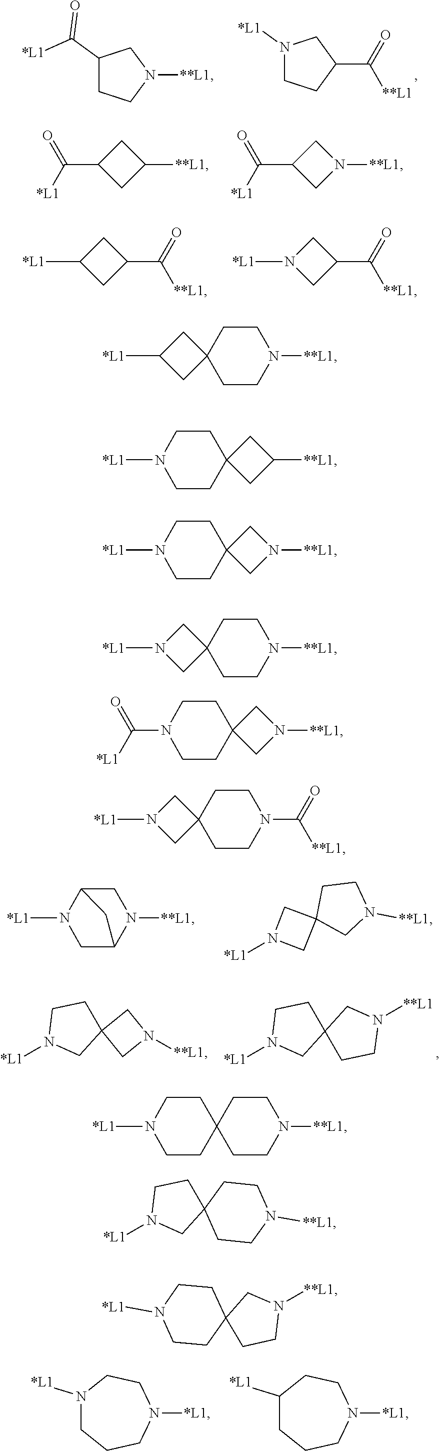 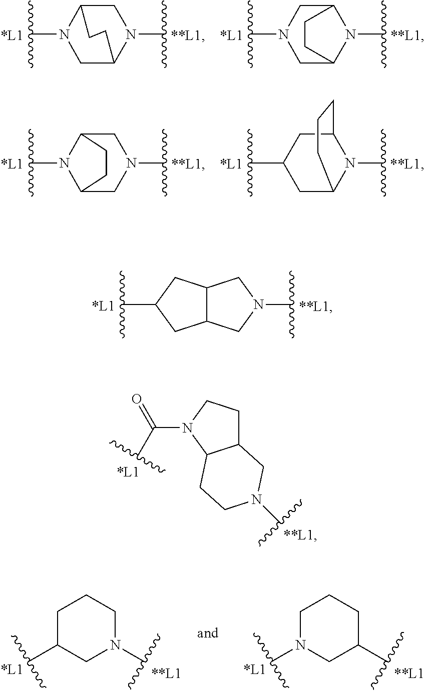
is optionally substituted with at least one R^{L1c};
each of said R^{L1c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L1c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, or octyl;
L² is selected from —O—, —C(O)—, —N(R^{a})—, *^{L2}—C(O)N(R^{a})—**^{L2}, *^{L2}—C(O)O—**^{L2}, *^{L2}—N(R^{a})C(O)—**^{L2}, *^{L2}—OC(O)—**^{L2}, 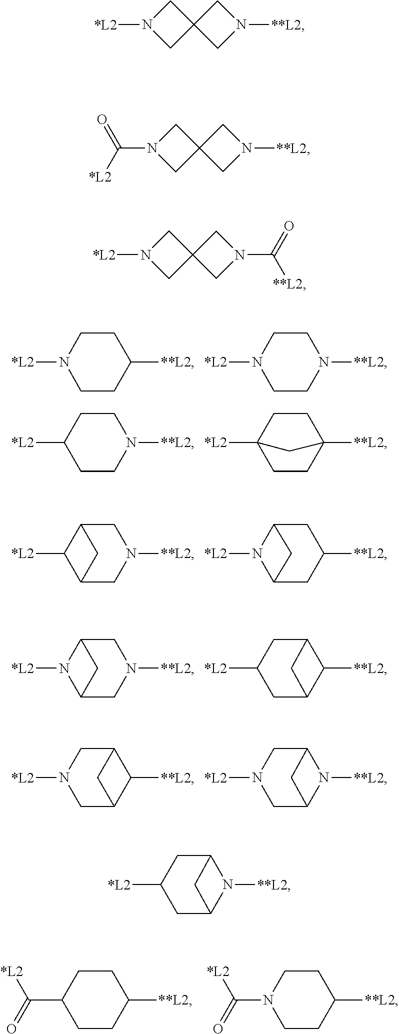 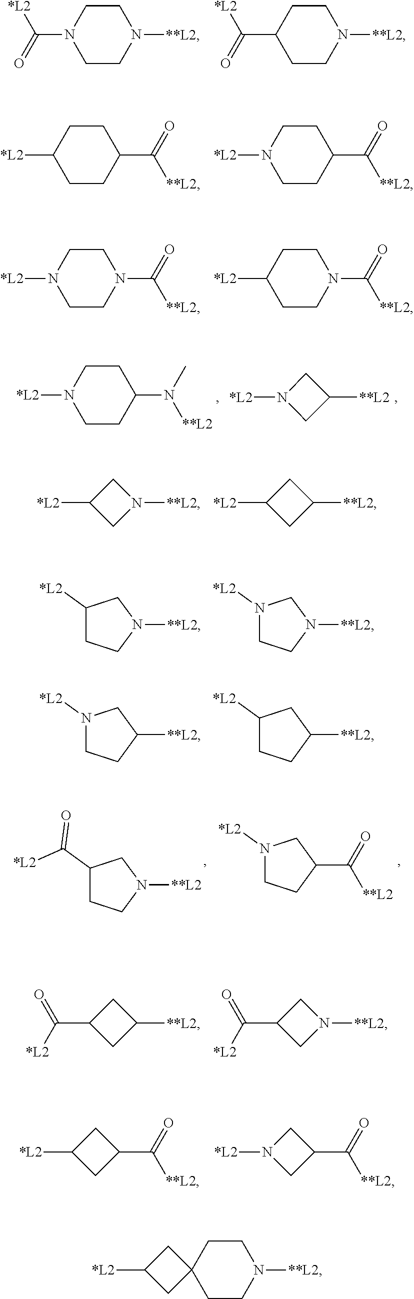 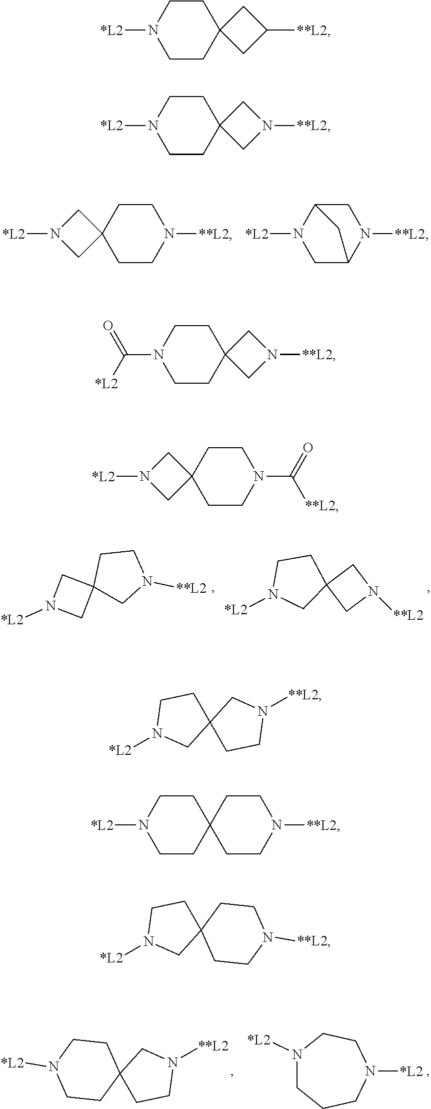 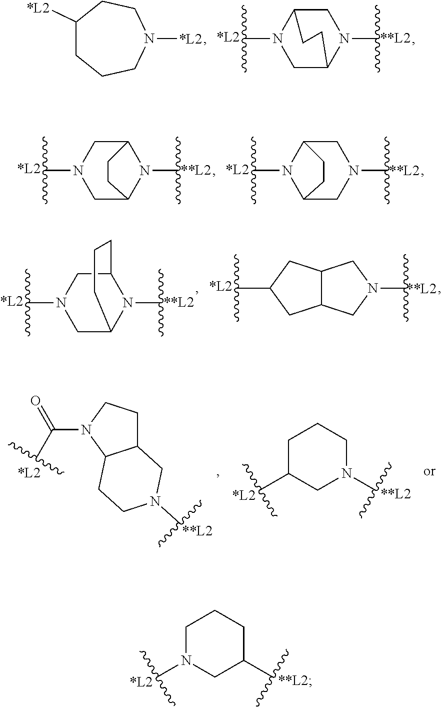
wherein each of said 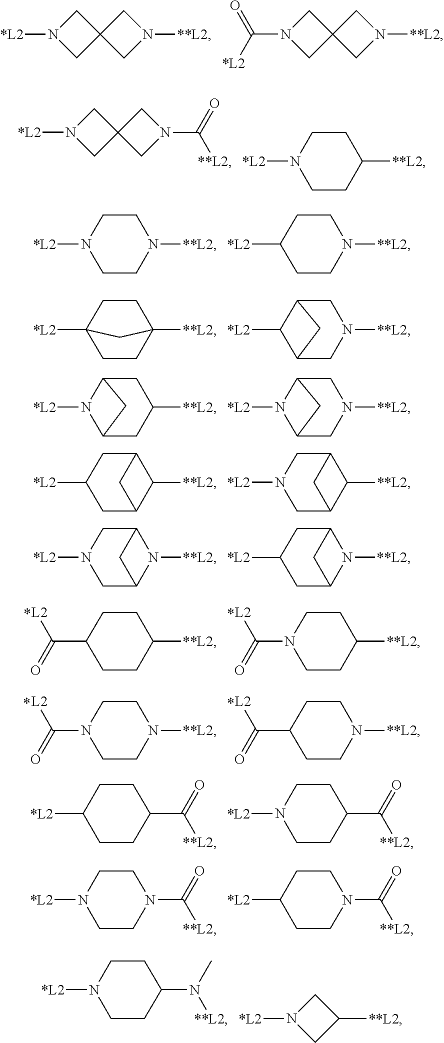 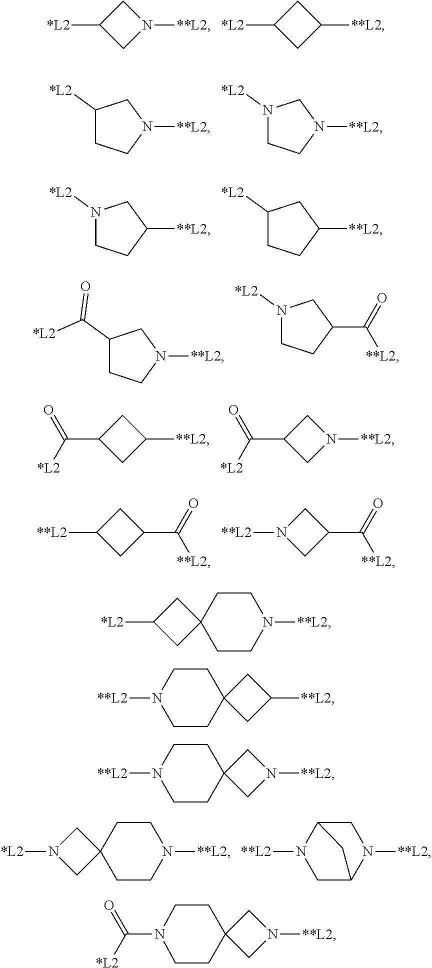 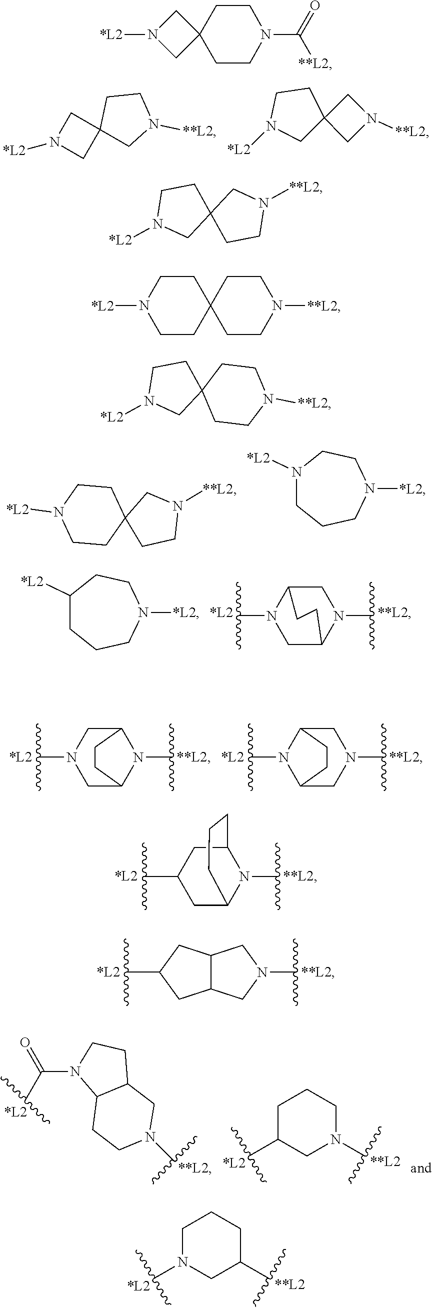
is optionally substituted with at least one R^{L2c};
each of said R^{L2c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L2c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl;
L³ is selected from —O—, —N(R^{a})—, —C(O)—, *^{L3}—C(O)N(R^{a})**^{L3}, *^{L3}C(O)O**^{L3}, *^{L3}—N(R^{a})C(O)—**^{L3}, *^{L3} OC(O)—**^{L3}, 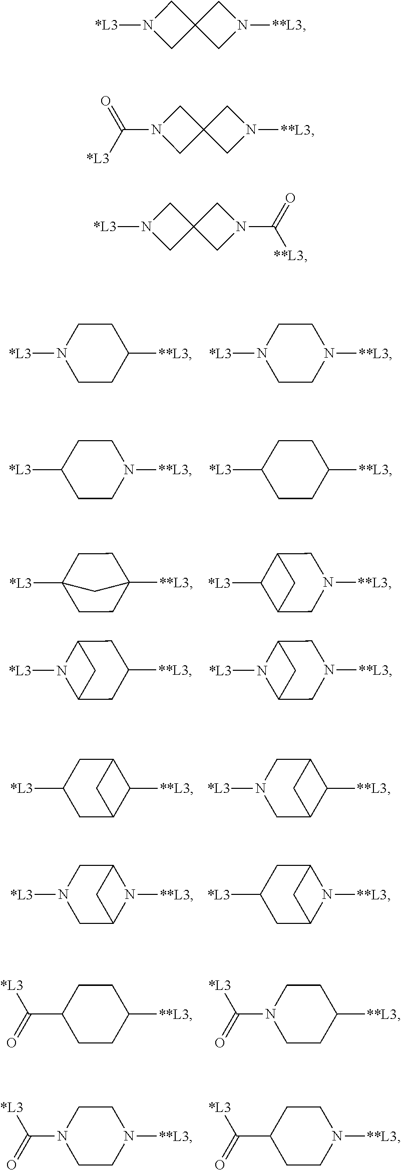 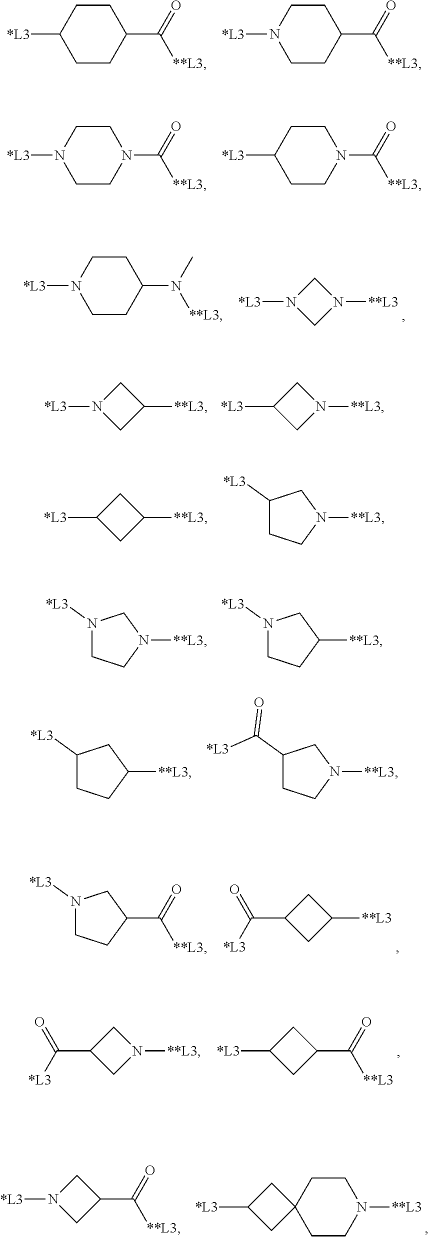 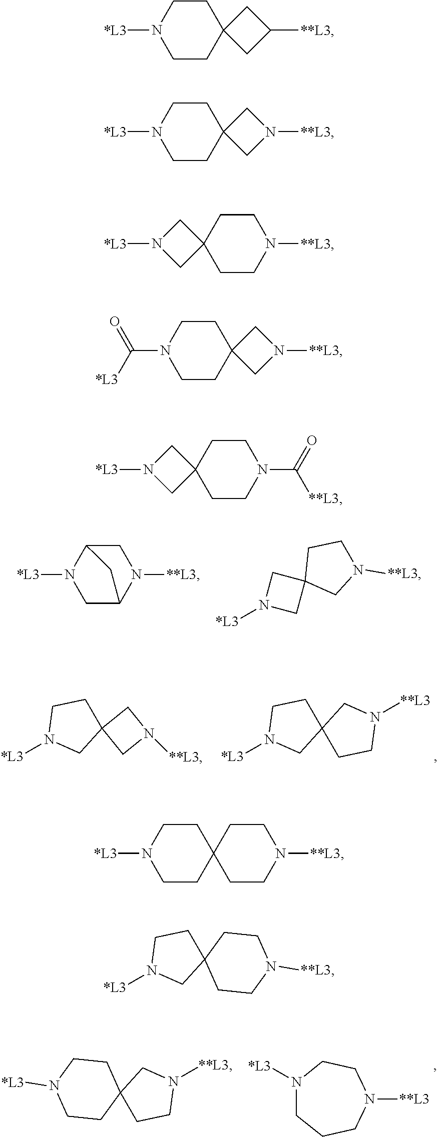 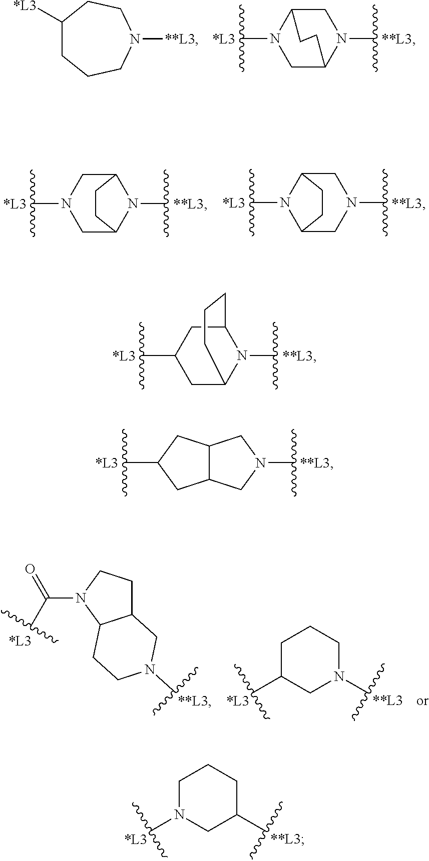
wherein each of said 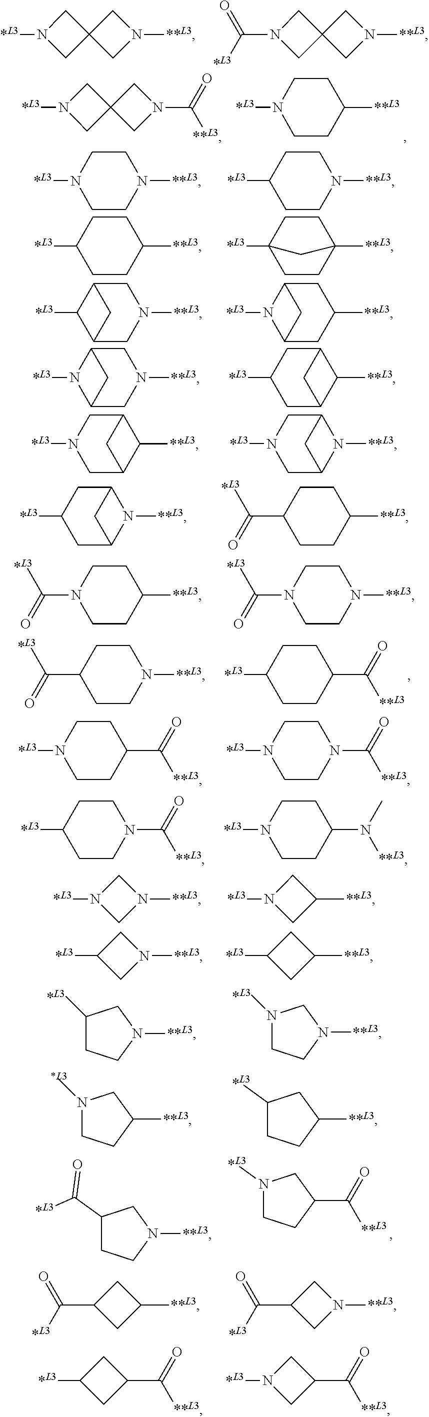 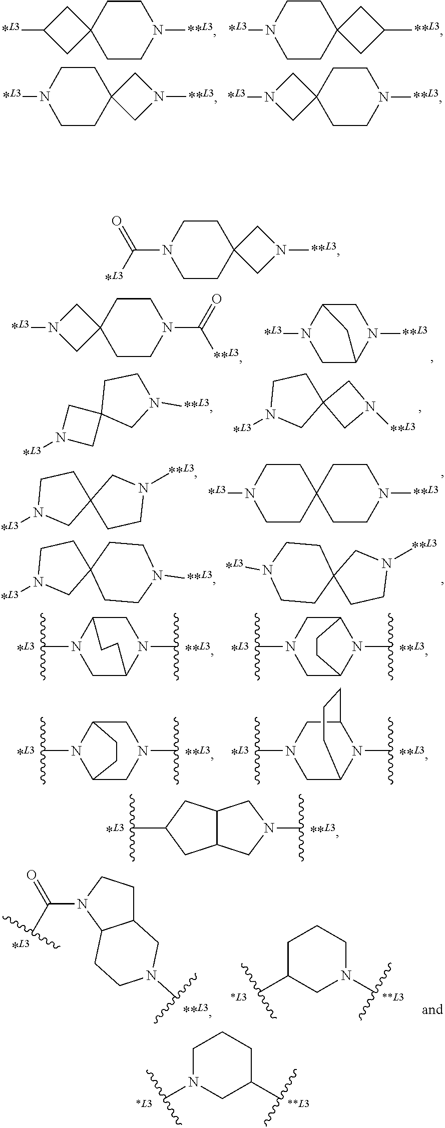
is optionally substituted with at least one R^{L3c};
each of said R^{L3c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L3c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl, each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

40. The compound of claim 33, wherein L¹ is selected from —O—, —N(CH₃)—, —C(O)—, —NH—, *^{L1}—C(O)N(CH₃)—**^{L1}, *^{L1}—C(O)NH—**^{L1}, *^{L1}—C(O)O—**^{L1}, *^{L1}—C(O)N(C₂H₅)—**^{L1}, *^{L1}—C(O)N(C₃H₇)—**^{L1}, *^{L1}—N(CH₃)C(O)—**^{L1}, *^{L1}—NHC(O)—**^{L1}, *^{L1}—OC(O)—**^{L1}, *^{L1}—N(C₂H₅)C(O)—**^{L1}, *^{L1}—N(C₃H₇)C(O)—**^{L1}, 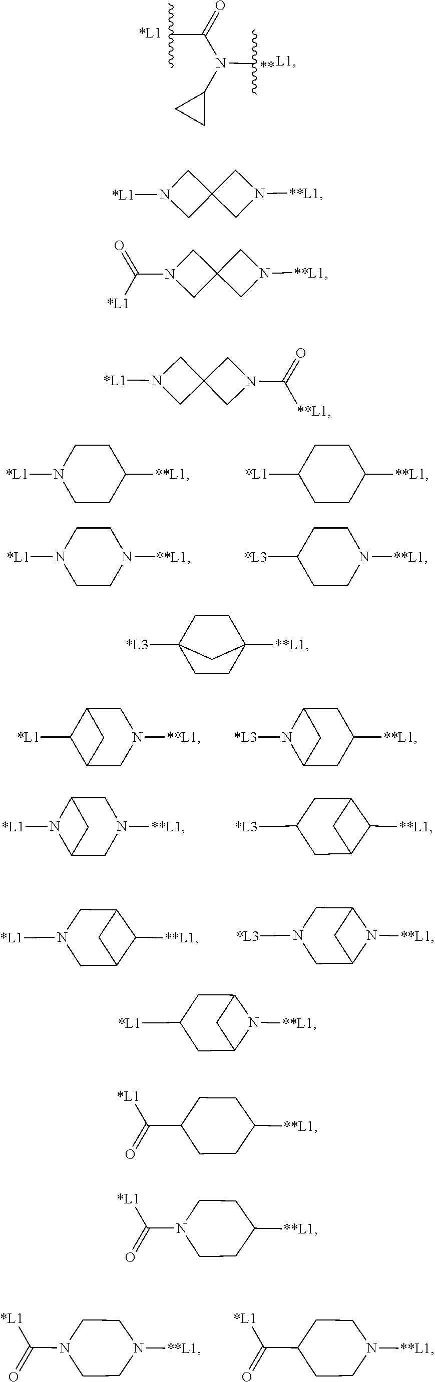 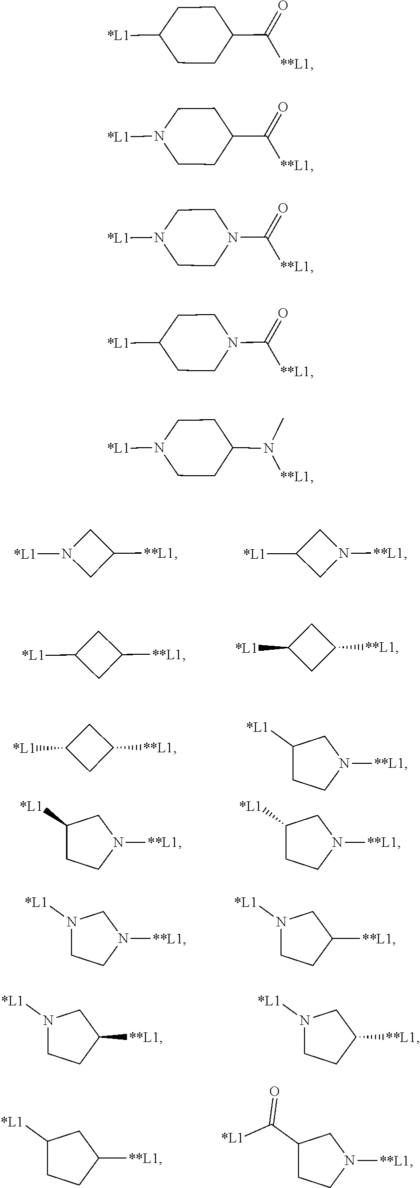 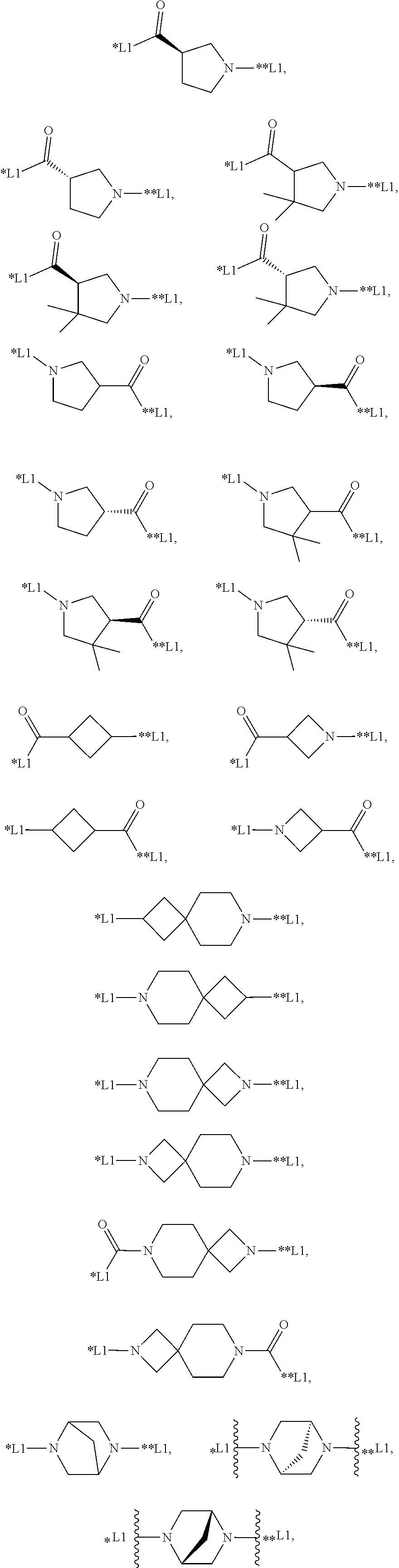 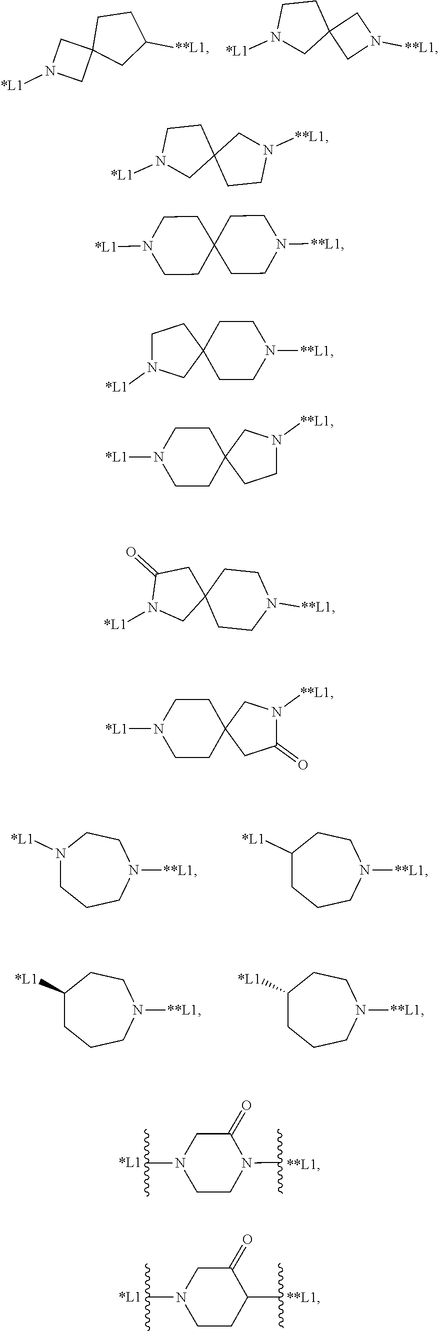 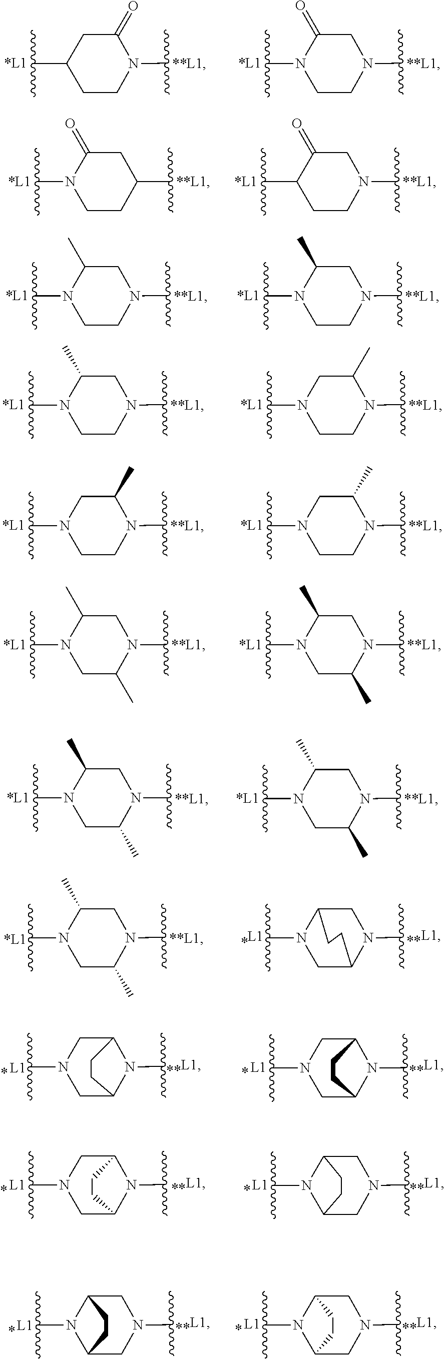 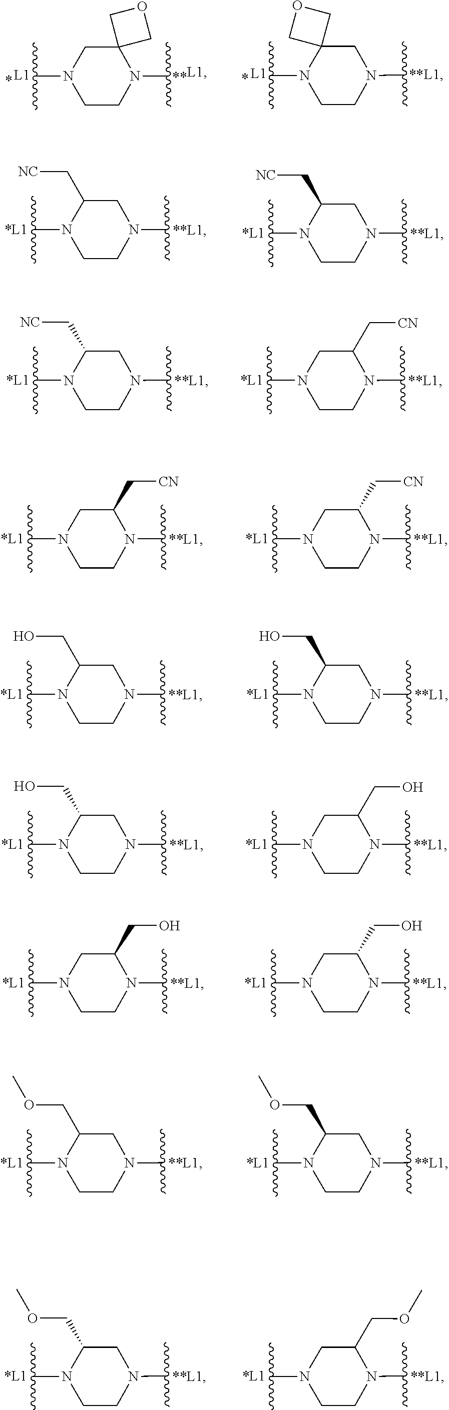 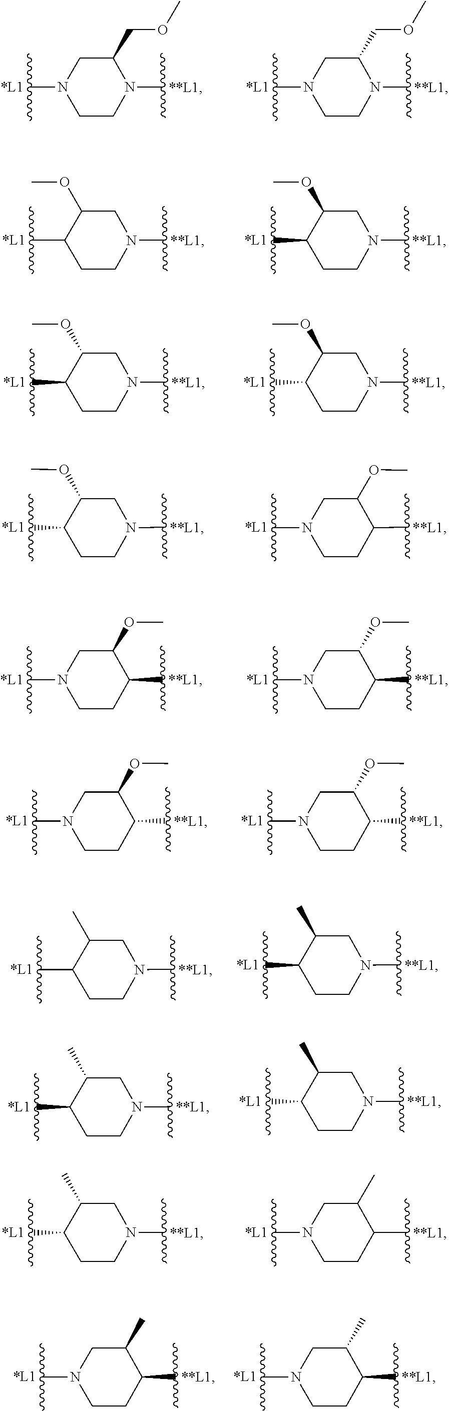 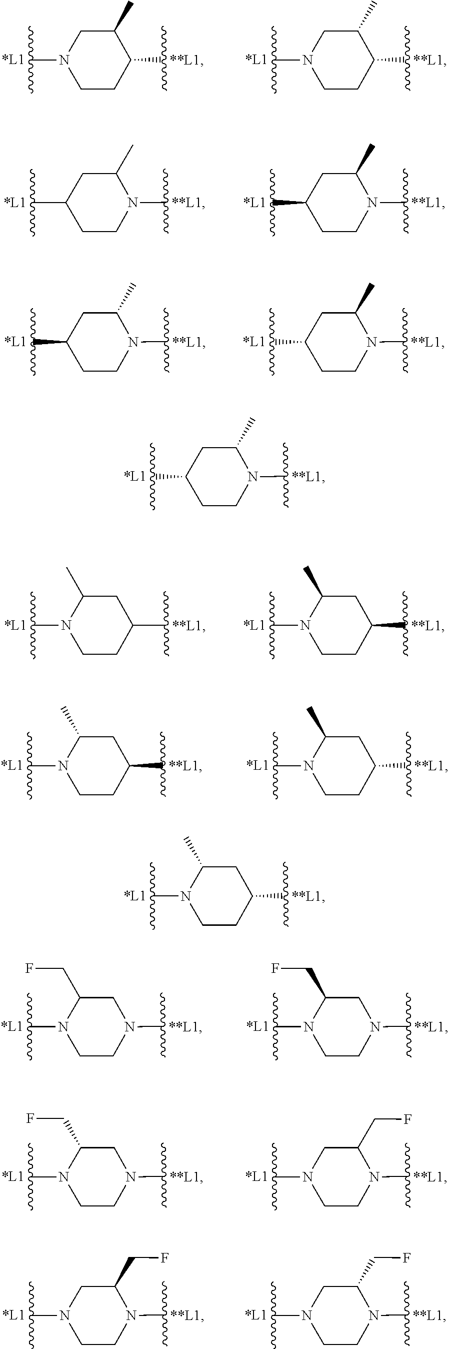 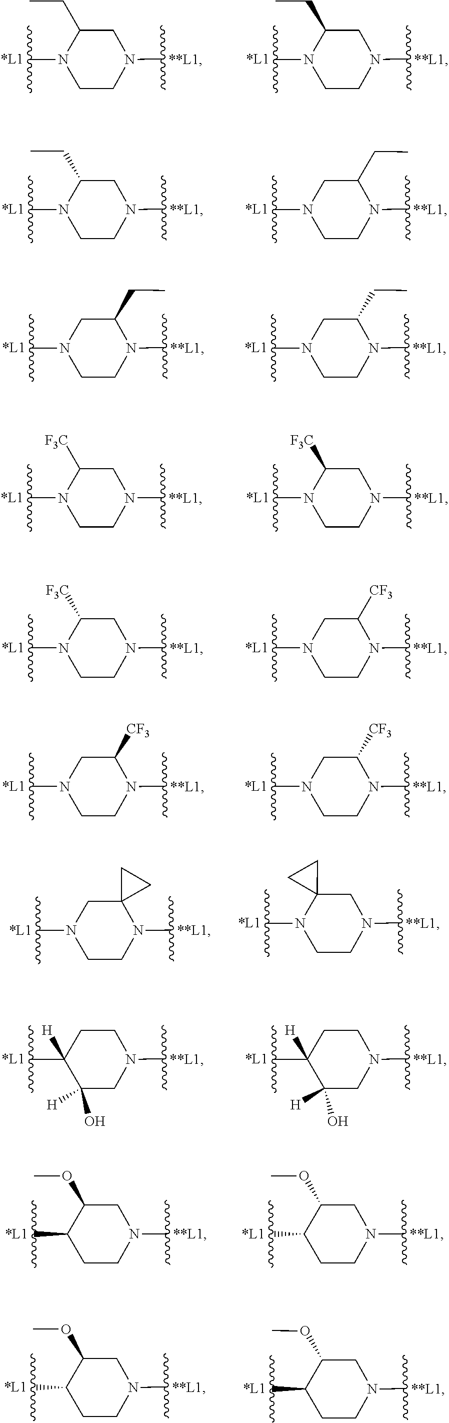 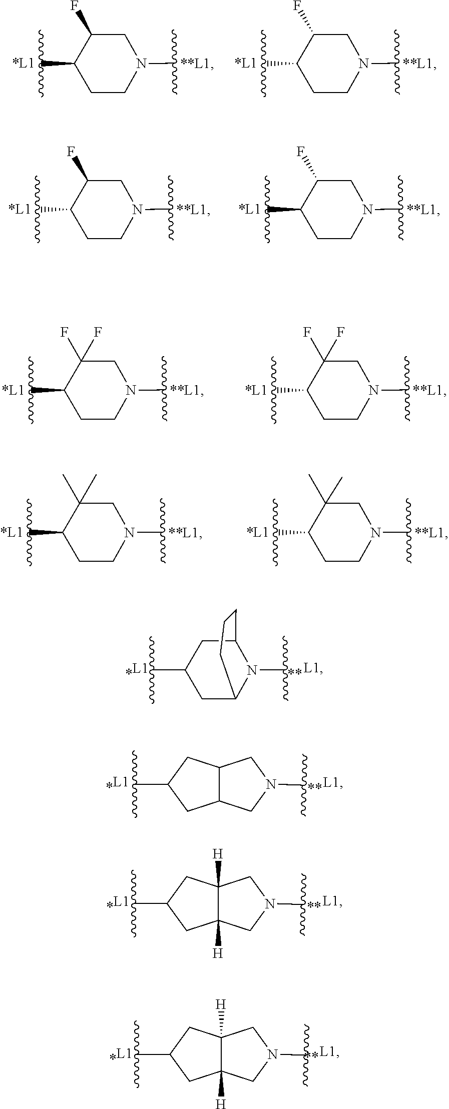 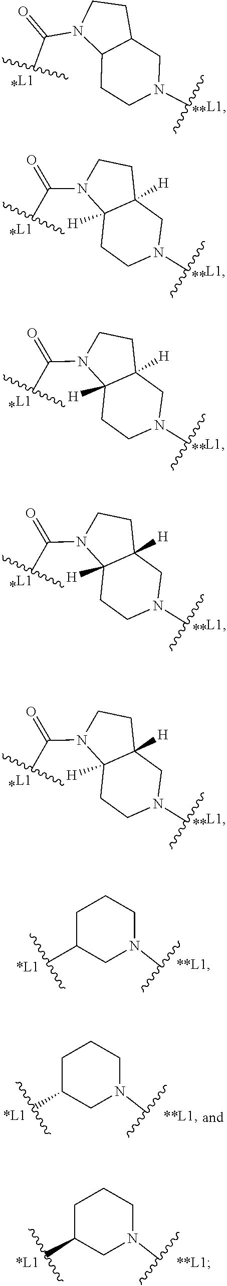
L² is selected from —O—, —N(CH₃)—, —C(O)—, —NH—, *^{L2}—C(O)N(CH₃)—**^{L2}, *^{L2}—C(O)NH—**^{L2}, *^{L2}—C(O)O—**^{L2}, *^{L2}—C(O)N(C₂H₅)—**^{L2}, *^{L2}—C(O)N(C₃H₇)—**^{L2}, *^{L2}—N(CH₃)C(O)—**^{L2}, *^{L2}—NHC(O)**^{L2}, *^{L2}-OC(O)—**^{L2}—N(C₂H₅)C(O)—**^{L2}, *^{L2}—N(C₃H₇)C(O)—**^{L2}, 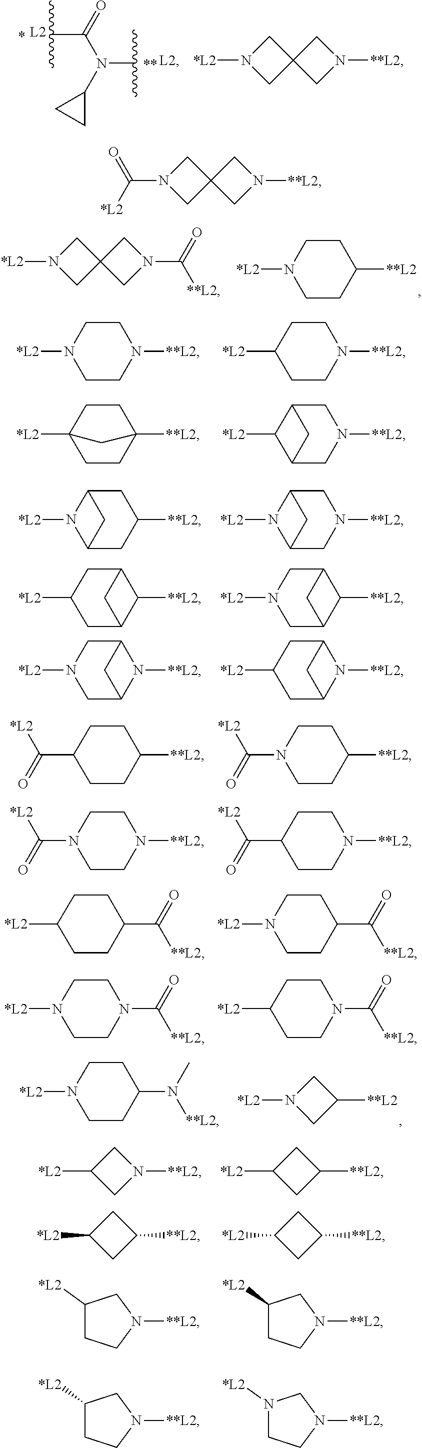 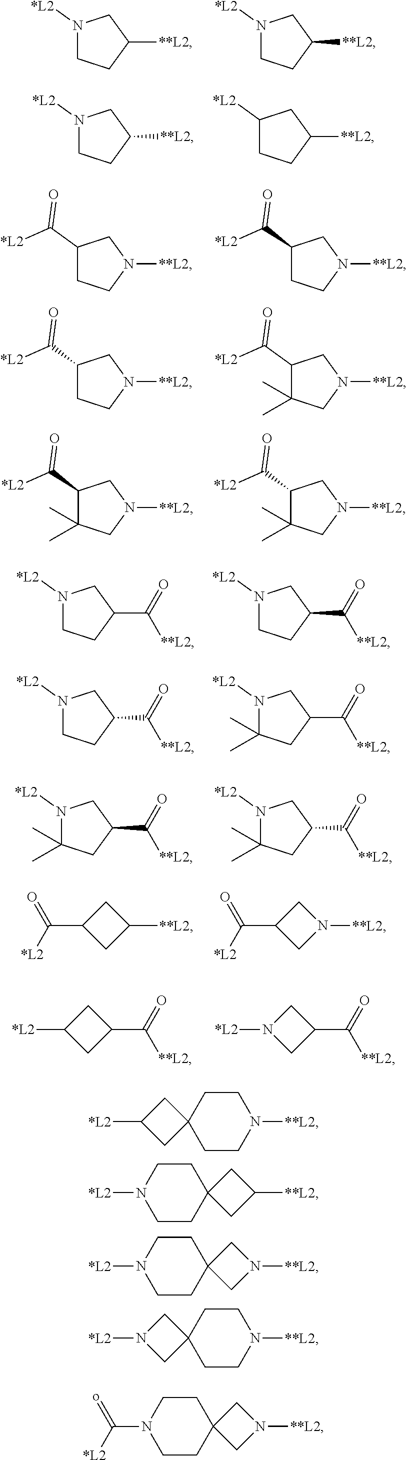 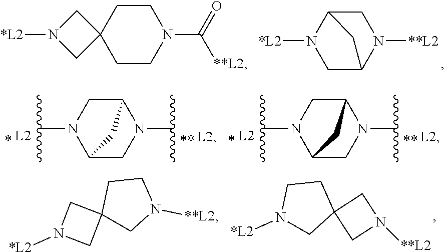 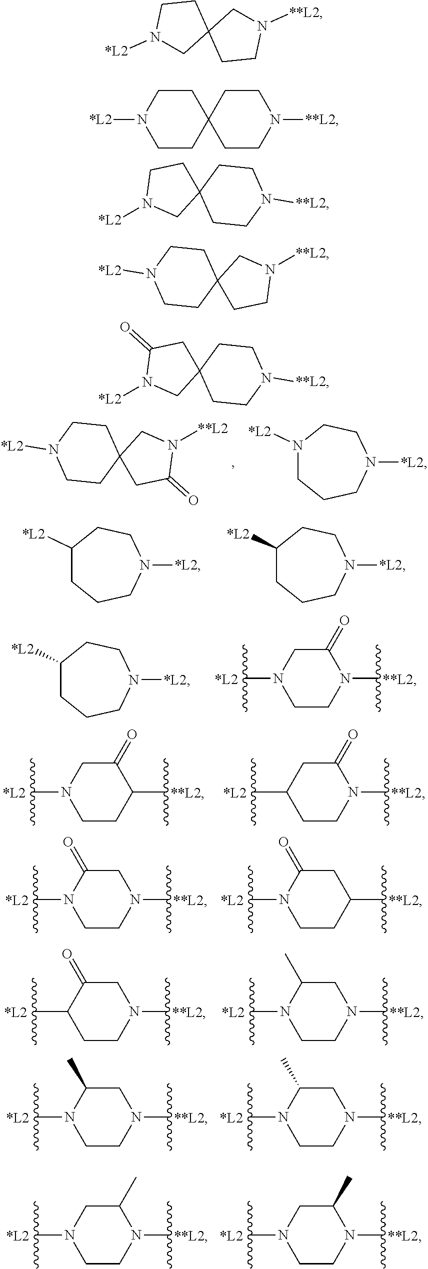 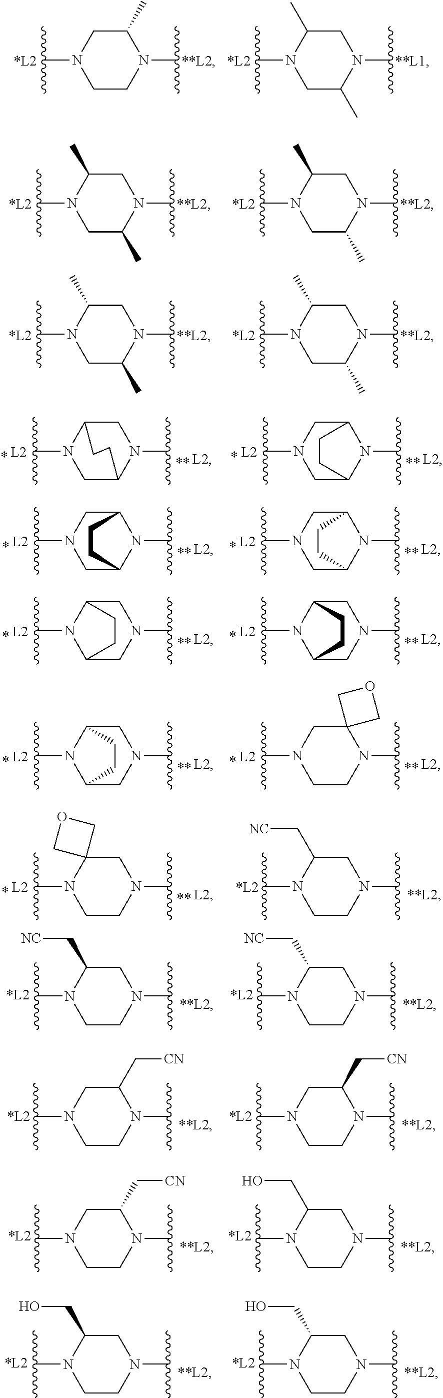 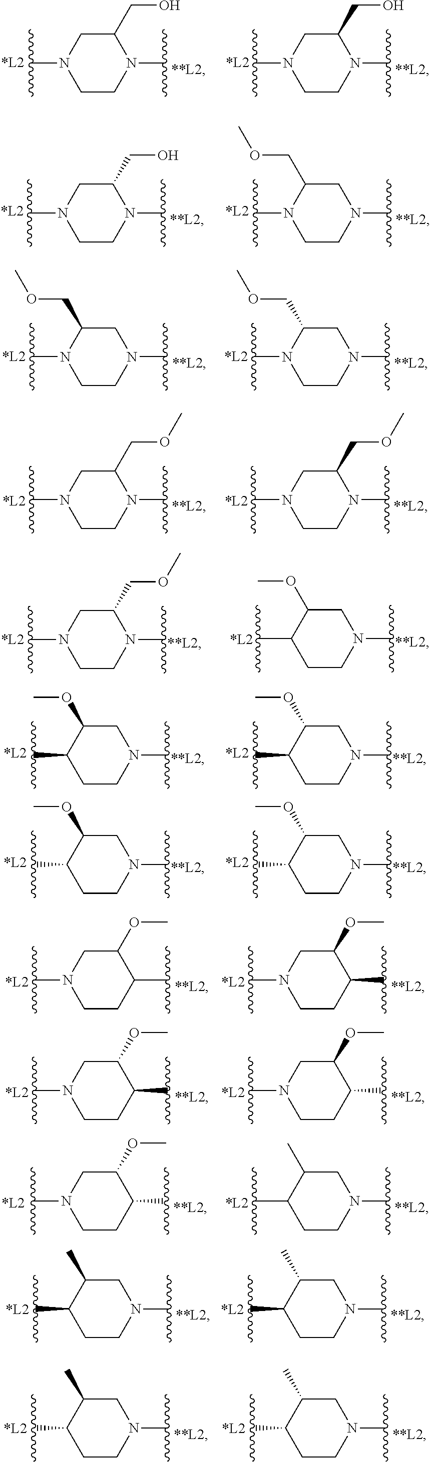 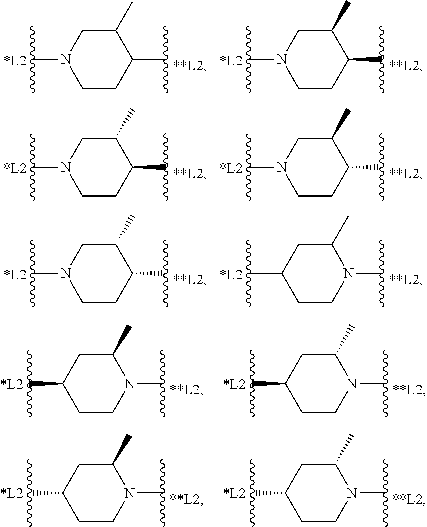 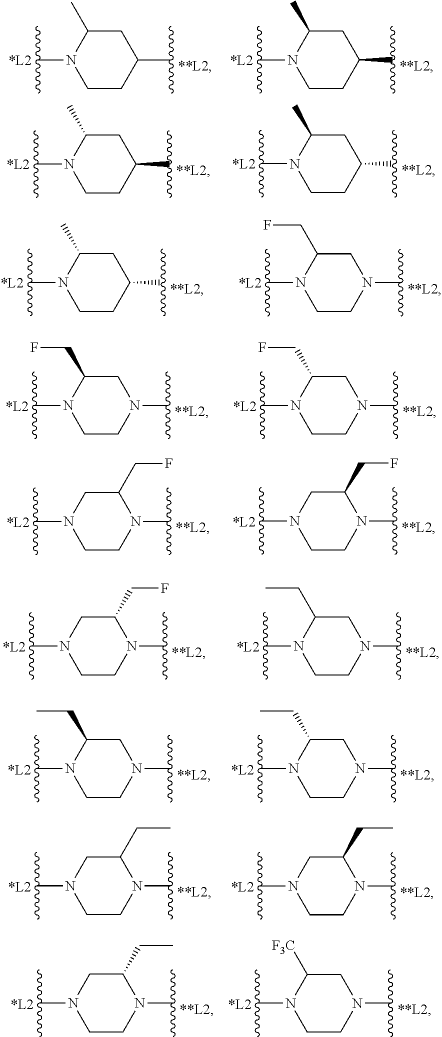 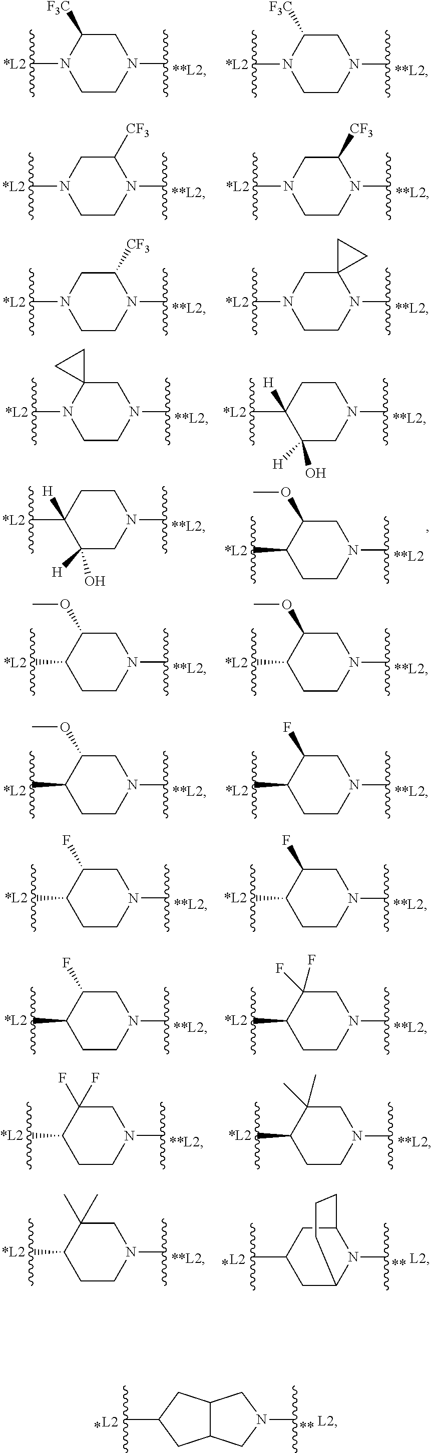 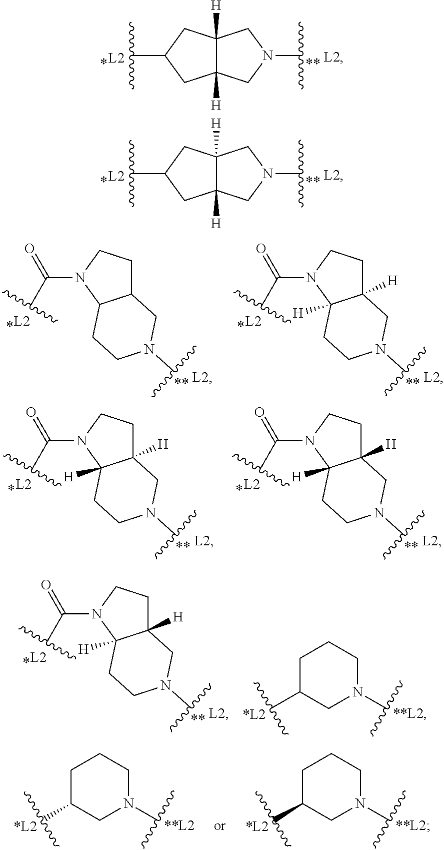
L³ is selected from —O—, —N(CH₃)—, —C(O)—, —NH—, *^{L3}—C(O)N(CH₃)—**^{L3}, *^{L3}—C(O)NH—**^{L3}, *^{L3}C(O)O—**^{L3}, *^{L3}—C(O)N(C₂H₅)—**^{L3}, **^{L3}—C(O)N(C₃H₇)—*^{L3}, *^{L3}—N(CH₃)C(O)—**^{L3}, *^{L3}—NHC(O)—**^{L3}, *^{L3}—OC(O)—**^{L3}, *^{L3}—N(C₂H₅)C(O)—**^{L3}, *^{L3}—N(C₃H₇)C(O)—**^{L3},

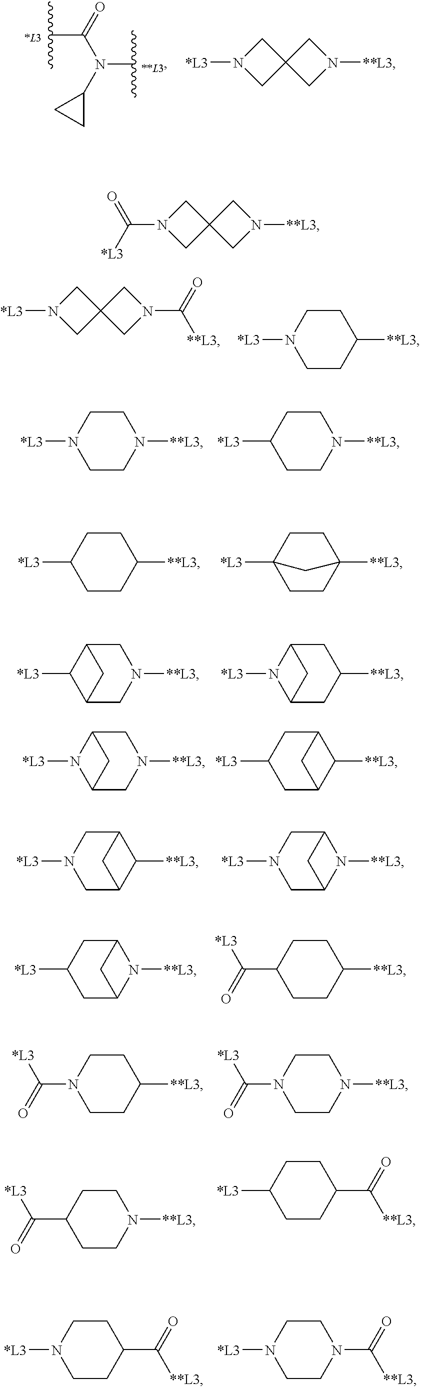

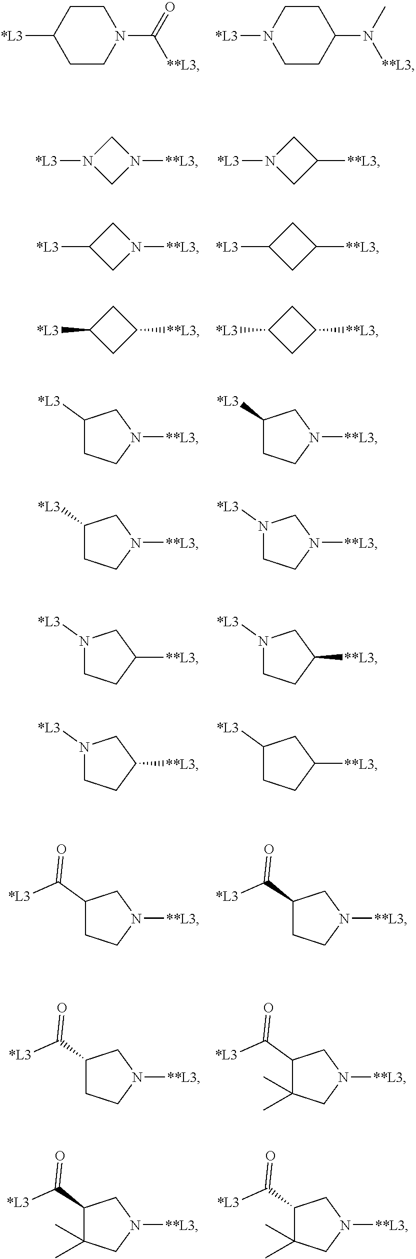

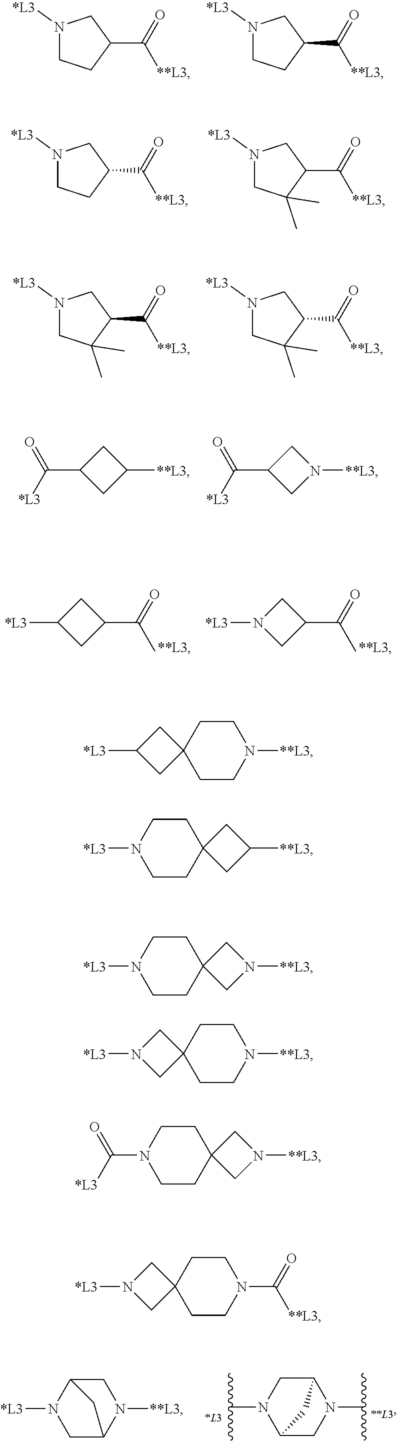

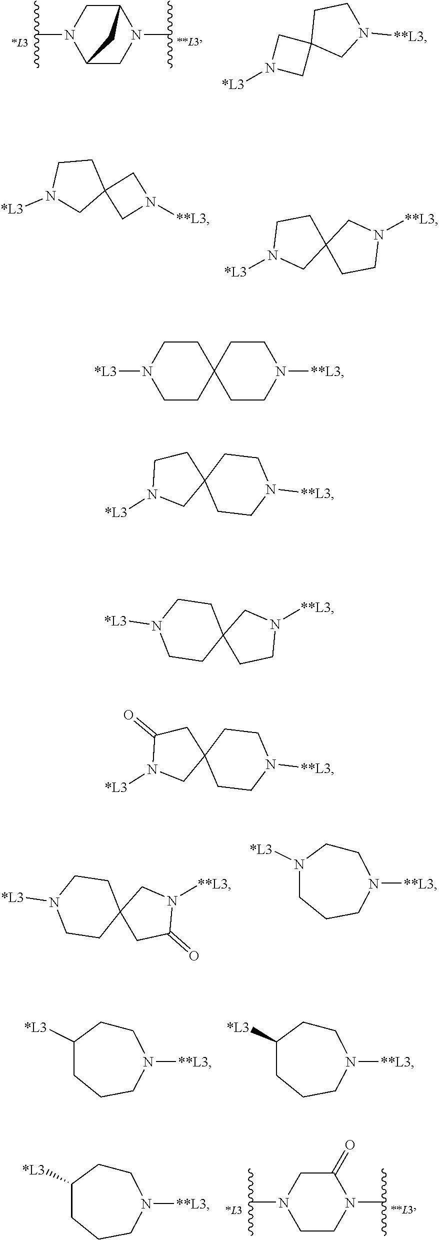

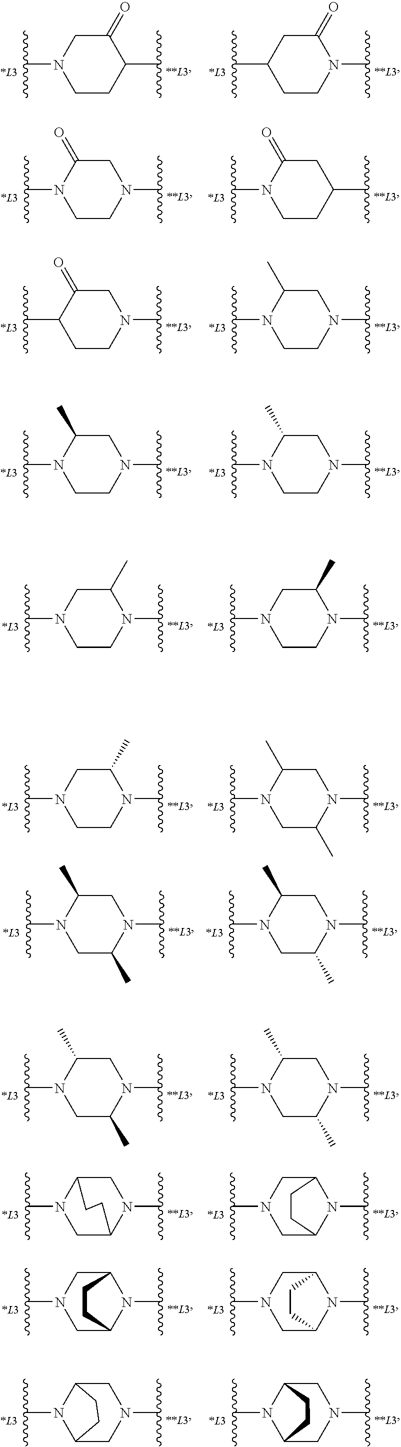

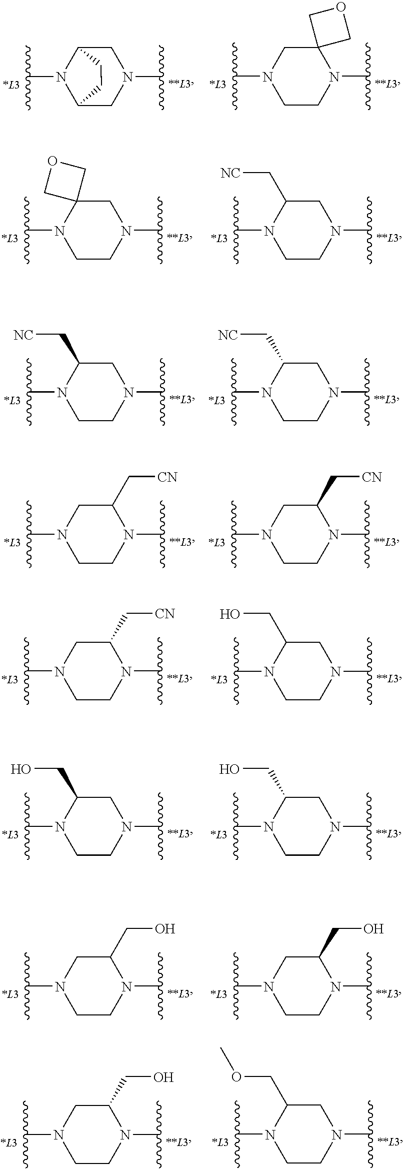

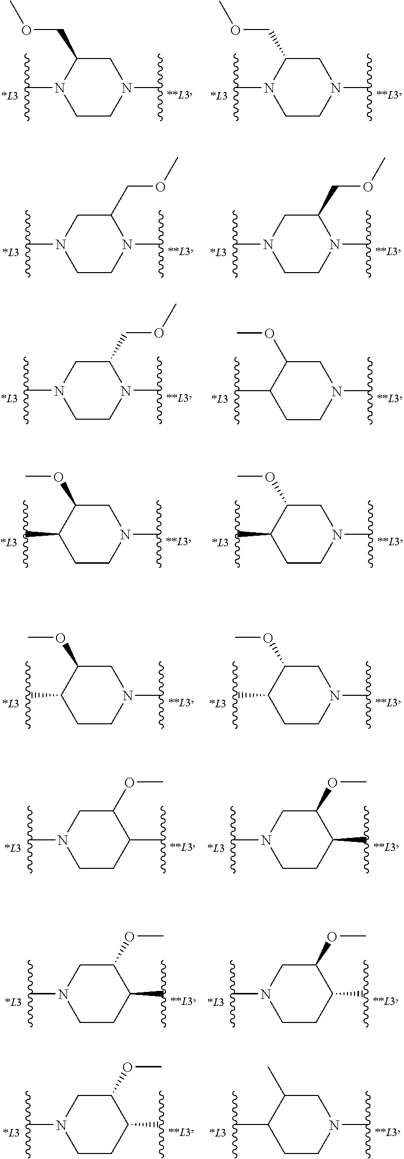

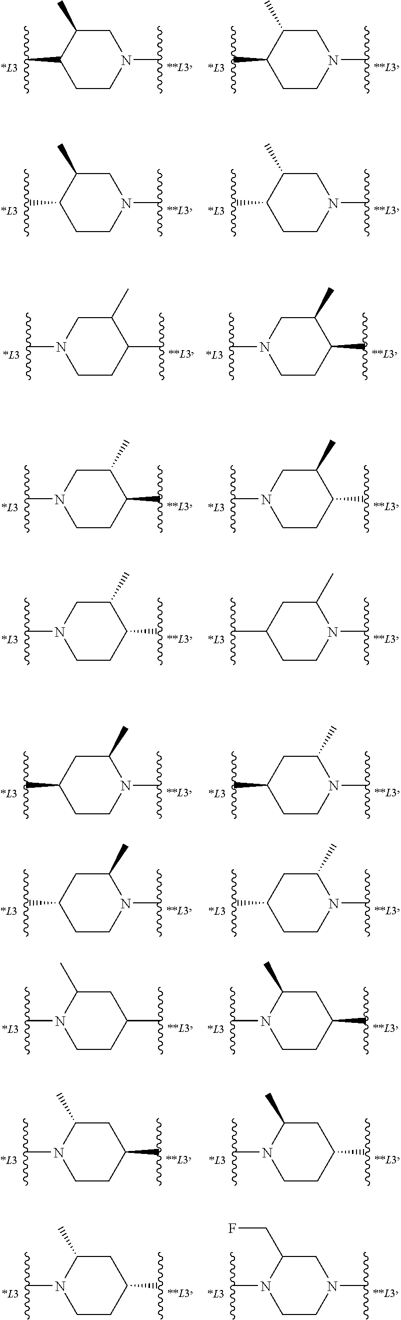

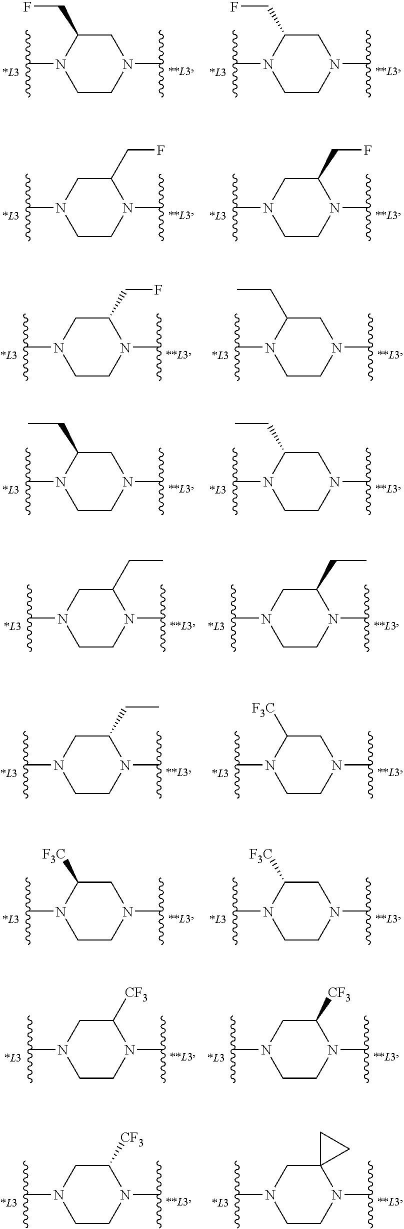

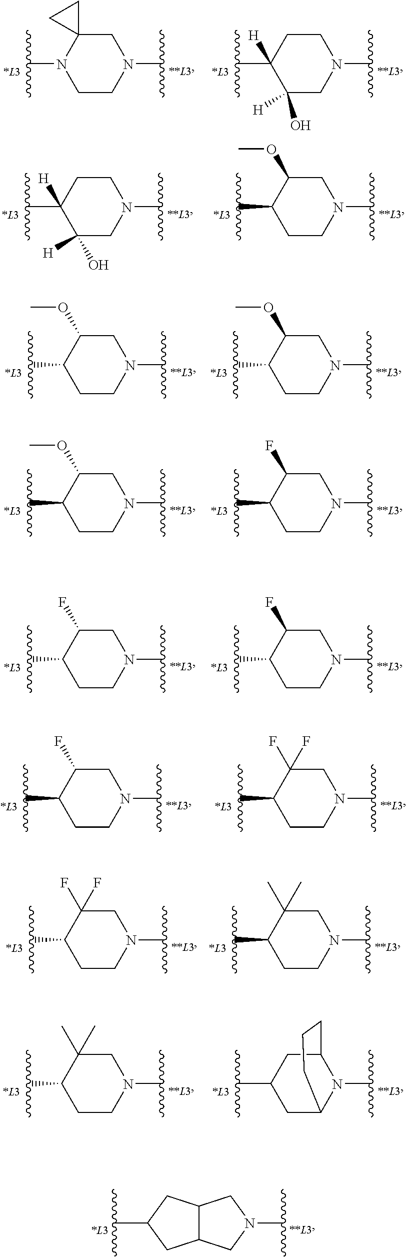

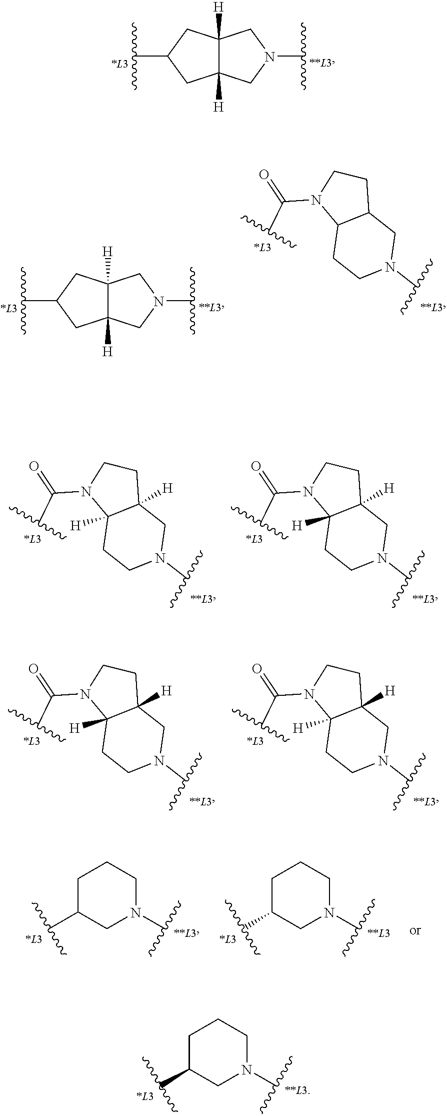

41. The compound of claim 33, wherein 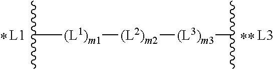
moiety is selected from

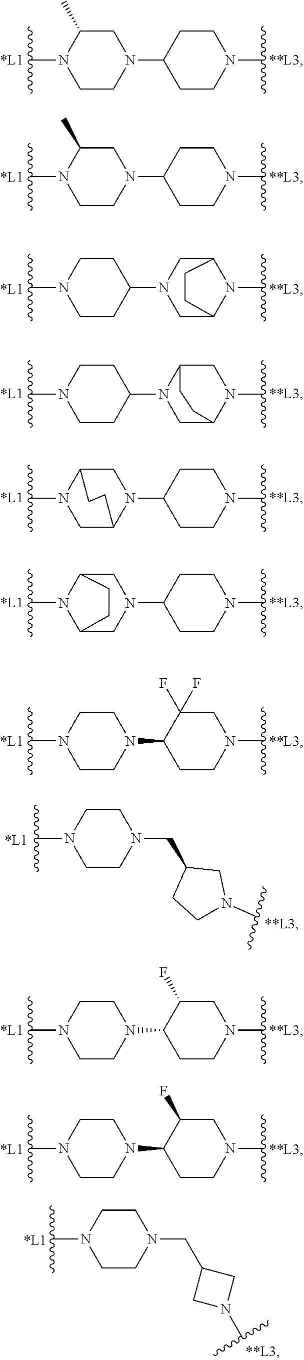

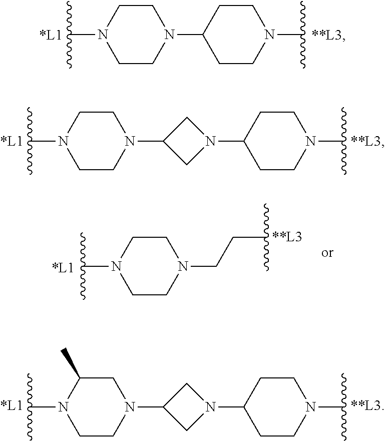 [SMILES: *N1CCC(N2CCN(*)CC2)CC1 ; *N1CCC(N2CC(N3CCN(*)CC3)C2)CC1 ; *CCN1CCN(*)CC1 ; *N1CCC(N2CC(N3CCN(*)[C@@H](C)C3)C2)CC1]

42. The compound of claim 33, wherein L⁴ is independently selected from a single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroary.

43. The compound of claim 33, wherein X⁷ is independently selected from —CR^{a}, or N;
R^{a} is independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl
X⁸ is independently selected from —NR^{a}—, —O—, —S— and —CR^{a}R^{b}—;
at each occurrence, R^{a} and R are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

44. The compound of claim 33, wherein X⁷ is independently selected from —CH, —C(CH₃), or N;
X⁸ is independently selected from —NH— and —CH₂—.

45. The compound of claim 33, wherein 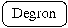
is selected from

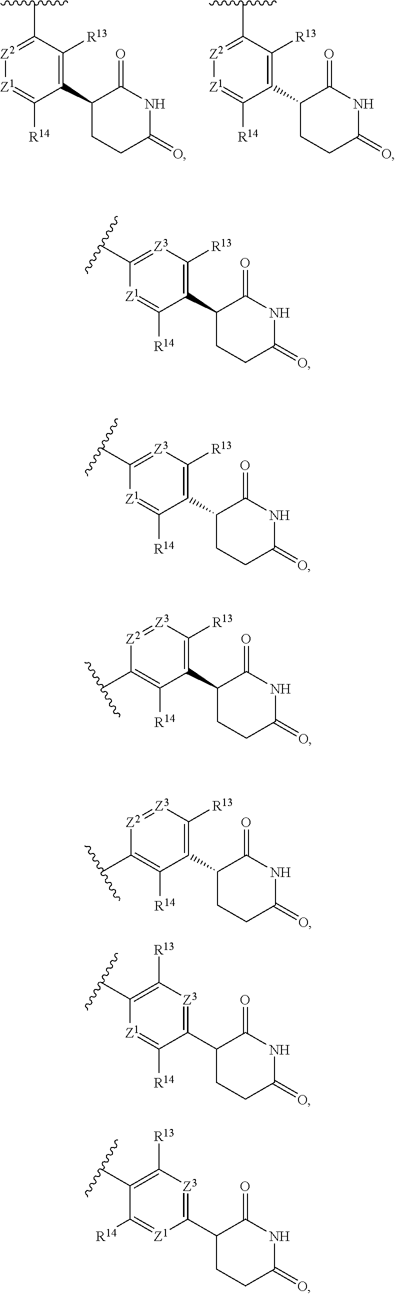

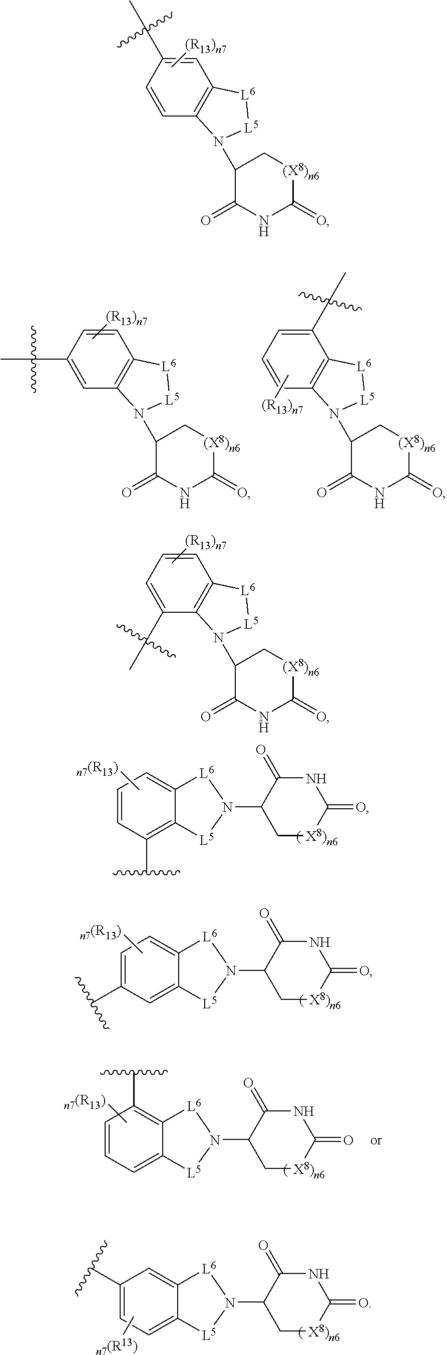

46. The compound of claim 33, wherein
Z¹, Z² and Z³ are each independently CR^{Z};
R^{z}, at each occurrence, is independently selected from hydrogen, —F, —Cl, —Br, —I, methyl, ethyl, propyl, butyl, pentyl, hexyl, heptyl, octyl, —NR^{Za}R^{Zb}, —OR^{Za}, —SR^{Za}, cyclopropyl, cyclobutyl, cyclopentyl, cyclohexyl, cycloheptyl, cyclooctyl, 3- to 8-membered heterocyclyl or CN; each of methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl is optionally substituted with at least one R^{Zc};
R^{Za} and R^{Zb} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{Zd};
R^{Zc} and R^{Zd} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl, or 5- to 12-membered heteroaryl;
R¹³ and R¹⁴ are each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}CO₂R^{13b}, or —NR^{13a}SO₂R^{13b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13a}, —NR^{13c}COR^{13a}, —NR^{13c}CO₂R^{13d}, or —NR^{13c}SO₂R^{13a};
R^{13a}, R^{13b}, R^{13c} and R^{13d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5- to 12-membered heteroaryl;
L⁵ and L⁶ are each independently selected from a single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
X⁸ is —CR^{a}R^{b}—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

47. The compound of claim 33, wherein
R^{z} is selected from H, —CH₃, —C₂H₅, F, —CH₂F, —CHF₂, —CF₃, —OCH₃, —OC₂H₅, —C₃H₇, —OCH₂F, —OCHF₂, —OCH₂CF₃, —OCF₃, —SCF₃, —CF₃ or —CH(OH)CH₃;
R¹³ and R¹⁴ are each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CN, —CH₂F, —CHF₂, —CF₃, —OCH₂F, —OCHF₂, —OCH₂CF₃, —OCF₃, —SCF₃, or phenyl;
L⁵ and L⁶ are each independently a single bond, 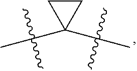 [SMILES: *C1(*)CC1]
—O—, —NH—, —NMe-, —N(CH₂CH₃)—, —CH₂—, —CHF—, —CF₂—, —C(CH₃)₂— or —CO—;
X⁸ is CH₂; and
n6 is 0 or 1;
n7 is 0, 1 or 2.

48. The compound of claim 33, wherein

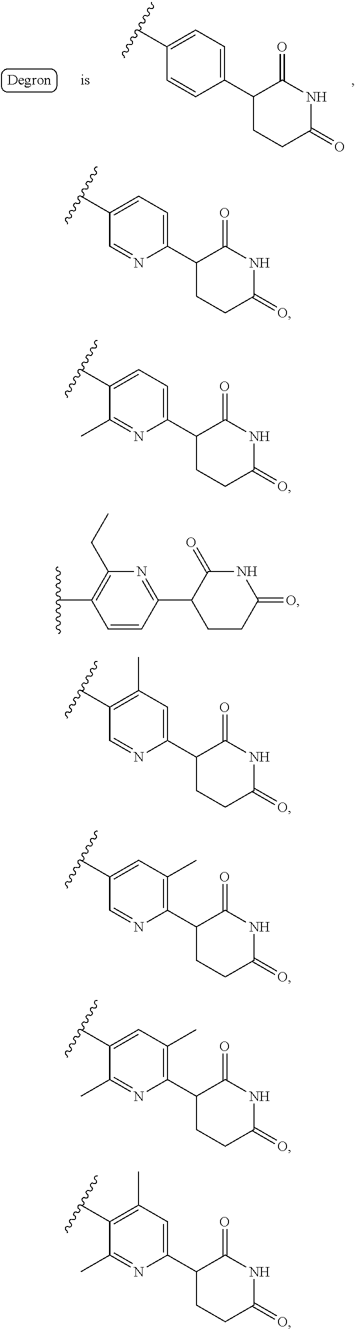

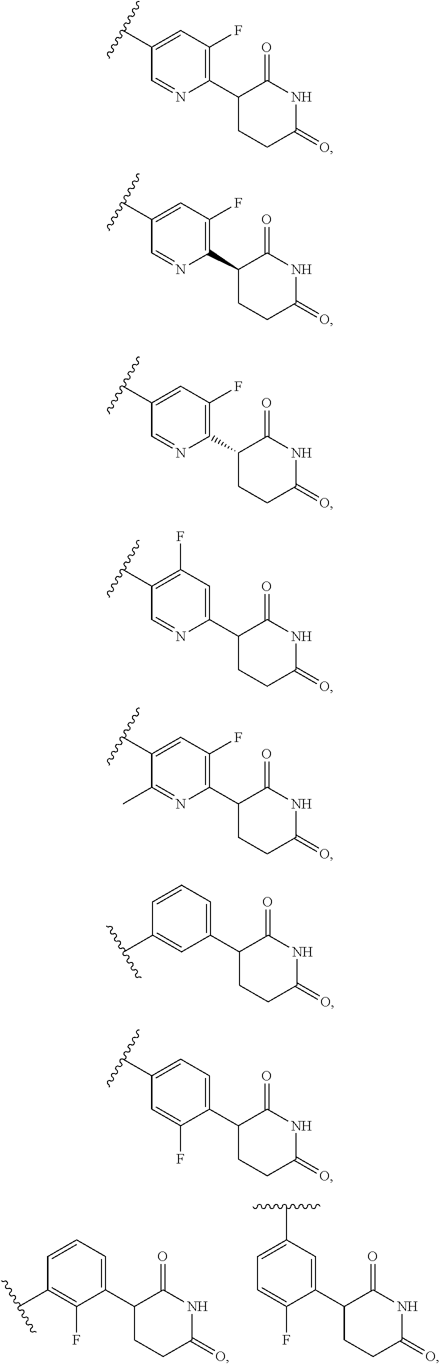

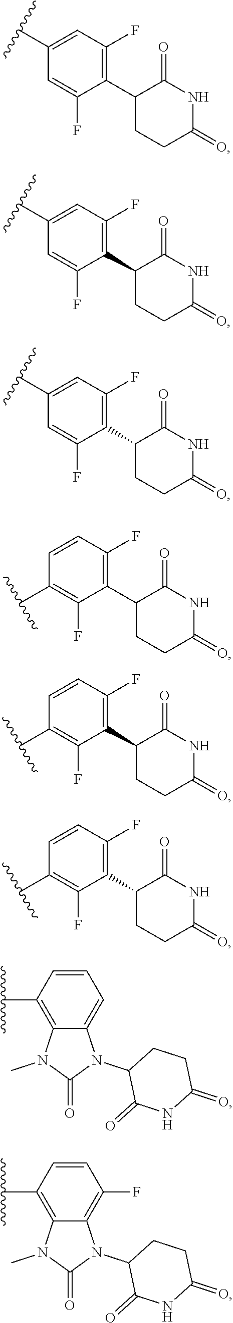

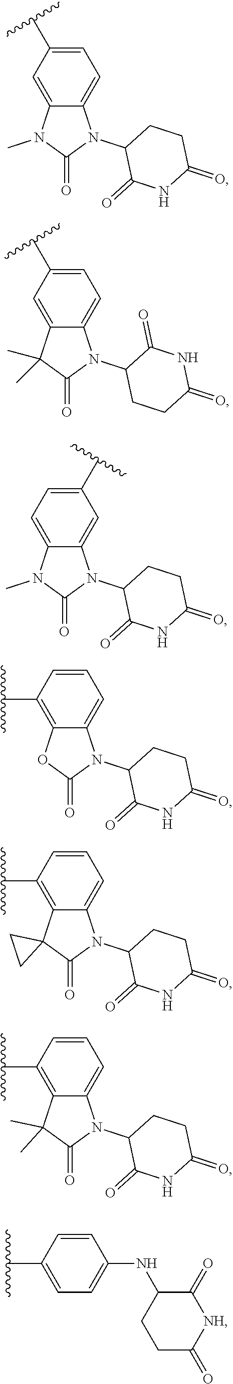

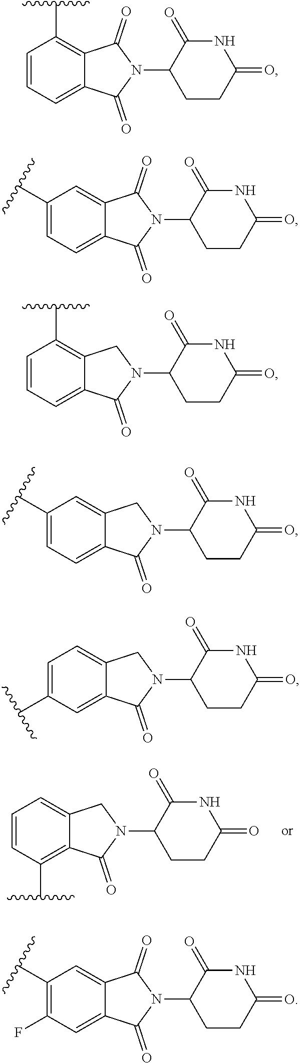

49. A compound, wherein the compound is selected from 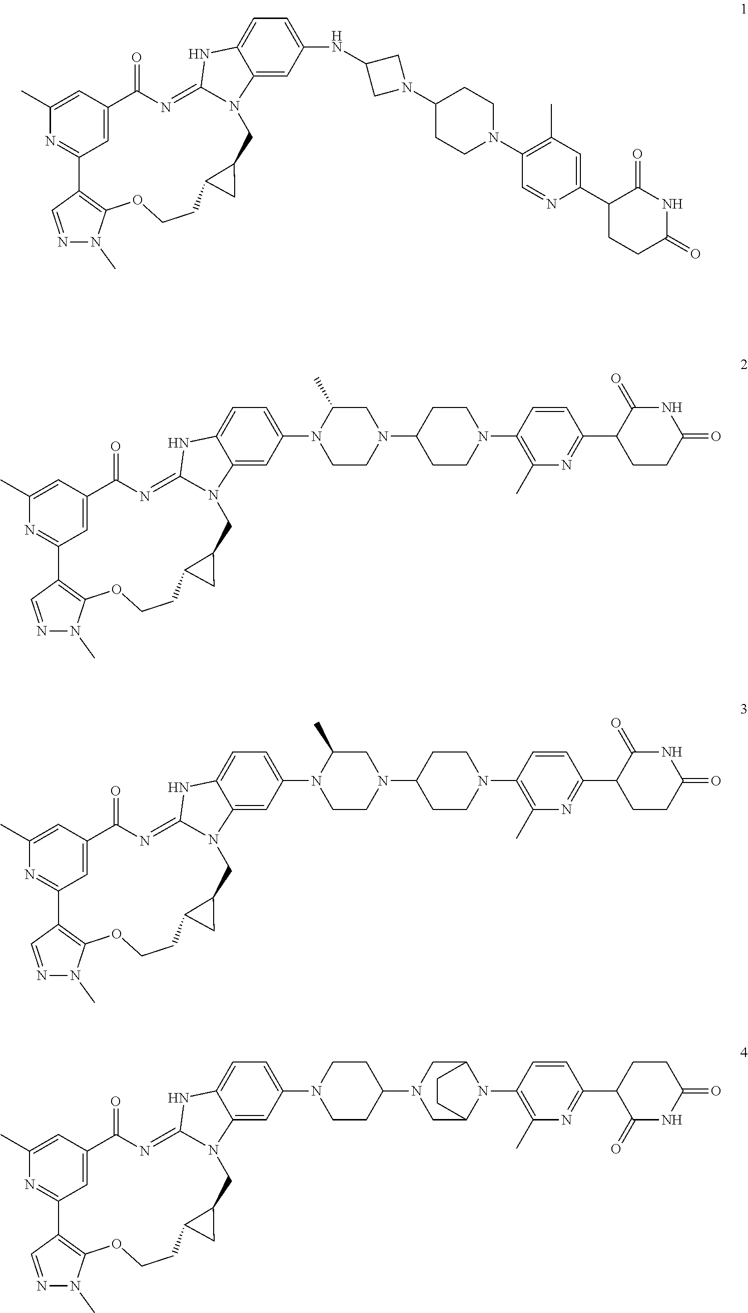 [SMILES: Cc1cc2cc(n1)-c1cnn(C)c1OCC[C@H]1C[C@@H]1CN1/C(=N/C2=O)Nc2ccc(NC3CN(C4CCN(c5cnc(C6CCC(=O)NC6=O)cc5C)CC4)C3)cc21 ; Cc1cc2cc(n1)-c1cnn(C)c1OCC[C@H]1C[C@@H]1CN1/C(=N/C2=O)Nc2ccc(N3CCN(C4CCN(c5ccc(C6CCC(=O)NC6=O)nc5C)CC4)C[C@H]3C)cc21 ; Cc1cc2cc(n1)-c1cnn(C)c1OCC[C@H]1C[C@@H]1CN1/C(=N/C2=O)Nc2ccc(N3CCN(C4CCN(c5ccc(C6CCC(=O)NC6=O)nc5C)CC4)C[C@@H]3C)cc21 ; Cc1cc2cc(n1)-c1cnn(C)c1OCC[C@H]1C[C@@H]1CN1/C(=N/C2=O)Nc2ccc(N3CCC(N4CC5CCC(C4)N5c4ccc(C5CCC(=O)NC5=O)nc4C)CC3)cc21] 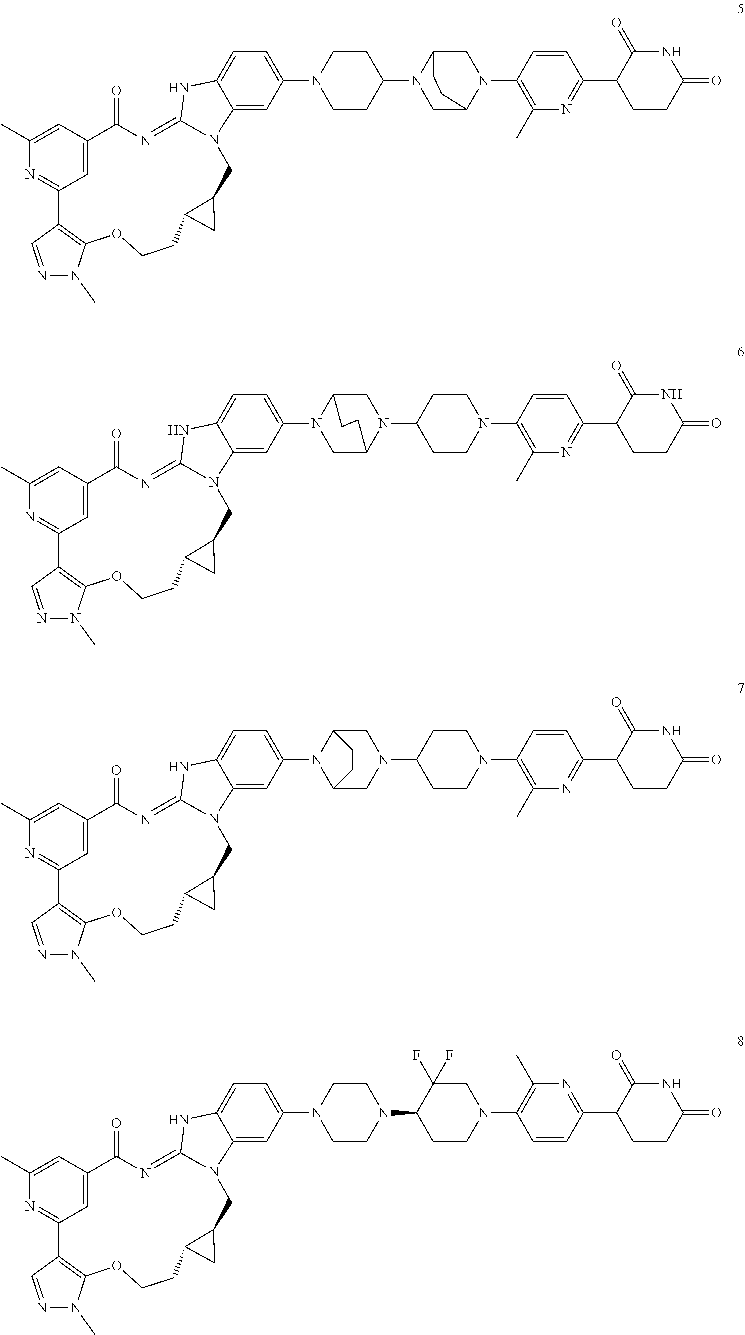 [SMILES: Cc1cc2cc(n1)-c1cnn(C)c1OCC[C@H]1C[C@@H]1CN1/C(=N/C2=O)Nc2ccc(N3CCC(N4CC5CCC4CN5c4ccc(C5CCC(=O)NC5=O)nc4C)CC3)cc21 ; Cc1cc2cc(n1)-c1cnn(C)c1OCC[C@H]1C[C@@H]1CN1/C(=N/C2=O)Nc2ccc(N3CC4CCC3CN4C3CCN(c4ccc(C5CCC(=O)NC5=O)nc4C)CC3)cc21 ; Cc1cc2cc(n1)-c1cnn(C)c1OCC[C@H]1C[C@@H]1CN1/C(=N/C2=O)Nc2ccc(N3C4CCC3CN(C3CCN(c5ccc(C6CCC(=O)NC6=O)nc5C)CC3)C4)cc21 ; Cc1cc2cc(n1)-c1cnn(C)c1OCC[C@H]1C[C@@H]1CN1/C(=N/C2=O)Nc2ccc(N3CCN([C@@H]4CCN(c5ccc(C6CCC(=O)NC6=O)nc5C)CC4(F)F)CC3)cc21] 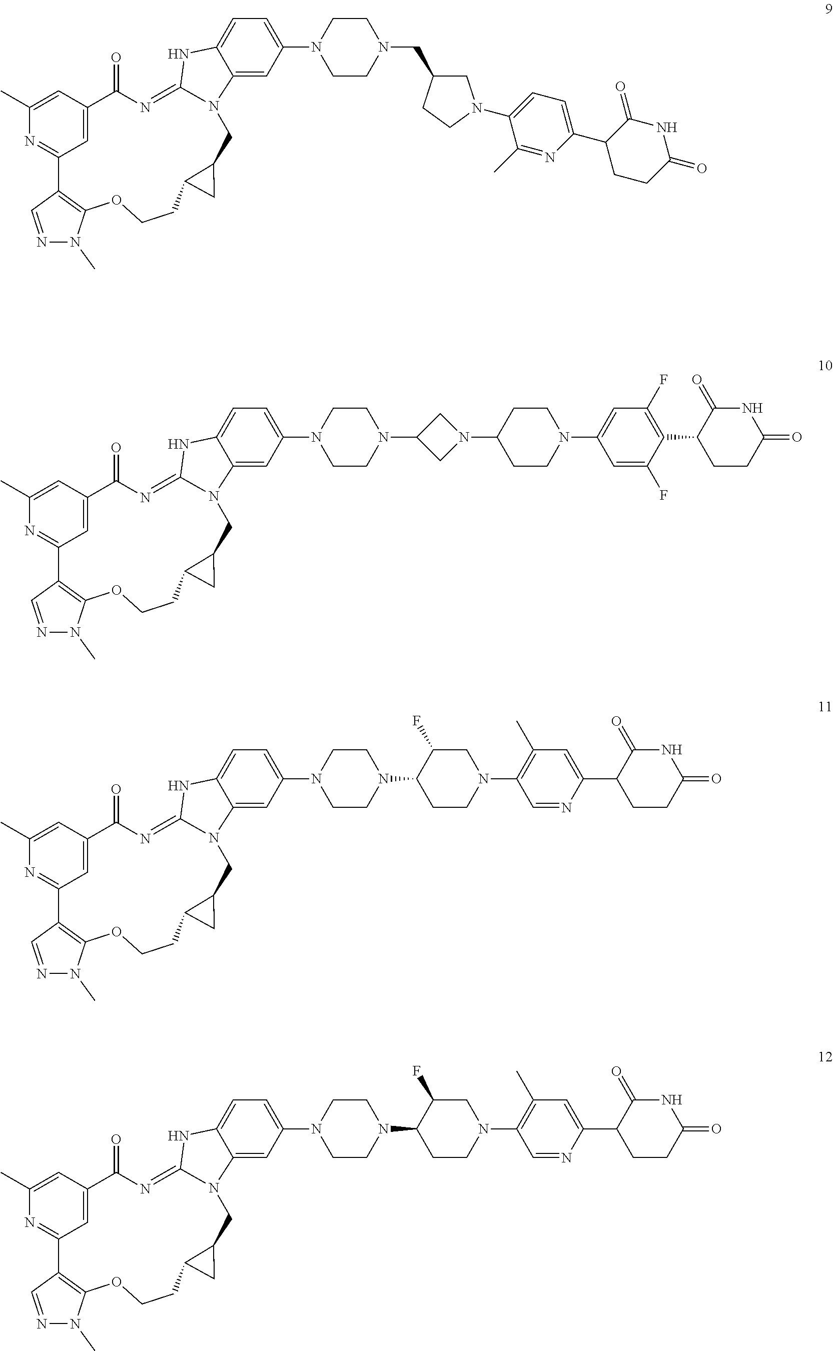 [SMILES: Cc1cc2cc(n1)-c1cnn(C)c1OCC[C@H]1C[C@@H]1CN1/C(=N/C2=O)Nc2ccc(N3CCN(C[C@@H]4CCN(c5ccc(C6CCC(=O)NC6=O)nc5C)C4)CC3)cc21 ; Cc1cc2cc(n1)-c1cnn(C)c1OCC[C@H]1C[C@@H]1CN1/C(=N/C2=O)Nc2ccc(N3CCN(C4CN(C5CCN(c6cc(F)c([C@H]7CCC(=O)NC7=O)c(F)c6)CC5)C4)CC3)cc21 ; Cc1cc2cc(n1)-c1cnn(C)c1OCC[C@H]1C[C@@H]1CN1/C(=N/C2=O)Nc2ccc(N3CCN([C@H]4CCN(c5cnc(C6CCC(=O)NC6=O)cc5C)C[C@H]4F)CC3)cc21 ; Cc1cc2cc(n1)-c1cnn(C)c1OCC[C@H]1C[C@@H]1CN1/C(=N/C2=O)Nc2ccc(N3CCN([C@@H]4CCN(c5cnc(C6CCC(=O)NC6=O)cc5C)C[C@@H]4F)CC3)cc21] 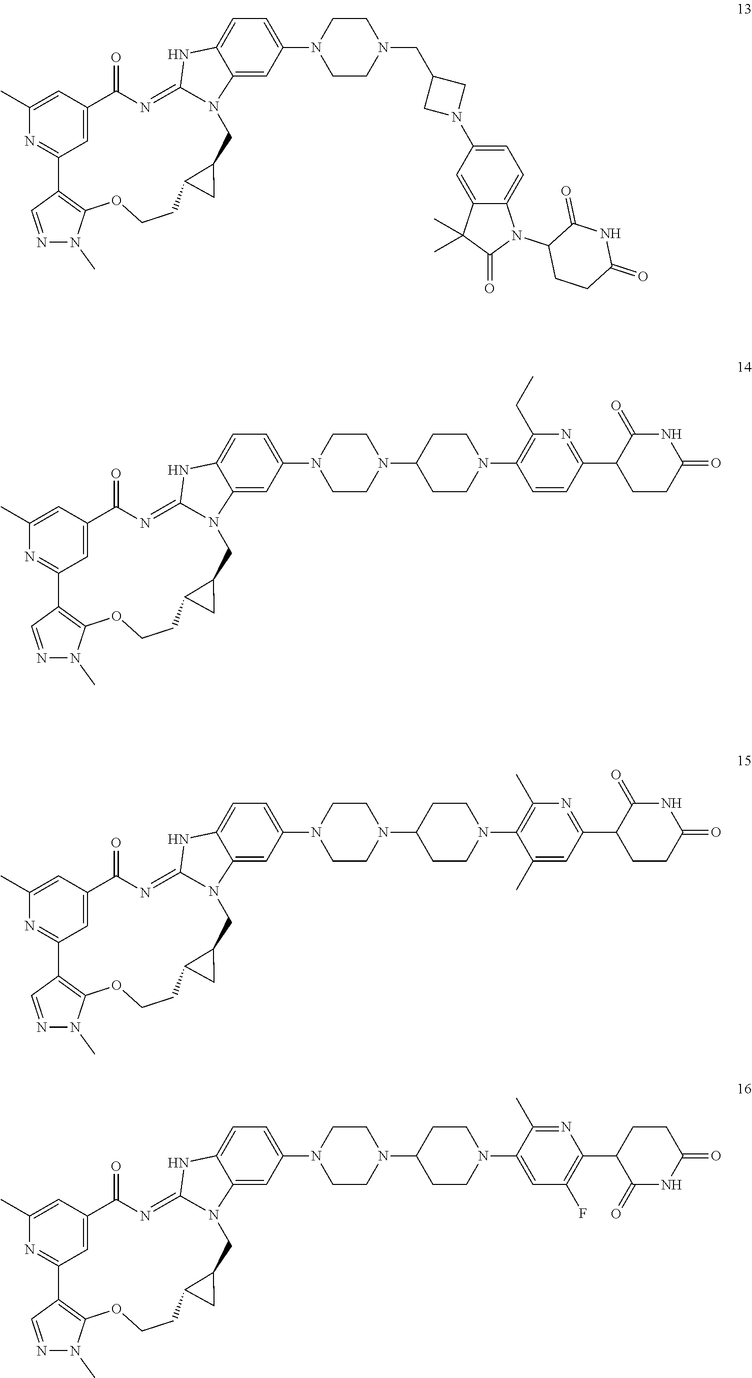 [SMILES: Cc1cc2cc(n1)-c1cnn(C)c1OCC[C@H]1C[C@@H]1CN1/C(=N/C2=O)Nc2ccc(N3CCN(CC4CN(c5ccc6c(c5)C(C)(C)C(=O)N6C5CCC(=O)NC5=O)C4)CC3)cc21 ; CCc1nc(C2CCC(=O)NC2=O)ccc1N1CCC(N2CCN(c3ccc4c(c3)N3C[C@H]5C[C@@H]5CCOc5c(cnn5C)-c5cc(cc(C)n5)C(=O)/N=C/3N4)CC2)CC1 ; Cc1cc2cc(n1)-c1cnn(C)c1OCC[C@H]1C[C@@H]1CN1/C(=N/C2=O)Nc2ccc(N3CCN(C4CCN(c5c(C)cc(C6CCC(=O)NC6=O)nc5C)CC4)CC3)cc21 ; Cc1cc2cc(n1)-c1cnn(C)c1OCC[C@H]1C[C@@H]1CN1/C(=N/C2=O)Nc2ccc(N3CCN(C4CCN(c5cc(F)c(C6CCC(=O)NC6=O)nc5C)CC4)CC3)cc21] 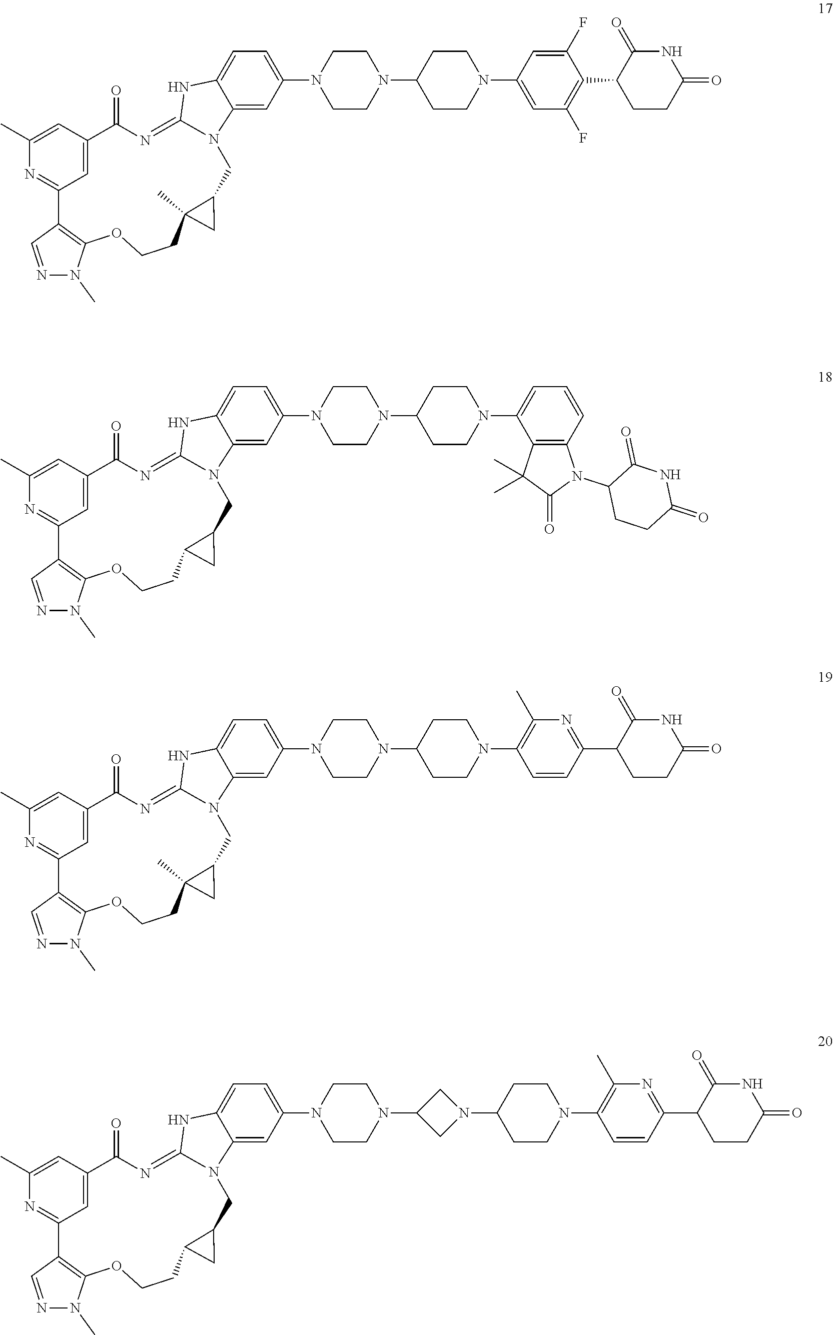 [SMILES: Cc1cc2cc(n1)-c1cnn(C)c1OCC[C@]1(C)C[C@H]1CN1/C(=N/C2=O)Nc2ccc(N3CCN(C4CCN(c5cc(F)c([C@H]6CCC(=O)NC6=O)c(F)c5)CC4)CC3)cc21 ; Cc1cc2cc(n1)-c1cnn(C)c1OCC[C@H]1C[C@@H]1CN1/C(=N/C2=O)Nc2ccc(N3CCN(C4CCN(c5cccc6c5C(C)(C)C(=O)N6C5CCC(=O)NC5=O)CC4)CC3)cc21 ; Cc1cc2cc(n1)-c1cnn(C)c1OCC[C@]1(C)C[C@H]1CN1/C(=N/C2=O)Nc2ccc(N3CCN(C4CCN(c5ccc(C6CCC(=O)NC6=O)nc5C)CC4)CC3)cc21 ; Cc1cc2cc(n1)-c1cnn(C)c1OCC[C@H]1C[C@@H]1CN1/C(=N/C2=O)Nc2ccc(N3CCN(C4CN(C5CCN(c6ccc(C7CCC(=O)NC7=O)nc6C)CC5)C4)CC3)cc21] 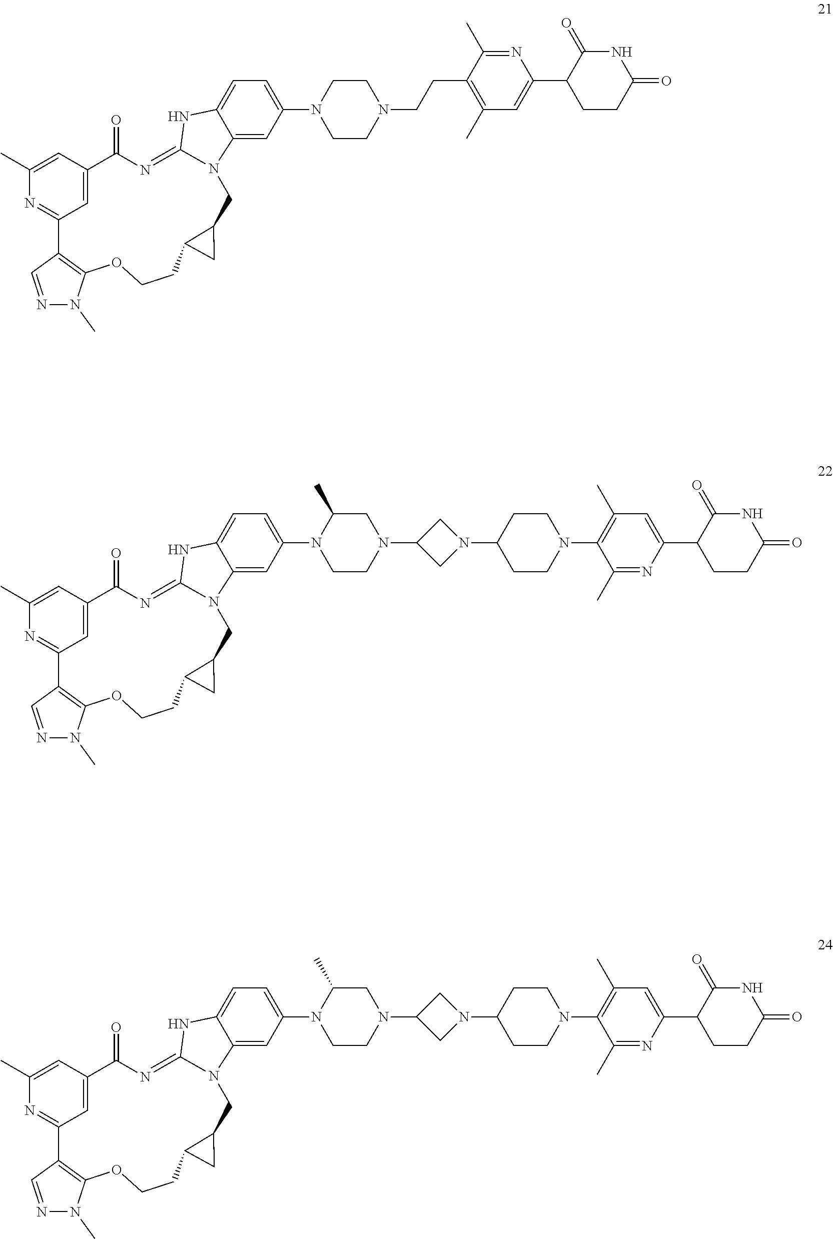 [SMILES: Cc1cc2cc(n1)-c1cnn(C)c1OCC[C@H]1C[C@@H]1CN1/C(=N/C2=O)Nc2ccc(N3CCN(CCc4c(C)cc(C5CCC(=O)NC5=O)nc4C)CC3)cc21 ; Cc1cc2cc(n1)-c1cnn(C)c1OCC[C@H]1C[C@@H]1CN1/C(=N/C2=O)Nc2ccc(N3CCN(C4CN(C5CCN(c6c(C)cc(C7CCC(=O)NC7=O)nc6C)CC5)C4)C[C@@H]3C)cc21 ; Cc1cc2cc(n1)-c1cnn(C)c1OCC[C@H]1C[C@@H]1CN1/C(=N/C2=O)Nc2ccc(N3CCN(C4CN(C5CCN(c6c(C)cc(C7CCC(=O)NC7=O)nc6C)CC5)C4)C[C@H]3C)cc21] 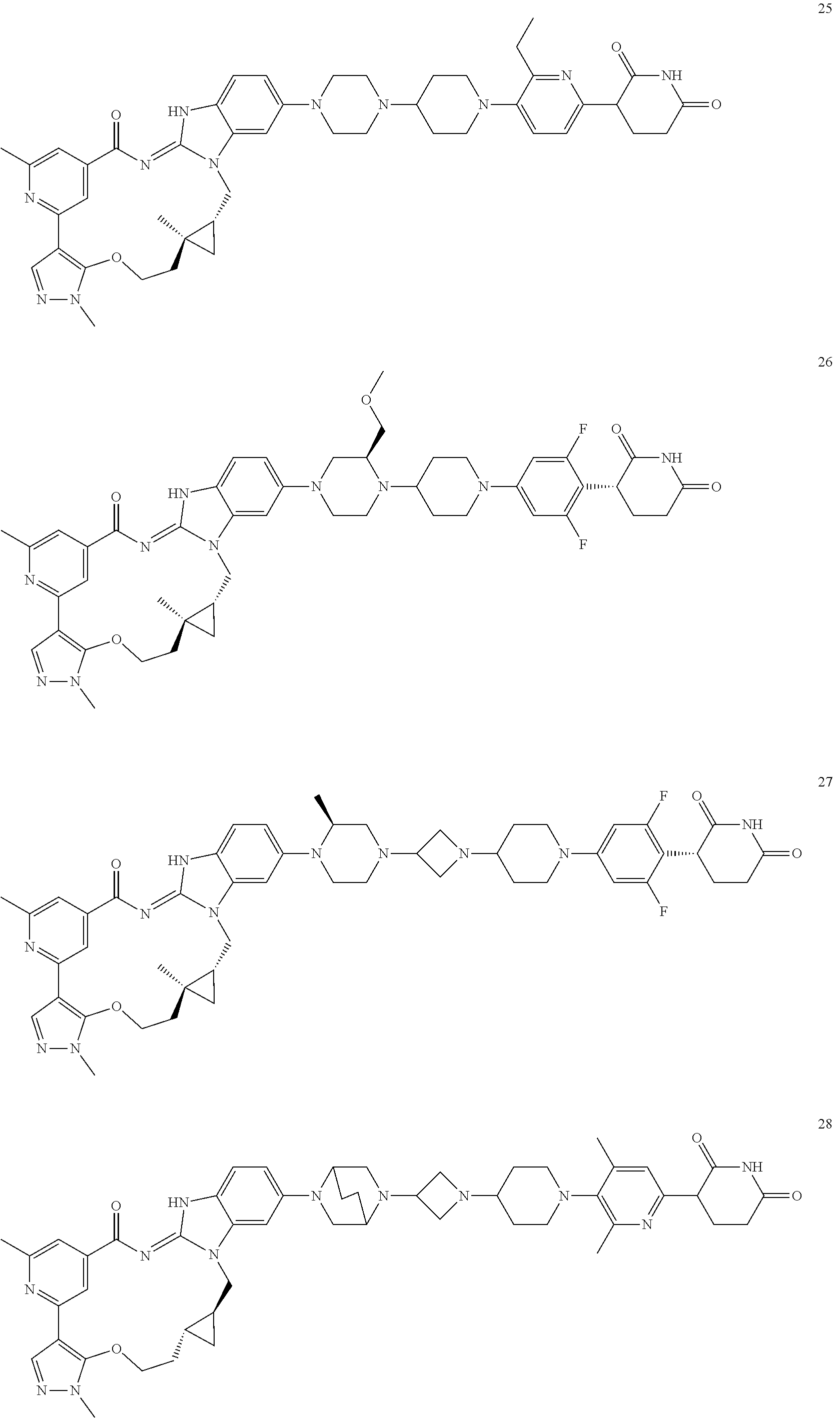 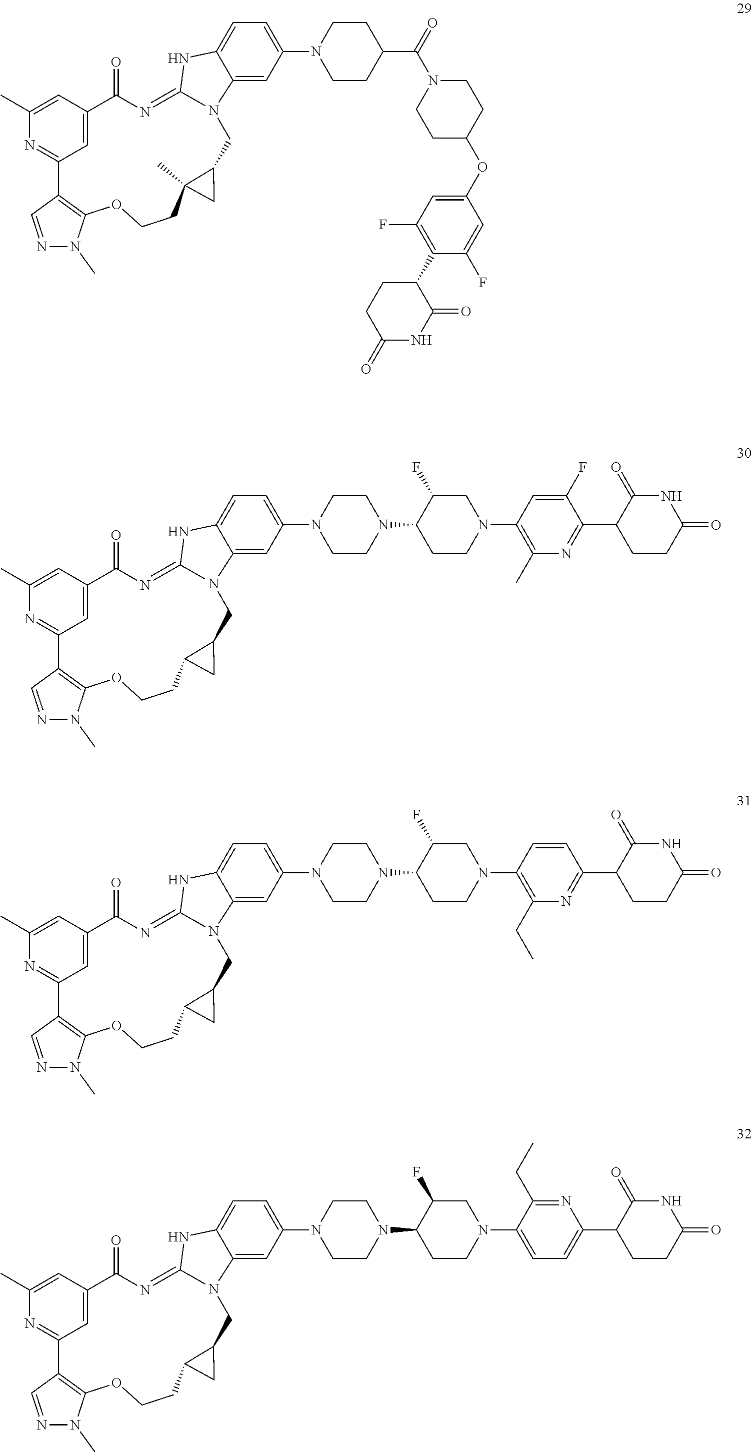 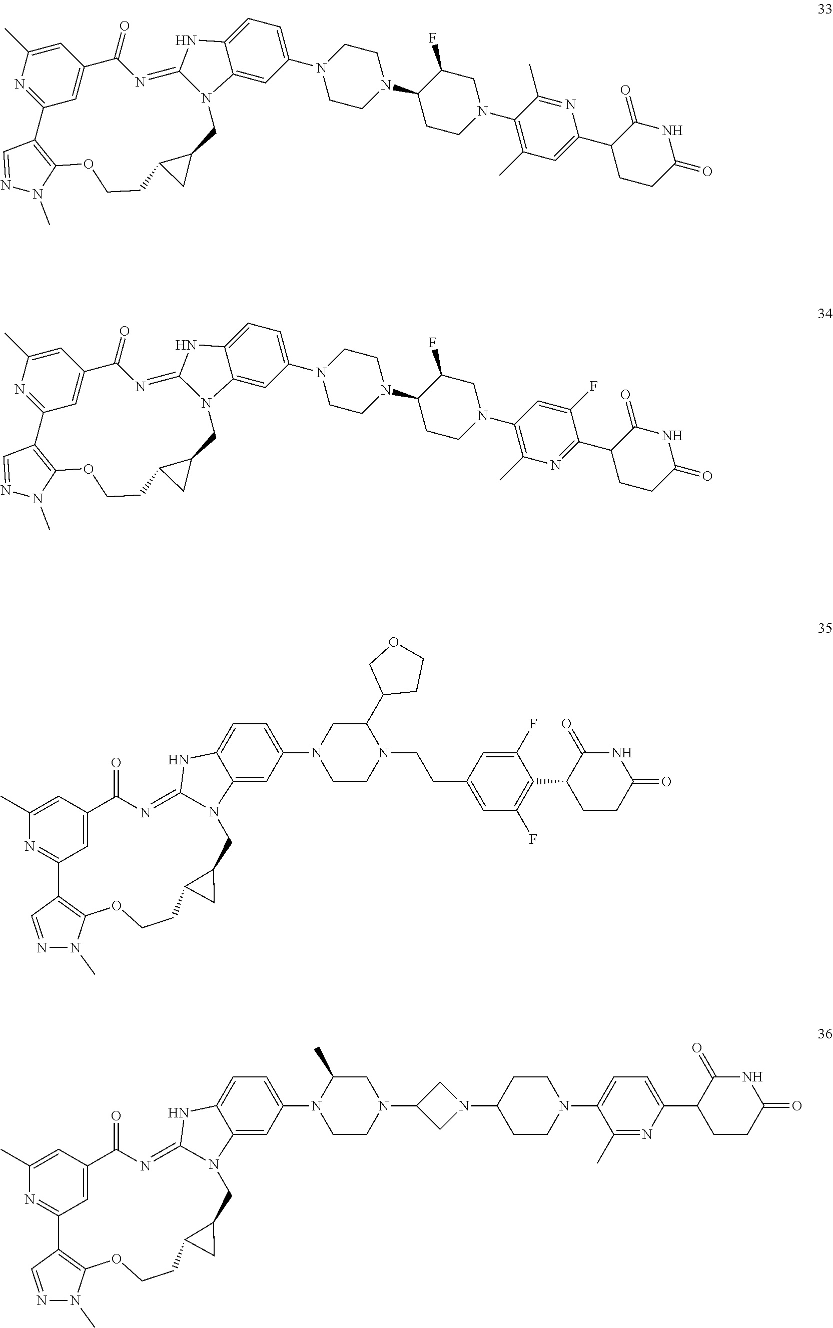 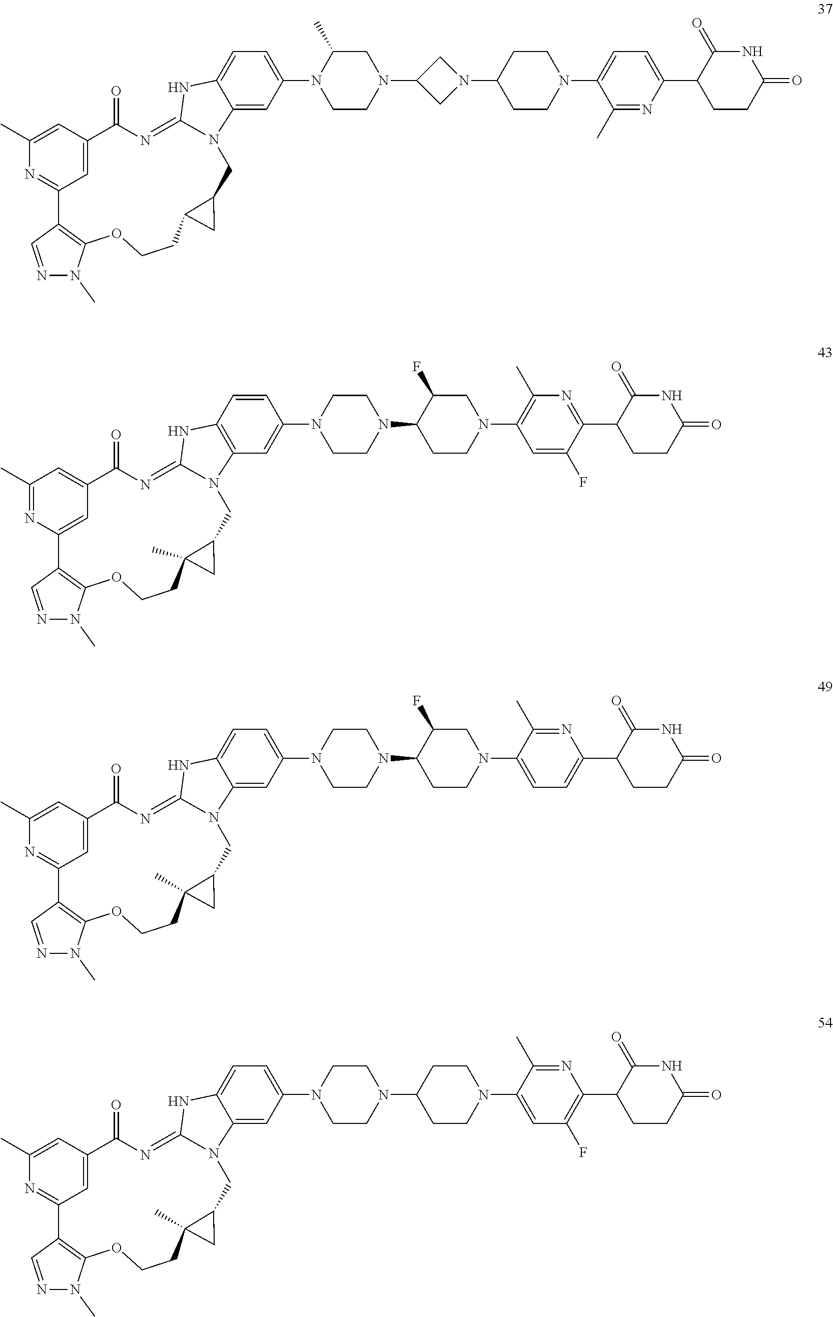 [SMILES: Cc1cc2cc(n1)-c1cnn(C)c1OCC[C@H]1C[C@@H]1CN1/C(=N/C2=O)Nc2ccc(N3CCN(C4CN(C5CCN(c6ccc(C7CCC(=O)NC7=O)nc6C)CC5)C4)C[C@H]3C)cc21 ; Cc1cc2cc(n1)-c1cnn(C)c1OCC[C@]1(C)C[C@H]1CN1/C(=N/C2=O)Nc2ccc(N3CCN([C@@H]4CCN(c5cc(F)c(C6CCC(=O)NC6=O)nc5C)C[C@@H]4F)CC3)cc21 ; Cc1cc2cc(n1)-c1cnn(C)c1OCC[C@]1(C)C[C@H]1CN1/C(=N/C2=O)Nc2ccc(N3CCN([C@@H]4CCN(c5ccc(C6CCC(=O)NC6=O)nc5C)C[C@@H]4F)CC3)cc21 ; Cc1cc2cc(n1)-c1cnn(C)c1OCC[C@]1(C)C[C@H]1CN1/C(=N/C2=O)Nc2ccc(N3CCN(C4CCN(c5cc(F)c(C6CCC(=O)NC6=O)nc5C)CC4)CC3)cc21] 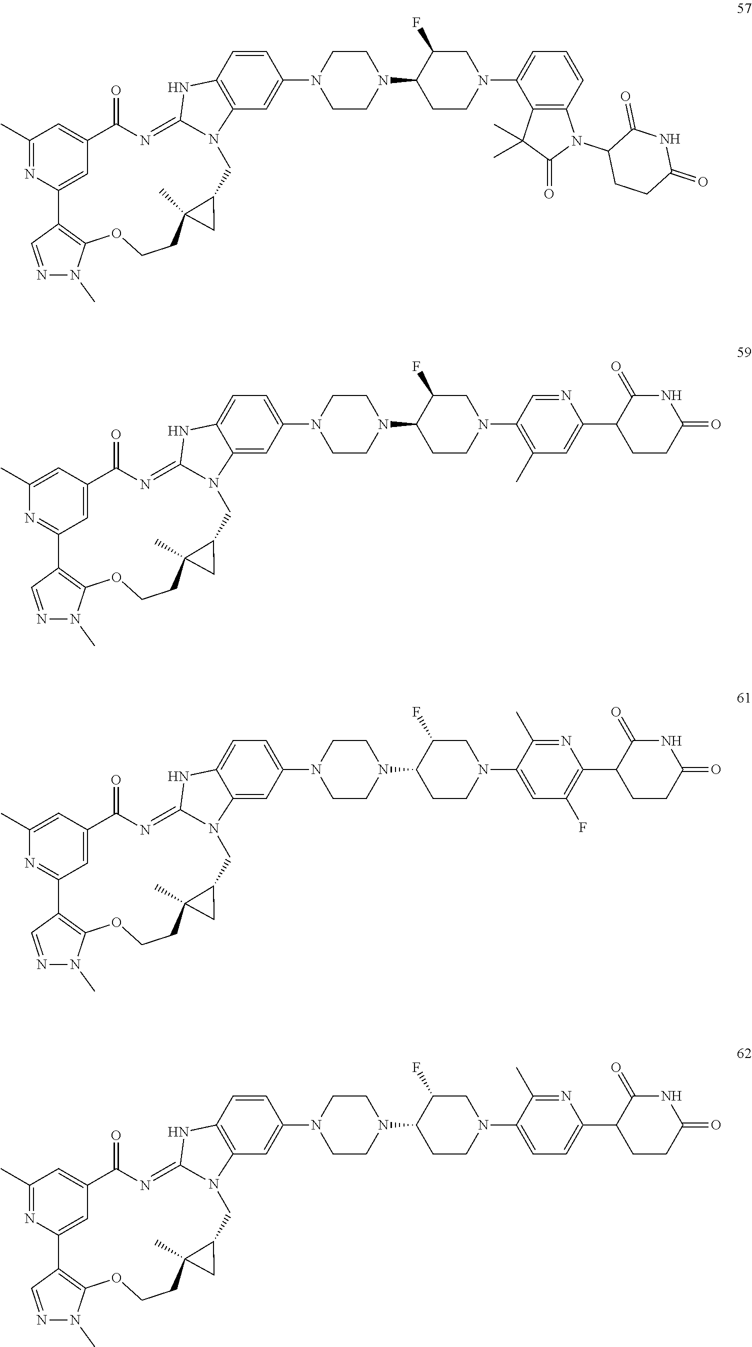
or a N-oxide thereof, or a pharmaceutically acceptable salt thereof, or a stereoisomer thereof, or a deuterated analog thereof, or a prodrug thereof.

50. A pharmaceutical composition comprising a compound of claim 33, or a pharmaceutically acceptable salt, stereoisomer, tautomer or prodrug thereof, together with a pharmaceutically acceptable excipient.

51. A method of treating a disease that can be affected by EGFR modulation, comprises administrating a subject in need thereof an effective amount of a compound of claim 33, or a pharmaceutically acceptable salt, stereoisomer, tautomer or prodrug thereof, wherein the disease is selected from cancer.

52. The method of claim 51, wherein the disease is selected from pancreatic cancer, breast cancer, glioblastoma multiforme, head and neck cancer, or non-small cell lung cancer.

53. The compound of claim 33, wherein R^{1a}, R^{1b}, R^{1c}, R^{1d}, R^{1e}, R^{1f}, R^{1g}, R^{1h}, R^{2a}, R², R³, R⁴ and R⁵ are each independently hydrogen, F, Cl, Br, I, methyl, ethyl, propyl, butyl, methoxy, ethoxy, propoxy, butoxy, cyclopropyl, cyclobutyl, cyclopentyl, cyclohexyl, —CF₃, —CHF₂, —CN, —CH₂OCH₃, —CH₂OCH₂CH₃, —CH₂CH₂OCH₃, —CH₂CH₂OCH₂CH₃.
